(19) United States
(12) Patent Application Publication
(10) Pub. No.: US 2026/0055428 A1
(43) Pub. Date: Feb. 26, 2026

(54) MODIFIED VIRUS-LIKE PARTICLES OF CMV

(71) Applicant: SAIBA ANIMAL HEALTH AG, Pfäffikon (CH)

(72) Inventors: Andris ZELTINS, Riga (LV); Kaspars TARS, Jaunolaine (LV)

(21) Appl. No.: 19/105,567

(22) PCT Filed: Aug. 30, 2023
(86) PCT No.: PCT/EP2023/073756

§ 371 Date: Feb. 21, 2025

(30) Foreign Application Priority Data
Aug. 30, 2022 (EP) 22192929.2
Sep. 23, 2022 (EP) 22197365.4

(51) Int. Cl. C12N 15/86; C12N 7/00

(52) U.S. Cl. CPC C12N 15/86; C12N 7/00; C12N 2710/16122; C12N 2710/16123; C12N 2710/16134; C12N 2710/16143

(57) ABSTRACT

The present invention relates to a modified virus-like particle (VLP) of cucumber mosaic virus (CMV) comprising at least one chimeric CMV polypeptide, wherein said at least one chimeric CMV polypeptide comprises, preferably consists of (i) a CMV polypeptide, wherein said CMV polypeptide comprises a coat protein of CMV or an amino acid sequence having a sequence identity of at least 75% with SEQ ID NO:48; and (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:48, as well as to compositions and pharmaceutical compositions comprising such modified VLPs to which antigens are linked, which compositions preferably serve as vaccine platform for generating immune responses, in particular antibody responses, against said antigens linked to the modified CMV VLPs.

The present invention relates to modified virus-like particles (VLPs) of Cucumber Mosaic Virus (CMV), and in particular to modified VLPs of CMV comprising chimeric CMV polypeptides which comprises a stretch of consecutive negative amino acids selected from aspartic acid or glutamic acid, as well as compositions and pharmaceutical compositions comprising such modified VLPs to which antigens are linked, which compositions preferably serve as vaccine platform for generating immune responses, in particular antibody responses, against said antigens linked to the modified CMV VLPs.

### RELATED ART

Virus-like particles (VLPs) have become an established and accepted vaccine technology, in particular as immunological carriers for inducing strong immune responses against conjugated antigens (Zeltins A, Mol Biotechnol (2013) 53:92-107; Jennings G T and Bachmann M F, Annu Rev Pharmacol Toxicol (2009) 49:303-26, Jennings G T and Bachmann M F, Biol Chem (2008) 389:521-536).

Recently, a vaccine platform based on Cucumber Mosaic Virus (CMV, family Bromoviridae, genus Cucumovirus) virus-like particles (CMV VLPs) has been described using chemical linker coupling technology to present different antigens including self-antigens such as cytokines on their surface, and elicit effective neutralizing antibody responses. These soluble and stable CMV VLPs serve as an excellent platform due to their intrinsic properties such as repetitive presentation of the target antigen to B cell receptors, nanoscale dimensions and geometry, as well as activation of innate immunity through activation of TLRs and provision of T cell help (WO2016/062720; Zeltins A et al. Vaccines 2 (2017) 30; Bachmann M F et al. Frontiers in Microbiology Vol. 9, Article 2522, October 2018; von Loga I S et al. Ann. Rheum Dis 2019, 78:672-675; WO2021/260131).

Despite the progress made in the course of the development of these versatile VLP based vaccines, there are challenges and requirements that have to be taken into account, in particular for eventual clinical trial testing, product registration, market launch and commercial supply needs. Hereby, controlling product characteristics such as stability, shelf-life, solubility, manufacturability including scalability, safety, potency, bioavailability and other pharmacological properties are particularly to be mentioned and are key elements of the chemistry, manufacturing and control (CMC) process necessary for the cost-effective provision of these products in sufficient amounts for such eventual clinical trial testing, product registration, market launch and commercial supply needs (Pham N G, Int J Pharm, 2020, 585:119523). In particular, the stability of these VLP platforms and VLP based vaccines even under various conditions required for an efficient CMC process is of relevance. A further undesired occurrence and problem negatively impacting product characteristics is the aggregation of biopharmaceuticals and vaccines, respectively (Roberts C J, Current Opinion in Biotechnology, 2014, 30:211-217). While an aggregated vaccine may still be capable of eliciting an immune response, provided its native structure is maintained, and even though it may thus still be suitable for some laboratory studies, it is not acceptable for GMP products produced for clinical studies and the market.

Therefore, despite the progress made in the course of the development of these versatile VLP based vaccines, there is still a need for development of modified VLP systems adapted to address challenges that can arise and meet the requirements for eventual product registration and market launch.

### SUMMARY OF THE INVENTION

We have surprisingly found that stretches of consecutive negative amino acids selected from glutamic acid and aspartic acid can be engineered within the vicinity of the βB-βC loop of cucumber mosaic virus (CMV) polypeptides and coat proteins, respectively, wherein the resulting chimeric CMV polypeptides are not only still capable of forming and assembling to stable modified virus-like particles (VLPs), but, in addition, said modified VLPs can serve as a highly immunogenic carrier platform, in particular a vaccine platform, for linked antigens to which immune responses are to be generated. Further surprisingly, the specific insertion of these stretches of consecutive negative amino acids selected from glutamic acid and aspartic acid into the CMV polypeptides and coat proteins unexpectedly led even to improvements in stability of the resulting modified CMV VLPs as compared to prior art CMV VLPs under conditions of elevated temperatures and higher ionic strengths. In particular, the improved stability in higher salt solution arising from the surface charge modifications to the CMV VLPs is highly beneficial or even essential for its processability and purification by ion-exchange chromatography. Thus, the improved stability and, as a consequence, processability and purification by ion-exchange chromatography, in particular anion exchange chromatography advantageously further allows readiness for scalable manufacturing of these modified CMV VLPs. The formation of stable modified CMV VLPs, let alone the even improved stability as compared to prior art CMV VLPs, was in particular surprising given that inclusion of additional negative charge within the coat protein can have deleterious effects on the formation of virus-like particles. Moreover, we have further surprisingly found that the inventive modified CMV VLPs maintain stability and structural integrity even, and in particular, upon linking antigens that result in aggregation and formation of aggregated conjugated CMV VLPs when linked to prior art CMV VLPs. Thus, the inventive modified CMV VLP vaccine platform comprising stretches of consecutive negative amino acids selected from glutamic acid and aspartic acid avoids such aggregation and formation of aggregated conjugated CMV VLPs which is highly undesired for drug development and product registration. In particular, it has been found that such undesired aggregation can be substantially reduced or avoided for antigens such as interleukins and growth factors. Specifically, preferred CMV VLPs of the present invention comprising a stretch of consecutive glutamic acid residues formed stable, soluble and highly immunogenic conjugates when a growth factor or interleukin such as canine or feline mature NGF, canine or feline IL-1β, feline IL-5 antigens were coupled thereto, while the CMV VLP counterparts not comprising said stretches of consecutive negative amino acids formed large aggregates that precipitated from solution.

Thus, in a first aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48; and
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48.

In a further aspect, the present invention provides a composition comprising
(a) a modified VLP of CMV as defined herein, wherein said modified VLP of CMV comprises at least one first attachment site;
(b) at least one antigen, wherein said antigen comprises at least one second attachment site; and
wherein (a) and (b) are linked through said at least one first and said at least one second attachment site via at least one covalent non-peptide bond.

Further aspects and embodiments of the present invention will become apparent as this description continues.

### BRIEF DESCRIPTION OF FIGURES

FIG. 1: Description of pET-CMVB2-Ntt-E8* plasmid map with single-cut restriction enzyme sites.

FIG. 2A: SDS-PAGE gel analysis of the purification of the VLP derived from the expression of CMV-Ntt830-E8*. M—protein size marker PageRuler (Thermo Fisher Scientific, #26620); S—soluble proteins in cell extract in E. coli C2566/pET-CMVB2-Ntt-E8*; P—insoluble proteins in cell extract; I—insoluble proteins after sucrose gradient (bottom of the tube); 2-6—sucrose gradient fractions (from 60% at the bottom of tube to 0% at the top). The asterisk (*) within the figure denotes the relative position of the corresponding CMV-Ntt830-E8* chimeric CMV polypeptide in SDS/PAGE gel.

FIG. 2B: Electron microscopy images of purified CMV-Ntt830-E8* VLPs. The horizontal bar corresponds to 500 nm.

FIG. 3: Description of pET-CMVB2-Ntt-E4 plasmid map with single-cut restriction enzyme sites.

FIG. 4: Description of pET-CMVB2-Ntt-E8 plasmid map with single-cut restriction enzyme sites.

FIG. 5: Description of pET-CMVB2-Ntt-E12 plasmid map with single-cut restriction enzyme sites.

FIG. 6: SDS-PAGE (left) and agarose gel (right) analysis of the purification of the VLP derived from the expression of CMV-Ntt830-E4. M1—protein size marker PageRuler (Thermo Fisher Scientific, #26620); M2—DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E4 chimeric CMV polypeptide in SDS/PAGE gel and typical VLP signal in agarose gel.

FIG. 7: SDS-PAGE (left) and agarose gel (right) analysis of the purification of the VLP derived from the expression of CMV-Ntt830-E8. M1—protein size marker PageRuler (Thermo Fisher Scientific, #26620); M2—DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E8 chimeric CMV polypeptide in SDS/PAGE gel and typical VLP signal in agarose gel.

FIG. 8: SDS-PAGE (left) and agarose gel (right) analysis of the purification of the VLPs derived from the expression of CMV-Ntt830-E12. M1—protein size marker PageRuler (Thermo Fisher Scientific, #26620); M2—DNA size marker (Thermo Fisher Scientific, #SM0311); T—total proteins in E. coli C2566 cells after 18 h cultivation at 20° C.; S—soluble proteins in cell extract after cell disruption before sucrose gradient (20-60%); P—insoluble proteins; 1-6—sucrose gradient fractions (from 60% at the bottom of tube to 0% at the top. The asterisk (*) within the figure denotes the relative position of the corresponding CMV-Ntt830-E12 chimeric CMV polypeptide in SDS/PAGE gel. A clear and distinct band corresponding to intact VLPs was not observed in the agarose gel.

FIG. 9: Electron microscopy images of purified CMV-Ntt830-E4 VLPs. Horizontal bar corresponds to 200 nm.

FIG. 10: Electron microscopy images of purified CMV-Ntt830-E8 VLPs. Horizontal bar corresponds to 200 nm.

FIG. 11: Comparison of thermal stability of CMV-Ntt830 VLPs and CMV-Ntt830-E4 VLPs. The structural changes in CMV-Ntt830 VLPs and CMV-Ntt830-E4 VLPs were monitored in the presence of Sypro-Orange dye using a DNA melting point determination program and a real-time PCR system. Curve 1 is for CMV-Ntt830-E4 VLPs), curve 2 is for CMV-Ntt830 VLPs and Curve 3 is for buffer control (5 mM Na phosphate 2 mM EDTA, pH 7.5). The respective 57° C. and 51° C. melting points are indicated by arrows.

FIG. 12: Stability of CMV-Ntt830 VLPs and CMV-Ntt830-E4 VLPs in solution in the presence of different NaCl concentrations. Samples of CMV-Ntt830 VLPs and CMV-Ntt830-E4 VLPs at 0.5 mg/ml were incubated at room temperature in 5 mM Na phosphate, 2 mM EDTA, pH 7.5 with different concentrations of NaCl (the molar concentration of NaCl in each sample is indicated at the bottom of the gels) for up to 2 hours. Samples were analysed by native agarose gel electrophoresis and ethidium bromide staining. Panels A and B show NAGE analysis of CMV-Ntt830 VLP and CMV-Ntt830-E4 VLPsamples respectively. M shows the lanes loaded with GeneRuler 1 kb DNA Ladder (SM0311, TFS). Black arrows indicate the position of loading wells within the gels and location of VLPs within the wells and gels. The presence of CMV-Ntt830 VLPs in the loading wells after electrophoresis (panel A) is due to the formation of VLP aggregates which are too large to enter the gel. Integral unaggreagted VLPs migrated into the gel.

FIG. 13: Analysis of CMV-Ntt830 VLPs subject to Anion Exchange Chromatography. 5 ml of 1 mg/ml CVMtt-VLPs in 5 mM Sodium Borate buffer pH 9.0 was loaded onto 1.0 ml Macro-Prep DEAE Bio-Rad anion exchange cartridge equilibrated with 5 mM Sodium Borate buffer and eluted step-wise with increasing concentrations of NaCl (0.1, 0.2, 0.3, 0.4. 0.5, 0.8, 1.0 and 2.0 M). Fractions were collected and analysed by nanodrop 260 nm for protein concertation and native agarose gel electrophoresis. Panel A shows the NaCl concentration and 260 nm absorbance values plotted against the respective fractions (1-25). Panel B is a NAGE analysis (ethidium bromide stained) of the principle fractions containing the highest protein concentrations. M shows the lanes loaded with GeneRuler 1 kb DNA Ladder (SM0311, TFS). Black arrows indicate the position of loading wells within the gels and location of VLPs within the wells and gels. The presence of CMV-Ntt830 VLPs in the loading wells after electrophoresis is due to the formation of VLP aggregates which are too large to enter the gel. Integral unaggreagted VLPs migrated into the gel.

FIG. 14: Analysis of CMV-Ntt830-E4 VLPs subject to Anion Exchange Chromatography. A biomass of E. coli cells expressing CMV-Ntt830-E4 VLPs was resuspended in 50 mM citrate, 5 mM Borate buffer pH 9.0 and cells were lysed using a microfluidizer LM-20. The soluble fraction was clarified by centrifugation and loaded onto a 60 ml Fracto-DEAE (XK 26/20). An elution buffer comprising 50 mM Citrate 5 mM Borate and 1M NaCl was applied in a continuous gradient manner to elute the bound VLPs. Panel A shows the protein elution and NaCl concentration gradient measured by A260 nm (mAU) and conductivity (mS/cm) respectively. The X-axis shows the elution volume and fraction numbers (4-11). The fractions collected from the Fracto-DEAE column were analysed by NAGE (panel B) and SDS-PAGE (panel C). In panel B, M indicates the lane loaded with a GeneRuler 1 kb DNA Ladder (SM0311, TFS), L is a sample of E. coli lysate before loading onto the Fracto DEAE, FT is the flow through collected from 0 to 150 ml and 4-10 represent the fraction numbers collected during elution. The black arrows from top to bottom indicate the position of the loading wells, position of integral CMV-Ntt830-E4 VLPs within the gel and contaminating nucleic acids from the clarified bacterial lysate respectively. In panel C, FT is the flow through collected from 0 to 150 ml and 4-10 represent the fraction numbers. The black arrow shows the position of the Coomassie blue stained CMV-Ntt830-E4 coat protein.

FIG. 15A: Purification and authenticity of recombinant canine mature NGF. SDS-PAGE analysis of the NGF purification process. M—marker, with molecular weights of bands shown in kDa; A—total cell lysate after expression, B—pooled fractions containing pro-NGF after refolding and partial purification; C—mature NGF after trypsin digestion and final purification. Arrows indicate pro-NGF in lanes A and B and mature NGF in lane C.

FIG. 15B: PC12 cells were grown for 5 days with recombinant human mature NGF produced in mouse myeloma cells (R&D systems) (black squares) or with canine mature NGF produced in E. coli as described herein (grey circles). Cells were grown in the presence of 100, 50, 25, 12.5 and 6.25 ng/ml of recombinant NGF and the percentage of cells with defined neurite outgrowth determined.

FIG. 16A: SDS-PAGE analysis of coupling of recombinant mature canine NGF (cNGF) to CMV-Ntt830 and CMV-Ntt830-E8* VLPs.
M—PageRuler™ Plus Prestained Protein Ladder, 10 to 250 kDa (Thermo Fisher Scientific, #26620) protein size marker; 1—Corresponding purified CMV-Ntt830 and CMV-Ntt830-E8* VLPs; 2—CMV VLPs after derivatization with 5×SMPH and removal of SMPH; 3—CMV VLPs coupled with equimolar amount of cNGF; 4—mixed samples of CMV-Ntt830-E8* and cNGF without SMPH derivatization; 5—purified cNGF. The asterixes denote the localization of observable CMV VLPs-NGF conjugate bands.

FIG. 16B: SDS-PAGE analysis of coupling of recombinant mature canine NGF (cNGF) to CMV-Ntt830-E4 and CMV-Ntt830-E8 VLPs.
M—PageRuler™ Plus Prestained Protein Ladder, 10 to 250 kDa (Thermo Fisher Scientific, #26620) protein size marker; 1—Corresponding purified CMV-Ntt830-E4 and CMV-Ntt830-E8 VLPs; 2—CMV VLPs after derivatization with 5×SMPH and removal of SMPH; 3—CMV VLPs coupled with equimolar amount of cNGF; 4—mixed samples of CMV-Ntt830-E4 or CMV-Ntt830-E8 and cNGF without SMPH derivatization; 5—purified cNGF. The asterixes denote the localization of observable CMV VLPs-cNGF conjugate bands.

FIG. 16C: Dynamic light scattering analysis of cNGF-CMV-Ntt830 VLPs. Because the vaccine precipitated, EM analysis could not be performed.

FIG. 16D: Dynamic light scattering analysis of cNGF-CMV-Ntt830-E4 VLPs comprising cNGF antigens of SEQ ID NO:31.

FIG. 16E: Dynamic light scattering analysis of cNGF-CMV-Ntt830-E8* VLPs comprising cNGF antigens of SEQ ID NO:174.

FIG. 16F: Dynamic light scattering analysis of cNGF-CMV-Ntt830-E8* VLPs.

FIG. 16G: Electromicroscopy of cNGF-CMV-Ntt830-E4 VLPs.

FIG. 16H: Electromicroscopy of cNGF-CMV-Ntt830-E8* VLPs.

FIG. 17A: Assessment of anti-NGF IgG antibodies from sera of mice immunized with cNGF-CMV-Ntt830-E8* VLP. Anti-NGF IgG titers in mice immunized twice (Day 0 and 14 indicated by arrows) with 15 ug with cNGF-CMV-Ntt830-E8* VLP with or without Quil A adjuvant (closed and open circles respectively) were measured by ELISA.

FIG. 17B: To test for neutralizing IgG antibodies generated in mice, PC12 cells were grown for 5 days in the presence of 12.5 ng/ml human mature NGF (or without as a negative control) in the presence of either anti-human NGF polyclonal antibody (from BioTechne) or purified IgG from naive mice (ms pIgG NAIVE) or mice immunized with cNGF-CMV-Ntt830-E8* VLP (serum pooled from study day 21, 28 and 35, ms pIgG NGF vacc) at the indicated concentrations. Data points represent sample replicates.

FIG. 18A: Assessment of anti-NGF IgG antibodies from sera of dogs immunized with cNGF-CMV-Ntt830-E8* VLP. Anti-NGF IgG titers of dogs from group 1 that received vaccine without adjuvant. Arrows indicate the injections of vaccine administered on day 0, 21 and 42.

FIG. 18B: Assessment of anti-NGF IgG antibodies from sera of dogs immunized with cNGF-CMV-Ntt830-E8* VLP. Anti-NGF IgG titers of 3 dogs from group 2 that received vaccine with adjuvant QuilA®. Arrows indicate the injections of vaccine administered on day 0, 21 and 42.

FIG. 18C: Assessment of anti-CMV IgG titers from sera of dogs immunized with cNGF-CMV-Ntt830-E8*VLP. Anti-CMV IgG titers of dogs from group 1 that received vaccine without adjuvant. Arrows indicate the injections of vaccine administered on day 0, 21 and 42.

FIG. 18D: Assessment of anti-CMV IgG titers from sera of dogs immunized with cNGF-CMV-Ntt830-E8* VLP. Anti-CMV IgG titers of dogs from group 2 that received vaccine with adjuvant QuilA®. Arrows indicate the injections of vaccine administered on day 0, 21 and 42.

FIG. 18E: Assessment of anti-NGF IgG antibodies from sera of dogs immunized with cNGF-CMV-Ntt830-E4 VLP in absence of adjuvant. 5 dogs were dosed with cNGF-CMV-Ntt830-E4 VLP on day 0 and 21. NGF-specific antibodies were assessed by ELISA in serum collected on days 0, 21, 42, 71 and 91.

FIG. 18F: Assessment of anti-NGF IgG antibodies from sera of dogs immunized with cNGF-CMV-Ntt830-E4 VLP in presence of aluminum hydroxide. 5 dogs were dosed with cNGF-CMV-Ntt830-E4 VLP with aluminum hydroxide on day 0 and 21. NGF-specific antibodies were determined by ELISA on days 0, 21, 42, 71 and 91.

FIG. 19A: Vaccination with cNGF-CMV-Ntt830-E8* VLP induces NGF neutralizing antibodies in dogs. Dogs (3 dogs per group) were immunized with 250 g cNGF-CMV-Ntt830-E8* VLP in presence or absence of adjuvant QuilA at day 0, day 21 and day 42. Sera were collected and tested for presence of neutralizing antibodies using a TF-1 based NGF bioactivity assay. Representation of titration of curves from one dog to determine neutralization capacity and 50% neutralization titers (NT50) of dog sera. 5 ng/mL human mature NGF was preincubated with increasing concentration of IgG purified from sera collected at indicated days after first administration of the vaccine. NT50 values, i.e. IgG concentration leading to 50% inhibition of cell proliferation, were determined using a 4PL sigmoidal curve fit model.

FIG. 19B: Vaccination with cNGF-CMV-Ntt830-E8* VLP induces NGF neutralizing antibodies in dogs. Dogs (3 dogs per group) were immunized with 250 g cNGF-CMV-Ntt830-E8* VLP in presence or absence of adjuvant QuilA at day 0, day 21 and day 42. Sera were collected and tested for presence of neutralizing antibodies using a TF-1 based NGF bioactivity assay. Total IgG were purified from dog sera. The capacity of 20 g/mL of purified total IgG to neutralize 5 ng human mature NGF/mL was assessed using the bioassay. Bars represent mean group values with standard deviation and symbols represent individual dogs (mean of assay duplicate). 2-way ANOVA with Tukey's multiple comparisons test was performed to compare group mean values using GraphPad Prism.* p<0.05, ** p<0.01, p<0.001, **** p<^{0.0001}.

FIG. 19C: Vaccination with cNGF-CMV-Ntt830-E8* VLP induces mature NGF neutralizing antibodies in dogs. Dogs (3 dogs per group) were immunized with 250 ug cNGF-CMV-Ntt830-E8* VLP in presence or absence of adjuvant QuilA at day 0, day 21 and day 42. Sera were collected and tested for presence of neutralizing antibodies using a TF-1 based NGF bioactivity assay. NT50 values were plotted versus OD50 values of anti-NGF IgG serum titers. Total IgG purified from serum with higher concentrations of NGF-specific antibodies were more potent to inhibit NGF mediated TF-1 cell proliferation than total IgG purified from sera of dogs with lower anti-NGF titers. Symbols represent individual dogs and sampling time points. Different symbols were allocated to different dogs. Closed symbols represent animals vaccinated in presence of adjuvant, whereas open symbols representing animals vaccinated without adjuvant.

FIG. 19D: Vaccination with cNGF-CMV-Ntt830-E4 VLP induces NGF neutralizing antibodies in dogs. cNGF-CMV-Ntt830-E4 VLP with aluminum hydroxide was adminstered to 5 dogs on day 0 and 21. Sera collected on day 42 were tested for presence of neutralizing antibodies using a TF-1 based NGF bioactivity assay. Bars represent mean group values with standard deviation and symbols represent individual dogs. The dotted line indicates detection limit of the assay.

FIG. 20: Description of pET42NBS-2×flIL5-C6Hcg plasmid map with single-cut restriction enzyme sites.

FIG. 21: SDS-PAGE (left) and Western blot (right) analysis of coupling of recombinant feline IL-5 dimer antigen to CMV-Ntt830-E8* VLPs. M—PageRuler™ Plus Prestained Protein Ladder, 10 to 250 kDa (TFS, Cat.Nr. 26620); 1—CMV-Ntt830-E8* VLP (1.5 mg/ml); 2—CMV-Ntt830-E8* VLP after 5×SMPH derivatization and unreacted SMPH removal; 3—CMV-Ntt830-E8* VLP after coupling reaction with recombinant feline IL-5 dimer antigen (10 xTCEP); 4—2×flIL5-CMV-Ntt830-E8* VLP after centrifugal ultrafiltration to remove uncoupled recombinant feline IL-5 dimer antigen; 5—recombinant feline IL-5 dimer antigen. For Western blot, feline IL-5 specific polyclonal IgGs were used (dilution 1:1000; Thermo Fisher Scientific, Cat. No. PA5-47994). * Denotes bands representing recombinant feline IL-5 dimer antigen covalently conjugated to either CMV VLP coat protein monomer or dimer which co-localize on SDS-PAGE and Western blot.

FIG. 22: Coomassie Blue G-250 stained Bolt-PAGE Gel of coupling reactions of recombinant feline IL-5 dimer antigens with modified CMV VLPs. 1—CMV-Ntt830 after coupling reaction with recombinant feline IL-5 dimer antigen; 2—CMV-Ntt830E8* after coupling reaction with recombinant feline IL-5 dimer antigen; 3—CMV-Ntt830-E4 after coupling reaction with recombinant feline IL-5 dimer antigen (10×TCEP); 4—recombinant feline IL-5 dimer antigen after treatment with 10×TCEP; 5—PageRuler™ Plus Prestained Protein Ladder, 10 to 250 kDa (TFS, Cat.Nr. 26620); 6—CMV-Ntt830 VLP (10 ug loaded); 7—CMV-Ntt830 VLP after derivatization with 5×SMPH. * Denotes bands representing recombinant feline IL-5 dimer antigen covalently conjugated to either CMV VLP coat protein monomer or dimer. System used: 10 well 1.0 mm Bolt 4-12% Bis-Tris Plus gel (TFS, Cat. No. NW04120BOX) in 1×MES SDS running buffer for 30 min at 200 V/125 mA.

FIG. 23A: Native Agarose Gel (0.8%) Electrophoresis of CMV-Ntt830, CMV-Ntt830-E8* and CMV-Ntt830-E4 VLPs from various stages of the coupling reaction. Agarose gels were run in parallel and stained with ethidium bromide. M1—GeneRuler 1 kb DNA Ladder (SM0311, TFS); 1—VLPs; 2—VLPs after 5×SMPH derivatization and removal of unreacted SMPH; 3—VLPs after coupling with recombinant feline IL-5 dimer antigen.

FIG. 23B: Native Agarose Gel (0.8%) Electrophoresis of CMV-Ntt830, CMV-Ntt830-E8* and CMV-Ntt830-E4 VLPs from various stages of the coupling reaction. Agarose gels were run in parallel and stained with Coomassie blue G250. M1—GeneRuler 1 kb DNA Ladder (SM0311, TFS); 1—VLPs; 2—VLPs after 5×SMPH derivatization and removal of unreacted SMPH; 3—VLPs after coupling with recombinant feline IL-5 dimer antigen.

FIG. 24A: Dynamic light scattering analysis of 2×flIL5-CMV-Ntt830 VLPs. Because the vaccine precipitated, EM analysis could not be performed.

FIG. 24B: Dynamic light scattering analysis of 2×flIL5-CMV-Ntt830-E8* VLPs FIG. 24C: Electromicroscopy of 2×flIL5-CMV-Ntt830-E4 VLPs.

FIG. 24D: Electromicroscopy of 2×flIL5-CMV-Ntt830-E8* VLPs.

FIG. 25A: Vaccination with modified CMV VLPs to which recombinant feline IL-5 dimer antigens are coupled induces anti-feline IL-5 antibodies in mice. Four Balb/c mice were immunized with 30 g recombinant feline IL-5 dimer antigen-CMV-E8C at day 0 and day 14. Sera were collected and tested for presence of feline IL-5 specific IgG determined by an ELISA assay. Bars represent mean group values with standard deviation and symbols represent individual mice.

FIG. 25B: Vaccination with CMV-Ntt830-E8* VLPs to which recombinant feline IL-5 dimer antigens are coupled_induces anti-feline IL-5 antibodies in mice. Four Balb/c mice were immunized with 30 g recombinant feline IL-5 dimer antigen-CMV-E8C at day 0 and day 14. Sera were collected at day 28 and tested for presence of neutralizing antibodies determined by a TF-1 based bioactivity assay. Bars represent mean group values with standard deviation and symbols represent individual mice.

FIGS. 26A-E: Vaccination with CMV-Ntt830-E8* VLPs to which recombinant feline IL-5 dimer antigens are coupled induces anti-feline IL-5 specific antibodies and leads to a reduction of eosinophils in the blood of vaccinated cats. Cats (n=3) were immunized with 250 g CMV-Ntt830-E8* VLPs to which recombinant feline IL-5 dimer antigens are coupled at day 0, day 21 and day 42. Sera were collected and tested for presence of feline IL-5 (FIG. 26A) as well as carrier CMV VLP (FIG. 26B) specific IgG antibodies and feline IL-5 neutralizing antibodies using a TF-1 based IL-5 bioactivity assay (FIG. 26C). Feline I L-5 specific neutralization titers positively correlated with target specific IgG titers detected in sera of cats collected at day 42, 63 and 105. Symbols represent individual cats and sampling time points. Different symbols were allocated to different study days (FIG. 26D). Eosinophils in the blood of cats included in the study were enumerated by differential blood counts at study day 0, 21, 42, 63 and 84 (FIG. 62E). Immunization with CMV-Ntt830-E8* VLPs to which recombinant feline IL-5 dimer antigens are coupled led to a significant reduction of blood eosinophils.

FIG. 27: Description of pET42NBS-cIL1b-C6Hcg plasmid map with single-cut restriction enzyme sites.

FIG. 28: SDS-PAGE (left) and Western blot (right) analysis of coupling of the canine IL-1β antigen to CMV-Ntt830-E8* VLPs. M—PageRuler™ Plus Prestained Protein Ladder, 10 to 250 kDa (Thermo Fisher Scientific, Cat.Nr. 26620); 1—CMV-Ntt830-E8* (1.5 mg/ml); 2—CMV-Ntt830-E8* after 5×SMPH derivatization and removal of unreacted SMPH; 3—CMV-Ntt830-E8* after coupling reaction with canine IL-1β antigen; 4—CMV-Ntt830-E8*+canine IL-1β antigen after removal of uncoupled canine IL-1β antigen; 5—the canine IL-1β antigen; For Western blot, monoclonal antibody against a His-tag was used (dilution 1:1000; Merck, Cat. No. 71840-3). * Denotes bands representing the canine IL-1β antigen covalently conjugated to either CMV-Ntt830-E8* coat protein monomer or dimer which co-localize on SDS-PAGE and Western blot.

FIG. 29: Coomassie Blue G-250 stained Bolt-PAGE Gel of coupling reactions of recombinant canine IL-1β antigens with modified CMV VLPs. M—PageRuler™ Plus Prestained Protein Ladder, 10 to 250 kDa (TFS, Cat.Nr. 26620); 1—CMV-Ntt830 VLP's after 5×SMPH derivatization and removal of unreacted SMPH; 2—CMV-Ntt830 VLPs after coupling reaction with canine IL-1β antigen; 3—CMV-Ntt830-E4 VLP's; 4—CMV-Ntt830-E4 VLP's after 5×SMPH derivatization and removal of unreacted SMPH; 5—CMV-Ntt830-E4 VLPs after coupling reaction with canine IL-1β antigen; 6—CMV-Ntt830-E8* VLP's; 7—CMV-Ntt830-E8* VLP's after 5×SMPH derivatization and removal of unreacted SMPH; 8—CMV-Ntt830-E8* VLPs after coupling reaction with canine IL-1β antigen; 9—the canine IL-1β antigen after reaction with 10×TCEP. * Denotes bands representing the canine IL-1β antigen covalently conjugated to either CMV VLP coat protein monomer or dimer. System used: 10 well 1.0 mm Bolt 4-12% Bis-Tris Plus gel (TFS, Cat. No. NWO4120BOX) in 1×MES SDS Running buffer for 30 min at 200 V/125 mA.

FIGS. 30A and 30B: Native Agarose Gel (0.8%) Electrophoresis of CMV-Ntt830, CMV-Ntt830-E8* and CMV-Nt830-E4 VLPs from various stages of the coupling reaction. Agarose gels were run in parallel and stained with ethidium bromide (FIG. 30A) or Coomassie blue G250 (FIG. 30B). M1—GeneRuler 1 kb DNA Ladder (SM0311, TFS); 1—CMV-Ntt830 VLPs; 2—CMV-Ntt830 VLPs after 5×SMPH derivatization and removal of unreacted SMPH; 3—CMV-Ntt830 VLPs after coupling with canine IL-1β antigen; 4—CMV-Ntt830-E4 VLPs; 5—CMV-Ntt830-E4 VLPs after 5×SMPH derivatization and removal of unreacted SMPH; 6—CMV-Ntt830-E4 VLPs after coupling with canine IL-1β antigen; 7—CMV-Ntt830-E4 VLPs after coupling with canine IL-1β antigen and centrifugation at 14′000 rpm; 8—CMV-Ntt830-E8* VLPs; 9—CMV-Ntt830-E8* VLPs after 5×SMPH derivatization and removal of unreacted SMPH; 10—CMV-Ntt830-E8* VLPs after coupling with the canine IL-1β antigen; 11—CMV-Ntt830-E8* VLPs after coupling with the canine IL-1β antigen and centrifugation at 14′000 rpm.

FIG. 31A: Dynamic light scattering analysis of cIL1b-CMV-Ntt830-E4 VLP conjugates.

FIG. 31B: Electromicroscopy of cIL1b-CMV-Ntt830-E4 VLP conjugates.

FIG. 32A: Dynamic light scattering analysis of cIL1b-CMV-Ntt830-E8* VLP conjugates.

FIG. 32B: Electromicroscopy of cIL1b-CMV-Ntt830-E8* VLP conjugates.

FIGS. 33A-C: Vaccination with cIL1b-CMV-Ntt830-Ntt830-E4 and cIL1b-CMV-Ntt830-E8* VLPs induce canine IL-1β-specific IgG and neutralizing antibodies. Balb/c mice (5 per group) were immunized with 30 g cIL1b-CMV-Nt830-E4 or cIL1b-CMV-Ntt830-E8* VLPs at day and day 14. Sera were collected and tested for presence of canine IL-1β-specific IgG antibodies (FIG. 33A), CMV VLP-specific IgG antibodies (FIG. 33B) and canine IL-1β neutralizing antibodies (FIG. 33C). Neutralization titers were determined using a bioassay based on secretion of IL-6 by HeLa cells in presence of 30 pg/mL IL-1β. Bars represent mean group values with standard deviation and symbols represent individual animals. Open squares are animals that were immunized with cIL1b-CMV-NT830-E4 VLPs, and closed circles received cIL1b-CMV-Ntt830-E8* VLPs.

FIG. 34: Description of pET42NBS-fIL1b-C6Hcg plasmid map with single-cut restriction enzyme sites.

FIG. 35A: SDS-PAGE analysis of coupling of the feline IL-1β antigen to CMV-Ntt830-E4 VLPs. M—PageRuler™ Plus Prestained Protein Ladder, 10 to 250 kDa (Thermo Fisher Scientific, Cat.Nr. 26620); 1—CMV-Ntt830-E4 (1.5 mg/ml); 2—CMV-Ntt830-E4 after 5×SMPH derivatization and removal of unreacted SMPH; 3—CMV-Ntt830-E4 after coupling reaction with feline IL-1β antigen; 4—soluble fraction of CMV-Ntt830-E4+feline IL-1β antigen after clarification by centrifugation; 5—insoluble fraction of CMV-Ntt830-E4+feline IL-1β antigen after clarification by centrifugation; 7 Soluble CMV-Ntt830-E4+feline IL-1β after size exclusion to deplete unconjugated feline IL-1β antigen and Amicon filtration to concentrate the sample and subsequent sterile filtration; 8 and 9 purified feline IL-1β antigen used for the conjugation; * Denotes bands representing the feline IL-10 antigen covalently conjugated to either CMV-Ntt830-E4 coat protein monomer or dimer.

FIG. 35B: SDS-PAGE analysis of coupling of the feline IL-1β antigen to CMV-Ntt830 VLPs. M—PageRuler™ Plus Prestained Protein Ladder, 10 to 250 kDa (Thermo Fisher Scientific, Cat.Nr. 26620); 1—CMV-Ntt830 (1.5 mg/ml); 2—CMV-Ntt830 after 5×SMPH derivatization and removal of unreacted SMPH; 3—CMV-Ntt830 after coupling reaction with feline IL-1β antigen; * Denotes bands representing the feline IL-1β antigen covalently conjugated to either CMV-Ntt830 coat protein monomer or dimer.

FIGS. 36A and 36B: Native Agarose Gel (0.8%) Electrophoresis of CMV-Nt830-E4 and CMV-Ntt830 VLPs from various stages of the coupling reaction. Agarose gels were run in parallel and stained with ethidium bromide (FIG. 32A) or Coomassie blue G250 (FIG. 32B). M—GeneRuler 1 kb DNA Ladder (SM0311, Thermo Fisher Scientific); 1—CMV-Ntt830-E4 VLPs; 2—CMV-Ntt830-E4 VLPs after 5×SMPH derivatization and removal of unreacted SMPH; 3—CMV-Ntt830-E4 VLPs after coupling with feline IL-1β antigen; 4—Soluble fraction of CMV-Ntt830-E4 VLPs after coupling with feline IL-1β antigen and clarification by centrifugation; 5—Soluble fraction of CMV-Ntt830-E4 VLPs after coupling with feline IL-1β antigen and subsequent size exclusion to deplete unconjugated feline IL-1β antigen and Amicon filtration to concentrate the sample; 6—CMV-Ntt830-E4 VLPs from 5 after sterile filtration; 7 feline IL-1β antigen: 8—CMV-Ntt830 VLPs; 9—CMV-Ntt830 VLPs after 5×SMPH derivatization and removal of unreacted SMPH; 10—CMV-Ntt830 VLPs after coupling with feline IL-1β antigen.

FIG. 37A: Dynamic light scattering analysis of fIL1b-CMV-Ntt830-E4 VLP conjugates.

FIG. 37B: Electromicroscopy of fIL1b-CMV-Ntt830-E4 VLP conjugates.

FIGS. 38A-C: Vaccination with fIL1b-CMV-Ntt830-E4 VLPs induced feline IL-1β-specific IgG and neutralizing antibodies. Balb/c mice (5 per group) were administered with g fIL1b-CMV-Ntt830-E4 VLPs or buffer control at day 0 and day 21. Sera were collected and tested for presence of feline IL-1β-specific IgG antibodies (FIG. 34A), CMV VLP-specific IgG antibodies (FIG. 34B) and feline IL-1β neutralizing antibodies (FIG. 34C). Neutralization titers were determined using a bioassay based on secretion of alkaline phosphatase by an IL-1β reporter cell line in presence of 500 pg/mL IL-1β. Bars represent geometric mean group values with error. Open squares are animals that were treated with buffer control, and closed circles received fIL1b-CMV-Ntt830-E4 VLPs.

### DETAILED DESCRIPTION OF THE INVENTION

Unless defined otherwise, all technical and scientific terms used herein have the same meanings as commonly understood by one of ordinary skill in the art to which this invention belongs. The herein described and disclosed embodiments, preferred embodiments and/or very preferred embodiments should apply to all aspects and other embodiments, preferred embodiments and/or very preferred embodiments irrespective of whether is specifically again referred to or irrespective of whether its repetition is avoided for the sake of conciseness. The articles “a” and “an”, as used herein, refer to one or to more than one (i.e., to at least one) of the grammatical object of the article. The term “or”, as used herein, should be understood to mean “and/or”, unless the context clearly indicates otherwise.

Virus-like particle (VLP): The term “virus-like particle (VLP)” as used herein, refers to a non-replicative or non-infectious, preferably a non-replicative and non-infectious virus particle, or refers to a non-replicative or non-infectious, preferably a non-replicative and non-infectious structure resembling a virus particle, preferably a capsid of a virus. The term “non-replicative”, as used herein, refers to being incapable of replicating the genome comprised by the VLP. The term “non-infectious”, as used herein, refers to being incapable of entering the host cell. A virus-like particle in accordance with the invention is non-replicative and non-infectious since it lacks all or part of the viral genome or genome function. A virus-like particle in accordance with the invention may contain nucleic acid distinct from their genome. Recombinantly produced virus-like particles typically contain host cell derived RNA. A typical and preferred embodiment of a virus-like particle in accordance with the present invention is a viral capsid composed of polypeptides of the invention. A virus-like particle is typically a macromolecular assembly composed of viral coat protein which typically comprises 60, 120, 180, 240, 300, 360, or more than 360 protein subunits per virus-like particle. Typically and preferably, the interactions of these subunits lead to the formation of viral capsid or viral-capsid like structure with an inherent repetitive organization. One feature of a virus-like particle is its highly ordered and repetitive arrangement of its subunits.

Modified virus-like particle (VLP) of CMV: The term “modified virus-like particle of CMV” refers to a virus-like particle comprising at least one chimeric CMV polypeptide as defined and as described herein. Typically and preferably, modified VLPs of CMV resemble the structure of the capsid of CMV. Modified VLPs of CMV are non-replicative and/or non-infectious, and lack at least the gene or genes encoding for the replication machinery of the CMV, and typically also lack the gene or genes encoding the protein or proteins responsible for viral attachment to or entry into the host. This definition includes also modified virus-like particles in which the aforementioned gene or genes are still present but inactive. Preferably, non-replicative and/or non-infectious modified virus-like particles are obtained by recombinant gene technology and typically and preferably do not comprise the viral genome. Preferably, a modified VLP of CMV is a macromolecular assembly composed of CMV polypeptides modified in accordance with the present invention, and typically and preferably comprising 180 of such protein subunits and chimeric polypeptides, respectively per VLP. Thus, in a preferred embodiment, said modified virus-like particle (VLP) of cucumber mosaic virus (CMV) comprises 180 chimeric CMV polypeptides.

Polypeptide: The term “polypeptide” as used herein refers to a polymer composed of amino acid monomers which are linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. The term “polypeptide” as used herein should also refer, typically and preferably to a polypeptide as defined before and encompassing modifications such as post-translational modifications, including but not limited to glycosylations. In a preferred embodiment, said term “polypeptide” as used herein should refer to a polypeptide as defined before and not encompassing modifications such as post-translational modifications such as glycosylations. In particular, for said biologically active peptides, said modifications such as said glycosylations can occur even in vivo thereafter, for example, by bacteria.

Cucumber Mosaic Virus (CMV) polypeptide, CMV polypeptide: The term “cucumber mosaic virus (CMV) polypeptide” as used herein refers to a polypeptide comprising or preferably consisting of: (i) an amino acid sequence of a coat protein of cucumber mosaic virus (CMV), or (ii) a mutated amino acid sequence, wherein said mutated amino acid sequence and said coat protein of CMV show a sequence identity of at least 90%, preferably of at least 91%, 92%, 93% or 94%, further preferably of at least 95%, again further preferably of at least 98% and further more preferably of at least 99%. Typically and preferably, the CMV polypeptide is capable of forming a virus-like particle of CMV upon expression by self-assembly.

Coat protein (CP) of cucumber mosaic virus (CMV): The term “coat protein (CP) of cucumber mosaic virus (CMV)”, as used herein, refers to a coat protein of the cucumber mosaic virus which occurs in nature. Due to extremely wide host range of the cucumber mosaic virus, a lot of different strains and isolates of CMV are known. The sequences of the coat proteins of said strains and isolates have been determined and are known to the skilled person in the art. The sequences of said coat proteins (CPs) of CMV are described in and retrievable from the known databases such as Genbank, www.dpvweb.net, or www.ncbi.nlm.nih.gov/protein/. Specific examples CPs of CMV are described in WO 2016/062720 at page 12, line 8 to page 13, line 25, the disclosure of which are explicitly incorporated herein by way of reference. A very preferred example and embodiment of a CMV coat protein is provided in SEQ ID NO:48. Thus, preferably, the term “coat protein of cucumber mosaic virus (CMV)”, as used herein, refers to an amino acid sequence of a coat protein of CMV, wherein said amino acid sequence comprises, or preferably consists of, SEQ ID NO:48 or an amino acid sequence having a sequence identity of at least 75%, preferably of at least 80%, more preferably of at least 85%, again further preferably of at least 90%, again further preferably of at least 91%, 92%, 93% or 94%, again more preferably of at least 95%, still further preferably of at least 96% or 97%, still further preferably of at least 98% and still again further more preferably of at least 99% of SEQ ID NO:48.

It is noteworthy that these strains and isolates have highly similar coat protein sequences at different protein domains, including the N-terminus of the coat protein. In particular, 98.1% of all completely sequenced CMV isolates share more than 85% sequence identity within the first 28 amino acids of their coat protein sequence, and still 79.5% of all completely sequenced CMV isolates share more than 90% sequence identity within the first 28 amino acids of their coat protein sequence.

Modified CMV polypeptide: The term “modified CMV polypeptide” as used herein refers to a CMV polypeptide comprising, or preferably consisting of, a CMV polypeptide, and a T helper cell epitope. Typically, the modified CMV polypeptide is capable of forming a virus-like particle of CMV upon expression by self-assembly. Preferably, the modified CMV polypeptide is a recombinant modified CMV polypeptide and is capable of forming a virus-like particle of CMV upon expression by self-assembly in E. coli.

Chimeric CMV polypeptide: The term “chimeric CMV polypeptide” as used herein refers to a polypeptide as defined herein and in accordance with the present invention, and comprising, preferably consisting of, a CMV polypeptide, wherein said CMV polypeptide is modified as defined and described herein, to comprise a polypeptide comprising, preferably consisting of, a stretch of consecutive negative amino acids independently selected from aspartic acid or glutamic acid, and optionally further to comprise a T helper cell epitope, all components as defined and described herein. Typically and preferably, the chimeric CMV polypeptide is capable of forming a modified virus-like particle of CMV upon expression by self-assembly. Thus, in a preferred embodiment, said chimeric CMV polypeptide is capable of forming a modified virus-like particle of CMV by self-assembly, typically and preferably by self-assembly upon expression. Preferably, the chimeric CMV polypeptide is a recombinant modified CMV polypeptide and is capable of forming a virus-like particle of CMV upon expression by self-assembly in E. coli. Typically and preferably, said T helper cell epitope replaces a N-terminal region of said CMV polypeptide, wherein said replaced N-terminal region of said CMV polypeptide consists of 5 to 15 consecutive amino acids. Preferably, said T helper cell epitope replaces a N-terminal region of said CMV polypeptide, and wherein said replaced N-terminal region of said CMV polypeptide consists of 5 to 15 consecutive amino acids, preferably of 9 to 14, 9 to 13 or 10 to 13 consecutive amino acids, more preferably of 11 to 13 consecutive amino acids, and most preferably of 11, 12 or 13 consecutive amino acids.

N-terminal region of the CMV polypeptide: The term “N-terminal region of the CMV polypeptide” as used herein, refers either to the N-terminus of said CMV polypeptide, and in particular to the N-terminus of a coat protein of CMV, or to the region of the N-terminus of said CMV polypeptide or said coat protein of CMV but starting with the second amino acid of the N-terminus of said CMV polypeptide or said coat protein of CMV if said CMV polypeptide or said coat protein comprises a N-terminal methionine residue. Preferably, in case said CMV polypeptide or said coat protein comprises a N-terminal methionine residue, from a practical point of view, the start-codon encoding methionine will usually be deleted and added to the N-terminus of the T helper (Th) cell epitope. Further preferably, one, two or three additional amino acids, preferably one amino acid, may be optionally inserted between the stating methionine and the Th cell epitope for cloning purposes.

Recombinant polypeptide: In the context of the invention the term “recombinant” when used in the context of a polypeptide refers to a polypeptide which is obtained by a process which comprises at least one step of recombinant DNA technology. Typically and preferably, a recombinant polypeptide is produced in a prokaryotic expression system. It is apparent for the artisan that recombinantly produced polypeptides which are expressed in a prokaryotic expression system such as E. coli may comprise an N-terminal methionine residue. The N-terminal methionine residue is typically cleaved off the recombinant polypeptide in the expression host during the maturation of the recombinant polypeptide. However, the cleavage of the N-terminal methionine may be incomplete. Thus, a preparation of a recombinant polypeptide may comprise a mixture of otherwise identical polypeptides with and without an N-terminal methionine residue. Typically and preferably, a preparation of a recombinant polypeptide comprises less than 10%, more preferably less than 5%, and still more preferably less than 1% recombinant polypeptide with an N-terminal methionine residue.

Recombinant modified virus-like particle: In the context of the invention the term “recombinant modified virus-like particle” refers to a modified virus-like particle (VLP) which is obtained by a process which comprises at least one step of recombinant DNA technology.

Mutated amino acid sequence: The term “mutated amino acid sequence” refers to an amino acid sequence which is obtained by introducing a defined set of mutations into an amino acid sequence to be mutated. In the context of the invention, said amino acid sequence to be mutated typically and preferably is an amino acid sequence of a coat protein of CMV. Thus, a mutated amino acid sequence differs from an amino acid sequence of a coat protein of CMV in at least one amino acid residue, wherein said mutated amino acid sequence and said amino acid sequence to be mutated show a sequence identity of at least 90%. Typically and preferably said mutated amino acid sequence and said amino acid sequence to be mutated show a sequence identity of at least 91%, 92%, 93% 94%, 95%, 96%, 97%, 98%, or 99%. Preferably, said mutated amino acid sequence and said sequence to be mutated differ in at most 11, 10, 9, 8, 7, 6, 4, 3, 2, or 1 amino acid residues, wherein further preferably said difference is selected from insertion, deletion and amino acid exchange. Preferably, the mutated amino acid sequence differs from an amino acid sequence of a coat protein of CMV in least one amino acid, wherein preferably said difference is an amino acid exchange.

The terms “corresponding, correspond or corresponds” when used herein to describe the relationship of specific positions of amino acid residue(s) within polypeptides and amino acid sequences, respectively, refers to the position of an amino acid residue(s) within an amino acid sequence, which corresponds to given and specific amino acid residue(s) of another amino acid sequence that can be identified by sequence alignment, typically and preferably by using the BLASTP algorithm, most preferably using the standard settings. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Sequence identity: The sequence identity of two given amino acid sequences is determined based on an alignment of both sequences. Algorithms for the determination of sequence identity are available to the artisan. Preferably, the sequence identity of two amino acid sequences is determined using publicly available computer homology programs such as the “BLAST” program (http://blast.ncbi.nlm.nih.gov/Blast.cgi) or the “CLUSTALW” (http://www.genome.jp/tools/clustalw/), and hereby preferably by the “BLAST” program provided on the NCBI homepage at http://blast.ncbi.nlm.nih.gov/Blast.cgi, using the default settings provided therein. Typical and preferred standard settings are: expect threshold: 10; word size: 3; max matches in a query range: 0; matrix: BLOSUM62; gap costs: existence 11, extension 1; compositional adjustments: conditional compositional score matrix adjustment.

Amino acid exchange: The term “amino acid exchange” refers to the exchange of a given amino acid residue in an amino acid sequence by any other amino acid residue having a different chemical structure, preferably by another proteinogenic amino acid residue. Thus, in contrast to insertion or deletion of an amino acid, the amino acid exchange does not change the total number of amino acids of said amino acid sequence.

The term “isoelectric point” as used herein and abbreviated as pI, refers to the pH at which a molecule carries no net electrical charge or is electrically neutral in the statistical mean. In particular, the term “isoelectric point” is used herein to refer to the pH at which antigens, used in the present invention and which are composed of amino acids, carries no net electrical charge or is electrically neutral in the statistical mean. At a pH below their pI, such antigens carry a net positive charge; above their pI they carry a net negative charge. Typically and preferably when referring to pI values, and in particular to pI values of antigens of the present invention and within the present disclosure, said pI values are determined by entering the primary amino acid sequence for a particular protein and antigen, respectively, into the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). Thus, if referred herein to the ExPASy Compute pI/MW tool is refers to the one described by Gasteiger et al. The tool calculates the theoretical isoelectric point pI and Mw of a specified Swiss-Prot/TrEMBL entry or a user-entered amino acid sequence. The pI of the protein is calculated using pK values of amino acids described in Bjellqvist et al., which were defined by examining polypeptide migration between pH 4.5 to 7.3 in an immobilised pH gradient gel environment with 9.2M and 9.8M urea at 15° C. or 25° C. (Bjellqvist, B. et al, 1993, Electrophoresis 14:1023-1031; Bjellqvist, B. er al, 1994, Electrophoresis 15:529-539).

Epitope: The term “epitope” refers to continuous or discontinuous portions of an a polypeptide or an antigen, wherein said portions can be specifically bound by an antibody or by a T-cell receptor within the context of an MHC molecule. With respect to antibodies, specific binding excludes non-specific binding but does not necessarily exclude cross-reactivity. An epitope typically comprise 5-20 amino acids in a spatial conformation which is unique to the antigenic site.

T helper (Th) cell epitope: The terms “T helper cell epitope or Th cell epitope, as interchangeably used” and as used herein, refer to an epitope that is capable of recognition by a helper Th cell. Typically and preferably, the term “Th cell epitope” as used herein refers to a Th cell epitope that is capable of binding to at least one, preferably more than one MHC class II molecules. The simplest way to determine whether a peptide sequence is a Th cell epitope is to measure the ability of the peptide to bind to individual MHC class II molecules. This may be measured by the ability of the peptide to compete with the binding of a known Th cell epitope peptide to the MHC class II molecule. A representative selection of HLA-DR molecules are described in e.g. Alexander J, et al., Immunity (1994) 1:751-761. Affinities of Th cell epitopes for MHC class II molecules should be at least 10⁻⁵M. A representative collection of MHC class II molecules present in different individuals is given in Panina-Bordignon P, et al., Eur J Immunol (1989) 19:2237-2242. As a consequence, the term “Th cell epitope” as used herein preferably refers to a Th cell epitope that generates a measurable T cell response upon immunization and boosting. Moreover, and again further preferred, the term “Th cell epitope” as used herein preferably refers to a Th cell epitope that is capable of binding to at least one, preferably to at least two, and even more preferably to at least three DR alleles selected from of DR1, DR2w2b, DR3, DR4w4, DR4w14, DR5, DR7, DR52a, DRw53, DR2w2a; and preferably selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. In an even again more preferable manner, the term “Th cell epitope” as used herein refers to a Th cell epitope that is capable of binding to at least one, preferably to at least two, and even more preferably to at least three DR alleles selected from DR1, DR2w2b, DR4w4, DR4w14, DR5, DR7, DRw53, DR2w2a, with an affinity at least 500 nM (as described in Alexander J, et al., Immunity (1994) 1:751-761 and references cited herein); a preferred binding assay to evaluate said affinities is the one described by Sette A, et al., J Immunol (1989) 142:35-40. Th cell epitopes are described, and known to the skilled person in the art, such as by Alexander J, et al., Immunity (1994) 1:751-761, Panina-Bordignon P, et al., Eur J Immunol (1989) 19:2237-2242, Calvo-Calle J M, et al., J Immunol (1997) 159:1362-1373, and Valmori D, et al., J Immunol (1992) 149:717-721.

Amino acid linker: The term “amino acid linker” as used herein, refers to a linker consisting exclusively of amino acid residues. The amino acid residues of the amino acid linker are composed of naturally occurring amino acids or unnatural amino acids known in the art, all-L or all-D or mixtures thereof. The amino acid residues of the amino acid linker are preferably naturally occurring amino acids, all-L or all-D or mixtures thereof. In a preferred embodiment, said amino acid linker consists of naturally occurring alpha amino acids, all in its L-configuration.

G-linker: The term “G-linker”, as used herein refers to an amino acid linker solely consisting of glycine amino acid residues. The G-linker in accordance with the present invention comprise at least two glycine residues and at most ten glycine residues.

GS-linker: The term “GS-linker”, as used herein refers to an amino acid linker solely consisting of glycine and serine amino acid residues. The GS-linker in accordance with the present invention comprise at least one glycine and at least one serine residue. Typically and preferably, the GS-linker has a length of at most 30 amino acids.

GS*-linker: The term “GS*-linker”, as used herein refers to an amino acid linker comprising at least one glycine, at least one serine and at least one amino acid residue selected from Thr, Ala, Lys, and Cys. Typically and preferably, the GS*-linker has a length of at most 30 amino acids.

The term “amino acid”, as used herein, refers to organic compounds containing the functional groups amine (—NH₂) and carboxylic acid (—COOH) and its zwitterions, typically and preferably, along with a side chain specific to each amino acid. The term “amino acid” typically and preferably includes amino acids that occur naturally, such as proteinogenic amino acids (produced by RNA-translation), non-proteinogenic amino acids (produced by other metabolic mechanisms, e.g. posttranslational modification), standard or canonical amino acids (that are directly encoded by the codons of the genetic code) and non-standard or non-canonical amino acids (not directly encoded by the genetic code). Naturally occurring amino acids include non-eukaryotic and eukaryotic amino acids. The term “amino acid”, as used herein, also includes unnatural amino acids that are chemically synthesized; alpha-(α-), beta-(β-), gamma-(γ-) and delta-(δ-) etc. amino acids as well as mixtures thereof in any ratio; and, if applicable such as for alpha-(α-) amino acids, any isomeric form of an amino acid, i.e. its D-stereoisomers and L-stereoisomers (alternatively addressed by the (R) and (S) nomenclature) as well as mixtures thereof in any ratio including in a racemic ratio of 1:1. The term “D-stereoisomer”, “L-stereoisomer”, “D-amino acid” or “L-amino acid” refers to the chiral alpha carbon of the amino acids. In a preferred embodiment, the term amino acid refers to an alpha amino acid, preferably to a naturally occurring alpha amino acid, further preferably to a naturally occurring alpha amino acid in its L-configuration.

Associated: The terms “associated” or “association” as used herein refer to all possible ways, preferably chemical interactions, by which two molecules are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds.

Attachment Site, First: As used herein, the phrase “first attachment site” refers to an element which is naturally occurring with the virus-like particle or which is artificially added to the virus-like particle, and to which the second attachment site may be linked. The first attachment site preferably is a protein, a polypeptide, an amino acid, a peptide, a sugar, a polynucleotide, a natural or synthetic polymer, a secondary metabolite or compound such as biotin, fluorescein, retinol, digoxigenin, metal ions, phenylmethylsulfonylfluoride, or a chemically reactive group such as an amino group, a carboxyl group, a sulfhydryl group, a hydroxyl group, a guanidinyl group, histidinyl group, or a combination thereof. A preferred embodiment of a chemically reactive group being the first attachment site is the amino group of an amino acid residue, preferably the amino group of the side chain of a lysine residue. The first attachment site is typically located on the surface, and preferably on the outer surface of the VLP. Multiple first attachment sites are present on the surface, preferably on the outer surface of the VLP, typically in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in a preferred embodiment the first attachment site is artificially added to the VLP. In a very preferred embodiment said first attachment site is the amino group of a lysine residue of the amino acid sequence of said VLP polypeptide.

Attachment Site, Second: As used herein, the phrase “second attachment site” refers to an element which is naturally occurring with or which is artificially added to the antigen and to which the first attachment site may be linked. The second attachment site of the antigen preferably is a protein, a polypeptide, a peptide, an amino acid, a sugar, a polynucleotide, a natural or synthetic polymer, a secondary metabolite or compound such as biotin, fluorescein, retinol, digoxigenin, metal ions, phenylmethylsulfonylfluoride, or a chemically reactive group such as an amino group, a carboxyl group, a sulfhydryl group, a hydroxyl group, a guanidinyl group, histidinyl group, or a combination thereof. A preferred embodiment of a chemically reactive group being the second attachment site is a sulfhydryl group, preferably the sulfhydryl group of a cysteine residue. The term “antigen with at least one second attachment site” refers, therefore, to a construct comprising the antigen and at least one second attachment site. However, in particular for a second attachment site, which is not naturally occurring within the antigen, such a construct typically and preferably further comprises a “linker”. In another preferred embodiment the second attachment site is associated with the antigen through at least one covalent bond, preferably through at least one peptide bond. In a further embodiment, the second attachment site is naturally occurring within the antigen. In another further preferred embodiment, the second attachment site is artificially added to the antigen through a linker, wherein said linker comprises or alternatively consists of a cysteine. Preferably, the linker is fused to the antigen by a peptide bond.

Linked: The terms “linked” or “linkage” as used herein, refer to all possible ways, preferably chemical interactions, by which the at least one first attachment site and the at least one second attachment site are joined together. Chemical interactions include covalent and non-covalent interactions. Typical examples for non-covalent interactions are ionic interactions, hydrophobic interactions or hydrogen bonds, whereas covalent interactions are based, by way of example, on covalent bonds such as ester, ether, phosphoester, carbon-phosphorus bonds, carbon-sulfur bonds such as thioether, or imide bonds. In certain preferred embodiments the first attachment site and the second attachment site are linked through at least one covalent bond, preferably through at least one non-peptide bond, and even more preferably through exclusively non-peptide covalent bond(s). The term “linked” as used herein, however, shall not only refer to a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker. In other embodiments the first attachment site and the second attachment site are linked through at least one covalent bond, preferably through at least one peptide bond, and even more preferably through exclusively peptide bond(s).

Linker: A “linker”, as used herein, either associates the second attachment site with the antigen or already comprises or consists of the second attachment site. Preferably, a “linker”, as used herein, already comprises the second attachment site, typically and preferably as one amino acid residue, preferably as a cysteine residue. A preferred linker is a linker containing at least one amino acid residue, or even more preferred is a linker consisting exclusively of amino acid residues. The amino acid residues of the linker are, preferably, composed of naturally occurring amino acids or unnatural amino acids known in the art, all-L or all-D or mixtures thereof. Further preferred embodiments of a linker in accordance with this invention are molecules comprising a sulfhydryl group or a cysteine residue and such molecules are, therefore, also encompassed within this invention. Further linkers useful for the present invention are molecules comprising a C1-6 alkyl-, a cycloalkyl such as a cyclopentyl or cyclohexyl, a cycloalkenyl, aryl or heteroaryl moiety. Moreover, linkers comprising preferably a C1-C6 alkyl-, cycloalkyl- (C5, C6), aryl- or heteroaryl-moiety and additional amino acid(s) can also be used as linkers for the present invention and shall be encompassed within the scope of the invention. Association of the linker with the antigen is preferably by way of at least one covalent bond, more preferably by way of at least one peptide bond.

Antigen: As used herein, the term “antigen” refers to a molecule capable of being bound by an antibody or a T-cell receptor (TCR) if presented by MHIC molecules. An antigen is additionally capable of being recognized by the immune system and/or being capable of inducing a humoral immune response and/or cellular immune response leading to the activation of B- and/or T-lymphocytes. An antigen can have one or more epitopes (B- and T-epitopes). An antigen as used herein may also be mixtures of several individual antigens.

Ordered and repetitive antigen array: As used herein, the term “ordered and repetitive antigen array” refers to a repeating pattern of antigen which typically and preferably is characterized by a high order of uniformity in spacial arrangement of the antigens with respect to the modified VLP of CMV. In one embodiment of the invention, the repeating pattern may be a geometric pattern. Certain embodiments of the invention, such as antigens linked to the modified VLP of CMV, are typical and preferred examples of suitable ordered and repetitive antigen arrays which, moreover, possess strictly repetitive paracrystalline orders of antigens, preferably with spacing of 1 to 30 nanometers, preferably 2 to 15 nanometers, even more preferably 2 to 10 nanometers, even again more preferably 2 to 8 nanometers, and further more preferably 1.6 to 7 nanometers.

Coupling efficiency: The coupling efficiency of a virus-like particle with a specific antigen is determined by SDS-PAGE of the coupling reactions. The intensities of Coomassie Blue-stained bands corresponding to components of the coupling reaction are determined by densitometry and used to calculate coupling efficiency. Coupling efficiency is defined as the ratio of (i) the amount of VLP polypeptides coupled to said antigen to (ii) the total amount of VLP polypeptides. Typically and preferably, said coupling efficiency is at least 5%, 10%, preferably at least 15%, further preferably at least 20%, 25% or at least 30%, and again further preferably of at least 35% or at least 40%. Coupling deficiency can also be expressed by the total number of antigens linked to the modified CMV VLP. Coupling deficiency can be dependent on the nature of the antigen, and the total numbers of antigens linked to the modified CMV VLP are typically and preferably at least 5, at least 7, at least 10, at least 15, at least 20, at least 25, at least 30, at least 40 and at least 50 antigens.

Peanut Allergen: The term “peanut allergen”, as used herein, refers to any protein of the Arachis hypogaea species, and isoforms thereof, suggested to cause an allergy for a human. Preferably, the term “peanut allergen”, as used herein, refers to any of the suggested peanut allergens, and isoforms thereof, as retrievable under www.allergen.org or a protein with an amino acid sequence of at least 90%, preferably of at least 92%, further preferably of at least 95%, and again further preferably of at least 98% amino acid sequence identity with such a peanut allergen and isoform thereof. More preferably, the term “peanut allergen”, as used herein, refers to any of the suggested currently 17 peanut allergens, and isoforms thereof, as retrievable under www.allergen.org or a protein with an amino acid sequence of at least 90%, preferably of at least 92%, further preferably of at least 95%, and again further preferably of at least 98% amino acid sequence identity with such a peanut allergen and isoform thereof. Again more preferably, the term “peanut allergen”, as used herein, refers to any one of the peanut allergens, and isoforms thereof, selected from Ara h1, Ara h2, Ara h3, Ara h4, Ara h5, Ara h6, Ara h7, Ara h8, Ara h9, Ara h10, Ara h11, Ara h12, Ara h13, Ara h14, Ara h15, Ara h16 and Ara h17, or a protein with an amino acid sequence of at least 90%, preferably of at least 92%, further preferably of at least 95%, and again further preferably of at least 98% amino acid sequence identity with such a peanut allergen and isoform thereof.

Fel d1 protein: The term “Fel d1 protein”, as used herein, refers to a protein comprising or alternatively consisting of chain 1 of Fel d1 and chain 2 of Fel d1. Preferably chain 1 of Fel d1 and chain 2 of Fel d1 are linked covalently. In one preferred embodiment, the chain 1 of Fel d1 and chain 2 of Fel d1 are linked via at least one disulfide bond. In another preferred embodiment, the chain 1 and chain 2 are fused either directly or via a spacer, in which case said Fel d1 protein further comprises or alternatively consists of a spacer. Preferably the Fel d1 protein, as defined herein, consists of at most 300, even more preferably at most 200 amino acids in total. Typically and preferably, Fel d1 protein, according to the invention, is capable of inducing in vivo the production of antibody specifically binding to either the naturally occurring Fel d1.

Chain 1 of Fel d1: The term “chain 1 of Fel d1”, as used herein, refers to a polypeptide comprising or alternatively consisting of an amino acid sequence as of SEQ ID NO:58 or a homologous sequence thereof. The term “homologous sequence of SEQ ID NO:58”, as used herein, refers to a polypeptide that has an sequence identity to SEQ ID NO:58 which is greater than 80%, more preferably greater than 90%, and even more preferably greater than 95%. The term “chain 1 of Fel d1”, as used herein, shall also refer to a polypeptide encompassing at least one post-translational modification, including but not limited to at least one glycosylation, of chain 1 of Fel d1, as defined herein. Preferably the chain 1 of Fel d1, as defined herein, consists of at most 130, even more preferably at most 100 amino acids in total.

Chain 2 of Fel d1: The term “chain 2 of Fel d1”, as used herein, refers to a polypeptide comprising or alternatively consisting of an amino acid sequence as of SEQ ID NO:59, SEQ ID NO:60 or SEQ ID NO:61, or a homologous sequence thereof. The term “homologous sequence of SEQ ID NO:59, SEQ ID NO:60 or SEQ ID NO:61, as used herein, refers to a polypeptide that has an identity to SEQ ID NO:59, SEQ ID NO:60 or SEQ ID NO:61 which is greater than 80%, more preferably greater than 90%, and even more preferably greater than 95%. The term “chain 2 of Fel d1”, as used herein, shall also refer to a polypeptide encompassing at least one post-translational modification, including but not limited to at least one glycosylation, of chain 2 of Fel d1, as defined herein Preferably the chain 2 of Fel d1, as defined herein, consists of at most 150, even more preferably at most 130, still more preferably at most 100 amino acids in total.

Receptor binding domain: The term “protein domain” and “receptor binding domain” as used herein, refers to parts of proteins that either occur alone or together with partner domains on the same protein chain. Most domains correspond to tertiary structure elements and are able to fold independently. All domains exhibit evolutionary conservation, and many either perform specific functions or contribute in a specific way to the function of their proteins (Forslund S K et al, Methods Mol Biol. (2019) 1910:469-504). Viral structural proteins, such as Coronavirus S proteins, can contain several functional domains, which are necessary for the cell infection process. One such domain in Coronavirus S protein is the receptor binding domain (RBD) which binds to corresponding cell receptor.

Receptor binding motif: The term “receptor binding motif (RBM)”, as used herein, is a part of receptor binding domain and represent a linear amino acid sequence and/or a 3D structure located on outer surface of the virus and making direct contact with target cell receptors (Sobhy H, Proteomes (2016) 4(1): 3). For Coronaviruses, the amino acids sequences of RBMs have low homology due to different target cellular receptors. For SARS-CoV2, 16 amino acids of RBM make direct contacts with human ACE2 receptor (Lan et al., Structure of the SARS-CoV-2 spike receptor-binding domain bound to the ACE2 receptor, Nature, 2020, 581, 215-220).

Adjuvant: The term “adjuvant” as used herein refers to stimulators of the immune response and/or substances that typically allow generation of a depot in the host which when combined with the composition, vaccine or pharmaceutical composition, respectively, of the present invention may provide for an more enhanced immune response. Adjuvants of varying types with different mechanisms of action are described and are able to enhance the antigen-specific antibody response (Pulendran B et al, 2021, Nature Reviews Drug Discovery 20:454-475). Typical and preferred adjuvants are mineral salts (e.g. Aluminum Hydroxide, Aluminum Phosphate), microcrystalline tyrosine, emulsions, microparticles, saponins (Quil A), cytokines, immune potentiators, microbial components/products, liposomes, complexes, and mucosal adjuvants which are known and as described such, and for example, in the Adjuvant Compendium NIAID and VAC (nih.gov) or by Aguilar et al, (Aguilar J C et al, 2007, Vaccine 25:3752-3762), Gerdts (Gerdts V, 2015, Berliner und Münchener Tierärztliche Wochenschrift 128:456-463) and Pasquale et al. (Pasquale et al. 2015, Vaccines 3:320-343). The term “adjuvant” as used herein may also comprise mixtures of adjuvants. Virus-like particles have sometimes been described as an adjuvant. However, the term “adjuvant”, as used within the context of this application, refers to an adjuvant not being the inventive modified virus-like particle. Rather “adjuvant” relates to an additional, distinct component of the inventive compositions, vaccines or pharmaceutical compositions.

Immunostimulatory substance: As used herein, the term “immunostimulatory substance” refers to a substance capable of inducing and/or enhancing an immune response. Immunostimulatory substances, as used herein, include, but are not limited to, toll-like receptor activating substances and substances inducing cytokine secretion. Toll-like receptor activating substances include, but are not limited to, immunostimulatory nucleic acids, peptideoglycans, lipopolysaccharides, lipoteichonic acids, imidazoquinoline compounds, flagellins, lipoproteins, and immunostimulatory organic substances such as taxol.

Immunostimulatory nucleic acid (ISS-NA): As used herein, the term “immunostimulatory nucleic acid” refers to a nucleic acid capable of inducing and/or enhancing an immune response. Immunostimulatory nucleic acids comprise ribonucleic acids and in particular deoxyribonucleic acids, wherein both, ribonucleic acids and deoxyribonucleic acids may be either double stranded or single stranded. Preferred ISS-NA are deoxyribonucleic acids, wherein further preferably said deoxyribonucleic acids are single stranded. Preferably, immunostimulatory nucleic acids contain at least one CpG motif comprising an unmethylated C. Very preferred immunostimulatory nucleic acids comprise at least one CpG motif, wherein said at least one CpG motif comprises or preferably consist of at least one, preferably one, CG dinucleotide, wherein the C is unmethylated. Preferably, but not necessarily, said CG dinucleotide is part of a palindromic sequence. The term immunostimulatory nucleic acid also refers to nucleic acids that contain modified bases, preferably 4-bromo-cytosine. Specifically preferred in the context of the invention are ISS-NA which are capable of stimulating IFN-alpha production in dendritic cells. Immunostimulatory nucleic acids useful for the purpose of the invention are described, for example, in WO2007/068747A1.

Oligonucleotide: As used herein, the term “oligonucleotide” refers to a nucleic acid sequence comprising two or more nucleotides, preferably about 6 to about 200 nucleotides, and more preferably 20 to about 100 nucleotides, and most preferably 20 to 40 nucleotides. Oligonucleotides are polyribonucleotides or polydeoxribonucleotides and are preferably selected from (a) unmodified RNA or DNA, and (b) modified RNA or DNA. The modification may comprise the backbone or nucleotide analogues. Oligonucleotides are preferably selected from the group consisting of (a) single- and double-stranded DNA, (b) DNA that is a mixture of single- and double-stranded regions, (c) single- and double-stranded RNA, (d) RNA that is mixture of single- and double-stranded regions, and (e) hybrid molecules comprising DNA and RNA that are single-stranded or, more preferably, double-stranded or a mixture of single- and double-stranded regions. Preferred nucleotide modifications/analogs are selected from the group consisting of (a) peptide nucleic acid, (b) inosin, (c) tritylated bases, (d) phosphorothioates, (e) alkylphosphorothioates, (f) 5-nitroindole desoxyribofliranosyl, (g) 5-methyldesoxycytosine, and (h) 5,6-dihydro-5,6-dihydroxydesoxythymidine. Phosphorothioated nucleotides are protected against degradation in a cell or an organism and are therefore preferred nucleotide modifications. Unmodified oligonucleotides consisting exclusively of phosphodiester bound nucleotides, typically are more active than modified nucleotides and are therefore generally preferred in the context of the invention. Most preferred are oligonucleotides consisting exclusively of phosphodiester bound oligonucleotides, wherein further preferably said oligonucleotides are single stranded. Further preferred are oligonucleotides capable of stimulating IFN-alpha production in cells, preferably in dendritic cells. Very preferred oligonucleotides capable of stimulating IFN-alpha production in cells are selected from A-type CpGs and C-type CpGs. Further preferred are RNA-molecules without a Cap.

CpG motif: As used herein, the term “CpG motif” refers to a pattern of nucleotides that includes an unmethylated central CpG, i.e. the unmethylated CpG dinucleotide, in which the C is unmethylated, surrounded by at least one base, preferably one or two nucleotides, flanking (on the 3′ and the 5′ side of) the central CpG. Typically and preferably, the CpG motif as used herein, comprises or alternatively consists of the unmethylated CpG dinucleotide and two nucleotides on its 5′ and 3′ ends. Without being bound by theory, the bases flanking the CpG confer a significant part of the activity to the CpG oligonucleotide.

Unmethylated CpG-containing oligonucleotide: As used herein, the term “unmethylated CpG-containing oligonucleotide” or “CpG” refers to an oligonucleotide, preferably to an oligodeoxynucleotide, containing at least one CpG motif Thus, a CpG contains at least one unmethylated cytosine, guanine dinucleotide. Preferred CpGs stimulate/activate, e.g. have a mitogenic effect on, or induce or increase cytokine expression by, a vertebrate bone marrow derived cell. For example, CpGs can be useful in activating B cells, NK cells and antigen-presenting cells, such as dendritic cells, monocytes and macrophages. Preferably, CpG relates to an oligodeoxynucleotide, preferably to a single stranded oligodeoxynucleotide, containing an unmethylated cytosine followed 3′ by a guanosine, wherein said unmethylated cytosine and said guanosine are linked by a phosphate bond, wherein preferably said phosphate bound is a phosphodiester bound or a phosphorothioate bound, and wherein further preferably said phosphate bond is a phosphodiester bound. CpGs can include nucleotide analogs such as analogs containing phosphorothioester bonds and can be double-stranded or single-stranded. Generally, double-stranded molecules are more stable in vivo, while single-stranded molecules have increased immune activity. Preferably, as used herein, a CpG is an oligonucleotide that is at least about ten nucleotides in length and comprises at least one CpG motif, wherein further preferably said CpG is 10 to 60, more preferably 15 to 50, still more preferably 20 to 40, still more preferably about 30, and most preferably exactly 30 nucleotides in length. A CpG may consist of methylated and/or unmethylated nucleotides, wherein said at least one CpG motif comprises at least one CG dinucleotide wherein the C is unmethylated. The CpG may also comprise methylated and unmethylated sequence stretches, wherein said at least one CpG motif comprises at least one CG dinucleotide wherein the C is unmethylated. Very preferably, CpG relates to a single stranded oligodeoxynucleotide containing an unmethylated cytosine followed 3′ by a guanosine, wherein said unmethylated cytosine and said guanosine are linked by a phosphodiester bound. The CpGs can include nucleotide analogs such as analogs containing phosphorothioester bonds and can be double-stranded or single-stranded. Generally, phosphodiester CpGs are A-type CpGs as indicated below, while phosphothioester stabilized CpGs are B-type CpGs. Preferred CpG oligonucleotides in the context of the invention are A-type CpGs.

A-type CpG: As used herein, the term “A-type CpG” or “D-type CpG” refers to an oligodeoxynucleotide (ODN) comprising at least one CpG motif. A-type CpGs preferentially stimulate activation of T cells and the maturation of dendritic cells and are capable of stimulating IFN-alpha production. In A-type CpGs, the nucleotides of the at least one CpG motif are linked by at least one phosphodiester bond. A-type CpGs comprise at least one phosphodiester bond CpG motif which may be flanked at its 5′ end and/or, preferably and, at its 3′ end by phosphorothioate bound nucleotides. Preferably, the CpG motif, and hereby preferably the CG dinucleotide and its immediate flanking regions comprising at least one, preferably two nucleotides, are composed of phosphodiester nucleotides. Preferred A-type CpGs exclusively consist of phosphodiester (PO) bond nucleotides. Typically and preferably, the poly G motif comprises or alternatively consists of at least one, preferably at least three, at least 4, 5, 6, 7, 8, 9, 10, 11, 12, 13, 14, or 15 G's (guanosines), most preferably by at least 10 G's. Preferably, the A-type CpG of the invention comprises or alternatively consists of a palindromic sequence.

Packaged: The term “packaged” as used herein refers to the state of a polyanionic macromolecule or immunostimulatory substances in relation to the core particle and VLP, respectively. The term “packaged” as used herein includes binding that may be covalent, e.g., by chemically coupling, or non-covalent, e.g., ionic interactions, hydrophobic interactions, hydrogen bonds, etc. The term also includes the enclosement, or partial enclosement, of a polyanionic macromolecule. Thus, the polyanionic macromolecule or immunostimulatory substances can be enclosed by the VLP without the existence of an actual binding, in particular of a covalent binding. In preferred embodiments, the at least one polyanionic macromolecule or immunostimulatory substances is packaged inside the VLP, most preferably in a non-covalent manner. In case said immunostimulatory substances is nucleic acid, preferably a DNA, the term packaged implies that said nucleic acid is not accessible to nucleases hydrolysis, preferably not accessible to DNAse hydrolysis (e.g. DNaseI or Benzonase), wherein preferably said accessibility is assayed as described in Examples 11-17 of WO2003/024481A2.

Effective amount: As used herein, the term “effective amount” refers to an amount necessary or sufficient to realize a desired biologic effect. An effective amount of the composition, or alternatively the pharmaceutical composition, would be the amount that achieves this selected result, and such an amount could be determined as a matter of routine by a person skilled in the art. The effective amount can vary depending on the particular composition being administered and the size of the subject. One of ordinary skill in the art can empirically determine the effective amount of a particular composition of the present invention without necessitating undue experimentation. Preferably, the term “effective amount” refers to an amount that (i) treats or prevents the particular disease or disorder, (ii) attenuates, ameliorates, or eliminates one or more symptoms of the particular disease or disorder, or (iii) prevents or delays the onset of one or more symptoms of the particular disease or disorder, described herein.

Animal: The term “animal”, as used herein and being the subject in need of treatment or prevention with the inventive modified CMV VLPs, may be an animal (e.g., a non-human animal), a vertebrate animal, a mammal, a rodent (e.g., a guinea pig, a hamster), a canine (e.g., a dog), a feline (e.g., a cat), a porcine (e.g., a pig), an equine (e.g., a horse), a primate, or a human. In the context of this invention, it is particularly envisaged that animals are to be treated which are economically, agronomically or scientifically important. Scientifically important organisms include, but are not limited to, mice, rats, and rabbits. Non-limiting examples of agronomically important animals are sheep, cattle and pigs, while, for example, cats, dogs and horses may be considered as economically important animals. Preferably, the subject is a mammal; more preferably, the subject is a human or a non-human mammal (such as, e.g., a dog, a cat, a horse, a sheep, cattle, or a pig). In a preferred embodiment said subject is a mammal, wherein said mammal is human or a non-human mammal, and wherein said non-human mammal is selected from a dog, a cat, a horse, a sheep, cattle, or a pig.

Treatment: As used herein, the terms “treatment”, “treat”, “treated” or “treating” refer to prophylaxis and/or therapy. In one embodiment, the terms “treatment”, “treat”, “treated” or “treating” refer to a therapeutic treatment. In another embodiment, the terms “treatment”, “treat”, “treated” or “treating” refer to a prophylactic treatment. Preferably, beneficial or desired clinical results of said treatment include, but are not limited to, alleviation of symptoms, diminishment of extent of disease or disorder, stabilized (i.e., not worsening) state of disease or disorder, delay or slowing of disease or disorder progression, amelioration or palliation of the disease or disorder state.

In a first aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48; and
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48.

In a further aspect, the present invention provides a composition comprising
(a) a modified VLP of CMV comprising at least one first attachment site;
(b) at least one antigen, wherein said antigen comprises at least one second attachment site;

wherein said modified VLP of CMV comprises at least one chimeric CMV polypeptide, and wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48; and
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48; and

wherein (a) and (b) are linked through said at least one first and said at least one second attachment site via at least one covalent non-peptide bond.

In a preferred embodiment, said chimeric CMV polypeptide further comprises a T helper cell epitope, wherein preferably said T helper cell epitope replaces a N-terminal region of said CMV polypeptide, and wherein further preferably said N-terminal region of said CMV polypeptide corresponds to amino acids 2-12 of SEQ ID NO:48, and wherein again further preferably said T helper cell epitope is derived from tetanus toxin or is a PADRE sequence, wherein very preferably, said Th cell epitope comprises, again further preferably consists of, the amino acid sequence of SEQ ID NO:50 or SEQ ID NO:51. In a further very preferred embodiment, said CMV polypeptide is a coat protein of CMV or an amino acid sequence having a sequence identity of at least 90%, preferably 95% with SEQ ID NO:48.

Thus, in another aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48, wherein preferably said CMV polypeptide is a coat protein of CMV or an amino acid sequence having a sequence identity of at least 90%, preferably 95% with SEQ ID NO:48;
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48; and
(iii) a T helper cell epitope, wherein said T helper cell epitope replaces a N-terminal region of said CMV polypeptide.

In a further very preferred embodiment, said stretch of consecutive negative amino acids comprises, preferably consists of SEQ ID NO:1 or SEQ ID NO:2.

Thus, in a further aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48, wherein preferably said CMV polypeptide is a coat protein of CMV or an amino acid sequence having a sequence identity of at least 90%, preferably 95% with SEQ ID NO:48; and
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48, and wherein said stretch of consecutive negative amino acids comprises, preferably consists of SEQ ID NO:1 or SEQ ID NO:2.

Thus, in another aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48, wherein preferably said CMV polypeptide is a coat protein of CMV or an amino acid sequence having a sequence identity of at least 90%, preferably 95% with SEQ ID NO:48;
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48, and wherein said stretch of consecutive negative amino acids comprises, preferably consists of SEQ ID NO:1 or SEQ ID NO:2; and
(iii) a T helper cell epitope, wherein said T helper cell epitope replaces a N-terminal region of said CMV polypeptide.

In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from the group consisting of (a.) a polyglycine linker (G-linker) having an amino acid sequence (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1 or an amino acid linker (GS*-linker) comprising at least one Gly, at least one Ser, and at least one amino acid selected from Thr, Ala, Lys, and Cys. In a further very preferred embodiment, said first amino acid linker comprises, preferably consists of, SEQ ID NO:8. In a further very preferred embodiment, said second amino acid linker comprises, preferably consists of, SEQ ID NO:4 or SEQ ID NO:9. In a further very preferred embodiment, said polypeptide comprises SEQ ID NO:62, SEQ ID NO:63 or SEQ ID NO:64. In a further very preferred embodiment, said polypeptide consists of SEQ ID NO:62, SEQ ID NO:63 or SEQ ID NO:64. In a further very preferred embodiment, said polypeptide comprising, preferably consisting of, a stretch of consecutive negative amino acids is inserted between amino acid residues of said CMV polypeptide corresponding to amino acid residues of position 84 and position 85 of SEQ ID NO:48.

Thus, in a further aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48, wherein preferably said CMV polypeptide is a coat protein of CMV or an amino acid sequence having a sequence identity of at least 90%, preferably 95% with SEQ ID NO:48; and
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48, and wherein said polypeptide comprises, preferably consists of, SEQ ID NO:62, SEQ ID NO:63 or SEQ ID NO:64, and wherein preferably said polypeptide is inserted between amino acid residues of said CMV polypeptide corresponding to amino acid residues of position 84 and position 85 of SEQ ID NO:48.

Thus, in another aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48, wherein preferably said CMV polypeptide is a coat protein of CMV or an amino acid sequence having a sequence identity of at least 90%, preferably 95% with SEQ ID NO:48;
(ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48, and wherein said polypeptide comprises, preferably consists of, SEQ ID NO:62, SEQ ID NO:63 or SEQ ID NO:64; and wherein preferably said polypeptide is inserted between amino acid residues of said CMV polypeptide corresponding to amino acid residues of position 84 and position 85 of SEQ ID NO:48;
(iii) a T helper cell epitope, wherein said T helper cell epitope replaces a N-terminal region of said CMV polypeptide.

In a further very preferred embodiment, said CMV polypeptide comprises, preferably consists of, the amino acid sequence of SEQ ID NO:5, SEQ ID NO:48 or SEQ ID NO:57, wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57.

Thus, in a further aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48;
(ii) a polypeptide comprising a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48; and
wherein said CMV polypeptide comprises, preferably consists of, the amino acid sequence of SEQ ID NO:5, SEQ ID NO:48 or SEQ ID NO:57, and wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57.

Thus, in a further aspect, the present invention provides a modified virus-like particle (VLP) of cucumber mosaic virus (CMV) comprising at least one chimeric CMV polypeptide, wherein said at least one chimeric CMV polypeptide comprises, preferably consists of,
(i) a CMV polypeptide, wherein said CMV polypeptide comprises a coat protein of CMV or an amino acid sequence having a sequence identity of at least 75% with SEQ ID NO:48;
(ii) a polypeptide comprising a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48;
(iii) a T helper cell epitope, wherein said T helper cell epitope replaces a N-terminal region of said CMV polypeptide; and
wherein said CMV polypeptide comprises, preferably consists of, the amino acid sequence of SEQ ID NO:5, SEQ ID NO:48 or SEQ ID NO:57, and wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57.

The herein described and disclosed embodiments, preferred embodiments and very preferred embodiments should apply to all aspects and other embodiments, preferred embodiments and very preferred embodiments irrespective of whether is specifically again referred to or its repetition is avoided for the sake of conciseness.

In a preferred embodiment, said CMV polypeptide comprises, preferably consists of, an amino acid sequence of a coat protein of CMV or a mutated amino acid sequence, wherein said mutated amino acid sequence and said coat protein of CMV show a sequence identity of at least 90%, preferably of at least 91%, 92%, 93, 94% or 95%, further preferably of at least 96%, 97% or 98% and again more preferably of at least 99%; wherein preferably said mutated amino acid sequence and said amino acid sequence to be mutated differ in least one and in at most 11, 10, 9, 8, 7, 6, 5, 4, 3, or 2 amino acid residues, and wherein further preferably these differences are selected from (i) insertion, (ii) deletion, (iii) amino acid exchange, and (iv) any combination of (i) to (iii).

In a preferred embodiment, said CMV polypeptide comprises a coat protein of CMV or an amino acid sequence having a sequence identity of at least 75% with SEQ ID NO:48.

In another preferred embodiment, said CMV polypeptide comprises a coat protein of CMV or an amino acid sequence having a sequence identity of at least 80% with SEQ ID NO:48.

In another preferred embodiment, said CMV polypeptide comprises a coat protein of CMV or an amino acid sequence having a sequence identity of at least 85% with SEQ ID NO:48.

In another preferred embodiment, said CMV polypeptide comprises a coat protein of CMV or an amino acid sequence having a sequence identity of at least 90% with SEQ ID NO:48.

In another preferred embodiment, said CMV polypeptide comprises a coat protein of CMV or an amino acid sequence having a sequence identity of at least 92% with SEQ ID NO:48.

In another preferred embodiment, said CMV polypeptide comprises a coat protein of CMV or an amino acid sequence having a sequence identity of at least 93% with SEQ ID NO:48.

In another preferred embodiment, said CMV polypeptide comprises a coat protein of CMV or an amino acid sequence having a sequence identity of at least 95% with SEQ ID NO:48. In another preferred embodiment, said CMV polypeptide comprises a coat protein of CMV or an amino acid sequence having a sequence identity of at least 96% with SEQ ID NO:48. In another preferred embodiment, said CMV polypeptide comprises a coat protein of CMV or an amino acid sequence having a sequence identity of at least 97% with SEQ ID NO:48. In another preferred embodiment, said CMV polypeptide comprises a coat protein of CMV or an amino acid sequence having a sequence identity of at least 98% with SEQ ID NO:48.

In another preferred embodiment, said CMV polypeptide comprises a coat protein of CMV or an amino acid sequence having a sequence identity of at least 99% with SEQ ID NO:48. In a preferred embodiment, said CMV polypeptide consists of a coat protein of CMV or an amino acid sequence having a sequence identity of at least 75% with SEQ ID NO:48. In another preferred embodiment, said CMV polypeptide consists of a coat protein of CMV or an amino acid sequence having a sequence identity of at least 80% with SEQ ID NO:48. In another preferred embodiment, said CMV polypeptide consists of a coat protein of CMV or an amino acid sequence having a sequence identity of at least 85% with SEQ ID NO:48. In another preferred embodiment, said CMV polypeptide consists of a coat protein of CMV or an amino acid sequence having a sequence identity of at least 90% with SEQ ID NO:48. In another preferred embodiment, said CMV polypeptide consists of a coat protein of CMV or an amino acid sequence having a sequence identity of at least 92% with SEQ ID NO:48. In another preferred embodiment, said CMV polypeptide consists of a coat protein of CMV or an amino acid sequence having a sequence identity of at least 93% with SEQ ID NO:48. In another preferred embodiment, said CMV polypeptide consists of a coat protein of CMV or an amino acid sequence having a sequence identity of at least 95% with SEQ ID NO:48. In another preferred embodiment, said CMV polypeptide consists of a coat protein of CMV or an amino acid sequence having a sequence identity of at least 96% with SEQ ID NO:48. In another preferred embodiment, said CMV polypeptide consists of a coat protein of CMV or an amino acid sequence having a sequence identity of at least 97% with SEQ ID NO:48. In another preferred embodiment, said CMV polypeptide consists of a coat protein of CMV or an amino acid sequence having a sequence identity of at least 98% with SEQ ID NO:48. In another preferred embodiment, said CMV polypeptide consists of a coat protein of CMV or an amino acid sequence having a sequence identity of at least 99% with SEQ ID NO:48.

In a preferred embodiment, said CMV polypeptide is a coat protein of CMV or an amino acid sequence having a sequence identity of at least 75%, preferably 85% with SEQ ID NO:48. In a preferred embodiment, said CMV polypeptide is a coat protein of CMV or an amino acid sequence having a sequence identity of at least 90%, preferably 95% with SEQ ID NO:48. In a preferred embodiment, said CMV polypeptide is a coat protein of CMV with SEQ ID NO:48. In a preferred embodiment, said coat protein of CMV comprises SEQ ID NO:48. In a preferred embodiment, said coat protein of CMV consists of SEQ ID NO:48. In a preferred embodiment, said CMV polypeptide comprises a coat protein of CMV. In a preferred embodiment, said CMV polypeptide consists of a coat protein of CMV. In a preferred embodiment, said CMV polypeptide comprises a coat protein of CMV, wherein said coat protein of CMV comprises SEQ ID NO:48. In a preferred embodiment, said CMV polypeptide comprises a coat protein of CMV, wherein said coat protein of CMV consists of SEQ ID NO:48. In a preferred embodiment, said CMV polypeptide consists of a coat protein of CMV, wherein said coat protein of CMV consists of SEQ ID NO:48.

In a preferred embodiment, said CMV polypeptide comprises SEQ ID NO:49 or an amino acid sequence region, wherein said amino acid sequence region has a sequence identity of at least 75% with SEQ ID NO:49. In a preferred embodiment, said CMV polypeptide comprises SEQ ID NO:49 or an amino acid sequence region, wherein said amino acid sequence region has a sequence identity of at least 80% with SEQ ID NO:49. In a preferred embodiment, said CMV polypeptide comprises SEQ ID NO:49 or an amino acid sequence region, wherein said amino acid sequence region has a sequence identity of at least 85% with SEQ ID NO:49. In a preferred embodiment, said CMV polypeptide comprises SEQ ID NO:49 or an amino acid sequence region, wherein said amino acid sequence region has a sequence identity of at least 90% with SEQ ID NO:49. In a preferred embodiment, said CMV polypeptide comprises SEQ ID NO:49 or an amino acid sequence region, wherein said amino acid sequence region has a sequence identity of at least 95% with SEQ ID NO:49. In a preferred embodiment, said CMV polypeptide comprises SEQ ID NO:49 or an amino acid sequence region, wherein said amino acid sequence region has a sequence identity of at least 98% with SEQ ID NO:49. In a preferred embodiment, said CMV polypeptide comprises SEQ ID NO:49 or an amino acid sequence region, wherein said amino acid sequence region has a sequence identity of at least 99% with SEQ ID NO:49.

In a preferred embodiment, said CMV polypeptide comprises, or preferably consists of, (i) an amino acid sequence of a coat protein of CMV, wherein said amino acid sequence comprises, or preferably consists of, SEQ ID NO:48; or (ii) an amino acid sequence having a sequence identity of at least 90% of SEQ ID NO:48; and wherein said amino sequence as defined in (i) or (ii) comprises SEQ ID NO:49 or an amino acid sequence region, wherein said amino acid sequence region has a sequence identity of at least 90% with SEQ ID NO:49. In a preferred embodiment, said CMV polypeptide comprises, or preferably consists of, (i) an amino acid sequence of a coat protein of CMV, wherein said amino acid sequence comprises, or preferably consists of, SEQ ID NO:48; or (ii) an amino acid sequence having a sequence identity of at least 95% of SEQ ID NO:48; and wherein said amino sequence as defined in (i) or (ii) comprises SEQ ID NO:49 or an amino acid sequence region, wherein said amino acid sequence region has a sequence identity of at least 95% with SEQ ID NO:49. In a preferred embodiment, said CMV polypeptide comprises, or preferably consists of, (i) an amino acid sequence of a coat protein of CMV, wherein said amino acid sequence comprises, or preferably consists of, SEQ ID NO:48; or (ii) an amino acid sequence having a sequence identity of at least 90% of SEQ ID NO:48; and wherein said amino sequence as defined in (i) or (ii) comprises SEQ ID NO:49.

In a preferred embodiment, the number of amino acids of said N-terminal region replaced is equal to or lower than the number of amino acids of which said T helper cell epitope consists. In a preferred embodiment, said replaced N-terminal region of said CMV polypeptide consists of 5 to 15 consecutive amino acids. In a preferred embodiment, said replaced N-terminal region of said CMV polypeptide consists of 9 to 14 consecutive amino acids. In a preferred embodiment, said replaced N-terminal region of said CMV polypeptide consists of 11 to 13 consecutive amino acids. In a preferred embodiment, said N-terminal region of said CMV polypeptide corresponds to amino acids 2-12 of SEQ ID NO:48. In a preferred embodiment, said N-terminal region of said CMV polypeptide comprises amino acids 2-12 of SEQ ID NO:48. In a preferred embodiment, said N-terminal region of said CMV polypeptide consists of amino acids 2-12 of SEQ ID NO:48. In a preferred embodiment, said T helper cell epitope consists of at most 20 amino acids.

In a preferred embodiment of the present invention, the Th cell epitope is selected from TT 830-843 (SEQ ID NO:50), PADRE (SEQ ID NO:51), HA 307-319 (SEQ ID NO:52), HBVnc 50-69 (SEQ ID NO:53), CS 378-398 (SEQ ID NO:54), MT 17-31 (SEQ ID NO:55), and TT 947-967 (SEQ ID NO:56). In a preferred embodiment, said Th cell epitope is a Th cell epitope derived from tetanus toxin or is a PADRE sequence. In a preferred embodiment, said T helper cell epitope is derived from a human vaccine. In a preferred embodiment, said Th cell epitope is a Th cell epitope derived from tetanus toxin. In a preferred embodiment, said Th cell epitope is a PADRE sequence. In a preferred embodiment, said Th cell epitope comprises the amino acid sequence of SEQ ID NO:50 or SEQ ID NO:51. In a very preferred embodiment, said Th cell epitope consists of the amino acid sequence of SEQ ID NO:50 or SEQ ID NO:51. In a very preferred embodiment, said Th cell epitope comprises the amino acid sequence of SEQ ID NO:50. In a preferred embodiment, said Th cell epitope consists of the amino acid sequence of SEQ ID NO:50. In a very preferred embodiment, said Th cell epitope comprises the amino acid sequence of SEQ ID NO:51. In a very preferred embodiment, said Th cell epitope consists of the amino acid sequence of SEQ ID NO:51.

In a preferred embodiment, said CMV polypeptide comprises, or preferably consists of, an amino acid sequence of a coat protein of CMV, wherein said amino acid sequence comprises, or preferably consists of, SEQ ID NO:48 or an amino acid sequence having a sequence identity of at least 95% of SEQ ID NO:48; and wherein said amino sequence comprises SEQ ID NO:49, and wherein said T helper cell epitope replaces the N-terminal region of said CMV polypeptide, and wherein said replaced N-terminal region of said CMV polypeptide consists of 11 to 13 consecutive amino acids, preferably of 11 consecutive amino acids, and wherein further preferably said N-terminal region of said CMV polypeptide corresponds to amino acids 2-12 of SEQ ID NO:48. In a preferred embodiment, said chimeric CMV polypeptide comprises, preferably consists of, the amino acid sequence of SEQ ID NO:5, in which said polypeptide is inserted between any amino acid residue of said CMV polypeptide corresponding to any amino acid residue between position 75 and position 85 of SEQ ID NO:48. In another preferred embodiment, said chimeric CMV polypeptide comprises the amino acid sequence of SEQ ID NO:57, in which said polypeptide is inserted between any amino acid residue of said CMV polypeptide corresponding to any amino acid residue between position 75 and position 85 of SEQ ID NO:48.

In a preferred embodiment, said stretch of consecutive negative amino acids has a length of 3 to 10 amino acids. In a further preferred embodiment, said stretch of consecutive negative amino acids has a length of 3, 4, 5, 6, 7, 8, 9 or 10 amino acids. In a further preferred embodiment, said stretch of consecutive negative amino acids has a length of 3 to 9 amino acids. In a further preferred embodiment, said stretch of consecutive negative amino acids has a length of 3 to 8 amino acids. In a further preferred embodiment, said stretch of consecutive negative amino acids has a length of 4 to 9 amino acids. In a further preferred embodiment, said stretch of consecutive negative amino acids has a length of 4 to 8 amino acids. In a further preferred embodiment, said stretch of consecutive negative amino acids has a length of 4, 5, 6, 7 or 8. In a further preferred embodiment, said stretch of consecutive negative amino acids has a length of 4 or 8 amino acids. In a further preferred embodiment, said stretch of consecutive negative amino acids has a length of 3 amino acids. In a further preferred embodiment, said stretch of consecutive negative amino acids has a length of 4 amino acids. In a further preferred embodiment, said stretch of consecutive negative amino acids has a length of 5 amino acids. In a further preferred embodiment, said stretch of consecutive negative amino acids has a length of 6 amino acids. In a further preferred embodiment, said stretch of consecutive negative amino acids has a length of 7 amino acids. In a further preferred embodiment, said stretch of consecutive negative amino acids has a length of 8 amino acids. In a further preferred embodiment, said stretch of consecutive negative amino acids has a length of 9 amino acids.

In a further preferred embodiment, said stretch of consecutive negative amino acids are independently selected from aspartic acid or glutamic acid, wherein said aspartic acid or said glutamic acid is independently in each occasion selected from its L-configuration or its D-configuration. In a further preferred embodiment, said stretch of consecutive negative amino acids comprises at least one aspartic acid in the L-configuration or in the D-configuration. In a further preferred embodiment, said stretch of consecutive negative amino acids comprises at least one aspartic acid in the L-configuration. In a further preferred embodiment, said stretch of consecutive negative amino acids comprises at least one aspartic acid in the D-configuration. In a further preferred embodiment, said stretch of consecutive negative amino acids comprises at least one glutamic acid in the L-configuration or the D-configuration. In a further preferred embodiment, said stretch of consecutive negative amino acids comprises at least one glutamic acid in the L-configuration. In a further preferred embodiment, said stretch of consecutive negative amino acids comprises at least one glutamic acid in the D-configuration.

In a further preferred embodiment, said stretch of consecutive negative amino acids comprises at least one aspartic acid in the L-configuration and at least one glutamic acid in the L-configuration. In a further preferred embodiment, said stretch of consecutive negative amino acids consists of aspartic acid and glutamic acid, all in the L-configuration. In a further preferred embodiment, said stretch of consecutive negative amino acids consists of aspartic acid or glutamic acid, all in the L-configuration.

In a further preferred embodiment, said stretch of consecutive negative amino acids comprises at least one aspartic acid or at least one glutamic acid. In a further preferred embodiment, said stretch of consecutive negative amino acids comprises at least two aspartic acid or at least two glutamic acid. In a further preferred embodiment, said stretch of consecutive negative amino acids comprises at least three aspartic acid or at least three glutamic acid. In a further preferred embodiment, said stretch of consecutive negative amino acids comprises at least four aspartic acid or at least four glutamic acid. In a further preferred embodiment, said stretch of consecutive negative amino acids comprises at least four aspartic acid. In a further preferred embodiment, said stretch of consecutive negative amino acids comprises at least four glutamic acid. In a further preferred embodiment, said stretch of consecutive negative amino acids comprises at least five glutamic acid. In a further preferred embodiment, said stretch of consecutive negative amino acids comprises at least six glutamic acid. In a further preferred embodiment, said stretch of consecutive negative amino acids comprises at least seven glutamic acid. In a further preferred embodiment, said stretch of consecutive negative amino acids comprises at least eight glutamic acid. In a further preferred embodiment, said stretch of consecutive negative amino acids consist solely of aspartic acid. In a further very preferred embodiment, said stretch of consecutive negative amino acids consists solely of glutamic acids.

In a further preferred embodiment, said stretch of consecutive negative amino acids comprises at least one aspartic acid or at least one glutamic acid, wherein said at least one aspartic acid or said at least one glutamic acid are in the L-configuration. In a further preferred embodiment, said stretch of consecutive negative amino acids comprises at least two aspartic acid or at least two glutamic acid, wherein at least two aspartic acid or at least two glutamic acid are in the L-configuration. In a further preferred embodiment, said stretch of consecutive negative amino acids comprises at least three aspartic acid or at least three glutamic acid, wherein said at least three aspartic acid or said at least three glutamic acid are in the L-configuration. In a further preferred embodiment, said stretch of consecutive negative amino acids comprises at least four aspartic acid or at least four glutamic acid, wherein said at least four aspartic acid or said at least four glutamic acid are in the L-configuration. In a further preferred embodiment, said stretch of consecutive negative amino acids comprises at least four aspartic acid, wherein said at least four aspartic acid are in the L-configuration. In a further preferred embodiment, said stretch of consecutive negative amino acids comprises at least four glutamic acid, wherein said at least four glutamic acid are in the L-configuration. In a further preferred embodiment, said stretch of consecutive negative amino acids comprises at least five glutamic acid, wherein said at least five glutamic acid are in the L-configuration. In a further preferred embodiment, said stretch of consecutive negative amino acids comprises at least six glutamic acid, wherein said at least six glutamic acid are in the L-configuration. In a further preferred embodiment, said stretch of consecutive negative amino acids comprises at least seven glutamic acid, wherein said at least seven glutamic acid are in the L-configuration. In a further preferred embodiment, said stretch of consecutive negative amino acids comprises at least eight glutamic acid, wherein said at least eight glutamic acid are in the L-configuration. In a further preferred embodiment, said stretch of consecutive negative amino acids consist solely of aspartic acids, wherein said aspartic acids are in the L-configuration. In a further very preferred embodiment, said stretch of consecutive negative amino acids consists solely of glutamic acids, wherein said glutamic acids are in the L-configuration.

In a preferred embodiment, said stretch of consecutive negative amino acids has a length of 3 to 10 amino acids, wherein said stretch of consecutive negative amino acids consists solely of glutamic acids. In a further preferred embodiment, said stretch of consecutive negative amino acids has a length of 3, 4, 5, 6, 7, 8, 9 or 10 amino acids, wherein said stretch of consecutive negative amino acids consists solely of glutamic acids. In a further preferred embodiment, said stretch of consecutive negative amino acids has a length of 3 to 9 amino acids, wherein said stretch of consecutive negative amino acids consists solely of glutamic acids. In a further preferred embodiment, said stretch of consecutive negative amino acids has a length of 3 to 8 amino acids, wherein said stretch of consecutive negative amino acids consists solely of glutamic acids. In a further preferred embodiment, said stretch of consecutive negative amino acids has a length of 4 to 9 amino acids, wherein said stretch of consecutive negative amino acids consists solely of glutamic acids. In a further preferred embodiment, said stretch of consecutive negative amino acids has a length of 4 to 8 amino acids, wherein said stretch of consecutive negative amino acids consists solely of glutamic acids. In a further preferred embodiment, said stretch of consecutive negative amino acids has a length of 4, 5, 6, 7 or 8, wherein said stretch of consecutive negative amino acids consists solely of glutamic acids. In a further preferred embodiment, said stretch of consecutive negative amino acids has a length of 4 to 8 amino acids, wherein said stretch of consecutive negative amino acids consists solely of glutamic acids. In a further preferred embodiment, said stretch of consecutive negative amino acids has a length of 4, 5, 6, 7 or 8, wherein said stretch of consecutive negative amino acids consists solely of glutamic acids. In a further preferred embodiment, said stretch of consecutive negative amino acids has a length of 4 or 8 amino acids, wherein said stretch of consecutive negative amino acids consists solely of glutamic acids. In a further preferred embodiment, said stretch of consecutive negative amino acids has a length of 3 amino acids, wherein said stretch of consecutive negative amino acids consists solely of glutamic acids. In a further preferred embodiment, said stretch of consecutive negative amino acids has a length of 4 amino acids, wherein said stretch of consecutive negative amino acids consists solely of glutamic acids. In a further preferred embodiment, said stretch of consecutive negative amino acids has a length of 5 amino acids, wherein said stretch of consecutive negative amino acids consists solely of glutamic acids. In a further preferred embodiment, said stretch of consecutive negative amino acids has a length of 6 amino acids, wherein said stretch of consecutive negative amino acids consists solely of glutamic acids. In a further preferred embodiment, said stretch of consecutive negative amino acids has a length of 7 amino acids, wherein said stretch of consecutive negative amino acids consists solely of glutamic acids. In a further preferred embodiment, said stretch of consecutive negative amino acids has a length of 8 amino acids, wherein said stretch of consecutive negative amino acids consists solely of glutamic acids. In a further preferred embodiment, said stretch of consecutive negative amino acids has a length of 9 amino acids, wherein said stretch of consecutive negative amino acids consists solely of glutamic acids.

In a preferred embodiment, said stretch of consecutive negative amino acids has a length of 3 to 10 amino acids, wherein said stretch of consecutive negative amino acids consists solely of glutamic acids, wherein said glutamic acids are in the L-configuration. In a further preferred embodiment, said stretch of consecutive negative amino acids has a length of 3, 4, 5, 6, 7, 8, 9 or 10 amino acids, wherein said stretch of consecutive negative amino acids consists solely of glutamic acids, wherein said glutamic acids are in the L-configuration. In a further preferred embodiment, said stretch of consecutive negative amino acids has a length of 3 to 9 amino acids, wherein said stretch of consecutive negative amino acids consists solely of glutamic acids, wherein said glutamic acids are in the L-configuration. In a further preferred embodiment, said stretch of consecutive negative amino acids has a length of 3 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4 to 9 amino acids, wherein said stretch of consecutive negative amino acids consists solely of glutamic acids, wherein said glutamic acids are in the L-configuration. In a further preferred embodiment, said stretch of consecutive negative amino acids has a length of 4 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4, 5, 6, 7 or 8, wherein said stretch of consecutive negative amino acids consists solely of glutamic acids, wherein said glutamic acids are in the L-configuration. In a further preferred embodiment, said stretch of consecutive negative amino acids has a length of 4 to 8 amino acids, wherein said stretch of consecutive negative amino acids consists solely of glutamic acids, wherein said glutamic acids are in the L-configuration. In a further preferred embodiment, said stretch of consecutive negative amino acids has a length of 4, 5, 6, 7 or 8, wherein said stretch of consecutive negative amino acids consists solely of glutamic acids, wherein said glutamic acids are in the L-configuration. In a further preferred embodiment, said stretch of consecutive negative amino acids has a length of 4 or 8 amino acids, wherein said stretch of consecutive negative amino acids consists solely of glutamic acids, wherein said glutamic acids are in the L-configuration. In a further preferred embodiment, said stretch of consecutive negative amino acids has a length of 3 amino acids, wherein said stretch of consecutive negative amino acids consists solely of glutamic acids, wherein said glutamic acids are in the L-configuration. In a further preferred embodiment, said stretch of consecutive negative amino acids has a length of 4 amino acids, wherein said stretch of consecutive negative amino acids consists solely of glutamic acids, wherein said glutamic acids are in the L-configuration. In a further preferred embodiment, said stretch of consecutive negative amino acids has a length of 5 amino acids, wherein said stretch of consecutive negative amino acids consists solely of glutamic acids, wherein said glutamic acids are in the L-configuration. In a further preferred embodiment, said stretch of consecutive negative amino acids has a length of 6 amino acids, wherein said stretch of consecutive negative amino acids consists solely of glutamic acids, wherein said glutamic acids are in the L-configuration. In a further preferred embodiment, said stretch of consecutive negative amino acids has a length of 7 amino acids, wherein said stretch of consecutive negative amino acids consists solely of glutamic acids, wherein said glutamic acids are in the L-configuration. In a further preferred embodiment, said stretch of consecutive negative amino acids has a length of 8 amino acids, wherein said stretch of consecutive negative amino acids consists solely of glutamic acids, wherein said glutamic acids are in the L-configuration. In a further preferred embodiment, said stretch of consecutive negative amino acids has a length of 9 amino acids, wherein said stretch of consecutive negative amino acids consists solely of glutamic acids, wherein said glutamic acids are in the L-configuration.

In a further very preferred embodiment, said stretch of consecutive negative amino acids comprises SEQ ID NO:1 or SEQ ID NO:2. In a further very preferred embodiment, said stretch of consecutive negative amino acids consists of SEQ ID NO:1 or SEQ ID NO:2. In a further very preferred embodiment, said stretch of consecutive negative amino acids comprises SEQ ID NO:1. In a further very preferred embodiment, said stretch of consecutive negative amino acids consists of SEQ ID NO:1. In a further very preferred embodiment, said stretch of consecutive negative amino acids comprises SEQ ID NO:2. In a further very preferred embodiment, said stretch of consecutive negative amino acids consists of SEQ ID NO:2.

In a preferred embodiment, said polypeptide comprising said stretch of consecutive negative amino acids further comprises a first amino acid linker, wherein said first amino acid linker is positioned at the N- or at the C-terminus of said stretch of consecutive negative amino acids. In a preferred embodiment, said polypeptide further comprises a first amino acid linker, wherein said first amino acid linker is positioned at the N-terminus of said stretch of consecutive negative amino acids. In a preferred embodiment, said polypeptide comprising said stretch of consecutive negative amino acids further comprises a first amino acid linker, wherein said first amino acid linker is positioned at the C-terminus of said stretch of consecutive negative amino acids. In a preferred embodiment, said polypeptide comprising said stretch of consecutive negative amino acids further comprises a second amino acid linker. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids.

In a preferred embodiment, said first amino acid linker has a length of at most 30 amino acids. In a preferred embodiment, said first amino acid linker has a length of at most 20, 19, 18, 17 or 16 amino acids. In a preferred embodiment, said first amino acid linker has a length of at most 15 amino acids. In a preferred embodiment, said first amino acid linker has a length of at most 14 amino acids. In a preferred embodiment, said first amino acid linker has a length of at most 13 amino acids In a preferred embodiment, said first amino acid linker has a length of at most 12 amino acids. In a preferred embodiment, said first amino acid linker has a length of at most 11 amino acids. In a preferred embodiment, said first amino acid linker has a length of at most 10 amino acids. In a preferred embodiment, said first amino acid linker has a length of at most 9 amino acids. In a preferred embodiment, said first amino acid linker has a length of at most 8 amino acids. In a preferred embodiment, said first amino acid linker has a length of at most 7 amino acids. In a preferred embodiment, said first amino acid linker has a length of at most 6 amino acids. In a preferred embodiment, said first amino acid linker has a length of at most 5 amino acids. In a preferred embodiment, said first amino acid linker has a length of at most 4 amino acids. In a preferred embodiment, said first amino acid linker has a length of at most 3 amino acids. In a preferred embodiment, said first amino acid linker has a length of at most 2 amino acids. In a preferred embodiment, said first amino acid linker consists of one amino acid. In a preferred embodiment, said second amino acid linker has a length of at most 30 amino acids. In a preferred embodiment, said second amino acid linker has a length of at most 20, 19, 18, 17 or 16 amino acids. In a preferred embodiment, said second amino acid linker has a length of at most 15 amino acids. In a preferred embodiment, said second amino acid linker has a length of at most 14 amino acids. In a preferred embodiment, said second amino acid linker has a length of at most 13 amino acids In a preferred embodiment, said second amino acid linker has a length of at most 12 amino acids. In a preferred embodiment, said second amino acid linker has a length of at most 11 amino acids. In a preferred embodiment, said second amino acid linker has a length of at most 10 amino acids. In a preferred embodiment, said second amino acid linker has a length of at most 9 amino acids. In a preferred embodiment, said second amino acid linker has a length of at most 8 amino acids. In a preferred embodiment, said second amino acid linker has a length of at most 7 amino acids. In a preferred embodiment, said second amino acid linker has a length of at most 6 amino acids. In a preferred embodiment, said second amino acid linker has a length of at most 5 amino acids. In a preferred embodiment, said second amino acid linker has a length of at most 4 amino acids. In a preferred embodiment, said second amino acid linker has a length of at most 3 amino acids. In a preferred embodiment, said second amino acid linker has a length of at most 2 amino acids. In a preferred embodiment, said second amino acid linker consists of one amino acid.

In a preferred embodiment, said polypeptide comprising said stretch of consecutive negative amino acids further comprises a first amino acid linker, wherein said first amino acid linker is positioned at the N- or at the C-terminus of said stretch of consecutive negative amino acids, and wherein said first amino acid linker is selected from the group consisting of: (a.) a polyglycine linker (Gly) of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys.

In a preferred embodiment, said polypeptide comprising said stretch of consecutive negative amino acids further comprises a second amino acid linker, wherein said second amino acid linker is positioned at the N- or at the C-terminus of said stretch of consecutive negative amino acids, and wherein said second amino acid linker is selected from the group consisting of: (a.) a polyglycine linker (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys. In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of (a.) a polyglycine linker (G-linker) having an amino acid sequence (Gly)ₙ of a length of n=2-10; (b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and (c.) an amino acid linker (GS*-linker) comprising at least one Gly, at least one Ser, and at least one amino acid selected from Thr, Ala, Lys, and Cys.

In a preferred embodiment, said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker are independently selected from a glycine-serine linker (GS-linker) comprising at least one glycine and at least one serine, wherein said GS linker has an amino acid sequence of (GS)ᵣ(GₛS)ₜ(GS)ᵤ with r=0 or 1, s=1-5, t=1-5 and u=0 or 1; and an amino acid linker (GS*-linker) comprising at least one Gly, at least one Ser, and at least one amino acid selected from Thr, Ala, Lys, and Cys.

In a preferred embodiment, said first amino acid linker is a polyglycine linker (Gly)ₙ of a length of n=2-10. In a preferred embodiment, said first amino acid linker is a glycine-serine linker (GS-linker) comprising at least one glycine and at least one serine. In a preferred embodiment, said first amino acid linker is a glycine-serine linker (GS-linker) comprising at least one glycine and at least one serine, and wherein said first amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, and u=0 or 1. In a further preferred embodiment, said GS-linker has a length of at most 15, 14, 13, 12, 11, preferably 10, 9, 8, 7, and further preferably a length of at most 6 amino acids. In a further preferred embodiment, said first amino acid linker is a glycine-serine linker (GS-linker), and said GS linker has an amino acid sequence of SEQ ID NO:8. In a further preferred embodiment, said first amino acid linker has an amino acid sequence of SEQ ID NO:8. In a preferred embodiment, said first amino acid linker is an amino acid linker (GS*-linker) comprising at least one Gly, at least one Ser, and at least one amino acid selected from Thr, Ala, Lys, and Cys.

In a preferred embodiment, said second amino acid linker is a polyglycine linker (Gly)ₙ of a length of n=2-10. In a preferred embodiment, said second amino acid linker is a glycine-serine linker (GS-linker) consisting of at least one glycine and at least one serine. In a preferred embodiment, said second amino acid linker is a glycine-serine linker (GS-linker) comprising at least one glycine and at least one serine, and wherein said second amino acid linker has a Gly-Ser at its N-terminus. In a further preferred embodiment, said second amino acid linker is a glycine-serine linker (GS-linker), said GS linker has an amino acid sequence of (GS)ᵣ(GₛS)ₜ(GS)ᵤ with r=0 or 1, s=3 or 4, t=1, 2 or 3, u=0 or 1. In a further preferred embodiment, said GS-linker has a length of at most 15, 14, 13, 12, 11, preferably 10, 9, 8, 7, and further preferably a length of at most 6 amino acids. In a further preferred embodiment, said second amino acid linker is a glycine-serine linker (GS-linker), and said GS linker has the amino acid sequence of SEQ ID NO:9.

In a preferred embodiment, said second amino acid linker is an amino acid linker (GS*-linker) comprising at least one Gly, at least one Ser, and at least one amino acid selected from Thr, Ala, Lys, and Cys. In a preferred embodiment, said second amino acid linker is an amino acid linker (GS*-linker) comprising at least one Gly, at least one Ser, and at least Cys. In a preferred embodiment, said second amino acid linker is an amino acid linker comprising at least one Gly, at least one Ser, and at least Cys (GS*-linker), and said second amino acid linker has a Gly-Ser at its N-terminus. In a further preferred embodiment, said second amino acid linker (GS*-linker) has a length of at most 15, 14, 13, 12, 11, preferably 10, 9, and further preferably a length of at most 7 or 6 amino acids. In a further preferred embodiment, said second amino acid linker is amino acid linker (GS*-linker), and said GS*-linker has the amino acid sequence of SEQ ID NO:4.

In a preferred embodiment, said first and said second amino acid linker are independently a polyglycine linker (Gly)ₙ of a length of n=2-10. In a preferred embodiment, said first and said second amino acid linker are independently a glycine-serine linker (GS-linker) comprising at least one glycine and at least one serine. In a preferred embodiment, said first and said second amino acid linker are independently an amino acid linker (GS*-linker) comprising at least one Gly, at least one Ser, and at least one amino acid selected from Thr, Ala, Lys, and Cys, and wherein said second amino acid linker has a Gly-Ser at its N-terminus. In a further preferred embodiment, said GS linker has an amino acid sequence of (GS)ᵣ(GₛS)ₜ(GS)ᵤ with r=0 or 1, s=1-5, t=1-5 and u=0 or 1. In a further preferred embodiment, said first and said second amino acid linker is independently a glycine-serine linker (GS-linker), said GS linker has an amino acid sequence of (GS)ᵣ(GₛS)ₜ(GS)ᵤ with r=0 or 1, s=2, 3 or 4, t=1, 2 or 3, u=0 or 1.

In a further preferred embodiment, said first amino acid linker and/or said second amino linker comprises, preferably consists of, of an amino acid sequence selected from SEQ ID NO:4, SEQ ID NO:8 and SEQ ID NO:9. In a further very preferred embodiment, said first amino acid linker comprises, preferably consists of, SEQ ID NO:8. In a further very preferred embodiment, said second amino acid linker comprises, preferably consists of, SEQ ID NO:4 or SEQ ID NO:9. In a further very preferred embodiment, said second amino acid linker comprises, preferably consists of, SEQ ID NO:4. In a further very preferred embodiment, said second amino acid linker comprises, preferably consists of, SEQ ID NO:9. In a further very preferred embodiment, said first amino acid linker comprises, preferably consists of, SEQ ID NO:8 and said second amino acid linker comprises, preferably consists of, SEQ ID NO:4 or SEQ ID NO:9. In a further very preferred embodiment, said first amino acid linker comprises, preferably consists of, SEQ ID NO:8 and said second amino acid linker comprises, preferably consists of, SEQ ID NO:4. In a further very preferred embodiment, said first amino acid linker comprises, preferably consists of, SEQ ID NO:8 and said second amino acid linker comprises, preferably consists of, or SEQ ID NO:9.

In a preferred embodiment, said polypeptide comprising, preferably consisting of, a stretch of consecutive negative amino acids has a length of at most 30 amino acids. In a preferred embodiment, said polypeptide has a length of at most 25, 24, 23, 22, or 21 amino acids. In a preferred embodiment, said polypeptide has a length of at most 20 amino acids. In a preferred embodiment, said polypeptide has a length of at most 19 amino acids. In a preferred embodiment, said polypeptide has a length of at most 18 amino acids. In a preferred embodiment, said polypeptide has a length of at most 17 amino acids. In a preferred embodiment, said polypeptide has a length of at most 16 amino acids. In a preferred embodiment, said polypeptide has a length of at most 15 amino acids. In a preferred embodiment, said polypeptide has a length of at most 14 amino acids. In a preferred embodiment, said polypeptide has a length of at most 13 amino acids In a preferred embodiment, said polypeptide has a length of at most 12 amino acids. In a preferred embodiment, said polypeptide has a length of at most 11 amino acids. In a preferred embodiment, said polypeptide has a length of at most 10 amino acids. In a preferred embodiment, said polypeptide has a length of at most 9 amino acids. In a preferred embodiment, said polypeptide has a length of at most 8 amino acids. In a preferred embodiment, said polypeptide has a length of at most 7 amino acids. In a preferred embodiment, said polypeptide has a length of at most 6 amino acids. In a preferred embodiment, said polypeptide has a length of at most 5 amino acids. In a preferred embodiment, said polypeptide has a length of at most 4 amino acids. In a further preferred embodiment, said polypeptide consists of said stretch of consecutive negative amino acids.

In a further very preferred embodiment, said polypeptide comprises SEQ ID NO:62, SEQ ID NO:63 or SEQ ID NO:64. In a further very preferred embodiment, said polypeptide consists of SEQ ID NO:62, SEQ ID NO:63 or SEQ ID NO:64. In a further very preferred embodiment, said polypeptide comprises SEQ ID NO:62. In a further very preferred embodiment, said polypeptide comprises SEQ ID NO:63. In a further very preferred embodiment, said polypeptide comprises SEQ ID NO:64. In a further very preferred embodiment, said polypeptide consists of SEQ ID NO:62. In a further very preferred embodiment, said polypeptide consists of SEQ ID NO:63. In a further very preferred embodiment, said polypeptide consists of SEQ ID NO:64.

In a further preferred embodiment, said polypeptide comprising, preferably consisting of, a stretch of consecutive negative amino acids is inserted between any amino acid residue of said CMV polypeptide corresponding to any amino acid residue between position 75 and position 85 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 75 and position 76 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 76 and position 77 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 77 and position 78 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 78 and position 79 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 79 and position 80 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 80 and position 81 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 75 and position 81 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 82 and position 83 of SEQ ID NO:48. In a further preferred embodiment, said polypeptide comprising, preferably consisting of, a stretch of consecutive negative amino acids is inserted between amino acid residues of said CMV polypeptide corresponding to amino acid residues of position 83 and position 84 of SEQ ID NO:48. In a further very preferred embodiment, said polypeptide comprising, preferably consisting of, a stretch of consecutive negative amino acids is inserted between amino acid residues of said CMV polypeptide corresponding to amino acid residues of position 84 and position 85 of SEQ ID NO:48.

In a very preferred embodiment, said CMV polypeptide comprises the amino acid sequence of SEQ ID NO:5, SEQ ID NO:48 or SEQ ID NO:57, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:48, or between amino acid residues of position 86 (Ser) and position 87 (Thr) of SEQ ID NO:57. In a very preferred embodiment, said CMV polypeptide consists of the amino acid sequence of SEQ ID NO:5, SEQ ID NO:48 or SEQ ID NO:57, wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57. In a very preferred embodiment, said CMV polypeptide comprises the amino acid sequence of SEQ ID NO:5, wherein said polypeptide is inserted between amino acid residues of position 88 and position 89 of SEQ ID NO:5. In a very preferred embodiment, said CMV polypeptide comprises the amino acid sequence of SEQ ID NO:48, wherein said polypeptide is inserted between amino acid residues of position 84 and position 85 of SEQ ID NO:48. In a very preferred embodiment, said CMV polypeptide comprises the amino acid sequence of SEQ ID NO:57, wherein said polypeptide is inserted between amino acid residues of position 86 and position 87 of SEQ ID NO:57. In a very preferred embodiment, said CMV polypeptide consists of the amino acid sequence of SEQ ID NO:5, wherein said polypeptide is inserted between amino acid residues of position 88 and position 89 of SEQ ID NO:5. In a very preferred embodiment, said CMV polypeptide consists of the amino acid sequence of SEQ ID NO:48, wherein said polypeptide is inserted between amino acid residues of position 84 and position 85 of SEQ ID NO:48. In a very preferred embodiment, said CMV polypeptide consists of the amino acid sequence of SEQ ID NO:57, wherein said polypeptide is inserted between amino acid residues of position 86 and position 87 of SEQ ID NO:57.

In a very preferred embodiment, said chimeric CMV polypeptide comprises the amino acid sequence of SEQ ID NO:5, SEQ ID NO:48 or SEQ ID NO:57, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:48, or between amino acid residues of position 86 (Ser) and position 87 (Thr) of SEQ ID NO:57. In a very preferred embodiment, said chimeric CMV polypeptide consists of the amino acid sequence of SEQ ID NO:5, SEQ ID NO:48 or SEQ ID NO:57, wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57. In a very preferred embodiment, said chimeric CMV polypeptide comprises the amino acid sequence of SEQ ID NO:5, wherein said polypeptide is inserted between amino acid residues of position 88 and position 89 of SEQ ID NO:5. In a very preferred embodiment, said chimeric CMV polypeptide comprises the amino acid sequence of SEQ ID NO:48, wherein said polypeptide is inserted between amino acid residues of position 84 and position 85 of SEQ ID NO:48. In a very preferred embodiment, said chimeric CMV polypeptide comprises the amino acid sequence of SEQ ID NO:57, wherein said polypeptide is inserted between amino acid residues of position 86 and position 87 of SEQ ID NO:57. In a very preferred embodiment, said chimeric CMV polypeptide consists of the amino acid sequence of SEQ ID NO:5, wherein said polypeptide is inserted between amino acid residues of position 88 and position 89 of SEQ ID NO:5. In a very preferred embodiment, said chimeric CMV polypeptide consists of the amino acid sequence of SEQ ID NO:48, wherein said polypeptide is inserted between amino acid residues of position 84 and position 85 of SEQ ID NO:48. In a very preferred embodiment, said chimeric CMV polypeptide consists of the amino acid sequence of SEQ ID NO:57, wherein said polypeptide is inserted between amino acid residues of position 86 and position 87 of SEQ ID NO:57.

In a very preferred embodiment, said CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:10, SEQ ID NO:11 or SEQ ID NO:12. In a very preferred embodiment, said chimeric CMV polypeptide comprises the amino acid sequence of SEQ ID NO: 10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO:12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO:12.

Thus, in another aspect, the present invention provides a modified virus-like particle (VLP) of cucumber mosaic virus (CMV) comprising at least one chimeric CMV polypeptide, wherein said at least one chimeric CMV polypeptide comprises, preferably consists of, the amino acid sequence of SEQ ID NO:10, SEQ ID NO:11 or SEQ ID NO:12. In a very preferred embodiment, said modified VLP of CMV comprises 180 identical chimeric CMV polypeptides, wherein said chimeric CMV polypeptide comprises, preferably consists of, the amino acid sequence of SEQ ID NO:10, SEQ ID NO:11 or SEQ ID NO:12.

The modified CMV VLPs of the invention may be expressed in prokaryotic or eukaryotic expression systems. Preferred systems are E. coli, yeast, insect cells as well as mammalian cell lines. Very preferred said modified VLP of CMV is obtained by expression of said chimeric CMV polypeptide in E. coli., and wherein preferably said expression is effected at temperatures of between 10° C. to 25° C., preferably at a temperature of 20° C. As indicated above, recombinantly produced polypeptides may comprise an N-terminal methionine residue. In one embodiment said chimeric CMV polypeptide therefore comprises an N-terminal methionine residue. However, typically and preferably said N-terminal methionine residue is cleaved off said chimeric CMV polypeptide.

In a further preferred embodiment said modified VLP of CMV further comprises at least one immunostimulatory substance. In a very preferred embodiment, said immunostimulatory substance is packaged into the modified VLPs of the invention. In another preferred embodiment, the immunostimulatory substance is mixed with the modified VLPs of the invention. Immunostimulatory substances useful for the invention are generally known in the art and are disclosed, inter alia, in WO2003/024481.

In another embodiment of the present invention, said immunostimulatory substance consists of DNA or RNA of non-eukaryotic origin. In a further preferred embodiment said immunostimulatory substance is selected from the group consisting of: (a) immunostimulatory nucleic acid; (b) peptidoglycan; (c) lipopolysaccharide; (d) lipoteichonic acid; (e) imidazoquinoline compound; (f) flagellin; (g) lipoprotein; and (h) any mixtures of at least one substance of (a) to (g). In a further preferred embodiment said immunostimulatory substance is an immunostimulatory nucleic acid, wherein said immunostimulatory nucleic acid is selected from the group consisting of: (a) ribonucleic acids; (b) deoxyribonucleic acids; (c) chimeric nucleic acids; and (d) any mixture of (a), (b) and/or (c). In a further preferred embodiment said immunostimulatory nucleic acid is a ribonucleic acid, and wherein said ribonucleic acid is bacteria derived RNA. In a further preferred embodiment said immunostimulatory nucleic acid is poly(IC) or a derivative thereof. In a further preferred embodiment said immunostimulatory nucleic acid is a deoxyribonucleic acid, wherein said deoxyribonucleic acid is an unmethylated CpG-containing oligonucleotide.

In a very preferred embodiment said immunostimulatory substance is an unmethylated CpG-containing oligonucleotide. In a further preferred embodiment said unmethylated CpG-containing oligonucleotide is an A-type CpG. In a further preferred embodiment said A-type CpG comprises a palindromic sequence. In a further preferred embodiment said palindromic sequence is flanked at its 5′-terminus and at its 3′-terminus by guanosine entities. In a further preferred embodiment said palindromic sequence is flanked at its 5′-terminus by at least 3 and at most 15 guanosine entities, and wherein said palindromic sequence is flanked at its 3′-terminus by at least 3 and at most 15 guanosine entities.

In another preferred embodiment, said immunostimulatory substance is an unmethylated CpG-containing oligonucleotide, and wherein preferably said unmethylated CpG-containing oligonucleotide comprises a palindromic sequence, and wherein further preferably the CpG motif of said unmethylated CpG-containing oligonucleotide is part of a palindromic sequence, and wherein again further preferably said palindromic sequence is SEQ ID NO:65. In a further preferred embodiment, said immunostimulatory nucleic acid is an unmethylated CpG containing oligonucleotide consisting of SEQ ID NO:66, wherein said unmethylated CpG-containing oligonucleotide consists exclusively of phosphodiester bound nucleotides.

In a further aspect, the present invention provides a composition comprising (a) modified VLP of CMV as defined herein, wherein said modified VLP of CMV comprises at least one first attachment site; and (b) at least one antigen, wherein said antigen comprises at least one second attachment site; wherein (a) and (b) are linked through said at least one first and said at least one second attachment site, typically and preferably via at least one covalent non-peptide bond. Methods for linking said modified VLP and said antigens via said first and said second attachment site are described, for example, in WO2002/056905, WO2004/084940 and WO2016/062720.

Thus, in a further aspect, the present invention provides a composition comprising (a) modified VLP of CMV, wherein said modified VLP of CMV comprises at least one first attachment site; and (b) at least one antigen, wherein said antigen comprises at least one second attachment site; wherein (a) and (b) are linked through said at least one first and said at least one second attachment site, typically and preferably via at least one covalent non-peptide bond, and wherein said modified VLP of CMV comprises at least one chimeric CMV polypeptide, wherein said at least one chimeric CMV polypeptide comprises, preferably consists of, (i) a CMV polypeptide, wherein said CMV polypeptide comprises a coat protein of CMV or an amino acid sequence having a sequence identity of at least 75% with SEQ ID NO:48; and (ii) a polypeptide comprising, preferably consisting of, a stretch of consecutive negative amino acids, wherein said negative amino acids are independently selected from aspartic acid or glutamic acid, and wherein said polypeptide is inserted between any amino acid residue of said CMV polypeptide corresponding to any amino acid residue between position 75 and position 85 of SEQ ID NO:48.

In a very preferred embodiment, said at least one first attachment site is not comprised or is not part of the polypeptide comprising said stretch of consecutive negative amino acids. In a very preferred embodiment, all of said first attachments sites are not comprised or are not part of the polypeptide comprising said stretch of consecutive negative amino acids. In a very preferred embodiment, said at least one first attachment site is not comprised or is not part of the stretch of consecutive negative amino acids. In a very preferred embodiment, all of said first attachments sites are not comprised or are not part of the stretch of consecutive negative amino acids. In a very preferred embodiment, said first attachment site and said second attachment site are linked solely via one or more covalent bonds. In a very preferred embodiment, said at least one antigen is linked to said modified VLP of CMV solely via one or more covalent bonds. In a very preferred embodiment, all of said antigens are linked to said modified VLP of CMV solely via one or more covalent bonds.

In a further preferred embodiment, said first attachment site is linked to said second attachment site via at least one covalent non-peptide bond. In a further preferred embodiment, all of said first attachment sites are linked to said second attachment sites via at least one covalent non-peptide bond. In a further very preferred embodiment, said first attachment site is an amino group, preferably an amino group of a lysine. In a further very preferred embodiment, all of said first attachment sites are an amino group, preferably an amino group of a lysine.

Attachment between modified virus-like particles and antigens by way of disulfide bonds are typically labile, in particular, to sulfhydryl-moiety containing molecules, and are, furthermore, less stable in serum than, for example, thioether attachments (Martin F J. and Papahadjopoulos D. (1982) J. Biol. Chem. 257: 286-288). Therefore, in a further very preferred embodiment of the present invention, the association or linkage of the modified VLP of CMV and the at least one antigen does not comprise a disulfide bond. Further preferred hereby, the at least one second attachment site comprise, or preferably is, a sulfhydryl group. Preferably, all of said second attachment sites comprise, or preferably are, a sulfhydryl group. In a further preferred embodiment, said at least one first attachment site is not or does not comprise a sulfhydryl group. In a further preferred embodiment, all of said first attachment sites are not or do not comprise a sulfhydryl group. In a preferred embodiment, said at least one first attachment site is not or does not comprise a sulfhydryl group of a cysteine. In a preferred embodiment, all of said first attachment sites are not or do not comprise a sulfhydryl group of a cysteine. In a further very preferred embodiment said second attachment site is a sulfhydryl group, preferably a sulfhydryl group of a cysteine. In a further very preferred embodiment, all of said second attachment sites are a sulfhydryl group, preferably a sulfhydryl group of a cysteine.

In a very preferred embodiment, the at least one first attachment site is an amino group, preferably an amino group of a lysine residue and the at least one second attachment site is a sulfhydryl group, preferably a sulfhydryl group of a cysteine residue or a sulfhydryl group that has been chemically attached to the antigen. In a very preferred embodiment, all of said first attachment sites are an amino group, preferably an amino group of a lysine residue and all of said second attachment sites are a sulfhydryl group, preferably a sulfhydryl group of a cysteine residue or a sulfhydryl group that has been chemically attached to the antigen. In a further preferred embodiment only one of said second attachment sites associates with said first attachment site through at least one non-peptide covalent bond leading to a single and uniform type of binding of said antigen to said modified VLP of CMV, wherein said only one second attachment site that associates with said first attachment site is a sulfhydryl group, and wherein said antigen and said modified VLP of CMV interact through said association to form an ordered and repetitive antigen array.

In one preferred embodiment of the invention, the antigen is linked to the modified VLP of CMV by way of chemical cross-linking, typically and preferably by using a heterobifunctional cross-linker. In preferred embodiments, the hetero-bifunctional cross-linker contains a functional group which can react with the preferred first attachment sites, preferably with the amino group, more preferably with the amino groups of lysine residue(s) of the modified VLP of CMV, and a further functional group which can react with the preferred second attachment site, i.e. a sulfhydryl group, preferably of cysteine(s) residue inherent of, or artificially added to the antigen, and optionally also made available for reaction by reduction. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, Sulfo-KMUS SVSB, SIA, and other cross-linkers available for example from the Pierce Chemical Company, and having one functional group reactive towards amino groups and one functional group reactive towards sulfhydryl groups. The above mentioned cross-linkers all lead to formation of an amide bond after reaction with the amino group and a thioether linkage with the sulfhydryl groups. In a very preferred embodiment, said hetero-bifunctional cross-linker is SMPH. Thus, in a preferred embodiment, the antigen is linked to the modified VLP of CMV by way of chemical cross-linking, typically and preferably by way of a heterobifunctional cross-linker through said at least one first and said at least one second attachment site via at least one covalent non-peptide bond, and wherein said hetero-bifunctional cross-linker is SMPH. Another class of cross-linkers suitable in the practice of the invention is characterized by the introduction of a disulfide linkage between the antigen and the modified VLP upon coupling. Preferred cross-linkers belonging to this class include, for example, SPDP and Sulfo-LC-SPDP (Pierce).

Thus, in a further aspect, the present invention provides a composition comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO:10, SEQ ID NO:11 or SEQ ID NO:12;
(b) at least one antigen, wherein said antigen comprises at least one second attachment site; and

wherein (a) and (b) are linked by way of a heterobifunctional cross-linker through said at least one first and said at least one second attachment site via at least one covalent non-peptide bond, preferably wherein said hetero-bifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group of a cysteine residue or a sulfhydryl group that has been chemically attached to the antigen. In a very preferred embodiment, said chimeric CMV polypeptide comprises the amino acid sequence of SEQ ID NO:10. In a very preferred embodiment, said chimeric CMV polypeptide comprises the amino acid sequence of SEQ ID NO: 11. In a very preferred embodiment, said chimeric CMV polypeptide comprises the amino acid sequence of SEQ ID NO:12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO:12.

Linking of the antigen to the modified VLP of CMV by using a hetero-bifunctional cross-linker allows linking of the antigen to the modified VLP of CMV in an oriented fashion. Other methods of linking the antigen to the modified VLP of CMV include methods wherein the antigen is cross-linked to the modified VLP of CMV, using the carbodiimide EDC, and NHS. The antigen may also be first thiolated through reaction, for example with SATA, SATP or iminothiolane. The antigen, after deprotection if required, may then be coupled to the modified VLP of CMV as follows. After separation of the excess thiolation reagent, the antigen is reacted with the modified VLP of CMV, previously activated with a hetero-bifunctional cross-linker comprising a cysteine reactive moiety, and therefore displaying at least one or several functional groups reactive towards cysteine residues, to which the thiolated antigen can react, such as described above. Optionally, low amounts of a reducing agent are included in the reaction mixture. In further methods, the antigen is attached to the modified VLP of CMV, using a homo-bifunctional cross-linker such as glutaraldehyde, DSG, BM[PEO]4, BS3, (Pierce) or other known homo-bifunctional cross-linkers with functional groups reactive towards amine groups or carboxyl groups of the modified VLP.

In very preferred embodiments of the invention, the antigen is linked via a cysteine residue, having been added to either the N-terminus or the C-terminus of, or a natural cysteine residue within the antigen, to lysine residues of the modified VLP of CMV. In a preferred embodiment, the composition of the invention further comprises a linker, wherein said linker associates said antigen with said second attachment site, and wherein preferably said linker comprises or alternatively consists of said second attachment site.

Engineering of a second attachment site onto the antigen is achieved by the association of a linker, typically and preferably containing at least one amino acid suitable as second attachment site according to the disclosures of this invention. Therefore, in a preferred embodiment of the present invention, a linker is associated to the antigen by way of at least one covalent bond, preferably, by at least one, preferably one peptide bond. Preferably, the linker comprises, or alternatively consists of, the second attachment site. In a further preferred embodiment, the linker comprises a sulfhydryl group, preferably of a cysteine residue. In another preferred embodiment, the linker comprises or is a cysteine residue. In a further preferred embodiment of the present invention, the linker consists of amino acids, wherein further preferably the linker consists at most 15 amino acids. In an again preferred embodiment of the invention, such amino acid linker contains 1 to 10 amino acids.

In again a further aspect, the present invention provides a composition comprising
(a) a modified VLP of CMV, wherein said modified VLP of CMV comprises at least one first attachment site, and wherein said modified VLP of CMV comprises at least one chimeric CMV polypeptide, wherein said at least one chimeric CMV polypeptide comprises, preferably consists of, the amino acid sequence of SEQ ID NO:10;
(b) at least one antigen, wherein said antigen comprises at least one second attachment site; and

wherein (a) and (b) are linked, preferably by way of a heterobifunctional cross-linker through said at least one first and said at least one second attachment site via at least one covalent non-peptide bond, wherein said preferred hetero-bifunctional cross-linker is SMPH, and wherein said at least one first attachment site is not comprised or is not part of the polypeptide comprising said stretch of consecutive negative amino acids, and wherein said at least one first attachment site is an amino group, hereby preferably an amino group of a lysine, and wherein the at least one second attachment site is a sulfhydryl group, hereby preferably a sulfhydryl group that has been chemically attached to the antigen. In a very preferred embodiment, said chimeric CMV polypeptide consists of the amino acid sequence of SEQ ID NO:10. In a very preferred embodiment, said modified VLP of CMV comprises 180 identical chimeric CMV polypeptides, wherein said chimeric CMV polypeptide comprises, preferably consists of, the amino acid sequence of SEQ ID NO:10. In a very preferred embodiment, said modified VLP of CMV comprises 180 identical chimeric CMV polypeptides, wherein said chimeric CMV polypeptide consists of the amino acid sequence of SEQ ID NO:10.

In a preferred embodiment, said antigen is a polypeptide derived from the group consisting of: (a) allergens; (b) viruses; (c) bacteria; (d) parasites; (e) tumors; (f) self-molecules; (g) hormones; (h) growth factors; (i) cytokines; (j) chemokines; and (k) biologically active peptides. In a preferred embodiment, said antigen is an allergen, a self antigen, a tumor antigen, a hormone, a cytokine, a growth factor, a chemokine, or a polypeptide of a viral, bacterial or pathogen In a preferred embodiment, said antigen is of bacterial, viral or mammalian origin. In a preferred embodiment, said antigen is an allergen, a polypeptide derived from a viral pathogen, a polypeptide derived from a bacterial pathogen, a tumor antigen, a self antigen, a polypeptide derived from a hormone, a polypeptide derived from a growth factor, a cytokine or a chemokine. In another preferred embodiment, said antigen is an allergen, a self antigen, a tumor antigen, or a polypeptide of a pathogen. In a preferred embodiment, said antigen is an allergen, a polypeptide derived from a viral pathogen, a polypeptide derived from a bacterial pathogen, a self antigen, a growth factor, a cytokine or a chemokine. In a preferred embodiment, said antigen is an allergen. In a preferred embodiment, said antigen is of viral origin. In a preferred embodiment, said antigen is a polypeptide derived from a virus. In a preferred embodiment, said antigen is of bacterial origin. In a preferred embodiment, said antigen is a polypeptide derived from a bacteria. In a preferred embodiment, said antigen is a polypeptide derived from a parasite. In a preferred embodiment, said antigen is a tumor antigen. In a preferred embodiment, said antigen is a self antigen. In a preferred embodiment, said antigen is a polypeptide derived from a parasite. In a preferred embodiment, said antigen is a hormone. In a preferred embodiment, said antigen is a growth factor. In a preferred embodiment, said antigen is cytokine. In a preferred embodiment, said antigen is chemokine. In a preferred embodiment, said antigen is biologically active peptide. In a preferred embodiment, said antigen is a growth factor or a cytokine.

In a further preferred embodiment said antigen is an allergen, wherein said allergen is derived from the group consisting of: (a) pollen extract; (b) dust extract; (c) dust mite extract; (d) fungal extract; (e) mammalian epidermal extract; (f) feather extract; (g) insect extract; (h) food extract; (i) hair extract; (j) saliva extract; and (k) serum extract. In a further preferred embodiment said antigen is an allergen, wherein said allergen is selected from the group consisting of: (a) trees; (b) grasses; (c) house dust; (d) house dust mite; (e) aspergillus; (f) animal hair; (g) animal feather; (h) bee venom; (i) animal products; (j) plant products; (k) animal dander and (1) peanut allergens.

In a further preferred embodiment said antigen is a recombinant polypeptide derived from an allergen selected from the group consisting of: (a) bee venom phospholipase A2; (b) ragweed pollen Amb a 1; (c) birch pollen Bet v I; (d) white faced hornet venom 5 DoI m V; (e) house dust mite Der p 1; (f) house dust mite Der f 2; (g) house dust mite Der p 2; (h) dust mite Lep d; (i) fungus allergen Alt a 1; (j) fungus allergen Asp f 1; (k) fungus allergen Asp f 16; (l) peanut allergens (m) cat allergen Fel d1; (n) Canine allergens Can f1, Can f2 (o) peanut-derived allergens; or (p) Japanese cedar allergen Cry J2.

In a further preferred embodiment said antigen is a recombinant allergen, wherein said allergen is selected from the group consisting of: (a) bee venom phospholipase A2; (b) ragweed pollen Amb a 1; (c) birch pollen Bet v I; (d) white faced hornet venom 5 DoI m V; (e) house dust mite Der p 1; (f) house dust mite Der f 2; (g) house dust mite Der p 2; (h) dust mite Lep d; (i) fungus allergen Alt a 1; (j) fungus allergen Asp f 1; (k) fungus allergen Asp f 16; (l) peanut allergens (m) cat allergen Fel d1; (n) Canine allergens Can f1, Can f2 (o) peanut-derived allergens; or (p) Japanese cedar allergen Cry J2.

In a further preferred embodiment, said antigen is an allergen derived from Japanese Cedar Cry J 2. Preferably, said antigen is derived from Japanese Cedar Cry J 2 of SEQ ID NO:67. Preferably, said antigen is derived from Japanese Cedar Cry J 2 and comprises, further preferably consists of the amino acid sequence of SEQ ID NO:67.

In a further preferred embodiment, said antigen is an allergen derived from ragweed pollen Amb al. Preferably, said antigen is derived from ragweed pollen Amb a 1 of SEQ ID NO:68. Preferably, said antigen is derived from ragweed pollen Amb al and comprises, preferably consist of the amino acid sequence of SEQ ID NO:68.

In a further preferred embodiment said antigen is a tumor antigen, wherein said tumor antigen is selected from the group consisting of: (a) a polypeptide of breast cancer cells; (b) a polypeptide of kidney cancer cells; (c) a polypeptide of prostate cancer cells; (d) a polypeptide of skin cancer cells; (e) a polypeptide of brain cancer cells; and (f) a polypeptide of leukemia cells.

In a further preferred embodiment said antigen is a tumor antigen selected from the group consisting of: (a) Her2; (b) ganglioside GD2; (c) EGF-R; (d) carcino embryonic antigen (CEA); (e) CD52; (f) CD21; (g) human melanoma gp100; (h) human melanoma melanA/MART-1; (i) Human melanoma melanA/MART-1 analogue; (j) tyrosinase; (k) NA17-A nt; (l) MAGE3; (m) p53 protein; and (n) antigenic fragments of any of the tumor antigens of (a) to (m).

In a further preferred embodiment said antigen is a polypeptide selected from the group consisting of: (a) IgE, (b) IL-6 (c) receptor activator of nuclear factor kB ligand (RANKL); (d) vascular endothelial growth factor (VEGF); (e) vascular endothelial growth factor receptor (VEGF-R); (f) hepatocyte growth factor (HGF) (g) interleukin-1α; (h) interleukin-1β; (i) interleukin-5; (j) interleukin-8; (k) interleukin-13; (l) interleukin-15; (m) interleukin-17; (n) IL-23; (o) Ghrelin; (p) angiotensin; (q) chemokine (C-C motif) (CCL21); (r) chemokine (C-X motif) (CXCL 12); (s) stromal cell derived factor 1 (SDF-I); (t) monocyte chemotactic protein 1 (MCP-I); (u) endoglin; (v) resistin; (w) gonadotropin releasing hormone (GnRH); (x) growth hormone releasing (GHRH); (y) lutenizing hormone releasing hormone (LHRH); (z) thyreotropin releasing hormone (TRH); (aa) macrophage migration inhibitory factor (MIF); (bb) glucose-dependent insulinotropic peptide (GIP); (cc) eotaxin; (dd) bradykinin; (ee) Des-Arg bradykinin; (ff) B-lymphocyte chemoattractant (BLC); (gg) macrophage colony stimulating factor M-CSF; (hh) tumor necrosis factor α (TNFα); (ii) amyloid beta peptide (Aβ1-42); (jj) amyloid beta peptide (Aβ-6); (kk) human IgE; (ll) CCR5 extracellular domain; (mm) CXCR4 extracellular domain; (nn) Gastrin; (oo) CETP; (pp) C5a; (qq) epidermal growth factor receptor (EGF-R); (rr) CGRP; (ss) α-synuclein; (tt) calcitonin gene-related peptide (CGRP) (uu) Amylin; (vv) myostatin; (ww) interleukin-4; (xx) thymic stromal lymphopoietin; (yy) interleukin-33; (zz) interleukin-25; (aaa) interleukin-31; (bbb) epidermal growth factor (EGF); (ccc) nerve growth factor (NGF); and (ddd) a fragment of any one of the polypeptides (a) to (ccc); and (eee) an antigenic mutant or fragment of any one of the polypeptides (a) to (ccc).

In a further preferred embodiment said antigen is a self antigen, wherein said self antigen is a polypeptide selected from the group consisting of: (a) IgE, (b) IL-6 (c) receptor activator of nuclear factor kB ligand (RANKL); (d) vascular endothelial growth factor (VEGF); (e) vascular endothelial growth factor receptor (VEGF-R); (f) hepatocyte growth factor (HGF) (g) interleukin-1α; (h) interleukin-1β; (i) interleukin-5; (j) interleukin-8; (k) interleukin-13; (l) interleukin-15; (m) interleukin-17; (n) IL-23; (o) Ghrelin; (p) angiotensin; (q) chemokine (C-C motif) (CCL21); (r) chemokine (C-X motif) (CXCL 12); (s) stromal cell derived factor 1 (SDF-I); (t) monocyte chemotactic protein 1 (MCP-I); (u) endoglin; (v) resistin; (w) gonadotropin releasing hormone (GnRH); (x) growth hormone releasing (GHRH); (y) lutenizing hormone releasing hormone (LHRH); (z) thyreotropin releasing hormone (TRH); (aa) macrophage migration inhibitory factor (MIF); (bb) glucose-dependent insulinotropic peptide (GIP); (cc) eotaxin; (dd) bradykinin; (ee) Des-Arg bradykinin; (ff) B-lymphocyte chemoattractant (BLC); (gg) macrophage colony stimulating factor M-CSF; (hh) tumor necrosis factor α (TNFα); (ii) amyloid beta peptide (Aβ1-42); (jj) amyloid beta peptide (Aβ-6); (kk) human IgE; (ll) CCR5 extracellular domain; (mm) CXCR4 extracellular domain; (nn) Gastrin; (oo) CETP; (pp) C5a; (qq) epidermal growth factor receptor (EGF-R); (rr) CGRP; (ss) α-synuclein; (tt) calcitonin gene-related peptide (CGRP) (uu) Amylin; (vv) myostatin; (ww) interleukin-4; (xx) thymic stromal lymphopoietin; (yy) interleukin-33; (zz) interleukin-25; (aaa) interleukin-31; (bbb) epidermal growth factor (EGF); (ccc) nerve growth factor (NGF); and (ddd) a fragment of any one of the polypeptides (a) to (ccc); and (eee) an antigenic mutant or fragment of any one of the polypeptides (a) to (ccc).

In a preferred embodiment, said antigen is selected from canine interleukin 17 (cIL-17), feline interleukin 17 (fIL-17), equine interleukin 17 (eIL-17), bovine interleukin 17 (bIL-17) and porcine interleukin 17 (pIL-17), preferably feline interleukin 17 (fIL-17). In a preferred embodiment, said antigen comprises, or preferably consists of, of an amino acid sequence selected from SEQ ID NO:69, SEQ ID NO:70, SEQ ID NO:71, SEQ ID NO:72 and SEQ ID NO:73, or an amino acid sequence having a sequence identity of at least 90%, preferably of at least 95%, with any of SEQ ID NO:69, SEQ ID NO:70, SEQ ID NO:71, SEQ ID NO:72 and SEQ ID NO:73. Preferably, said antigen comprises. In another preferred embodiment, said antigen consists of SEQ ID NO:70. Furthermore, the inventive modified CMV VLPs are used in a method of treating an inflammatory disease, preferably a chronic inflammatory disease in an animal, further preferably in a cat.

In another preferred embodiment, said antigen is IL-5, preferably human, canine, feline, equine, bovine or porcine IL-5. In a preferred embodiment, said antigen is selected from human interleukin 5, canine interleukin 5 (cTL-5), feline interleukin 5 (fIL-5), equine interleukin 5 (eTL-5), bovine interleukin 5 (bTL-5) and porcine interleukin 5 (pTL-5), preferably canine interleukin 5 (cTL-5) or feline interleukin 5 (fIL-5), further preferably feline interleukin 5 (fIL-5). In a preferred embodiment, said antigen comprises, or preferably consists of, of an amino acid sequence selected from any of SEQ ID NO:35, SEQ ID NO:41, SEQ ID NO:42, SEQ ID NO:74, SEQ ID NO:75, SEQ ID NO:76, SEQ ID NO:77, SEQ ID NO:78, SEQ ID NO:79, SEQ ID NO:80, SEQ ID NO:81, and SEQ ID NO:82, or an amino acid sequence having a sequence identity of at least 90%, preferably of at least 95%, with any of SEQ ID NO:35, SEQ ID NO:41, SEQ ID NO:42, and SEQ ID NO:74 to SEQ ID NO:82.

In another preferred embodiment, said antigen is human IL-5. In again a further preferred embodiment, said antigen comprises, or preferably consists of SEQ ID NO:74. Furthermore, the inventive modified CMV VLPs comprising antigens of IL-5 are used in a method of treating an inflammatory disease, preferably a chronic inflammatory disease in an animal or human. Preferably, said inflammatory disease is selected from RA, MS, Psoriasis, asthma, Crohns, Colitis, COPD, diabetes, neurodermatitis (allergic dermatitis), eosinophilic granulomatosis, feline atopic skin syndrome and insect bite hypersensitivity.

In another preferred embodiment, said antigen is canine IL-5 (cTL-5). In again a further preferred embodiment, said antigen comprises, or preferably consists of, SEQ ID NO:75 or SEQ ID NO:76, or an amino acid sequence having a sequence identity of at least 90%, preferably of at least 95%, with SEQ ID NO:75 or SEQ ID NO:76. Preferably, said antigen comprises SEQ ID NO:75 or SEQ ID NO:76. In another preferred embodiment, said antigen consists of SEQ ID NO:75 or Seq ID NO:76.

In another preferred embodiment, said antigen is feline IL-5 (fIL-5). In a preferred embodiment, said antigen comprises, or preferably consists of, SEQ ID NO:35, SEQ ID NO:41, SEQ ID NO:42, SEQ ID NO:77, SEQ ID NO:78, SEQ ID:79 or an amino acid sequence having a sequence identity of at least 90%, preferably of at least 92%, further preferably of at least 95%, and again further preferably of at least 98% amino acid sequence identity with SEQ ID NO:35, SEQ ID NO:41, SEQ ID NO:42, SEQ ID NO:77, SEQ ID NO:78, SEQ ID:79. In a further preferred embodiment, said antigen comprises SEQ ID NO:35, SEQ ID NO:41, SEQ ID NO:42, SEQ ID NO:77, SEQ ID NO:78 or SEQ ID:79. In a further preferred embodiment, said antigen consists of SEQ ID NO:35, SEQ ID NO:41, SEQ ID NO:42, SEQ ID NO:77, SEQ ID NO:78 or SEQ ID:79. In a further preferred embodiment, said antigen comprises, or preferably consists of, SEQ ID NO:35 or an amino acid sequence having a sequence identity of at least 90%, preferably of at least 92%, further preferably of at least 95%, and again further preferably of at least 98% amino acid sequence identity with SEQ ID NO:35. In a further preferred embodiment, said antigen comprises, or preferably consists of, SEQ ID NO:41 or an amino acid sequence having a sequence identity of at least 90%, preferably of at least 92%, further preferably of at least 95%, and again further preferably of at least 98% amino acid sequence identity with SEQ ID NO:41. In a further very preferred embodiment, said antigen comprises SEQ ID NO:41. In a further very preferred embodiment, said antigen consists of SEQ ID NO:41. In a further preferred embodiment, said antigen comprises, or preferably consists of, SEQ ID NO:42 or an amino acid sequence having a sequence identity of at least 90%, preferably of at least 92%, further preferably of at least 95%, and again further preferably of at least 98% amino acid sequence identity with SEQ ID NO:42. In a further very preferred embodiment, said antigen comprises SEQ ID NO:42. In a further very preferred embodiment, said antigen consists of SEQ ID NO:42. In a further preferred embodiment, said antigen comprises, or preferably consists of, SEQ ID NO:77 or an amino acid sequence having a sequence identity of at least 90%, preferably of at least 92%, further preferably of at least 95%, and again further preferably of at least 98% amino acid sequence identity with SEQ ID NO:77.

In another preferred embodiment, said antigen is equine IL-5 (eIL-5). In a preferred embodiment, said antigen comprises, or preferably consists of, SEQ ID NO:80 or an amino acid sequence having a sequence identity of at least 90%, preferably of at least 92%, further preferably of at least 95%, and again further preferably of at least 98% amino acid sequence identity with SEQ ID NO:80.

In another preferred embodiment, said antigen is IL-4, preferably human Il-4. In again a further preferred embodiment, said antigen comprises SEQ ID NO:83. In another preferred embodiment, said antigen consists of SEQ ID NO:83.

In another preferred embodiment, said antigen is canine IL-4. In again a further preferred embodiment, said antigen comprises, or preferably consists of, SEQ ID NO:84 or an amino acid sequence having a sequence identity of at least 90%, preferably of at least 95%, with SEQ ID NO:84. Preferably, said antigen comprises SEQ ID NO:84. In another preferred embodiment, said antigen consists of SEQ ID NO:84.

In another preferred embodiment, said antigen is feline IL-4. In again a further preferred embodiment, said antigen comprises, or preferably consists of SEQ ID NO:85 or an amino acid sequence having a sequence identity of at least 90%, preferably of at least 95%, with SEQ ID NO:85. Preferably, said antigen comprises SEQ ID NO:85. In another preferred embodiment, said antigen consists of SEQ ID NO:85.

In another preferred embodiment, said antigen is equine IL-4.

In another preferred embodiment, said antigen is IL-13, preferably human IL-13. In a preferred embodiment, said antigen is selected from human interleukin 13, canine interleukin 13 (cIL-13), feline interleukin 13 (fIL-13), equine interleukin 13 (eIL-13), bovine interleukin 13 (bIL-13) and porcine interleukin 13 (pIL-13), preferably canine interleukin 13 (cIL-13) or feline interleukin 13 (fIL-13), further preferably feline interleukin 13 (fIL-13). In a preferred embodiment, said antigen comprises, or preferably consists of, of an amino acid sequence selected from any of SEQ ID NO:86, SEQ ID NO:87, SEQ ID NO:88, SEQ ID NO:89, SEQ ID NO:90, SEQ ID NO:91, SEQ ID NO:92 and SEQ ID NO:93, or an amino acid sequence having a sequence identity of at least 90%, preferably of at least 95%, with any of SEQ ID NO:86 to SEQ ID NO:93. Furthermore, the inventive modified CMV VLPs comprising antigens of IL-13 are used in a method of treating an inflammatory disease, preferably an allergic inflammation, allergic lung disease, asthma or atopic dermatitis.

In again a further preferred embodiment, said antigen comprises, or preferably consists of SEQ ID NO:86. In another preferred embodiment, said antigen consists of SEQ ID NO:86.

In another preferred embodiment, said antigen is canine IL-13. In again a further preferred embodiment, said antigen comprises, or preferably consists of SEQ ID NO:87 or an amino acid sequence having a sequence identity of at least 90%, preferably of at least 95%, with SEQ ID NO:87. Preferably, said antigen comprises, or preferably consists of SEQ ID NO:87. In another preferred embodiment, said antigen consists of SEQ ID NO:87.

In another preferred embodiment, said antigen is feline IL-13. In again a further preferred embodiment, said antigen comprises, or preferably consists of SEQ ID NO:88, SEQ ID NO:89, or an amino acid sequence having a sequence identity of at least 90%, preferably of at least 95%, with SEQ ID NO:88 or SEQ ID NO:89. Preferably, said antigen comprises, or preferably consists of SEQ ID NO:88 or SEQ ID NO: 89. In another preferred embodiment, said antigen consists of SEQ ID NO:88 or SEQ ID NO: 89.

In another preferred embodiment, said antigen is equine IL-13. In again a further preferred embodiment, said antigen comprises, or preferably consists of SEQ ID NO:90, SEQ ID NO:91 or an amino acid sequence having a sequence identity of at least 90%, preferably of at least 95%, with SEQ ID NO:90 or SEQ ID NO:91. Preferably, said antigen comprises, or preferably consists of SEQ ID NO:90 or SEQ ID NO:91. In another preferred embodiment, said antigen consists of SEQ ID NO:90 or SEQ ID NO:91.

In a further preferred embodiment, said antigen is TNFα. Furthermore, the inventive modified CMV VLPs comprising antigens of TNFα are used in a method of treating an inflammatory disease, preferably multisystem inflammatory diseases, rheumatoid arthritis, inflammatory bowel disease, Crohn's disease, ulcerative colitis, psoriasis, psoriatic arthritis, juvenile idiopathic arthritis or ankylosing spondylitis.

In another preferred embodiment, said antigen is IL-1α, preferably human IL-1α. In a preferred embodiment, said antigen is selected from human interleukin 1α, canine interleukin 1α (cIL-1α), feline interleukin 1α (fIL-1α), equine interleukin 1α (eIL-1α), bovine interleukin 1α (bIL-1α) and porcine interleukin 1α (pIL-1α), preferably canine interleukin 1α (cIL-1α) or feline interleukin 1α (fIL-1α), further preferably feline interleukin 1α (fIL-1α). In a preferred embodiment, said antigen comprises, or preferably consists of, of an amino acid sequence selected from any of SEQ ID NO:94, SEQ ID NO:95, SEQ ID NO:96, SEQ ID NO:97, SEQ ID NO:98, SEQ ID NO:99, SEQ ID NO:100, SEQ ID NO:101 and SEQ ID NO:102, or an amino acid sequence having a sequence identity of at least 90%, preferably of at least 95%, with any of SEQ ID NO:94 to SEQ ID NO:102. In again a further preferred embodiment, said antigen comprises SEQ ID NO:94 or SEQ ID NO:95. In another preferred embodiment, said antigen consists of SEQ ID NO:94 or SEQ ID NO:95.

In another preferred embodiment, said antigen is canine IL-1α. In again a further preferred embodiment, said antigen comprises, or preferably consists of SEQ ID NO:96 or an amino acid sequence having a sequence identity of at least 90%, preferably of at least 95%, with SEQ ID NO:96. Preferably, said antigen comprises SEQ ID NO:96. In another preferred embodiment, said antigen consists of SEQ ID NO:96.

In another preferred embodiment, said antigen is feline IL-1α. In again a further preferred embodiment, said antigen comprises, or preferably consists of SEQ ID NO:97 or SEQ ID NO:98, or an amino acid sequence having a sequence identity of at least 90%, preferably of at least 95%, with SEQ ID NO:97 or SEQ ID NO:98. Preferably, said antigen comprises SEQ ID NO:97 or SEQ ID NO:98. In another preferred embodiment, said antigen consists of SEQ ID NO:97 or SEQ ID NO:98.

In another preferred embodiment, said antigen is equine IL-1α. In again a further preferred embodiment, said antigen comprises, or preferably consists of SEQ ID NO:99 or SEQ ID NO:100, or an amino acid sequence having a sequence identity of at least 90%, preferably of at least 95%, with SEQ ID NO:99 or SEQ ID NO:100. Preferably, said antigen comprises SEQ ID NO:99 or SEQ ID NO:100. In another preferred embodiment, said antigen consists of SEQ ID NO:99 or SEQ ID NO:100.

In another preferred embodiment, said antigen is IL-33, preferably human IL-33. Furthermore, the inventive modified CMV VLPs comprising antigens of IL-33 are used in a method of treating an inflammatory disease, preferably atopic dermatitis, asthma, a cardiovascular disease, a musculoskeletal disease, inflammatory bowel disease, or an allergy such as food allergy, or cancer or Alzheimer disease. In again a further very preferred embodiment, said antigen comprises, or preferably consists of SEQ ID NO: 103 or SEQ ID NO:104. In another preferred embodiment, said antigen consists of SEQ ID NO:103 or SEQ ID NO: 104.

In another preferred embodiment, said antigen is canine IL-33. In a preferred embodiment, said antigen comprises, or preferably consists of any one of SEQ ID NO:105, SEQ ID NO:106, SEQ ID NO:107, SEQ ID NO:108 or SEQ ID NO:109, or an amino acid sequence having a sequence identity of at least 90%, preferably of at least 95%, with any one of SEQ ID NO:105, SEQ ID NO:106, SEQ ID NO:107, SEQ ID NO:108 or SEQ ID NO:109. Preferably, said antigen comprises any one of SEQ ID NO:105, SEQ ID NO: 106, SEQ ID NO: 107, SEQ ID NO:108 or SEQ ID NO: 109. In another preferred embodiment, said antigen consists any one of SEQ ID NO:105, SEQ ID NO:106, SEQ ID NO: 107, SEQ ID NO:108 or SEQ ID NO: 109. In a preferred embodiment, said antigen comprises, or preferably consists of SEQ ID NO:105 or an amino acid sequence having a sequence identity of at least 90%, preferably of at least 95%, with SEQ ID NO:105. Preferably, said antigen comprises SEQ ID NO:105. In another preferred embodiment, said antigen consists of SEQ ID NO:105. In a preferred embodiment, said antigen comprises, or preferably consists of SEQ ID NO:108 or an amino acid sequence having a sequence identity of at least 90%, preferably of at least 95%, with SEQ ID NO:108. Preferably, said antigen comprises SEQ ID NO: 108. In another preferred embodiment, said antigen consists of SEQ ID NO:108. In a preferred embodiment, said antigen comprises, or preferably consists of SEQ ID NO: 109 or an amino acid sequence having a sequence identity of at least 90%, preferably of at least 95%, with SEQ ID NO:109. Preferably, said antigen comprises SEQ ID NO:109. In another preferred embodiment, said antigen consists of SEQ ID NO:109.

In another preferred embodiment, said antigen is feline IL-33. In a preferred embodiment, said antigen comprises, or preferably consists of SEQ ID NO:110 or SEQ ID NO:111 or an amino acid sequence having a sequence identity of at least 90%, preferably of at least 95%, with SEQ ID NO:110 or SEQ ID NO:111. Preferably, said antigen comprises SEQ ID NO:110 or SEQ ID NO:111. In another preferred embodiment, said antigen consists of SEQ ID NO:110 or SEQ ID NO:111.

In another preferred embodiment, said antigen is equine IL-33. In a preferred embodiment, said antigen comprises, or preferably consists of SEQ ID NO: 112 or an amino acid sequence having a sequence identity of at least 90%, preferably of at least 95%, with SEQ ID NO: 112. Preferably, said antigen comprises SEQ ID NO:112. In another preferred embodiment, said antigen consists of SEQ ID NO: 112.

In another preferred embodiment, said antigen is IL-25, preferably human IL-25. In again a further preferred embodiment, said antigen comprises SEQ ID NO:113, or an amino acid sequence having a sequence identity of at least 90%, preferably of at least 95%, with SEQ ID NO:113. Preferably, said antigen comprises SEQ ID NO:113. In another preferred embodiment, said antigen consists of SEQ ID NO: 113.

In another preferred embodiment, said antigen is canine IL-25. In again a further preferred embodiment, said antigen comprises, or preferably consists of SEQ ID NO: 114 or an amino acid sequence having a sequence identity of at least 90%, preferably of at least 95%, with SEQ ID NO:114. Preferably, said antigen comprises SEQ ID NO:114. In another preferred embodiment, said antigen consists of SEQ ID NO:114.

In another preferred embodiment, said antigen is feline IL-25. In again a further preferred embodiment, said antigen comprises, or preferably consists of SEQ ID NO: 115 or an amino acid sequence having a sequence identity of at least 90%, preferably of at least 95%, with SEQ ID NO:115. Preferably, said antigen comprises SEQ ID NO:115. In another preferred embodiment, said antigen consists of SEQ ID NO:115.

In another preferred embodiment, said antigen is equine IL-25. In again a further preferred embodiment, said antigen comprises, or preferably consists of SEQ ID NO: 116 or an amino acid sequence having a sequence identity of at least 90%, preferably of at least 95%, with SEQ ID NO:116. Preferably, said antigen comprises SEQ ID NO:116. In another preferred embodiment, said antigen consists of SEQ ID NO:116.

In a further preferred embodiment, said antigen is IL-1β, preferably human IL-1β, canine IL-1β, feline IL-1β and equine IL-1β. Furthermore, the inventive modified CMV VLPs comprising antigens of IL-1β are used in a method of treating an inflammatory disease, preferably multisystem inflammatory diseases associated with inflammasome dysregulation including osteoarthritis, juvenile idiopathic arthritis, Familial Mediterranean Fever, cryopyrin associated periodic syndrome, Muckle-Wells Syndrome, hyperimmunoglobulin D syndrome, Stills disease, gouty arthritis, rheumatoid arthritis, chronic obstructive pulmonary disease and coronary artery disease.

In another preferred embodiment, said antigen is IL-1β, preferably human, canine, feline, equine, bovine or porcine IL-1β. In a preferred embodiment, said antigen is selected from human interleukin 1β, canine interleukin 1β (cIL-1β), feline interleukin 1β (fIL-1β), equine interleukin 1β (eIL-1β), bovine interleukin 1β (bIL-1β) and porcine interleukin 1β (pIL-1β), preferably canine interleukin 1β (cIL-1β) or feline interleukin 1β (fIL-1β), further preferably canine interleukin 1β (cIL-1β). In a preferred embodiment, said antigen comprises, or preferably consists of, of an amino acid sequence selected from any of SEQ ID NO:44, SEQ ID NO:45, SEQ ID NO:117, SEQ ID NO:118, SEQ ID NO:119, SEQ ID NO: 120, SEQ ID NO:121, SEQ ID NO:122, SEQ ID NO:123, SEQ ID NO:124, SEQ ID NO:171, or an amino acid sequence having a sequence identity of at least 90%, preferably of at least 95%, with any of SEQ ID NO:44, SEQ ID NO:45, SEQ ID NO:117 to SEQ ID NO:124 or SEQ ID NO:171.

In a preferred embodiment, said antigen is human 1β. In a preferred embodiment, said antigen comprises SEQ ID NO:117 or SEQ ID NO:118. In again a further preferred embodiment, said antigen consists of SEQ ID NO:117 or SEQ ID NO:118.

In a further very preferred embodiment, said antigen is canine IL-1β. In a very preferred embodiment, said antigen comprises, or preferably consists of, SEQ ID NO:44, SEQ ID NO:45, SEQ ID NO:119, SEQ ID NO:120 or an amino acid sequence having a sequence identity of at least 90%, preferably of at least 92%, further preferably of at least 95%, and again further preferably of at least 98% amino acid sequence identity with SEQ ID NO:44, SEQ ID NO:45, SEQ ID NO:119, SEQ ID NO:120. In a further preferred embodiment, said antigen comprises SEQ ID NO:44, SEQ ID NO:45, SEQ ID NO:119 or SEQ ID NO:120. In a further preferred embodiment, said antigen consists of SEQ ID NO:44, SEQ ID NO:45, SEQ ID NO:119 or SEQ ID NO:120. In a further very preferred embodiment, said antigen comprises, or preferably consists of, SEQ ID NO:44 or an amino acid sequence having a sequence identity of at least 90%, preferably of at least 92%, further preferably of at least 95%, and again further preferably of at least 98% amino acid sequence identity with SEQ ID NO:44. In a further very preferred embodiment, said antigen comprises SEQ ID NO:44. In a further very preferred embodiment, said antigen consists of SEQ ID NO:44. In a further preferred embodiment, said antigen comprises, or preferably consists of, SEQ ID NO:45 or an amino acid sequence having a sequence identity of at least 90%, preferably of at least 92%, further preferably of at least 95%, and again further preferably of at least 98% amino acid sequence identity with SEQ ID NO:45. In a further very preferred embodiment, said antigen comprises SEQ ID NO:45. In a further very preferred embodiment, said antigen consists of SEQ ID NO:45. In a further preferred embodiment, said antigen comprises, or preferably consists of, SEQ ID NO: 119 or an amino acid sequence having a sequence identity of at least 90%, preferably of at least 92%, further preferably of at least 95%, and again further preferably of at least 98% amino acid sequence identity with SEQ ID NO:119. In a further very preferred embodiment, said antigen comprises SEQ ID NO:119. In a further very preferred embodiment, said antigen consists of SEQ ID NO:119. In a further very preferred embodiment, said antigen comprises, or preferably consists of, SEQ ID NO:120 or an amino acid sequence having a sequence identity of at least 90%, preferably of at least 92%, further preferably of at least 95%, and again further preferably of at least 98% amino acid sequence identity with SEQ ID NO:120. In a further very preferred embodiment, said antigen comprises SEQ ID NO:120. In a further very preferred embodiment, said antigen consists of SEQ ID NO: 120.

In a further very preferred embodiment, said antigen is feline IL-1β. In a further very preferred embodiment, said antigen comprises, or preferably consists of, SEQ ID NO:121, SEQ ID NO:171 or an amino acid sequence having a sequence identity of at least 90%, preferably of at least 92%, further preferably of at least 95%, and again further preferably of at least 98% amino acid sequence identity with SEQ ID NO:121 or SEQ ID NO:171. In a further very preferred embodiment, said antigen comprises, or preferably consists of, SEQ ID NO:121 or an amino acid sequence having a sequence identity of at least 90%, preferably of at least 92%, further preferably of at least 95%, and again further preferably of at least 98% amino acid sequence identity with SEQ ID NO:121. In a further very preferred embodiment, said antigen comprises, or preferably consists of, SEQ ID NO:171 or an amino acid sequence having a sequence identity of at least 90%, preferably of at least 92%, further preferably of at least 95%, and again further preferably of at least 98% amino acid sequence identity with SEQ ID NO:171. In a further very preferred embodiment, said antigen comprises SEQ ID NO:121 or SEQ ID NO:171. In a further very preferred embodiment, said antigen consists of SEQ ID NO:121 or SEQ ID NO:171. In a further very preferred embodiment, said antigen comprises SEQ ID NO:121. In a further very preferred embodiment, said antigen consists of SEQ ID NO:121. In a further very preferred embodiment, said antigen comprises SEQ ID NO:171. In a further very preferred embodiment, said antigen consists of SEQ ID NO:171.

In a further very preferred embodiment, said antigen is equine IL-1β. In a further very preferred embodiment, said antigen comprises, or preferably consists of, SEQ ID NO:122 or an amino acid sequence having a sequence identity of at least 90%, preferably of at least 92%, further preferably of at least 95%, and again further preferably of at least 98% amino acid sequence identity with SEQ ID NO:122. In a further very preferred embodiment, said antigen comprises SEQ ID NO: 122. In a further very preferred embodiment, said antigen consists of SEQ ID NO:122.

In a further preferred embodiment, said antigen is bovine IL-1β. In a further very preferred embodiment, said antigen comprises, or preferably consists of, SEQ ID NO:123 or an amino acid sequence having a sequence identity of at least 90%, preferably of at least 92%, further preferably of at least 95%, and again further preferably of at least 98% amino acid sequence identity with SEQ ID NO:123. In a further very preferred embodiment, said antigen comprises SEQ ID NO: 123. In a further very preferred embodiment, said antigen consists of SEQ ID NO:123.

In a further very preferred embodiment, said antigen is porcine IL-1β. In a further very preferred embodiment, said antigen comprises, or preferably consists of, SEQ ID NO:124 or an amino acid sequence having a sequence identity of at least 90%, preferably of at least 92%, further preferably of at least 95%, and again further preferably of at least 98% amino acid sequence identity with SEQ ID NO:124. In a further very preferred embodiment, said antigen comprises SEQ ID NO: 124. In a further very preferred embodiment, said antigen consists of SEQ ID NO:124.

In a further preferred embodiment, said antigen is IL-12/23, preferably human IL-12/23. In a further preferred embodiment, said antigen is canine IL-12/23. In a further preferred embodiment, said antigen is feline IL-12/23. In a further preferred embodiment, said antigen is equine IL-12/23.

In another preferred embodiment, said antigen is IL-31, preferably human, canine and feline IL-31. Furthermore, the inventive modified CMV VLPs comprising antigens of IL-31 are used in a method of treating an inflammatory disease, preferably atopic dermatitis, bullous pemphigoid, chronic urticaria or asthma. In a preferred embodiment, said antigen is selected from human interleukin 31, canine interleukin 31 (cIL-31), feline interleukin 1β (fIL-31), equine interleukin 31 (eIL-31), bovine interleukin 31 (bIL-31) and porcine interleukin 31 (pIL-31), preferably canine interleukin 31 (cIL-31) or feline interleukin 31 (fIL-31), further preferably canine interleukin 31 (cIL-31). In a preferred embodiment, said antigen comprises, or preferably consists of, of an amino acid sequence selected from any of SEQ ID NO:125, SEQ ID NO:126, SEQ ID NO:127, SEQ ID NO:128, SEQ ID NO:129, SEQ ID NO:130, SEQ ID NO:131 and SEQ ID NO:132, or an amino acid sequence having a sequence identity of at least 90%, preferably of at least 95%, with any of SEQ ID NO:125 to SEQ ID NO:132.

In another preferred embodiment, said antigen is human IL-31. In again a further preferred embodiment, said antigen comprises, or preferably consists of SEQ ID NO: 125 or an amino acid sequence having a sequence identity of at least 90%, preferably of at least 95%, with SEQ ID NO:125. Preferably, said antigen comprises SEQ ID NO:125. In another preferred embodiment, said antigen consists of SEQ ID NO:125.

In another preferred embodiment, said antigen is canine IL-31. In again a further preferred embodiment, said antigen comprises, or preferably consists of SEQ ID NO: 126 or an amino acid sequence having a sequence identity of at least 90%, preferably of at least 95%, with SEQ ID NO:126. Preferably, said antigen comprises SEQ ID NO:126. In another preferred embodiment, said antigen consists of SEQ ID NO:126.

In another preferred embodiment, said antigen is feline IL-31. In again a further preferred embodiment, said antigen comprises, or preferably consists of SEQ ID NO: 127, SEQ ID NO:128 or an amino acid sequence having a sequence identity of at least 90%, preferably of at least 95%, with SEQ ID NO:127, SEQ ID NO:128. Preferably, said antigen comprises, or preferably consists of SEQ ID NO: 127, SEQ ID NO:128. In another preferred embodiment, said antigen consists of SEQ ID NO: 127, SEQ ID NO:128.

In another preferred embodiment, said antigen is equine IL-31. In again a further preferred embodiment, said antigen comprises, or preferably consists of SEQ ID NO: 129, SEQ ID NO:130 or an amino acid sequence having a sequence identity of at least 90%, preferably of at least 95%, with SEQ ID NO:129, SEQ ID NO:130. Preferably, said antigen comprises, or preferably consists of SEQ ID NO: 129, SEQ ID NO:130. In another preferred embodiment, said antigen consists of SEQ ID NO: 129, SEQ ID NO:130.

In another preferred embodiment, said antigen is bovine IL-31. In again a further preferred embodiment, said antigen comprises, or preferably consists of SEQ ID NO: 131 or an amino acid sequence having a sequence identity of at least 90%, preferably of at least 95%, with SEQ ID NO:131. Preferably, said antigen comprises SEQ ID NO:131. In another preferred embodiment, said antigen consists of SEQ ID NO:131.

In another preferred embodiment, said antigen is porcine IL-31. In again a further preferred embodiment, said antigen comprises, or preferably consists of SEQ ID NO: 132 or an amino acid sequence having a sequence identity of at least 90%, preferably of at least 95%, with SEQ ID NO:132. Preferably, said antigen comprises SEQ ID NO:132. In another preferred embodiment, said antigen consists of SEQ ID NO:132.

In another preferred embodiment, said antigen is thymic stromal lymphopoietin (TLSP), preferably human, canine and feline thymic stromal lymphopoietin (TLSP).

In another preferred embodiment, said antigen is human TLSP. In again a further preferred embodiment, said antigen comprises, or preferably consists of SEQ ID NO: 133 or an amino acid sequence having a sequence identity of at least 90%, preferably of at least 95%, with SEQ ID NO:133. Preferably, said antigen comprises SEQ ID NO:133. In another preferred embodiment, said antigen consists of SEQ ID NO:133.

In another preferred embodiment, said antigen is canine TLSP. In again a further preferred embodiment, said antigen comprises, or preferably consists of SEQ ID NO: 134 or an amino acid sequence having a sequence identity of at least 90%, preferably of at least 95%, with SEQ ID NO:134. Preferably, said antigen comprises SEQ ID NO:134. In another preferred embodiment, said antigen consists of SEQ ID NO:134.

In another preferred embodiment, said antigen is feline TLSP. In again a further preferred embodiment, said antigen comprises, or preferably consists of SEQ ID NO: 135, SEQ ID NO:136 or an amino acid sequence having a sequence identity of at least 90%, preferably of at least 95%, with SEQ ID NO:135, SEQ ID NO:136. Preferably, said antigen comprises, or preferably consists of SEQ ID NO:135, SEQ ID NO:136. In another preferred embodiment, said antigen consists of SEQ ID NO:135, SEQ ID NO:136.

In another preferred embodiment, said antigen is equine TLSP. In again a further preferred embodiment, said antigen comprises, or preferably consists of SEQ ID NO: 137 or an amino acid sequence having a sequence identity of at least 90%, preferably of at least 95%, with SEQ ID NO:137. Preferably, said antigen comprises SEQ ID NO:137. In another preferred embodiment, said antigen consists of SEQ ID NO:137.

In another very preferred embodiment, said antigen is nerve growth factor (NGF), preferably human, canine, feline, equine, bovine or porcine NGF. In a preferred embodiment, said antigen is selected from human NGF, canine NGF (cNGF), feline NGF (fNGF), equine NGF (eNGF), bovine NGF (bNGF) and porcine NGF (pNGF), preferably canine NGF (cNGF) or feline NGF (fNGF), and wherein further preferably said antigen is canine NGF (cNGF). In a preferred embodiment, said antigen comprises, or preferably consists of, of an amino acid sequence selected from any of SEQ ID NO:30, SEQ ID NO:31, SEQ ID NO:138, SEQ ID NO:139, SEQ ID NO:140, SEQ ID NO:141, and SEQ ID NO:142, or an amino acid sequence having a sequence identity of at least 90%, preferably of at least 95%, with any of SEQ ID NO:30, SEQ ID NO:31, SEQ ID NO:138, SEQ ID NO:139, SEQ ID NO:140, SEQ ID NO:141, and SEQ ID NO:142.

In a further preferred embodiment, said antigen is human NGF. In a further very preferred embodiment, said antigen comprises, or preferably consists of, SEQ ID NO:138 or an amino acid sequence having a sequence identity of at least 90%, preferably of at least 92%, further preferably of at least 95%, and again further preferably of at least 98% amino acid sequence identity with SEQ ID NO:138. In a further very preferred embodiment, said antigen comprises SEQ ID NO:138. In a further very preferred embodiment, said antigen consists of SEQ ID NO:138.

In a further very preferred embodiment, said antigen is canine NGF. In a very preferred embodiment, said antigen comprises, or preferably consists of, SEQ ID NO:30, SEQ ID NO:31, or an amino acid sequence having a sequence identity of at least 90%, preferably of at least 92%, further preferably of at least 95%, and again further preferably of at least 98% amino acid sequence identity with SEQ ID NO:30 or SEQ ID NO:31. In a further preferred embodiment, said antigen comprises SEQ ID NO:30 or SEQ ID NO:31. In a further preferred embodiment, said antigen consists of SEQ ID NO:30 or SEQ ID NO:31. In a further very preferred embodiment, said antigen comprises, or preferably consists of, SEQ ID NO:30 or an amino acid sequence having a sequence identity of at least 90%, preferably of at least 92%, further preferably of at least 95%, and again further preferably of at least 98% amino acid sequence identity with SEQ ID NO:30. In a further very preferred embodiment, said antigen comprises SEQ ID NO:30. In a further very preferred embodiment, said antigen consists of SEQ ID NO:30. In a further preferred embodiment, said antigen comprises, or preferably consists of, SEQ ID NO:31 or an amino acid sequence having a sequence identity of at least 90%, preferably of at least 92%, further preferably of at least 95%, and again further preferably of at least 98% amino acid sequence identity with SEQ ID NO:31. In a further very preferred embodiment, said antigen comprises SEQ ID NO:31. In a further very preferred embodiment, said antigen consists of SEQ ID NO:31.

In a further very preferred embodiment, said antigen is feline NGF. In a further very preferred embodiment, said antigen comprises, or preferably consists of, SEQ ID NO:139 or an amino acid sequence having a sequence identity of at least 90%, preferably of at least 92%, further preferably of at least 95%, and again further preferably of at least 98% amino acid sequence identity with SEQ ID NO:139. In a further very preferred embodiment, said antigen comprises SEQ ID NO: 139. In a further very preferred embodiment, said antigen consists of SEQ ID NO:139.

In a further preferred embodiment, said antigen is equine NGF. In a further very preferred embodiment, said antigen comprises, or preferably consists of, SEQ ID NO:140 or an amino acid sequence having a sequence identity of at least 90%, preferably of at least 92%, further preferably of at least 95%, and again further preferably of at least 98% amino acid sequence identity with SEQ ID NO:140. In a further very preferred embodiment, said antigen comprises SEQ ID NO: 140. In a further very preferred embodiment, said antigen consists of SEQ ID NO:140.

In a further preferred embodiment, said antigen is bovine NGF. In a further very preferred embodiment, said antigen comprises, or preferably consists of, SEQ ID NO:141 or an amino acid sequence having a sequence identity of at least 90%, preferably of at least 92%, further preferably of at least 95%, and again further preferably of at least 98% amino acid sequence identity with SEQ ID NO:141. In a further very preferred embodiment, said antigen comprises SEQ ID NO:141. In a further very preferred embodiment, said antigen consists of SEQ ID NO:141.

In a further very preferred embodiment, said antigen is porcine NGF. In a further very preferred embodiment, said antigen comprises, or preferably consists of, SEQ ID NO:142 or an amino acid sequence having a sequence identity of at least 90%, preferably of at least 92%, further preferably of at least 95%, and again further preferably of at least 98% amino acid sequence identity with SEQ ID NO:142. In a further very preferred embodiment, said antigen comprises SEQ ID NO: 142. In a further very preferred embodiment, said antigen consists of SEQ ID NO:142.

In again a further preferred embodiment, said antigen is IgE or a peptide or domain comprised in IgE.

In again a further preferred embodiment, said antigen is a peptide derived the N-terminus from Aβ-1-42 (SEQ ID NO:143), in particular a fragment of Aβ-1-42 (SEQ ID NO:143) of at most 7 consecutive amino acids in length, preferably a fragment of Aβ-1-42 (SEQ ID NO:143) of at most 6 consecutive amino acids in length. Thus, in a further preferred embodiment, said antigen is selected from Aβ-1-6 (SEQ ID NO: 144), Aβ-1-7 (SEQ ID NO:145), Aβ-3-6 (SEQ ID NO:146), Aβ-1-5 (SEQ ID NO:147), Aβ-2-6 (SEQ ID NO:148), or Aβ-3-7 (SEQ ID NO:149).

In another preferred embodiment, said antigen is α-synuclein or a peptide derived from α-synuclein, and wherein preferably said peptide consists of 6 to 14 amino acids, and wherein further preferably said antigen is a peptide derived from α-synuclein selected from any one of SEQ D NO:150, SEQ ID NO:151, SEQ ID NO:152 and SEQ ID NO:153. Further preferred peptides derived from α-synuclein are disclosed in WO 2011/020133, which is incorporated herein by way of reference.

Alpha-synuclein (α-Syn), a small protein with multiple physiological and pathological functions, is one of the dominant proteins found in Lewy Bodies, a pathological hallmark of Lewy body disorders, including Parkinson's disease (PD). More recently, α-Syn has been found in body fluids, including blood and cerebrospinal fluid, and is likely produced by both peripheral tissues and the central nervous system. Exchange of α-Syn between the brain and peripheral tissues could have important pathophysiologic and therapeutic implications (Gardai S J et al., PLoS ONE (2013) 8(8): e71634). The evidence implicating alpha-synuclein (a-syn) in the pathogenesis of Parkinson's Disease (PD) is overwhelming.

Thus, in a further preferred embodiment, said antigen is selected from any one of the sequences selected from SEQ D NO:150, SEQ ID NO:151, SEQ ID NO:152 and SEQ ID NO:153. In a further preferred embodiment, said antigen is SEQ D NO:150. In a further preferred embodiment, said antigen is SEQ D NO:151. In a further preferred embodiment, said antigen is SEQ D NO:152. In a further preferred embodiment, said antigen is SEQ D NO:153.

In again a further preferred embodiment, said antigen is Amylin.

In a further preferred embodiment, said antigen is derived from African Swine Fever (ASF) protein useful to address African Swine Fever infections. In a preferred embodiment, said antigen comprises, preferably is, SEQ ID NO: 154.

In a further preferred embodiment, said antigen is Gonadotropin Releasing Hormone (GnRH). In one preferred embodiment, the antigen is GnRH or a fragment thereof. Such fragments useful in the production of modified CMV VLPs and vaccines in accordance with the present invention are disclosed in WO2006/027300, which is incorporated herein by reference in its entirety. In a preferred embodiment, said antigen comprises, preferably is, SEQ ID NO:155 or SEQ ID NO:156. In a further preferred embodiment, the N-terminal glutamic acid of SEQ ID NO:155 is a pyroglutamic acid (pGlu or pE).

This modified CMV VLP comprising antigens derived from GnRH can be used to address boar taint, fertility and behavior management. Thus, this modified CMV VLP comprising antigens derived from GnRH can be administered to a mammal, such as pig to prevent the boar taint in the meat. This modified CMV VLP comprising GnRH can be administered to an animal, such as dog, cat, sheep, cattle, horse to control their behaviour and/or to reduce their reproductivity. This modified CMV VLP comprising GnRH can be administered to human having gonadal steroid hormone dependent cancers. Moreover, this modified CMV VLP comprising GnRH can be administered to an animal or human to lower steroid hormone, preferably testosterone, levels in an animal or human.

In a preferred embodiment, said antigen is angiotensin I or a peptide derived from angiotensin I. In another preferred embodiment, said antigen is angiotensin II or a peptide derived from angiotensin II.

Modified CMV VLP comprising angiotensin derived antigens are useful for the treatment of diseases or disorders associated with the renin-activated angiotensin system, and in particular for the treatment of diseases selected from the group consisting of hypertension and high blood pressure, stroke, infarction, congestive heart failure, kidney failure, preferably cat chronic kidney disease, and retinal hemorrhage. Such angiotensin derived antigens are disclosed in WO03031466, which is incorporated herein by reference in its entirety. In a preferred embodiment, said antigen comprises, preferably is, SEQ ID NO:157, SEQ ID NO:158 or SEQ ID NO:159.

In a further preferred embodiment, said antigen is eotaxin.

In another preferred embodiment, said antigen is myostatin, preferably cow myostatin. In again a further preferred embodiment, said antigen comprises, or preferably consists of SEQ ID NO:160 or an amino acid sequence having a sequence identity of at least 90%, preferably of at least 95%, with SEQ ID NO:160. Preferably, said antigen comprises SEQ ID NO:160. In another preferred embodiment, said antigen consists of SEQ ID NO:160.

In a further preferred embodiment said antigen is a polypeptide of a parasite, wherein preferably said pathogen is selected from the group consisting of: (a) Toxoplasma spp.; (b) Plasmodium falciparum; (c) Plasmodium vivax; (d) Plasmodium ovale; (e) Plasmodium malariae; (f) Leishmania; (g) Schistosoma and (h) Nematodes. Preferably, said antigen is derived from Plasmodium falciparum or Plasmodium Vivax (SEQ ID NO: 161).

In a further preferred embodiment, said antigen is a polypeptide of a bacterium, wherein preferably said bacterium is selected from the group consisting of: (a) Chlamydia (b) Streptococcus; (c) Pneumococcus; (d) Staphylococcus; (e) Salmonella; (f) Mycobacteria; (g) Clostridia (h) Vibrio (i) Yersinia (k) Meningococcus (1) Borrelia.

Lyme disease is the most prevalent tick-born disease in Europe and North America, with about 400,000 registered cases annually. Disease may have different complications—pain in joints, neurological disorders, symptoms like multiple sclerosis and arthritis. Although the disease can be cured with antibiotics, symptoms may persist for years even after antibiotic treatment. Currently, no vaccine against Lyme disease is available in the market. In 1998, SmithKline Beecham Biologicals (now part of GlaxoSmithKline) developed LYMErix anti-Lyme vaccine, but it was removed from the market due to complaints about side-effects and multiple lawsuit cases. Therefore, at a global scale there is a need for a new, efficient and safe anti-Lyme vaccine. Borrelia genus bacteria, which cause Lyme disease, have many different proteins, located on their surface, creating an immune response against which may kill the pathogen. This approach was used in the Lymerix vaccine, which consisted of outer surface protein OspA. Since then, several other surface proteins of Borrelia burgdorferi have been tried as vaccine candidates, but none of them have reached the market so far. Borrelia species produce a number of surface proteins, which help to evade the destruction of bacteria by the complement system of the host. So-called CRASPs (complement regulator-acquiring proteins) are able bind complement regulator factor H (CFH) and CFH-like protein-1 (CFHL-1), which both inhibit complement activation and formation of membrane attack complex. CspZ is one of CRASPs, being able to bind both CFH and CFHL-1. Therefore, anti-CspZ antibodies would not only mark the surface of bacteria for attack of the immune system, but also reduce the ability of bacteria to avoid the complement.

Thus, in another preferred embodiment, said antigen is CspZ protein from Borrelia burgdorferi. In a preferred embodiment, said antigen comprises, or preferably consists of SEQ ID NO:162 or an amino acid sequence having a sequence identity of at least 90%, preferably of at least 95%, with SEQ ID NO:162. Preferably, said antigen comprises, or preferably consists of SEQ ID NO: 162. In another very preferred embodiment, said antigen consists of SEQ ID NO:162.

In a preferred embodiment, said antigen comprises, or preferably consists of SEQ ID NO:163 or an amino acid sequence having a sequence identity of at least 90%, preferably of at least 95%, with SEQ ID NO:163. Preferably, said antigen comprises, or preferably consists of SEQ ID NO:163. In another preferred embodiment, said antigen consists of SEQ ID NO:163.

As a consequence, the inventive modified virus-like particle of CMV comprising CspZ protein as the antigen can be useful as vaccine for protecting from Lyme borreliosis.

In a further preferred embodiment said antigen is a viral antigen, wherein preferably said viral antigen is a polypeptide derived from a virus selected from the group consisting of: (a) Retrovirus, preferably HIV; (b) Influenza virus, preferably influenza A M2 extracellular domain or HA or HA globular domain; (c) a polypeptide of Hepatitis B virus, preferably preSl; (d) Hepatitis C virus; (e) HPV, preferably HPV16E7; (f) RSV; (g) Coronavirus, preferably SARS-CoV-1, SARS-CoV-2, MERS, further preferably SARS-CoV-2; (h) Flavivirus, preferably Dengue virus, Zika Virus, West Nile Virus and Hand Foot and Mouth Disease Virus, and further preferably ectodomain III (ED3) from E protein of Dengue fever virus serotype 1; (i) Alphavirus, preferably Chikungunya; (k) Herpesvirus, preferably CMV; (1) Rotavirus. In a further preferred embodiment, said antigen is the derived from RSV.

In a further very preferred embodiment, said antigen is the derived from Dengue virus. Dengue fever is a vector-borne tropical disease, caused by Dengue fever virus. Each year about 390 million cases occur worldwide. Symptoms include fever, headache, vomiting, pain in joints and muscle and characteristic skin rash. In rare cases illness progresses to Dengue haemorrhagic fever, which is a life threatening condition, causing around 40,000 deaths worldwide annually. The first and only dengue vaccine that successfully completed clinical development has been withdrawn from the market in many countries due to safety concerns. Therefore, there still is a need for a safe dengue vaccine.

Envelope (E) protein is found on the surface of mature dengue virus particles and it is composed of three ectodomains EDI, EDII, EDIII (ED3) and a transmembrane region. It has been shown previously that ED3 alone results in production of high levels of EDIII-specific neutralizing antibodies. Therefore, ED3 could be used in fusion with the tandem dimers leading to the inventive modified VLPs as an efficient vaccine.

Thus, in another preferred embodiment, said antigen is derived, preferably is, from ectodomain III (ED3) from E protein of Dengue fever virus. In another preferred embodiment, said antigen is derived from ectodomain III (ED3) from E protein of Dengue fever virus serotype 1. Thus, in another preferred embodiment, said antigen is ectodomain III (ED3) from E protein of Dengue fever virus serotype 1. In a preferred embodiment, said antigen comprises, or preferably consists of SEQ ID NO: 164 or an amino acid sequence having a sequence identity of at least 90%, preferably of at least 95%, with SEQ ID NO:164. Preferably, said antigen comprises, or preferably consists of SEQ ID NO:164. In another very preferred embodiment, said antigen consists of SEQ ID NO:164.

In a preferred embodiment, said antigen comprises, or preferably consists of SEQ ID NO:165 or an amino acid sequence having a sequence identity of at least 90%, preferably of at least 95%, with SEQ ID NO:165. Preferably, said antigen comprises, or preferably consists of SEQ ID NO:165. In another preferred embodiment, said antigen consists of SEQ ID NO:165.

In a further preferred embodiment, said antigen comprises, or preferably consists of, position 9 to 99, position 9 to 109 or position 9 to 112 of SEQ ID NO:165 or an amino acid sequence having a sequence identity of at least 90%, preferably of at least 95%, with SEQ ID NO:165. Preferably, said antigen comprises, or preferably consists of, position 9 to 99, position 9 to 109 or position 9 to 112 of SEQ ID NO:165. In another preferred embodiment, said antigen consists of position 9 to 99, position 9 to 109 or position 9 to 112 of SEQ ID NO:165.

In a preferred embodiment, said antigen is the extracellular domain of Influenza A virus M2 protein, or an antigenic fragment thereof. In a preferred embodiment said antigen comprises or preferably consists of the extracellular domain of the Influenza A virus M2 protein, wherein preferably said extracellular domain of the Influenza A virus M2 protein is SEQ ID NO:166. In another preferred embodiment, said antigen is the globular domain of Influenza virus. In another preferred embodiment, said antigen comprises the protease cleavage site of HA Influenza virus.

In a preferred embodiment, said antigen is a receptor binding domain (RBD) of a coronavirus (CoV), or a fragment thereof. In another preferred embodiment, said antigen is the receptor binding domain (RBD), preferably the receptor binding motif (RBM), of a spike (S) protein of a human coronavirus (HCoV), or a fragment thereof, wherein said HCoV is selected from SARS-CoV-2, SARS-CoV, MERS-CoV, HCoV-229E, HCoV-NL63, HCoV-OC43 and HCoV-HKU1, preferably from SARS-CoV-2, SARS-CoV and MERS-CoV, and again further preferably from SARS-CoV-2.

In a preferred embodiment, said antigen comprises, or preferably consists of the amino acid sequence selected from SEQ ID NO:167, SEQ ID NO:168, SEQ ID NO:169, SEQ ID NO:170, and an amino acid sequence having a sequence identity of at least 80%, preferably of at least 90%, further preferably of at least 95% with any of SEQ ID NO:167, SEQ ID NO:168, SEQ ID NO:169 and SEQ ID NO:170.

In a preferred embodiment, said antigen comprises, or preferably consists of SEQ ID NO:167 or an amino acid sequence having a sequence identity of at least 90%, preferably of at least 95%, with SEQ ID NO:167. Preferably, said antigen comprises, or preferably consists of SEQ ID NO:167. In another very preferred embodiment, said antigen consists of SEQ ID NO:167.

In a preferred embodiment, said antigen comprises, or preferably consists of SEQ ID NO:168 or an amino acid sequence having a sequence identity of at least 90%, preferably of at least 95%, with SEQ ID NO:168. Preferably, said antigen comprises, or preferably consists of SEQ ID NO:168. In another very preferred embodiment, said antigen consists of SEQ ID NO:168.

In a preferred embodiment, said antigen is a growth factor or a cytokine, wherein said growth factor is selected from vascular endothelial growth factor (VEGF), vascular endothelial growth factor receptor, hepatocyte growth factor (HGF), epidermal growth factor (EGF), epidermal growth factor receptor (EGF-R) and nerve growth factor (NGF), preferably said growth factor is nerve growth factor (NGF), and wherein said cytokine is selected from interleukin-6, interleukin-1α, interleukin-1β, interleukin-5, interleukin-8, interleukin-13 interleukin-15, interleukin-17, interleukin-23, chemokine (C-C motif) (CCL21), chemokine (C-X motif) (CXCL 12), interleukin-4, interleukin-33, interleukin-25 and interleukin-31, preferably said cytokine is selected from interleukin-1α, interleukin-1β, interleukin-5, interleukin-13, interleukin-17, and interleukin-31, and further preferably said cytokine is selected from interleukin-1β and interleukin-5.

In a preferred embodiment, said antigen is a growth factor or an interleukin, wherein said growth factor is selected from vascular endothelial growth factor (VEGF), vascular endothelial growth factor receptor, hepatocyte growth factor (HGF), epidermal growth factor (EGF), epidermal growth factor receptor (EGF-R) and nerve growth factor (NGF), preferably said growth factor is nerve growth factor (NGF), and wherein said interleukin is selected from interleukin-6, interleukin-1α, interleukin-1β, interleukin-5, interleukin-8, interleukin-13 interleukin-15, interleukin-17, interleukin-23, interleukin-4, interleukin-33, interleukin-25 and interleukin-31, preferably said interleukin is selected from interleukin-1α, interleukin-1β, interleukin-5, interleukin-13, interleukin-17, and interleukin-31, and further preferably said interleukin is selected from interleukin-1p and interleukin-5, again further preferably said interleukin is interleukin-1p.

Without being bound, we believe that undesired aggregation and formation of aggregated conjugated CMV VLPs can in particular be reduced and avoided for antigens having a higher isoelectric point, and thus for antigens, which under the conditions used for conjugation would have an overall positive charge. In a preferred embodiment, said antigen has an isoelectric point of above 6.5. In a preferred embodiment, said antigen has an isoelectric point above 6.5 and below 13.0, preferably below 12.5, and further preferably below 12.0. In a preferred embodiment, said antigen has an isoelectric point above 6.5, as determined by the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). In a preferred embodiment, said antigen has an isoelectric point above 6.5 and below 13.0, preferably below 12.5, and further preferably below 12.0, as determined by the ExPASy Compute pI/MW tool described by Gasteiger et al (Gasteiger, E., Hoogland, C., Gattiker, A., Duvaud, S., Wilkins, M. R., Appel, R. D., & Bairoch, A., Protein Identification and Analysis Tools on the ExPASy Server, (In) John M. Walker (ed): The Proteomics Protocols Handbook, Humana Press (2005). In a preferred embodiment, said antigen has an isoelectric point of above 6.6, 6.7, 6.8 or 6.9. In a preferred embodiment, said antigen has an isoelectric point above 6.6, 6.7, 6.8 or 6.9 and below 13.0, preferably below 12.5, and further preferably below 12.0. In a preferred embodiment, said antigen has an isoelectric point of above 6.6, 6.7, 6.8 or 6.9, as determined by the ExPASy Compute pI/MW tool. In a preferred embodiment, said antigen has an isoelectric point above 6.6, 6.7, 6.8 or 6.9 and below 13.0, preferably below 12.5, and further preferably below 12.0, as determined by the ExPASy Compute pI/MW tool. In a preferred embodiment, said antigen has an isoelectric point of equal to or above 7.0. In a preferred embodiment, said antigen has an isoelectric point equal or above 7.0 and below 13.0, preferably below 12.5, and further preferably below 12.0. In a preferred embodiment, said antigen has an isoelectric point equal to or above 7.0, as determined by the ExPASy Compute pI/MW tool. In a preferred embodiment, said antigen has an isoelectric point equal or above 7.0 and below 13.0, preferably below 12.5, and further preferably below 12.0, as determined by the ExPASy Compute pI/MW tool. In a preferred embodiment, said antigen has an isoelectric point equal to or above 7.1, 7.2, 7.3 or 7.4. In a preferred embodiment, said antigen has an isoelectric point equal to or above 7.1, 7.2, 7.3 or 7.4 and below 13.0, preferably below 12.5, and further preferably below 12.0. In a preferred embodiment, said antigen has an isoelectric point equal to or above 7.1, 7.2, 7.3 or 7.4, as determined by the ExPASy Compute pI/MW tool. In a preferred embodiment, said antigen has an isoelectric point equal to or above 7.1, 7.2, 7.3 or 7.4 and of below 13.0, preferably below 12.5, and further preferably below 12.0, as determined by the ExPASy Compute pI/MW tool. In a preferred embodiment, said antigen has an isoelectric point equal to or above 7.5. In a preferred embodiment, said antigen has an isoelectric point equal to or above 7.5 and below 13.0, preferably below 12.5, and further preferably below 12.0. In a preferred embodiment, said antigen has an isoelectric point equal to or above 7.5, as determined by the ExPASy Compute pI/MW tool. In a preferred embodiment, said antigen has an isoelectric point equal to or above 7.5 and below 13.0, preferably below 12.5, and further preferably below 12.0, as determined by the ExPASy Compute pI/MW tool. In a preferred embodiment, said antigen has an isoelectric point of equal or above 7.6, 7.7, 7.8 or 7.9. In a preferred embodiment, said antigen has an isoelectric point equal or above 7.6, 7.7, 7.8 or 7.9 and below 13.0, preferably below 12.5, and further preferably below 12.0. In a preferred embodiment, said antigen has an isoelectric point of equal or above 7.6, 7.7, 7.8 or 7.9, as determined by the ExPASy Compute pI/MW tool. In a preferred embodiment, said antigen has an isoelectric point equal or above 7.6, 7.7, 7.8 or 7.9 and below 13.0, preferably below 12.5, and further preferably below 12.0, as determined by the ExPASy Compute pI/MW tool. In a preferred embodiment, said antigen has an isoelectric point of equal or above 8.0. In a preferred embodiment, said antigen has an isoelectric point equal or above 8.0. and below 13.0, preferably below 12.5, and further preferably below 12.0. In a preferred embodiment, said antigen has an isoelectric point of equal or above 8.0, as determined by the ExPASy Compute pI/MW tool. In a preferred embodiment, said antigen has an isoelectric point equal or above 8.0 and below 13.0, preferably below 12.5, and further preferably below 12.0, as determined by the ExPASy Compute pI/MW tool. In a preferred embodiment, said antigen has an isoelectric point of equal or above 8.1, 8.2, 8.3 or 8.4. In a preferred embodiment, said antigen has an isoelectric point equal or above 8.1, 8.2, 8.3 or 8.4. and below 13.0, preferably below 12.5, and further preferably below 12.0. In a preferred embodiment, said antigen has an isoelectric point of equal or above 8.1, 8.2, 8.3 or 8.4, as determined by the ExPASy Compute pI/MW tool. In a preferred embodiment, said antigen has an isoelectric point equal to or above 8.1, 8.2, 8.3 or 8.4 and below 13.0, preferably below 12.5, and further preferably below 12.0, as determined by the ExPASy Compute pI/MW tool. In a preferred embodiment, said antigen has an isoelectric point equal to or above 8.5. In a preferred embodiment, said antigen has an isoelectric point equal to or above 8.5. and below 13.0, preferably below 12.5, and further preferably below 12.0. In a preferred embodiment, said antigen has an isoelectric point of equal or above 8.5, as determined by the ExPASy Compute pI/MW tool. In a preferred embodiment, said antigen has an isoelectric point equal to or above 8.5 and below 13.0, preferably below 12.5, and further preferably below 12.0, as determined by the ExPASy Compute pI/MW tool.

In a very preferred embodiment, said polypeptide comprising said stretch of consecutive negative amino acids comprises SEQ ID NO:62, SEQ ID NO:63 or SEQ ID NO:64. In a further very preferred embodiment, said polypeptide consists of SEQ ID NO:62, SEQ ID NO:63 or SEQ ID NO:64. In a further very preferred embodiment, said polypeptide comprises SEQ ID NO:62. In a further very preferred embodiment, said polypeptide comprises SEQ ID NO:63. In a further very preferred embodiment, said polypeptide comprises SEQ ID NO:64. In a further very preferred embodiment, said polypeptide consists of SEQ ID NO:62. In a further very preferred embodiment, said polypeptide consists of SEQ ID NO:63. In a further very preferred embodiment, said polypeptide consists of SEQ ID NO:64.

In a very preferred embodiment, said CMV polypeptide comprises the amino acid sequence of SEQ ID NO:5, SEQ ID NO:48 or SEQ ID NO:57, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:48, or between amino acid residues of position 86 (Ser) and position 87 (Thr) of SEQ ID NO:57. In a very preferred embodiment, said CMV polypeptide consists of the amino acid sequence of SEQ ID NO:5, SEQ ID NO:48 or SEQ ID NO:57, wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57. In a very preferred embodiment, said CMV polypeptide comprises the amino acid sequence of SEQ ID NO:5, wherein said polypeptide is inserted between amino acid residues of position 88 and position 89 of SEQ ID NO:5. In a very preferred embodiment, said CMV polypeptide comprises the amino acid sequence of SEQ ID NO:48, wherein said polypeptide is inserted between amino acid residues of position 84 and position 85 of SEQ ID NO:48. In a very preferred embodiment, said CMV polypeptide comprises the amino acid sequence of SEQ ID NO:57, wherein said polypeptide is inserted between amino acid residues of position 86 and position 87 of SEQ ID NO:57. In a very preferred embodiment, said CMV polypeptide consists of the amino acid sequence of SEQ ID NO:5, wherein said polypeptide is inserted between amino acid residues of position 88 and position 89 of SEQ ID NO:5. In a very preferred embodiment, said CMV polypeptide consists of the amino acid sequence of SEQ ID NO:48, wherein said polypeptide is inserted between amino acid residues of position 84 and position 85 of SEQ ID NO:48. In a very preferred embodiment, said CMV polypeptide consists of the amino acid sequence of SEQ ID NO:57, wherein said polypeptide is inserted between amino acid residues of position 86 and position 87 of SEQ ID NO:57.

In a very preferred embodiment, said chimeric CMV polypeptide comprises the amino acid sequence of SEQ ID NO:5, SEQ ID NO:48 or SEQ ID NO:57, wherein said polypeptide comprising said stretch of consecutive negative amino acids is inserted between amino acid residues of position 88 (Ser) and position 89 (Thr) of said SEQ ID NO:5, between amino acid residues of position 84 (Ser) and position 85 (Thr) of SEQ ID NO:48, or between amino acid residues of position 86 (Ser) and position 87 (Thr) of SEQ ID NO:57. In a very preferred embodiment, said chimeric CMV polypeptide consists of the amino acid sequence of SEQ ID NO:5, SEQ ID NO:48 or SEQ ID NO:57, wherein said polypeptide comprising said stretch of consecutive negative amino acids is inserted between amino acid residues of position 88 and position 89 of said SEQ ID NO:5, between amino acid residues of position 84 and position 85 of SEQ ID NO:48, or between amino acid residues of position 86 and position 87 of SEQ ID NO:57. In a very preferred embodiment, said chimeric CMV polypeptide comprises the amino acid sequence of SEQ ID NO:5, wherein said polypeptide is inserted between amino acid residues of position 88 and position 89 of SEQ ID NO:5. In a very preferred embodiment, said chimeric CMV polypeptide comprises the amino acid sequence of SEQ ID NO:48, wherein said polypeptide is inserted between amino acid residues of position 84 and position 85 of SEQ ID NO:48. In a very preferred embodiment, said chimeric CMV polypeptide comprises the amino acid sequence of SEQ ID NO:57, wherein said polypeptide is inserted between amino acid residues of position 86 and position 87 of SEQ ID NO:57. In a very preferred embodiment, said chimeric CMV polypeptide consists of the amino acid sequence of SEQ ID NO:5, wherein said polypeptide is inserted between amino acid residues of position 88 and position 89 of SEQ ID NO:5. In a very preferred embodiment, said chimeric CMV polypeptide consists of the amino acid sequence of SEQ ID NO:48, wherein said polypeptide is inserted between amino acid residues of position 84 and position 85 of SEQ ID NO:48. In a very preferred embodiment, said chimeric CMV polypeptide consists of the amino acid sequence of SEQ ID NO:57, wherein said polypeptide is inserted between amino acid residues of position 86 and position 87 of SEQ ID NO:57.

In a very preferred embodiment, said CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine, or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:5 and said polypeptide comprising said stretch of consecutive negative amino acids is inserted between amino acid residues 88 (Ser) and amino acid residue 89 (Thr) of SEQ ID NO:5, and said polypeptide comprising said stretch of consecutive negative amino acids further comprises a first and a second amino acid linker, wherein said first and said second amino acid linker is independently a glycine-serine linker (GS-linker) comprising at least one glycine and at least one serine or an amino acid linker (GS*-linker) comprising at least one Gly, at least one Ser, and at least one amino acid selected from Thr, Ala, Lys, and Cys, wherein said first and/or said second amino acid linker has a Gly-Ser sequence at its N-terminus.

In a very preferred embodiment, said chimeric CMV polypeptide comprises the amino acid sequence of SEQ ID NO:10, SEQ ID NO:11 or SEQ ID NO:12. In a very preferred embodiment, said chimeric CMV polypeptide comprises the amino acid sequence of SEQ ID NO: 10. In a very preferred embodiment, said chimeric CMV polypeptide comprises the amino acid sequence of SEQ ID NO:11. In a very preferred embodiment, said chimeric CMV polypeptide comprises the amino acid sequence of SEQ ID NO:12. In a very preferred embodiment, said chimeric CMV polypeptide consists of the amino acid sequence of SEQ ID NO:10, SEQ ID NO:11 or SEQ ID NO:12. In a very preferred embodiment, said chimeric CMV polypeptide consists of the amino acid sequence of SEQ ID NO:10. In a very preferred embodiment, said chimeric CMV polypeptide consists of the amino acid sequence of SEQ ID NO:11. In a very preferred embodiment, said chimeric CMV polypeptide consists of the amino acid sequence of SEQ ID NO:12.

In a very preferred embodiment, said modified VLP of CMV comprises 180 copies of said chimeric CMV polypeptide comprising the amino acid sequence of SEQ ID NO:10, SEQ ID NO:11 or SEQ ID NO:12. In a very preferred embodiment, said modified VLP of CMV comprises 180 copies of said chimeric CMV polypeptide comprising the amino acid sequence of SEQ ID NO:10. In a very preferred embodiment, said modified VLP of CMV comprises 180 copies of said chimeric CMV polypeptide comprising the amino acid sequence of SEQ ID NO:11. In a very preferred embodiment, said modified VLP of CMV comprises 180 copies of said chimeric CMV polypeptide comprising the amino acid sequence of SEQ ID NO:12. In a very preferred embodiment, said modified VLP of CMV comprises 180 copies of said chimeric CMV polypeptide consisting of the amino acid sequence of SEQ ID NO:10, SEQ ID NO:11 or SEQ ID NO:12. In a very preferred embodiment, said modified VLP of CMV comprises 180 copies of said chimeric CMV polypeptide consisting of the amino acid sequence of SEQ ID NO:10. In a very preferred embodiment, said modified VLP of CMV comprises 180 copies of said chimeric CMV polypeptide consisting of the amino acid sequence of SEQ ID NO:11. In a very preferred embodiment, said modified VLP of CMV comprises 180 copies of said chimeric CMV polypeptide consisting of the amino acid sequence of SEQ ID NO:12.

In a further very preferred embodiment, said antigen is canine IL-1β. In a very preferred embodiment, said antigen comprises, or preferably consists of, SEQ ID NO:44, SEQ ID NO:45, SEQ ID NO:119, SEQ ID NO:120 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:44, SEQ ID NO:45, SEQ ID NO:119, SEQ ID NO:120. In a further preferred embodiment, said antigen comprises SEQ ID NO:44, SEQ ID NO:45, SEQ ID NO:119 or SEQ ID NO:120. In a further preferred embodiment, said antigen consists of SEQ ID NO:44, SEQ ID NO:45, SEQ ID NO:119 or SEQ ID NO:120. In a further very preferred embodiment, said antigen comprises, or preferably consists of, SEQ ID NO:44 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:44. In a further very preferred embodiment, said antigen comprises SEQ ID NO:44. In a further very preferred embodiment, said antigen consists of SEQ ID NO:44. In a further preferred embodiment, said antigen comprises, or preferably consists of, SEQ ID NO:45 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:45. In a further very preferred embodiment, said antigen comprises SEQ ID NO:45. In a further very preferred embodiment, said antigen consists of SEQ ID NO:45. In a further preferred embodiment, said antigen comprises, or preferably consists of, SEQ ID NO:119 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:119. In a further very preferred embodiment, said antigen comprises SEQ ID NO:119. In a further very preferred embodiment, said antigen consists of SEQ ID NO:119. In a further very preferred embodiment, said antigen comprises, or preferably consists of, SEQ ID NO:120 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:120. In a further very preferred embodiment, said antigen comprises SEQ ID NO:120. In a further very preferred embodiment, said antigen consists of SEQ ID NO:120.

In a further very preferred embodiment, said antigen is canine NGF. In a very preferred embodiment, said antigen comprises, or preferably consists of, SEQ ID NO:30, SEQ ID NO:31, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:30 or SEQ ID NO:31. In a further preferred embodiment, said antigen comprises SEQ ID NO:30 or SEQ ID NO:31. In a further preferred embodiment, said antigen consists of SEQ ID NO:30 or SEQ ID NO:31. In a further very preferred embodiment, said antigen comprises, or preferably consists of, SEQ ID NO:30 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:30. In a further very preferred embodiment, said antigen comprises SEQ ID NO:30. In a further very preferred embodiment, said antigen consists of SEQ ID NO:30. In a further preferred embodiment, said antigen comprises, or preferably consists of, SEQ ID NO:31 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:31. In a further very preferred embodiment, said antigen comprises SEQ ID NO:31. In a further very preferred embodiment, said antigen consists of SEQ ID NO:31.

In a further very preferred embodiment, said antigen is feline IL-5. In a very preferred embodiment, said antigen comprises, or preferably consists of, SEQ ID NO:35, SEQ ID NO:41, SEQ ID NO:42, SEQ ID NO:77, SEQ ID NO:78, SEQ ID:79 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:35, SEQ ID NO:41, SEQ ID NO:42, SEQ ID NO:77, SEQ ID NO:78, SEQ ID:79. In a further preferred embodiment, said antigen comprises SEQ ID NO:35, SEQ ID NO:41, SEQ ID NO:42, SEQ ID NO:77, SEQ ID NO:78, SEQ ID:79. In a further preferred embodiment, said antigen consists of SEQ ID NO:35, SEQ ID NO:41, SEQ ID NO:42, SEQ ID NO:77, SEQ ID NO:78, SEQ ID:79. In a further very preferred embodiment, said antigen comprises, or preferably consists of, SEQ ID NO:35 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:35. In a further very preferred embodiment, said antigen comprises SEQ ID NO:35. In a further very preferred embodiment, said antigen consists of SEQ ID NO:35. In a further preferred embodiment, said antigen comprises, or preferably consists of, SEQ ID NO:41 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:41. In a further very preferred embodiment, said antigen comprises SEQ ID NO:41. In a further very preferred embodiment, said antigen consists of SEQ ID NO:41. In a further preferred embodiment, said antigen comprises, or preferably consists of, SEQ ID NO:42 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:42. In a further very preferred embodiment, said antigen comprises SEQ ID NO:42. In a further very preferred embodiment, said antigen consists of SEQ ID NO:42. In a further very preferred embodiment, said antigen comprises, or preferably consists of, SEQ ID NO:77 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:77. In a further very preferred embodiment, said antigen comprises SEQ ID NO:77. In a further very preferred embodiment, said antigen consists of SEQ ID NO:77. In a further very preferred embodiment, said antigen comprises, or preferably consists of, SEQ ID NO:78 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:78. In a further very preferred embodiment, said antigen comprises SEQ ID NO:78. In a further very preferred embodiment, said antigen consists of SEQ ID NO:78. In a further very preferred embodiment, said antigen comprises, or preferably consists of, SEQ ID NO:79 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:79. In a further very preferred embodiment, said antigen comprises SEQ ID NO:79. In a further very preferred embodiment, said antigen consists of SEQ ID NO:79.

In a further very preferred embodiment, said antigen is feline IL-1b. In a very preferred embodiment, said antigen comprises, or preferably consists of, SEQ ID NO:30, SEQ ID NO:31, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:121 or SEQ ID NO:171. In a further preferred embodiment, said antigen comprises SEQ ID NO:121 or SEQ ID NO:171. In a further preferred embodiment, said antigen consists of SEQ ID NO:121 or SEQ ID NO:171. In a further very preferred embodiment, said antigen comprises, or preferably consists of, SEQ ID NO:121 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:121. In a further very preferred embodiment, said antigen comprises SEQ ID NO:121. In a further very preferred embodiment, said antigen consists of SEQ ID NO:121. In a further preferred embodiment, said antigen comprises, or preferably consists of, SEQ ID NO:171 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:171. In a further very preferred embodiment, said antigen comprises SEQ ID NO:171. In a further very preferred embodiment, said antigen consists of SEQ ID NO:171.

In a very preferred embodiment, said modified VLP of CMV comprises at least one, preferably 180 copies of said chimeric CMV polypeptide comprising the amino acid sequence of SEQ ID NO:10, SEQ ID NO:11 or SEQ ID NO:12, and said antigen comprises, or preferably consists of, SEQ ID NO:44 or SEQ ID NO:45 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:44 or SEQ ID NO:45, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:10, said antigen comprises, or preferably consists of, SEQ ID NO:44, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:44, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:10, said antigen comprises, or preferably consists of, SEQ ID NO:45, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:45, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:44, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:44, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:45, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:45, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:44, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:44, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:45, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:45, and preferably all of said first attachments sites are not comprised or are not part of the polypeptide comprising said stretch of consecutive negative amino acids.

In a very preferred embodiment, said modified VLP of CMV comprises at least one, preferably 180 copies of said chimeric CMV polypeptide comprising the amino acid sequence of SEQ ID NO:10, SEQ ID NO:11 or SEQ ID NO:12, and said antigen comprises, or preferably consists of, SEQ ID NO:30 or SEQ ID NO:31 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:30 or SEQ ID NO:31, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:30, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:30, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:31, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:31, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:30, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:30, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:31, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:31, and preferably all of said first attachments sites are not comprised or are not part of the polypeptide comprising said stretch of consecutive negative amino acids.

In a very preferred embodiment, said modified VLP of CMV comprises at least one, preferably 180 copies of said chimeric CMV polypeptide comprising the amino acid sequence of SEQ ID NO:10, SEQ ID NO:11 or SEQ ID NO:12, and said antigen comprises, or preferably consists of, SEQ ID NO:41 or SEQ ID NO:42 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:41 or SEQ ID NO:42, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:10, said antigen comprises, or preferably consists of, SEQ ID NO:41, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:41, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:10, said antigen comprises, or preferably consists of, SEQ ID NO:42, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:42, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:41, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:41, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:42, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:42, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:41, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:41, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:42, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:42, and preferably all of said first attachments sites are not comprised or are not part of the polypeptide comprising said stretch of consecutive negative amino acids.

In a very preferred embodiment, said modified VLP of CMV comprises at least one, preferably 180 copies of said chimeric CMV polypeptide comprising the amino acid sequence of SEQ ID NO: 10, SEQ ID NO:11 or SEQ ID NO:12, and said antigen comprises, or preferably consists of, SEQ ID NO:121 or SEQ ID NO:171 or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:121 or SEQ ID NO:171, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:121, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:121, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:11, said antigen comprises, or preferably consists of, SEQ ID NO:171, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:171, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:121, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:121, and preferably all of said first attachments sites are not comprised or are not part of the polypeptide comprising said stretch of consecutive negative amino acids. In a very preferred embodiment, said modified VLP of CMV comprises at least one, preferably 180 copies of said chimeric CMV polypeptide comprising the amino acid sequence of SEQ ID NO:12, said antigen comprises, or preferably consists of, SEQ ID NO:171, or an amino acid sequence having a sequence identity of at least 90%, preferably of at least 91% or 92%, further preferably of at least 93%, 94% or at least 95%, and again further preferably of at least 96%, 97% or at least 98% or at least 99% amino acid sequence identity with SEQ ID NO:171, and preferably all of said first attachments sites are not comprised or are not part of the polypeptide comprising said stretch of consecutive negative amino acids.

The modified VLPs of the invention can be prepared in prokaryotic or eukaryotic expression systems. Preferred systems are E. coli, yeast, insect cells as well as mammalian cell lines. Very preferred said modified VLP of CMV or said VLP of CMV is obtained by expression of said chimeric CMV polypeptide in E. coli., and wherein preferably said expression is effected at temperatures of between 10° C. to 35° C.

Therefore, in another aspect, the present invention provides for a modified virus-like particle (VLP) of cucumber mosaic virus (CMV) comprising at least one chimeric CMV polypeptide, wherein said at least one chimeric CMV polypeptide comprises, preferably consists of (i) a CMV polypeptide, wherein said CMV polypeptide comprises a coat protein of CMV or an amino acid sequence having a sequence identity of at least 75% with SEQ ID NO:48; and (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:48, and wherein said modified VLP of CMV is obtained by expression of said chimeric CMV polypeptide in E. coli., and wherein preferably said expression is effected at temperatures of between 10° C. to 35° C.

In another aspect, the present invention provides for a process for the purification of a modified virus-like particle (VLP) of cucumber mosaic virus (CMV) from a recombinant bacterial host expressing said modified VLP of CMV, wherein said modified VLP of CMV comprises at least one chimeric CMV polypeptide, wherein said at least one chimeric CMV polypeptide comprises, preferably consists of (i) a CMV polypeptide, wherein said CMV polypeptide comprises a coat protein of CMV or an amino acid sequence having a sequence identity of at least 75% with SEQ ID NO:48; and (ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:48; and wherein the process comprises the steps of: (a) lysing said bacterial host; (b) clarifying the lysate obtained by said lysis; (c) purifying said modified VLP of CMV from the clarified lysate by anion exchange chromatography (AEX); wherein said steps are performed in the given order.

In a preferred embodiment, said composition comprises an adjuvant. Typical and preferred adjuvants are mineral salts (e.g. Aluminum Hydroxide, Aluminum Phosphate), microcrystalline tyrosine, emulsions, microparticles, saponins (Quil A), cytokines, immune potentiators, microbial components/products, liposomes, complexes, and mucosal adjuvants which are known and as described such, and for example, in the Adjuvant Compendium NIAID and VAC (nih.gov) or by Aguilar et al, (Aguilar J C et al, 2007, Vaccine 25:3752-3762), Gerdts (Gerdts V, 2015, Berliner und Münchener Tierärztliche Wochenschrift 128:456-463) and Pasquale et al. (Pasquale et al. 2015, Vaccines 3:320-343). In another preferred embodiment, said composition is devoid of an adjuvant.

In a further aspect, the present invention provides a vaccine comprising, or alternatively consisting of, (i) the modified VLP of CMV of the invention as described herein, or (ii) the inventive composition comprising said modified VLP of CMV and at least one antigen as described herein. Encompassed are vaccines wherein said modified VLPs of CMV, and/or said inventive composition comprise any one of the technical features disclosed herein, either alone or in any possible combination. In a preferred embodiment, the vaccine further comprises an adjuvant. In a further preferred embodiment the vaccine is devoid of an adjuvant. In a preferred embodiment said vaccine comprises an effective amount of the composition of the invention.

In a further aspect, the invention relates to a pharmaceutical composition comprising: (a) the modified VLP of CMV as described herein, the inventive composition as described herein, or the vaccine of the invention as described herein; and (b) a pharmaceutically acceptable carrier, diluent and/or excipient. Said diluent includes sterile aqueous (e.g., physiological saline) or non-aqueous solutions and suspensions. Pharmaceutical compositions of the invention may be in a form which contain salts, buffers, adjuvants, or other substances which are desirable for improving the efficacy of the conjugate. In a preferred embodiment, said pharmaceutical composition comprises an effective amount of the vaccine of the invention. In a preferred embodiment, said pharmaceutical composition comprises an adjuvant.

A further aspect of the present invention is a method of immunization comprising administering the modified VLP of CMV as described herein, the inventive composition as described herein, the vaccine of the invention as described herein, or the pharmaceutical composition as described herein, to an animal or a human. In a preferred embodiment said method comprises administering the inventive composition as described herein, the vaccine of the invention as described herein, or the pharmaceutical composition as described herein, to an animal or a human. In a preferred embodiment said method comprises administering an effective amount of said modified VLP of CMV, said inventive composition, said vaccine, or said pharmaceutical composition to said animal or said human.

In a further aspect, the present invention provides the modified VLP of CMV as described herein, the inventive composition as described herein, the vaccine of the invention as described herein, or the pharmaceutical composition as described herein for use in a method of immunization an animal or a human, wherein said method comprises administering an effective amount of said modified VLP of CMV, said inventive composition, said vaccine, or said pharmaceutical composition to said animal or said human.

A further aspect of the invention is a method of treating or preventing a disease, disorder or condition in an animal or in a human, said method comprises administering the modified VLP of CMV as described herein, the inventive composition as described herein, the vaccine of the invention as described herein, or the pharmaceutical composition as described herein, to an animal or a human. In a further preferred embodiment, said disease, disorder or condition is selected from the group consisting of an allergy, a cancer, an autoimmune disease, an inflammatory disease or an infectious disease.

In a further aspect, the present invention provides the modified VLP of CMV as described herein, the inventive composition as described herein, the vaccine of the invention as described herein, or the pharmaceutical composition as described herein for use in a method of treating or preventing a disease, disorder or condition in an animal or in a human, wherein said method comprises administering an effective amount of said modified VLP of CMV, said inventive composition, said vaccine, or said pharmaceutical composition to said animal or said human. In a further preferred embodiment, said disease, disorder or condition is selected from the group consisting of an allergy, a cancer, an autoimmune disease, an inflammatory disease or an infectious disease.

In another aspect, the present invention provides for the use of the modified VLP of CMV as described herein, the inventive composition as described herein, the vaccine of the invention as described herein, or the pharmaceutical composition in the manufacture of a medicament for the treatment of a disease, disorder or condition in an animal or a human. In a further aspect the invention provides the modified VLP of CMV for use as a medicament. In a further aspect the invention provides the inventive composition comprising said modified VLP of CMV and at least one antigen as described herein for use as a medicament.

### EXAMPLES

### Example 1

### Construction and Production of Surface Charge Modified CMV VLPs

Different chimeric CMV polypeptides in accordance with the present invention were prepared, and subsequently expressed leading to the inventive modified CMV VLPs.

Towards this end, chimeric CMV polypeptides comprising, in particular, different polypeptides of contiguous negative amino acids, namely polypeptides consisting of either 4, 8, or 12 glutamic acid residues (“E4”—SEQ ID NO:1; “E8”—SEQ ID NO:2; “E12”-SEQ ID NO:3) were prepared such that said glutamic acid residues were inserted between amino acid residues Ser(88) and Tyr(89) of the modified CMV polypeptide CMV-Ntt830 (SEQ ID NO:5). Said modified CMV polypeptide CMV-Ntt830 comprises the T helper cell epitope derived from tetanus toxoid TT830 (SEQ ID NO:6). The corresponding nucleic acid sequence (SEQ ID NO:7) coding for said modified CMV polypeptide CMV-Ntt830 was prepared as described in Example 3 of WO2016/062720A1.

The prepared chimeric CMV polypeptides further comprise linkers flanking the introduced E4, E8 and E12 polypeptides at both termini. In detail, said prepared chimeric CMV polypeptides either comprise a GGS-linker or a GGGS-linker (SEQ ID NO:8) directly at the N-terminus of the introduced E4, E8, and E12 polypeptides, and either a GGGSGS-linker (SEQ ID NO:9) or a CGGGSGS-linker (SEQ ID NO:4) directly at the C-terminus of the introduced E4, E8, and E12 polypeptides.

The resulting amino acid sequences of said prepared chimeric CMV polypeptides are named “CMV-Ntt830-E4”, “CMV-Ntt830-E8”, “CMV-Ntt830-E8*” and “CMV-Ntt830-E12” and have the amino acid sequences as follows:

SEQ ID NO: 10
“CMV-Ntt830-E4”:;
SEQ ID NO: 11
“CMV-Ntt830-E8”:;
SEQ ID NO: 12
“CMV-Ntt830-E8*”:;
SEQ ID NO: 13
“CMV-Ntt830-E12”:.

The corresponding nucleotide sequences of said preferred chimeric CMV polypeptides are as follows:

SEQ ID NO: 14
“CMV-Ntt830-E4”:;
SEQ ID NO: 15
″CMV-Ntt830-E8”:;
SEQ ID NO: 16
“CMV-Ntt830-E8*”:;
SEQ ID NO: 17
“CMV-Ntt830-E12”:.

First, the chimeric CMV polypeptide CMV-Ntt830-E8* was prepared. Hereby and in a first step the incorporation of the coding sequence for E8 including the flanking linkers into the modified CMV using PCR mutagenesis was effected. The PCR fragment coding for the E8 sequence including the flanking linkers as well as the 3′ end fragment of the modified CMV was amplified in two step PCR using the following oligonucleotides:

Forward:
(SEQ ID NO: 18)
E8*-1F
Forward:
(SEQ ID NO: 19)
E8*-2F
Reverse:
(SEQ ID NO: 20)
CMcpR.

Thus, a PCR reaction was carried out using E8*-1F/CMcpR oligonucleotides and pET-CMV-Ntt830 plasmid as template. The template pET-CMV-Ntt830 was prepared as described in Example 3 of WO2016/062720A1. The target PCR product was obtained after a second PCR using oligonucleotides E8*-2F/CMcpR and the PCR product from the first PCR. The resulting PCR product was cloned into helper vector pTZ57 (InsTAclone PCR Cloning Kit, Fermentas #K1214). PCR product-containing plasmid was amplified in E. coli XL1-Blue cells, and plasmid DNA was purified and sequenced using BigDye cycle-sequencing kit and an ABI Prism 3100 Genetic Analyzer (Applied Biosystems). As a result, the helper plasmid pTZ-CMV-E8*, without PCR errors, was obtained.

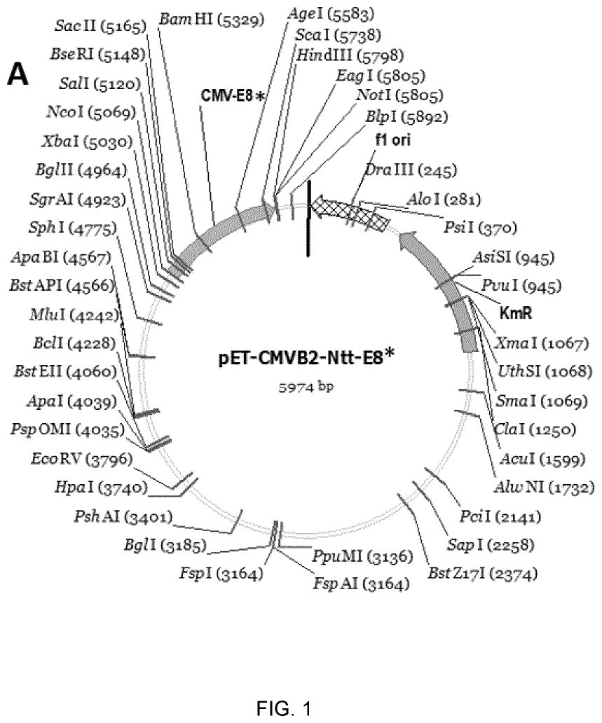

As a next step, the BamHI/HindIII fragment of pTZ-CMV-E8* was cloned back into the pET-CMV-Ntt830B helper vector using the same restriction sites, resulting in the expression vector pET-CMVB2-Ntt-E8C (FIG. 1).

The helper vector pET-CMV-Ntt830B was used for introduction of polypeptides comprising a stretch of consecutive negative amino acids coding DNA sequences in the corresponding CMV DNA sequence of CMV-Ntt830, BamHI site-containing sequence was introduced at the corresponding position for subsequent cloning. The CMV-Ntt830 coding nucleic acid sequence was prepared as described in Example 3 of WO2016/062720A1 and corresponds to SEQ ID NO:14 of WO2016/062720A1.

The BamHI site was introduced by two-step PCR mutagenesis using below listed oligonucleotides and previously constructed pET-CMV-Ntt830 as a template. As indicated, the template pET-CMV-Ntt830 was prepared as described in Example 3 of WO2016/062720A1

1^{st} PCR:
Forward-
(SEQ ID NO: 21)
pET-90 primer (anneals pET28a+)
Reverse-
(SEQ ID NO: 22)
RGSYrev
2nd PCR
Forward-
(SEQ ID NO: 23)
RGSYdir
Reverse-
(SEQ ID NO: 24)
CMV-AgeR

After purification of both PCR products, the next PCR was carried out to join the PCR fragments (5 cycles without primers then 25 cycles using primers pET-90 and CMV-AgeR).

After amplification of the gene, the obtained PCR product was directly cloned into the pTZ57R/T vector (InsTAclone PCR Cloning Kit, Fermentas #K1214). E. coli XL1-Blue cells were used as a host for cloning and plasmid amplification.

To avoid RT-PCR errors, several CMV-Ntt830 gene-containing pTZ57 plasmid clones were sequenced using a BigDye cycle sequencing kit and an ABI Prism 3100 Genetic analyzer (Applied Biosystems). After sequencing, pTZ-plasmid clone without sequence errors containing CMV-Ntt830B gene with introduced BamHI site was cut with NcoI and AgeI enzymes. Then the fragment was subcloned into the NcoI/AgeI sites of the pET-CMV-Ntt830, resulting in the helper vector pET-CMV-Ntt830B.

CMV-Ntt830-E8* VLPs were produced in E. coli C2566 cells (New England Biolabs, USA). The VLPs were produced using, E. coli cell cultivation, biomass treatment and purification methods as follows:
1) suspend 3 g biomass in 20 ml of 50 mM Na citrate, 5 mM Na borate, 5 mM EDTA, 5 mM mercaptoethanol, pH 9.0, treat the suspension with ultrasound (Hielscher sonicator UP200S, 16 min, amplitude 70%, cycle 0.5);
2) Centrifuge the lysate at 11000 rpm for 20 min, at +4° C.;
3) Prepare sucrose gradient (20-60%) in 35 ml tubes, in buffer containing 50 mM Na citrate, 5 mM Na borate, 2 mM EDTA, 0.5% TX-100;
4) Overlay 5 ml of the VLP sample over the sucrose gradient;
5) Centrifuge 6 h using SW32 rotor, Beckman (25000 rpm, at +18° C.).
6) Divide the content of each gradient tube in 6 ml fractions. Pool corresponding fractions;
7) Analyse gradient fractions on SDS.

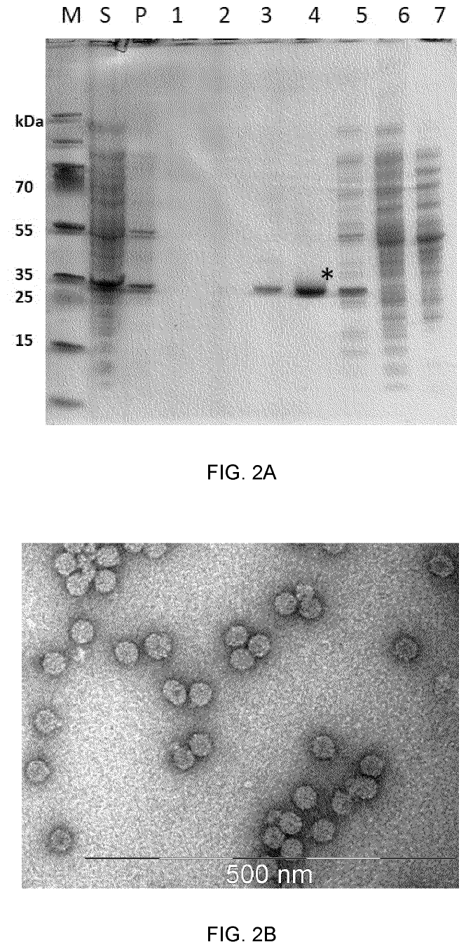

SDS-PAGE analysis of the VLPs after sucrose gradient purification demonstrates homogeneous CMV-Ntt830-E8* coat protein monomer (FIG. 2A) and electron microscopy shows intact VLPs (FIG. 2B).

The chimeric CMV polypeptides CMV-Ntt830-E4, CMV-Ntt830-E8 and CMV-Ntt830-E12 were prepared accordingly and as follows. The first step was the incorporation of the poly-glutamate coding sequences including the flanking linkers into the modified CMV using PCR mutagenesis. The PCR fragments coding for poly-glutamate sequences including the flanking linkers as well as the 3′ end fragment of the modified CMV were amplified by PCR using the following pairs of oligonucleotides and plasmid pET-CMVB2-Ntt-E8* as a template:

1)
Forward:
(SEQ ID NO: 25)
E4-F
Reverse:
(SEQ ID NO: 20)
CMcpR;
2)
Forward:
(SEQ ID NO: 26)
E8-F
Reverse:
(SEQ ID NO: 20)
CMcpR;
3)
Forward:
(SEQ ID NO: 27)
E12-F
Reverse:
(SEQ ID NO: 20)
CMcpR.

The resulting PCR products were cloned into helper vector pTZ57 (InsTAclone PCR Cloning Kit, Fermentas #K1214). PCR product containing plasmids were amplified in E. coli XL1-Blue cells, and plasmid DNAs purified and sequenced using BigDye cycle-sequencing kit and an ABI Prism 3100 Genetic Analyzer (Applied Biosystems). Thus the helper plasmids pTZ-CMV-E4, pTZ-CMV-E8 and pTZ-CMV-E12 without PCR errors were obtained.

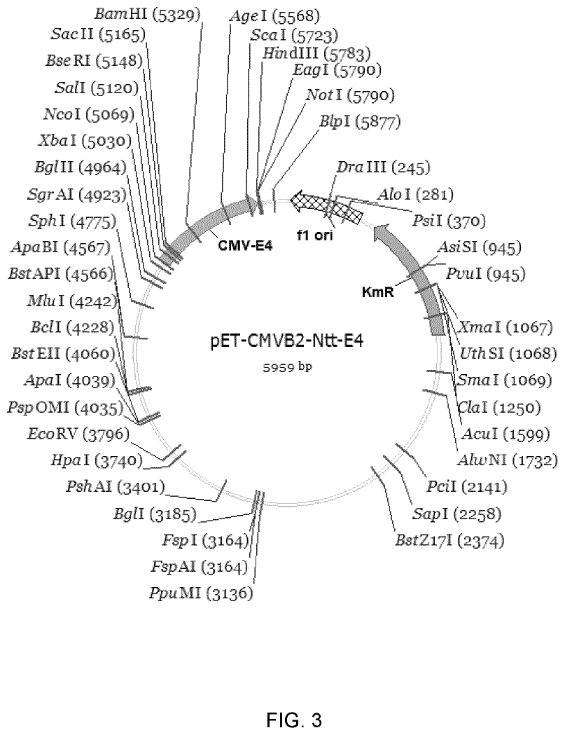

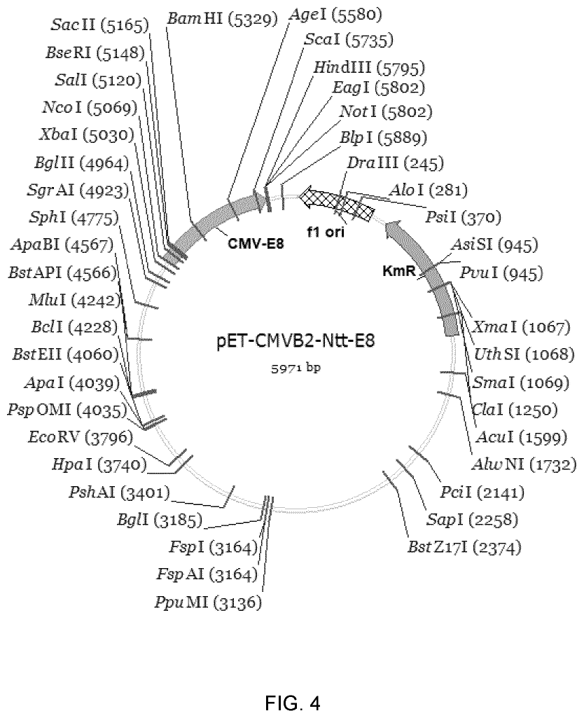

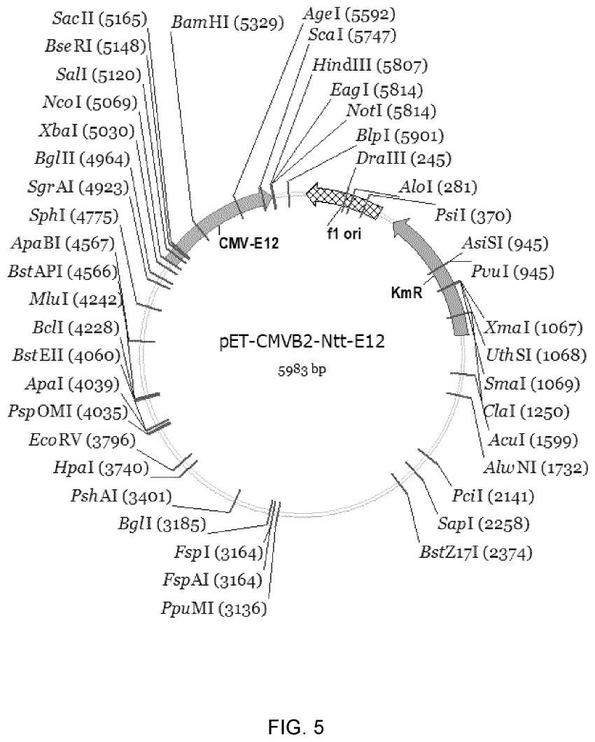

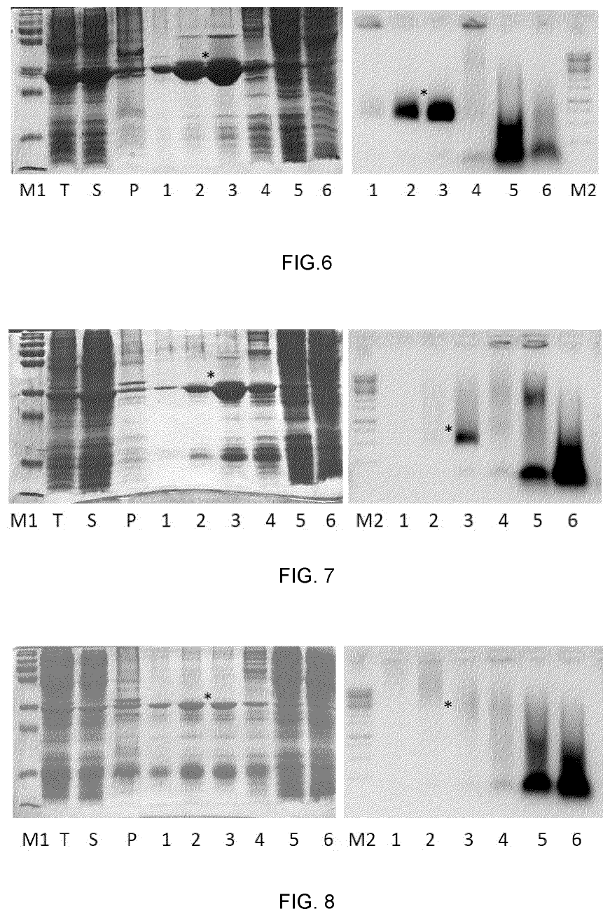

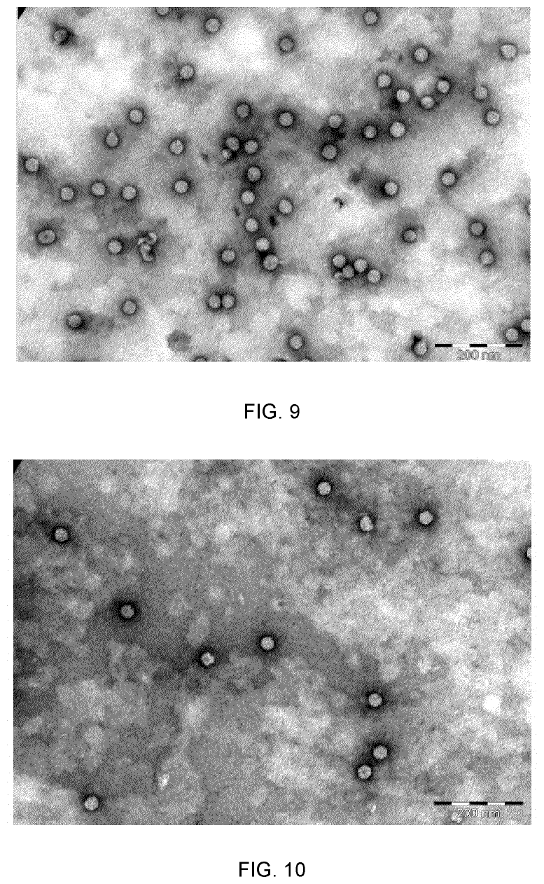

Next, the BamHI/HindIII digested fragments of pTZ-CMV-E4, pTZ-CMV-E8 and pTZ-CMV-E12 were cloned back into the pET-CMV-Ntt830B (see above) using the same restriction sites. The expression vectors pET-CMVB2-Ntt-E4 (FIG. 3) pET-CMVB2-Ntt-E8 (FIG. 4), and pET-CMVB2-Ntt-E12 (FIG. 5) were thus obtained. The expression vectors were transformed into E. coli C2566 cells (New England Biolabs, USA). VLPs were produced using, E. coli cell-cultivation, biomass-treatment and purification methods as described above for CMV-Ntt830-E8* VLPs. SDS-PAGE analyses of the VLPs after sucrose gradient purification demonstrated near homogeneous CMV-coat protein monomer was obtained for all 3 poly-glutamate constructs (FIG. 6, FIG. 7, FIG. 8). However, agarose gel analysis showed integral particles were only formed with CMV-Ntt830-E4 and CMV-Ntt830-E8 but not with the CMV-E12 (FIG. 6, FIG. 7, FIG. 8). Electron microscopy showed that CMV-Ntt830-E4 and CMV-Ntt830-E8 formed intact VLPs (FIG. 9, FIG. 10).

### Example 2

### Improved Stability of the Inventive Surface Charge Modified CMV VLPs as Compared to Prior Art CMV VLPs

### Thermal Stability

Increased thermal stability of the inventive surface charge modified CMV VLPs was demonstrated by measuring denaturation of the prior art CMV-Ntt830 VLPs, which were prepared as described in Example 3 and Example 4 of WO2016/062720A1, and of the inventive CMV-Ntt830-E4 VLPs as a function of increasing temperature and determining the respective melting points.

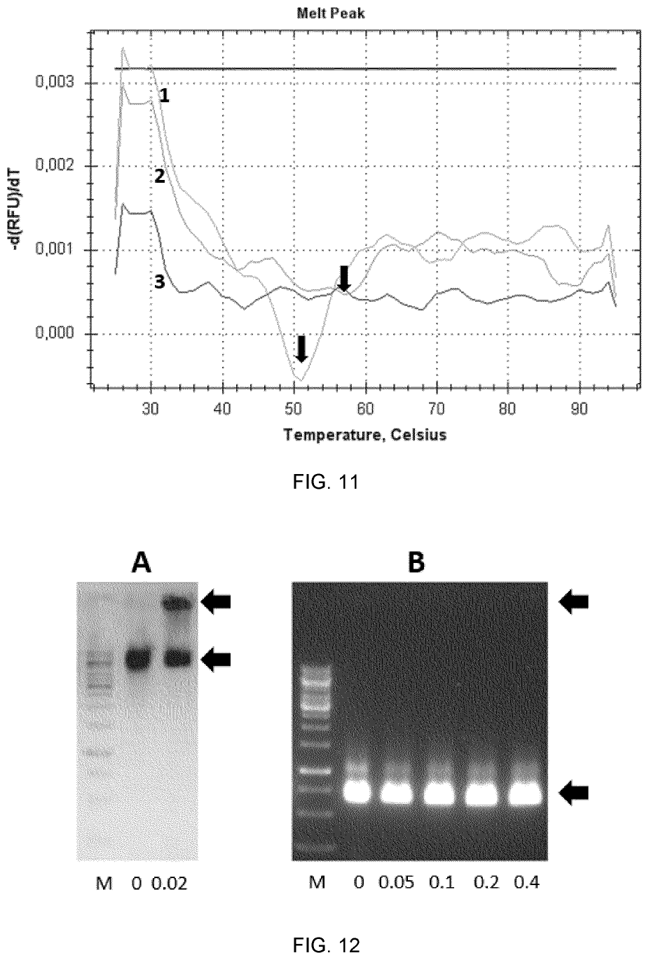

A thermal shift assay involving temperature-induced denaturation and the fluorescent dye SYPRO® Orange (Sigma, Saint Louis, USA) was used for this purpose. The dye is a naturally quenched in solution but as the VLPs denature with increasing temperatures, SYPRO® Orange interacts with exposed hydrophobic amino acids and cores and emits a fluorescent signal, which is measured by fluorometry. From the resultant melting curve (fluorescent signal vs temperature), the melt peak curves and melting temperature were determined. Solutions containing 0.5 mg/ml of sucrose density gradient purified (as described in Example 1 above) CMV-Ntt830 VLPs or CMV-Ntt830-E4 VLPs in 5 mM Na phosphate, 2 mM EDTA, pH 7.5 were assayed with a real-time PCR system MJ Mini (Bio-Rad, Hercules, USA) using a DNA melting point determination program. Data were analysed using Opticon Monitor Software and melting curves processed at a smooth setting of four. FIG. 11 shows the melt peak curves for purified CMV-Ntt830 VLPs and CMV-Ntt830-E4 VLPs.

The respective melting temperatures were estimated to be 51° C. and 57° C. evidencing an increased thermal stability of the surface charge modified CMV VLPs in accordance with the invention as compared to the prior art CMV-Ntt830 VLPs.

### Ionic Strength/Salt Stability

Ionic strength is important for capsid stability. Salts in solution interact with charged residues on the coat proteins and VLP surfaces, influence the water shell and disfavour hydrophobic exposure and thereby influence overall VLP stability.

The relative stabilities of CMV-Ntt830 VLPs and CMV-Ntt830-E4 VLPs to NaCl were tested by incubating purified VLPs (0.5 mg/ml in 5 mM Na phosphate, 2 mM EDTA, pH 7.5) at room temperature with various NaCl concentrations. After 2 hours in the presence of 20 mM NaCl, the CMV-Ntt830 VLPs were relatively unstable and formed aggregates in significant proportion that were both visible to the eye and demonstrable by native gel electrophoresis (FIG. 12). In contrast, there was no evidence of aggregate formation for CMV-Ntt830-E4 VLPs even with NaCl concentrations up to 0.4 M (FIG. 12).

The improved stability in higher salt solution arising from the surface charge modifications of the inventive modified CMV VLPs is important for its processability by ion-exchange chromatography as described in Example 3.

### Example 3

### Improved Purification Potential of the Inventive Surface Charge Modified CMV VLPs as Compared to Prior Art CMV VLPs

The sucrose gradient/cushion ultra-centrifugation purification step, which was used in the laboratory-scale CMV VLP manufacture process as described in the prior art such as in Examples 2-4 of WO2016/062720A1 and for the preparation of the inventive modified CMV VLPs as described in Example 1 above, provides CMV VLPs of suitable yield and purity for subsequent conjugation, vaccine manufacture and preclinical evaluation. However, this method cannot be simply and cost effectively used to produce vaccine for commercial purposes.

Ion exchange chromatography (IEX) is typically readily scalable and used in downstream processes for the commercial production of biologics. It is based on reversible ionic interactions between charged molecules/macromolecules in solution and an immobilized oppositely charged chromatography resin. An example is anion-exchange chromatography (AEX) where the stationary phase (resin) is positively charged and negatively charged molecules such as proteins are bound. The interaction of the resin and sample can be disrupted by application of a counter ion such as Cl⁻. IEX is commonly used in bind/elute mode to provide rapid capture, high-resolution purification and concentration of the desired sample. It can be employed in the initial (e.g. after lysate clarification), intermediate or penultimate stages of a downstream process.

For CMV VLPs to be effectively bound and eluted by IEX, it is necessary that the CMV VLP is stable to the ionic environment encountered during the binding and elution phases. Both the charge on the ion-exchange resin and elution salt contribute to the ionic environment.

The prior art CMV-Ntt830 VLPs as well as the inventive modified CMV VLPs such as CMV-Ntt830-E4, CMV-Ntt830-E8 and CMV-Ntt830-E8* have a net negative charge at about pH's of 9 and below, as demonstrated by their migration towards the positively charged electrode in NAGE. Thus, anion-exchange chromatography (AIX) is a technique that would have been expected to work for both CMV VLP particles.

However, this is not the case because the CMV-Ntt830 VLPs, as described above in Example 2, are relatively unstable in solution in the presence of already 20 mM NaCl and form aggregates, which precipitate. In contrast, the inventive modified CMV VLPs such as the CMV-Ntt830-E4 VLP do not form aggregates at NaCl concentrations up to 0.4 M (FIG. 12, Panel B). The improved stability in higher salt solution arising from the surface charge modifications to the VLP is essential for its processability by ion-exchange chromatography.

### Improved Purification by Anion Exchange Chromatography (AEX).

To test the processability of prior art CMV-Ntt830 VLPs with anion exchange chromatography (AEX), sucrose gradient purified VLPs were prepared as described in Examples 2-4 of WO2016/062720A1. Five mls of CMV-Ntt830 VLPs (1 mg/ml) were buffer exchanged into 5 mM sodium borate pH 9 and loaded onto a 1.0 ml Macro-Prep DEAE Bio-Rad anion exchange cartridge equilibrated with the same buffer. After the loading step, the concentration of NaCl in the elution buffer was increased in step-wise manner (0.1, 0.2, 0.3, 0.4. 0.5, 0.8., 1.0 and 2.0 M). Fractions were collected and measured at 260 nm using Nanodrop spectrophotometer to measure protein and subjected to native agarose gel electrophoresis (NAGE).

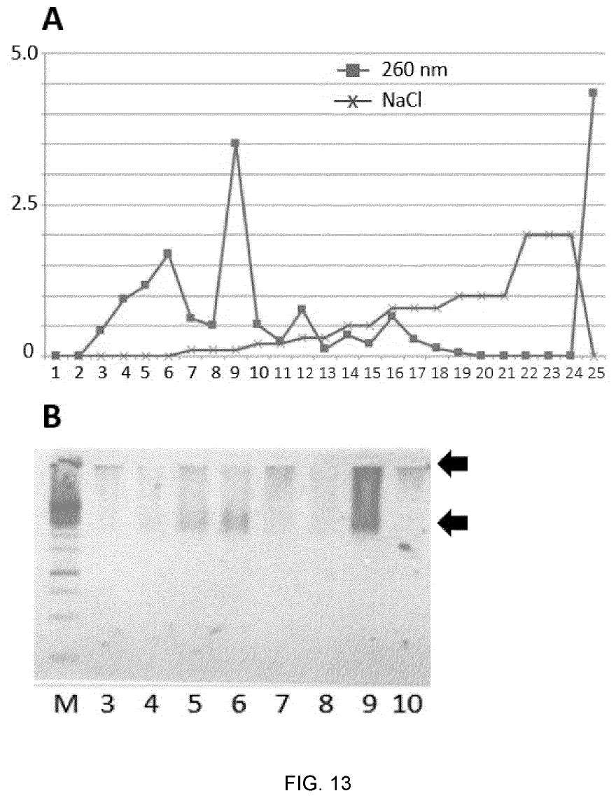

The resultant chromatogram of protein elution and NaCl concentrations plotted against the corresponding fraction (FIG. 13, panel A) shows the CMV-Ntt830 VLPs did not elute as a single peak as is typical for AIX. Instead, CMV-Ntt830 VLPs eluted in a broad non-specific manner during the loading (at 0 M NaCl) and subsequent elution steps over a range of NaCl concentrations, principally 0.2 to 0.8 M. Critically, the VLP-containing fractions after elution from the column were turbid and contained a significant proportion of aggregated VLPs, as demonstrated by the presence of ethidium bromide stained VLPs in the loading wells following NAGE (FIG. 13, panel B). The propensity of the CMV-Ntt830 VLPs to aggregate and elute in a non-discrete manner precludes the ready use of this methodology for scale-up manufacture.

In contrast, non-aggregated CMV-Ntt830-E4 VLPs could be readily purified from a crude lysate using AEX. Clarified lysate prepared from E. coli expressing CMV-Ntt830-E4 VLPs (as described in Example 1) in 50 mM citrate, 5 mM Borate buffer pH 9.0 was loaded onto 60 ml of Fracto-DEAE (Merck) in an XK 26/20 column equilibrated with the same buffer and eluted by applying a continuous NaCl gradient from 0 to 1.0 M in the same buffer. The eluate was monitored at A260 nm to measure protein and conductivity measured to monitor salt concentration. The clarified lysate, flow-through and fractions were collected and subjected to NAGE and SDS-PAGE.

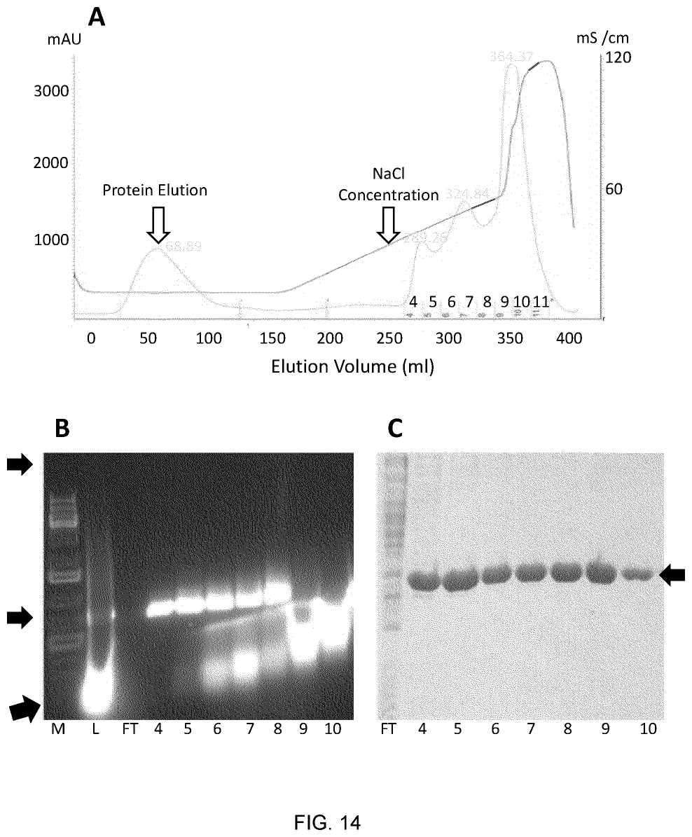

The resultant chromatogram, SDS-PAGE and NAGE analyses (FIG. 14) show that the CMV-Ntt830-E4 VLPs were not present in the flow-through and entirely bound to the Fracto-DEAE. The VLPs were subsequently eluted over a relatively narrow concentration range of 0.2-0.5M NaCl. Moreover, there was no evidence of aggregated VLPs in the loading wells of the native agarose gel. The Coomassie blue stained SDS-polyacrylamide gel showed highly pure VLP coat protein was obtained from the crude bacterial lysate.

### Example 4

### Cloning, Expression and Purification of Recombinant Mature NGF

### Cloning of Recombinant NGF

A cDNA construct (SEQ ID NO:28) consisting of full-length feline NGF pro-peptide sequence, canine mature NGF sequence and a C-terminal glycine-cysteine-glycine motif was synthesized de novo and cloned into pBHA vector (BIONEER Company). The canine NGF sequence was codon optimized. The resulting amino acid sequence of the full-length feline NGF pro-peptide is provided in SEQ ID NO:29 comprising the canine mature NGF sequence of SEQ ID NO:30. The amino acid sequence of canine mature NGF to which said C-terminal glycine-cysteine-glycine motif is attached is provided in SEQ ID NO:31.

Analogously, a cDNA construct (SEQ ID NO:173) consisting of full-length feline NGF pro-peptide sequence, canine mature NGF sequence, a C-terminal glycine-cysteine-glycine motif and a his-tag was synthesized de novo and cloned into pBHA vector (BIONEER Company). The included his-tag does not fulfil any roles for purification, but its presence increased refolding efficiency in downstream processes. The resulting amino acid sequence is provided in SEQ ID NO:174 comprising the canine mature NGF sequence of SEQ ID NO:30 as well as the His6-tag (SEQ ID NO:175).

The constructs were sub-cloned into an expression vector by PCR. Briefly, the NGF-pBHA plasmid was used as a template with an NGF forward primer (SEQ ID NO:32), and an NGF reverse primer (SEQ ID NO:33), containing XbaI and HindIII sites respectively.

The NGF PCR product was subject to 1% agarose gel electrophoresis in TAE buffer and then NGF fragment extracted with GeneJet DNA elution kit (Thermo Fisher Scientific) according to the manufacturer's protocol. The NGF fragment was digested with FastDigest XbaI and HindIII (Thermo Fisher Scientific) restriction enzymes for 30 min in 1× FastDigest buffer at +37° C. according to the manufacturer's protocol. pET42a plasmid (Novagen) was digested in the same manner. The NGF and vector digested DNA fragments were analysed with agarose gel electrophoresis and extracted as above. The NGF fragment was ligated in the pET42a vector using T4 ligase overnight in room temperature according to manufacturer's protocol.

The NGF-pET42a construct was transformed in chemically competent E. coli DH5a cells by the heat shock method. The cells were suspended in 1 ml of LB medium and incubated at +37° C. with shaking for 1 hour and plated onto LB agar containing 60 μg/ml kanamycin and incubated overnight at 37° C. Individual colonies were seeded into LB medium, containing 30 μg/ml kanamycin and incubated overnight at +37° C. with shaking. DNA was extracted from individual clone cultures with GeneJet plasmid miniprep kit (Thermo Fisher Scientific) according to manufacturer protocol.

The correct sequence of the NGF constructs of SEQ ID NO:28 and SEQ ID NO:173 were confirmed by Sanger sequencing using a BigDye Terminator v3.1 Cycle Sequencing Kit (Thermo Fisher Scientific) according to manufacturer's protocol.

### Expression and Purification of Recombinant Canine Mature NGF

The NGF-pET42a plasmid was transformed into chemically competent E. coli BL21-DE3 (Sigma-Aldrich) cells. The cells were suspended in 1 ml of LB medium and incubated at +37° C. with shaking for 1 hour. The cells were plated onto LB agar containing 60 μg/ml kanamycin and incubated overnight at 37° C. Several colonies of NGF-pET42 transformed BL21-DE3 cells were seeded into LB medium containing 30 μg/ml kanamycin, and incubating overnight at 37° C. and then added to 2×TY medium containing 30 μg/ml kanamycin and grown at 37° C. with shaking until OD_{540 nm} of 0.7 units was reached. Recombinant protein expression was induced by addition of IPTG to a final concentration of 1 mM and cells grown for an additional 4 hours at 37° C. with shaking. The biomass was collected by centrifugation at 5000 g for 15 minutes, frozen and stored at −70° C.

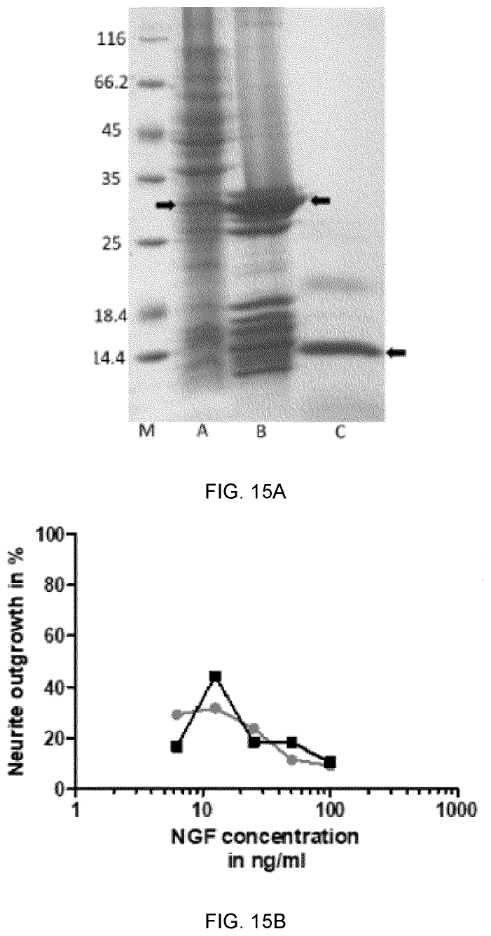

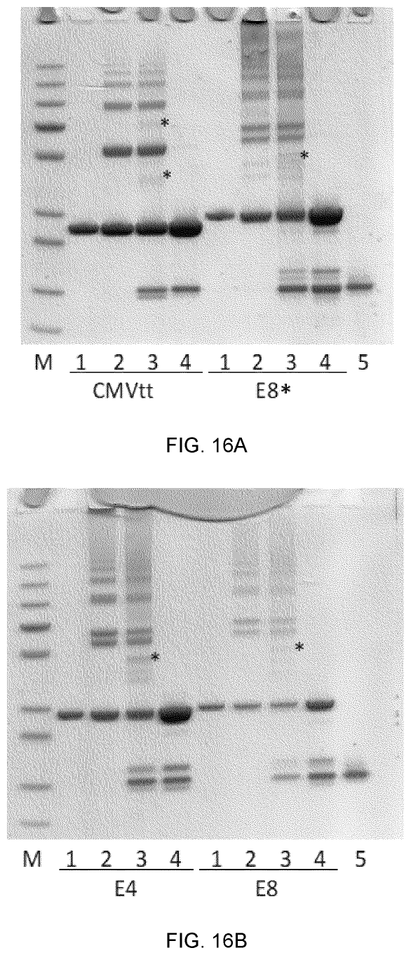

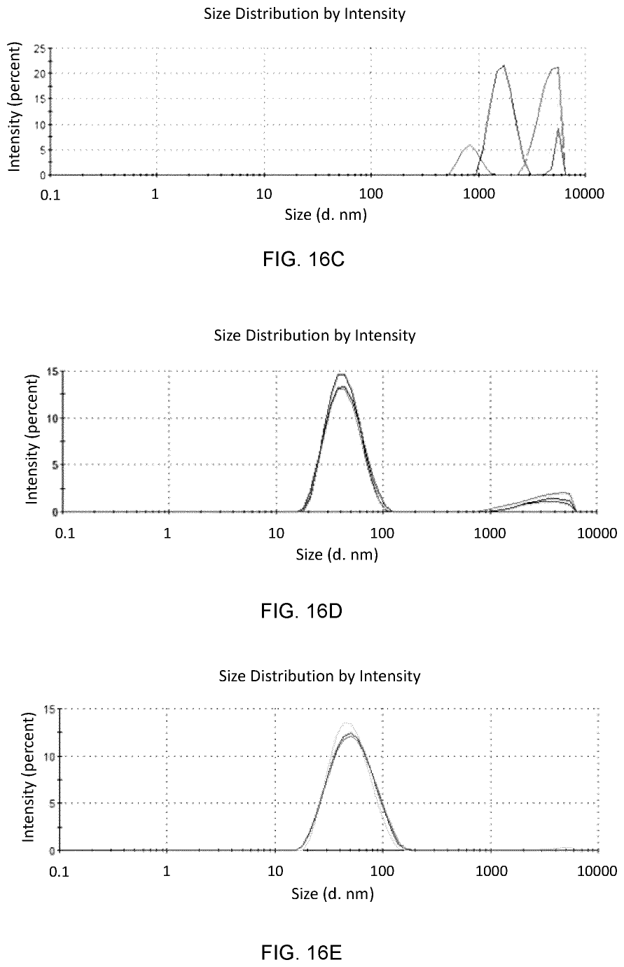

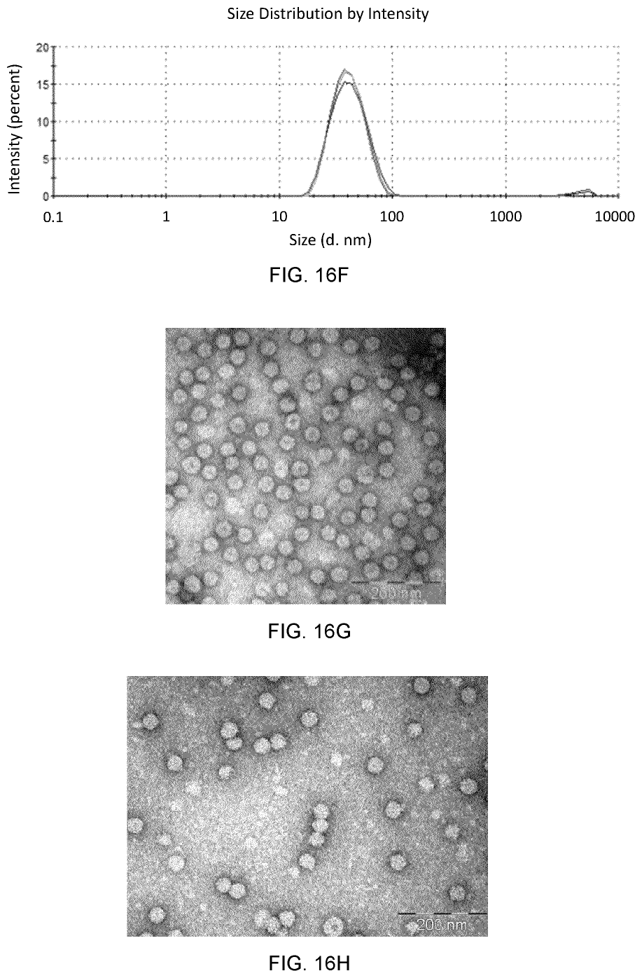

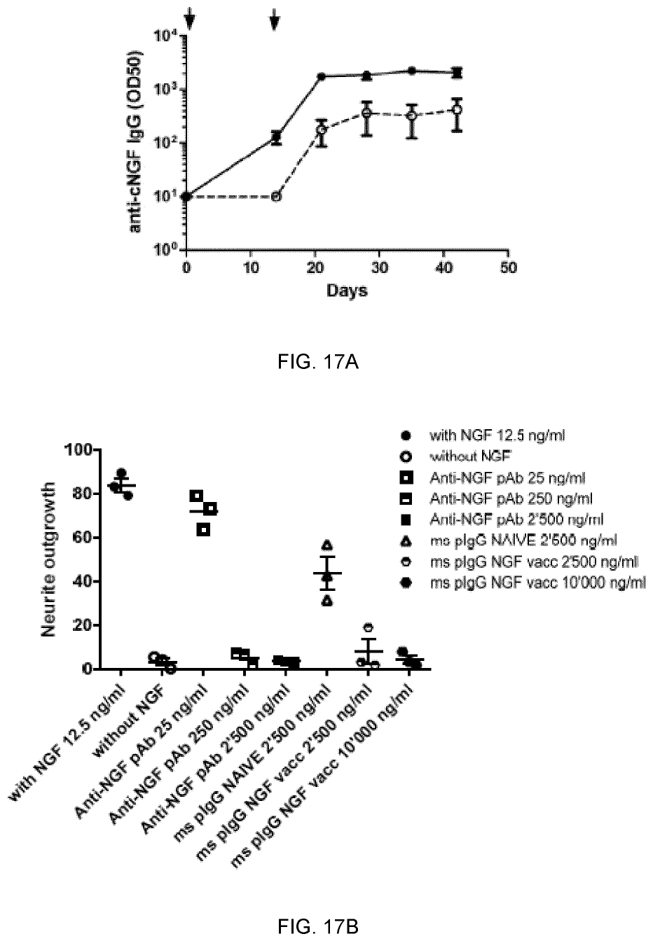

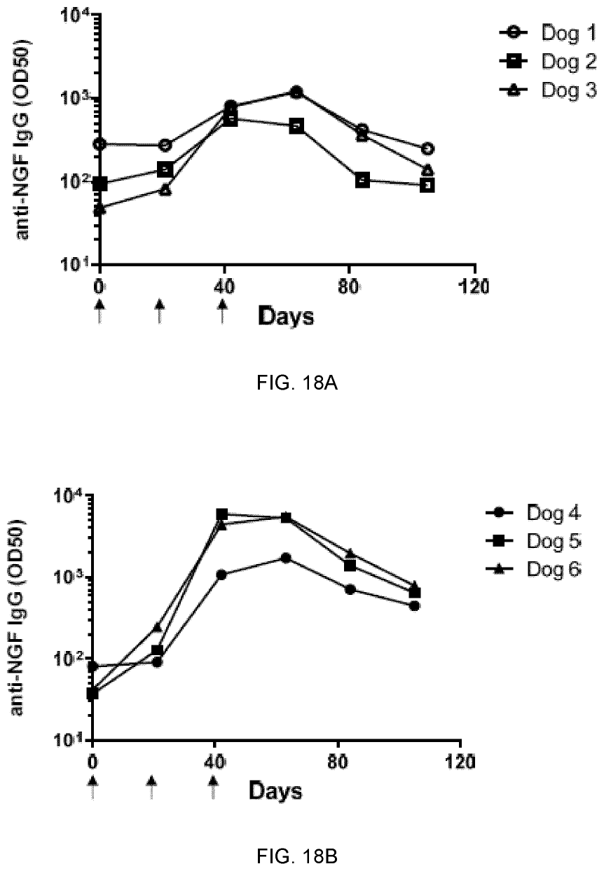

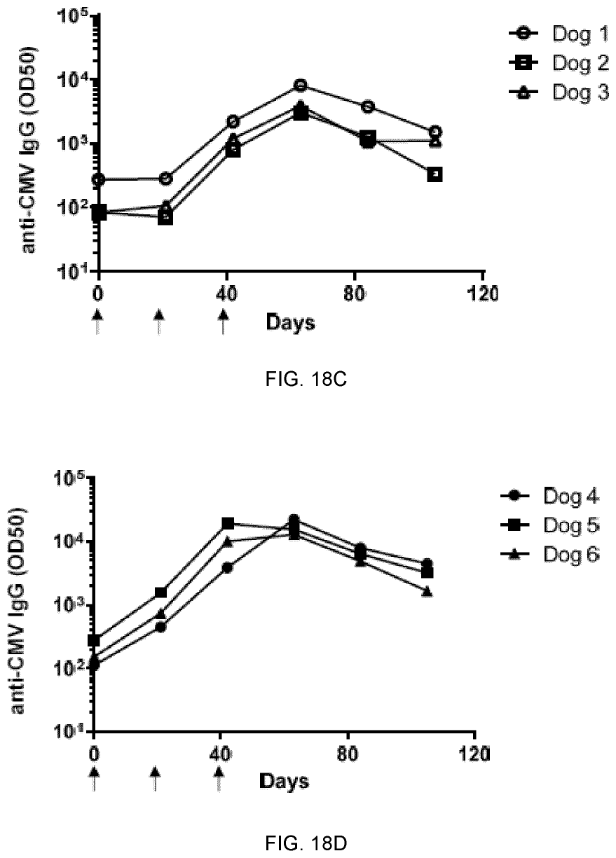

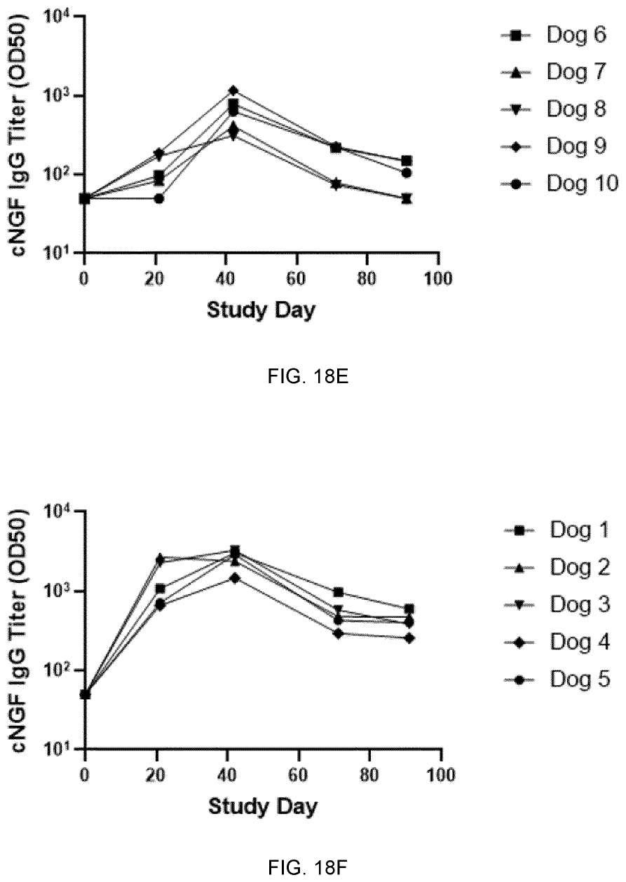

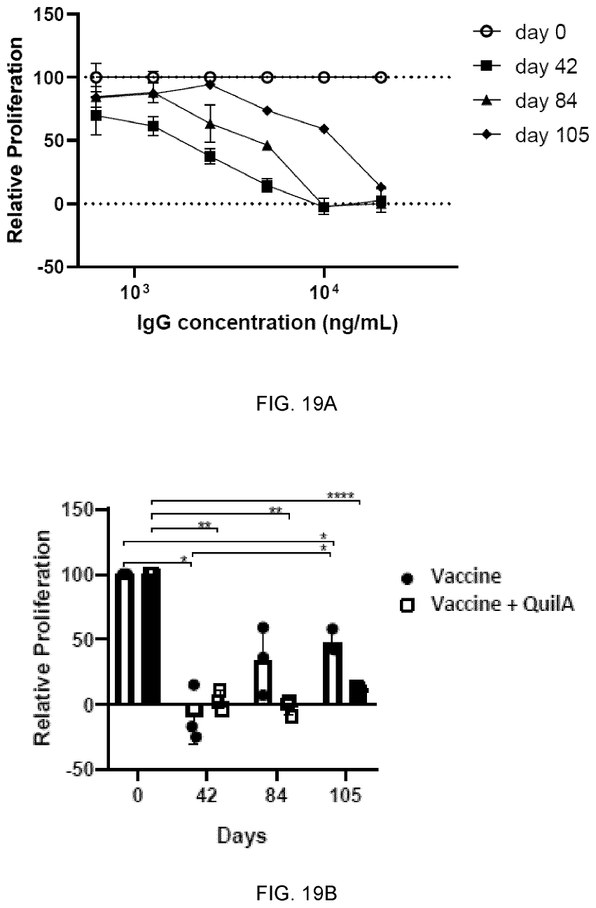

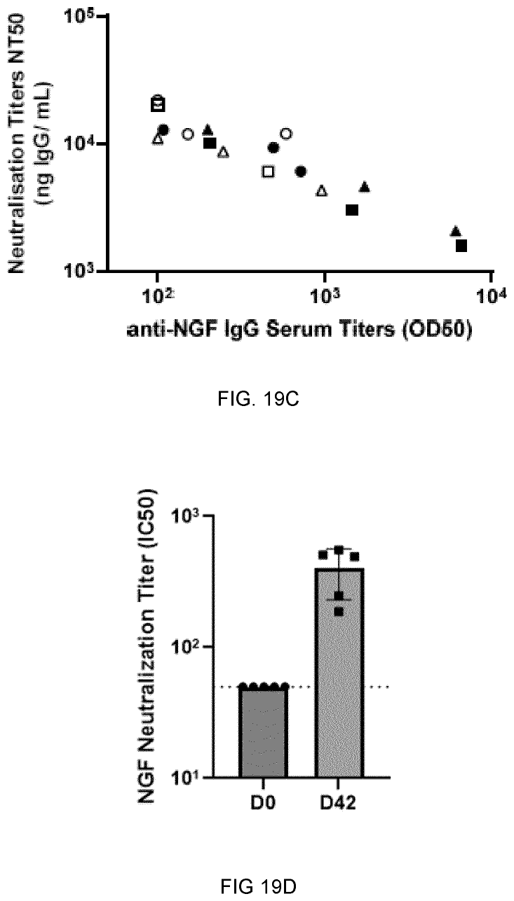

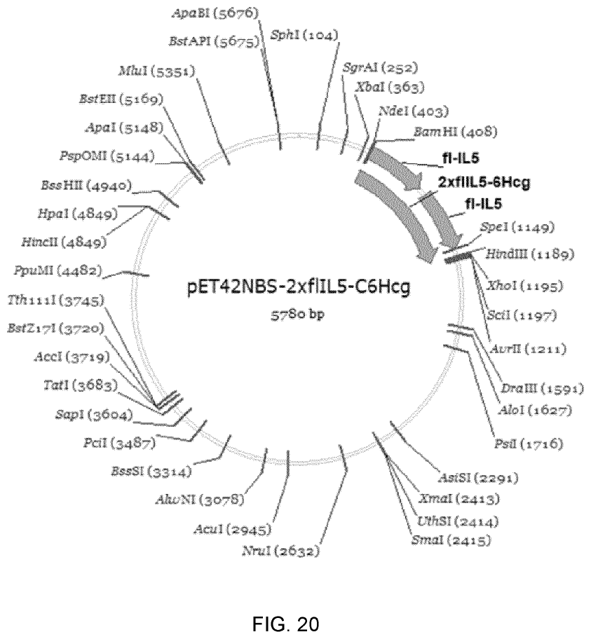

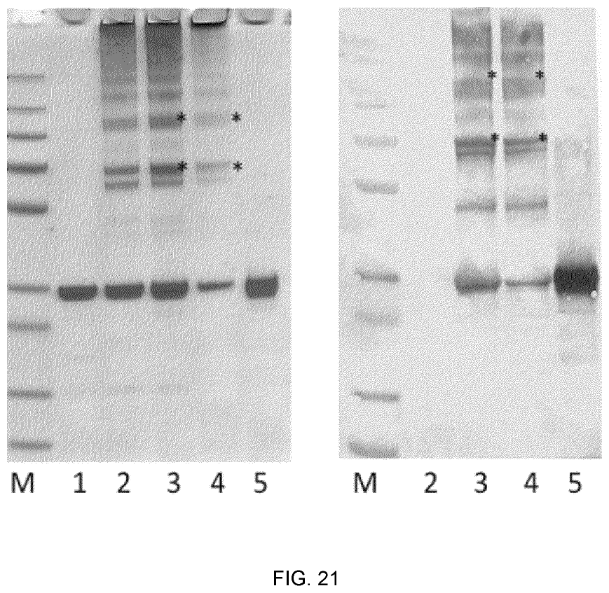

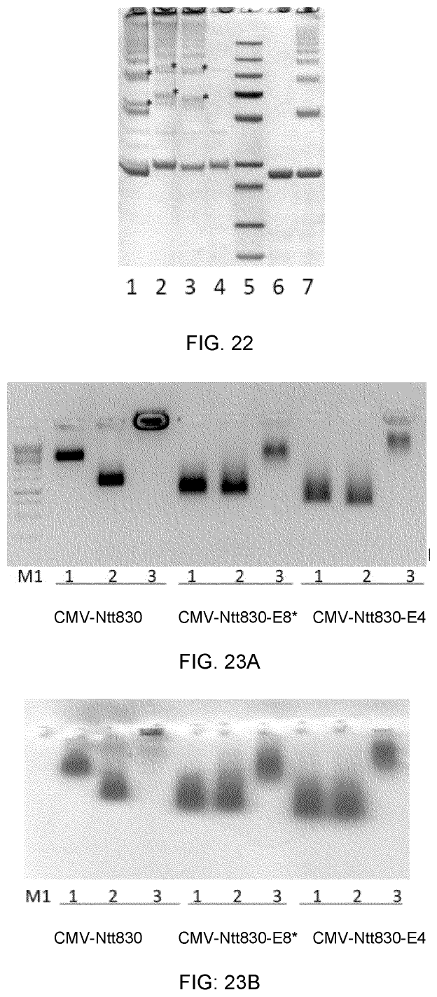

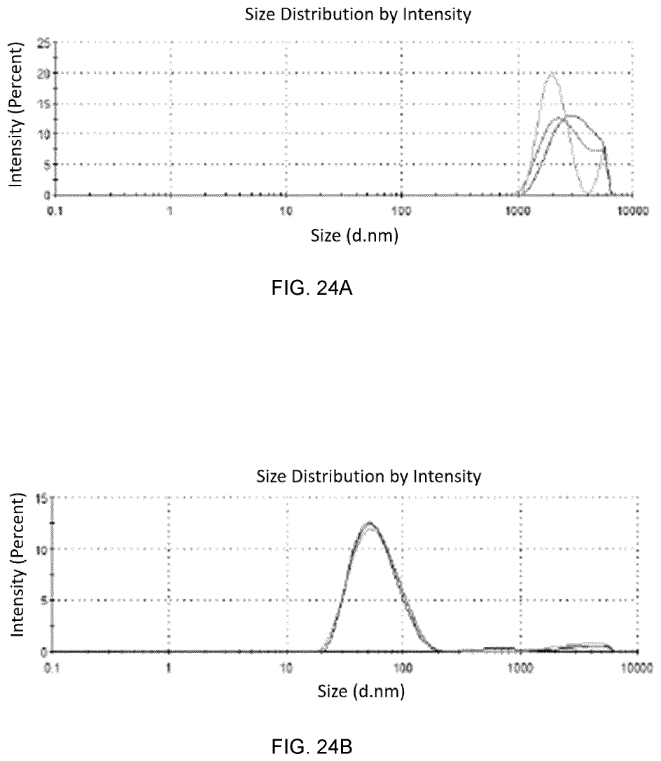

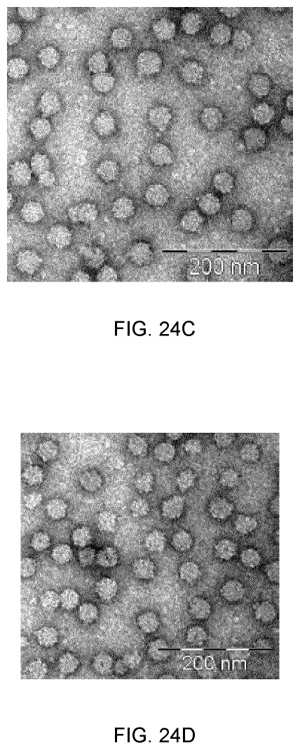

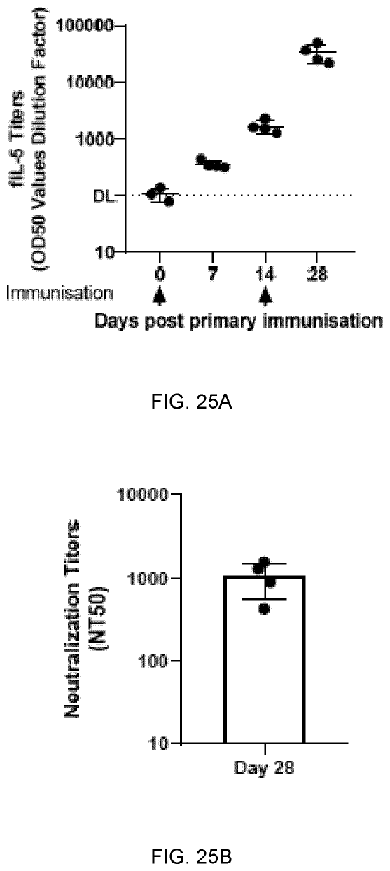

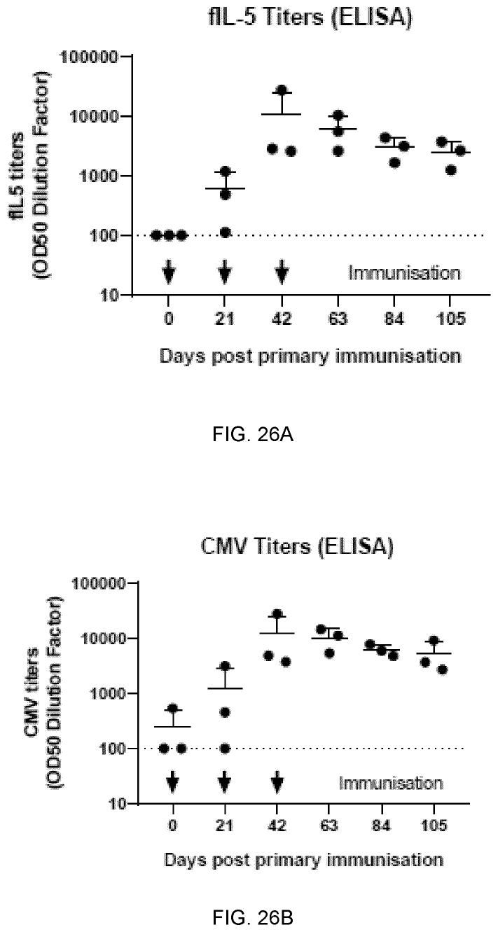

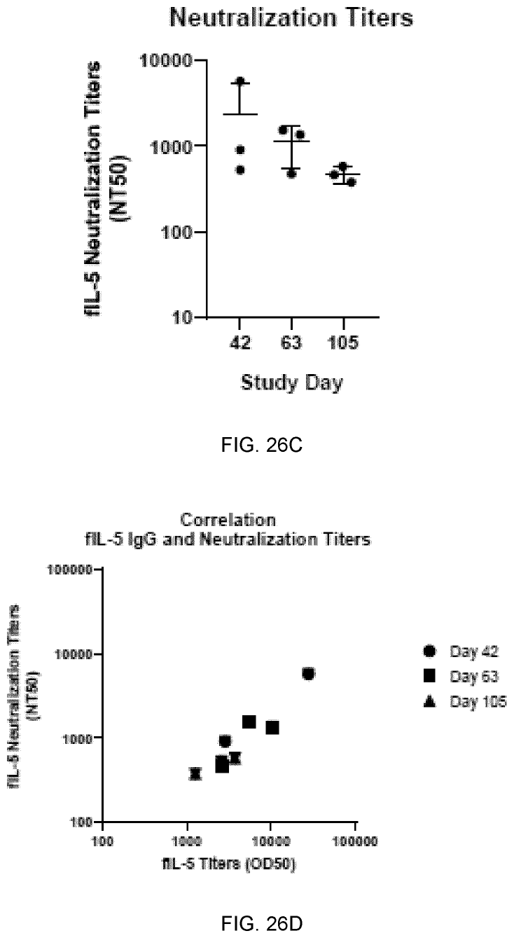

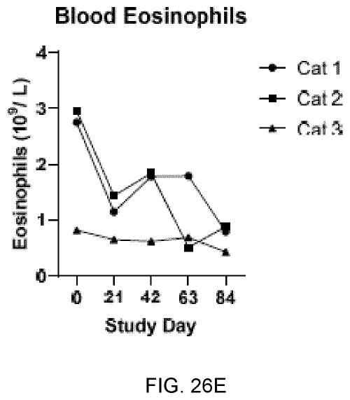

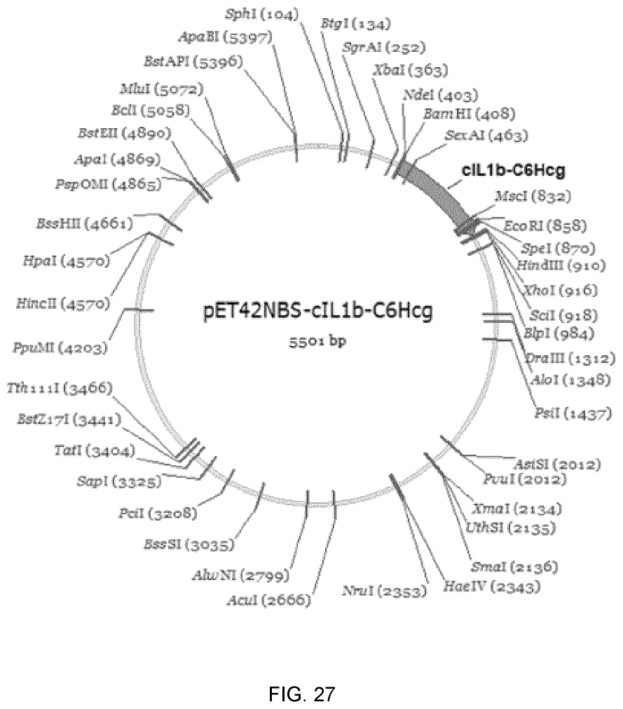

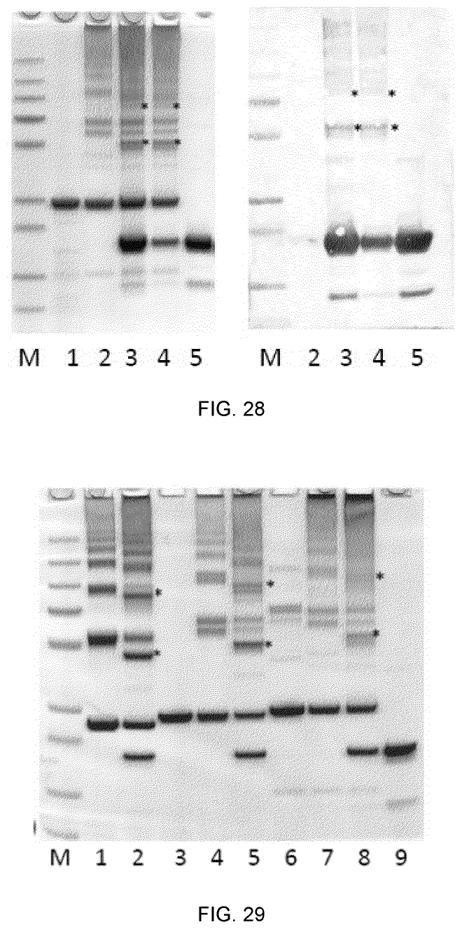

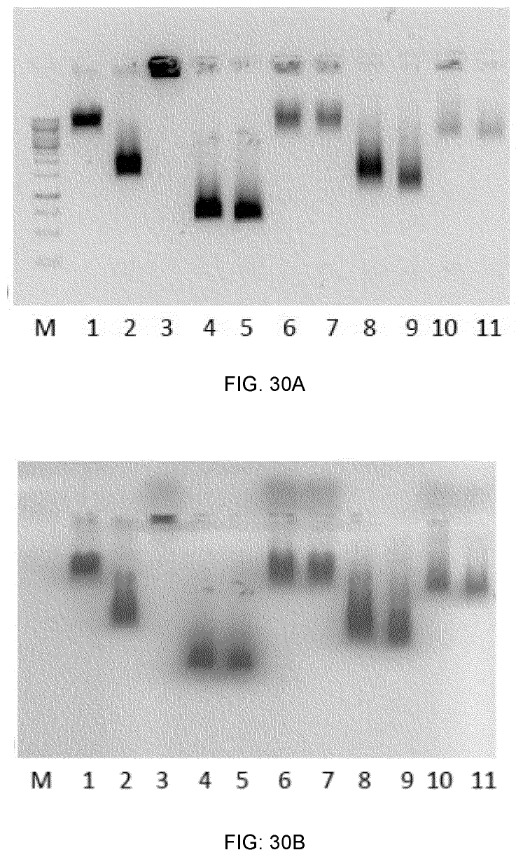

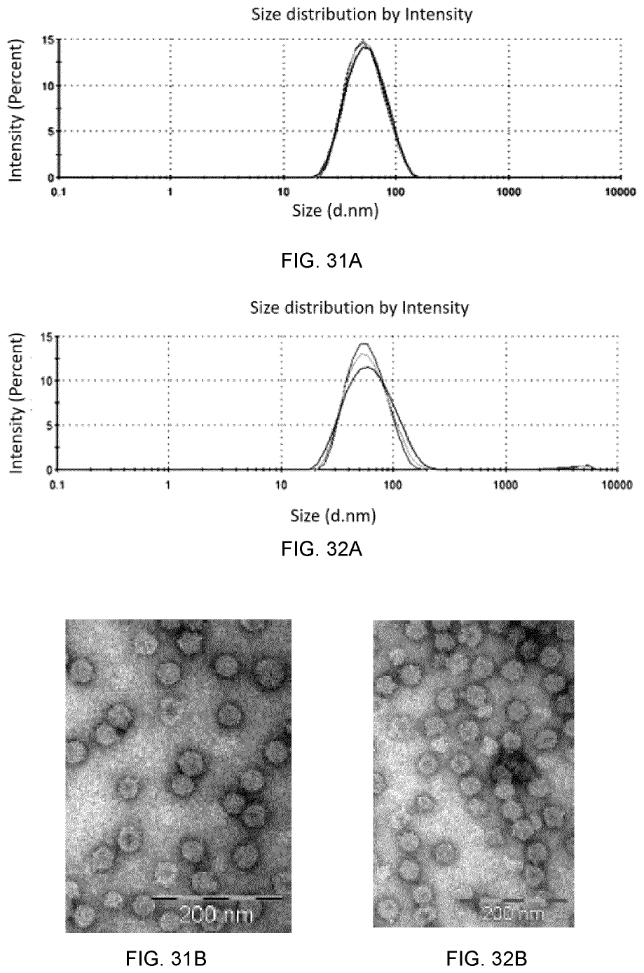

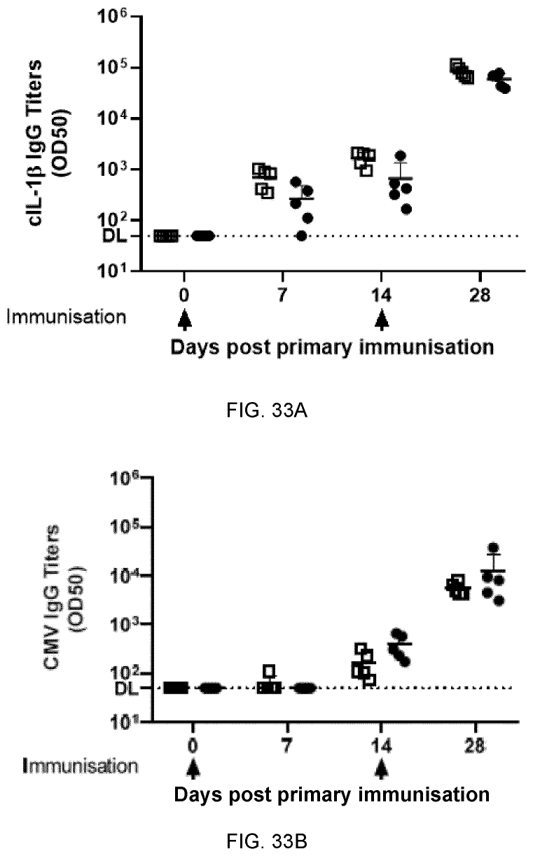

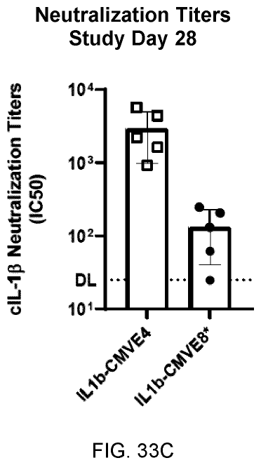

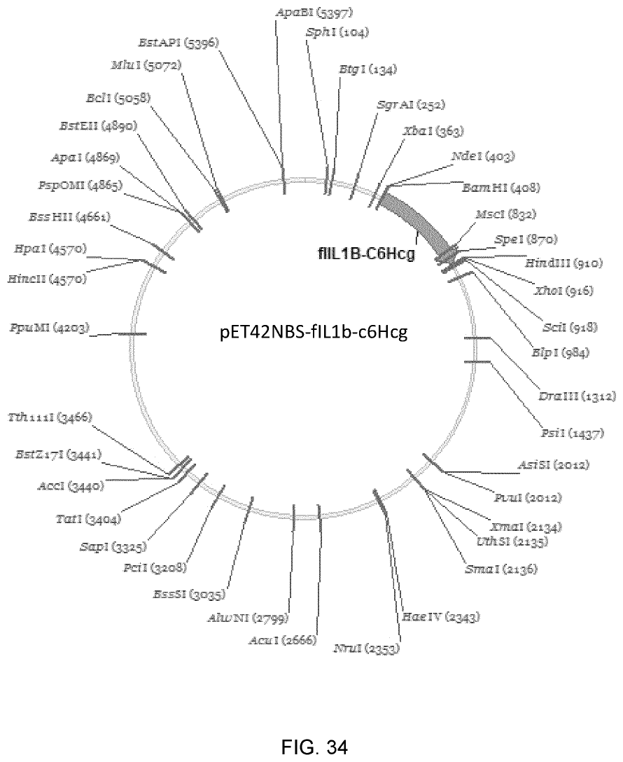

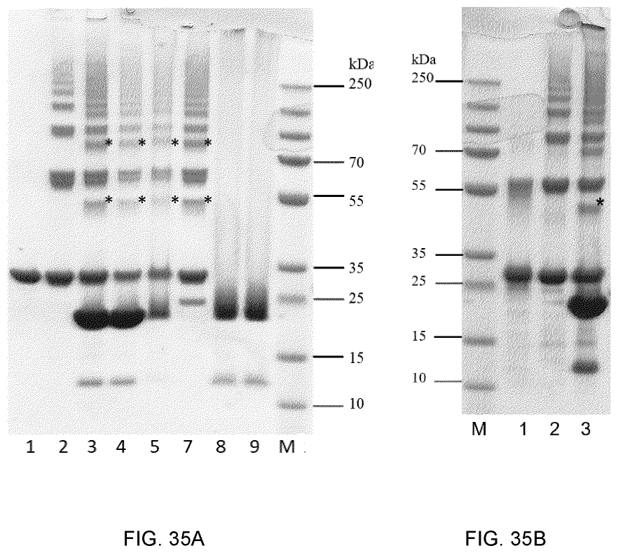

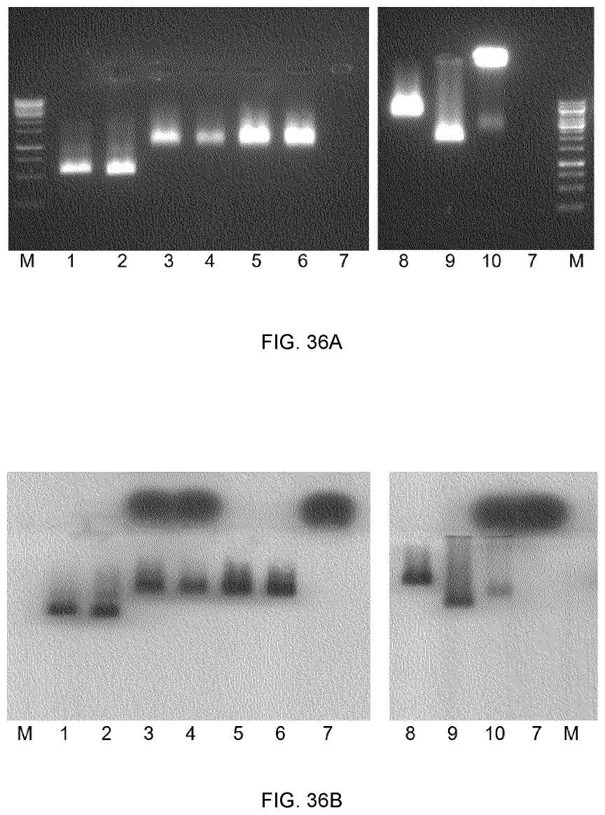

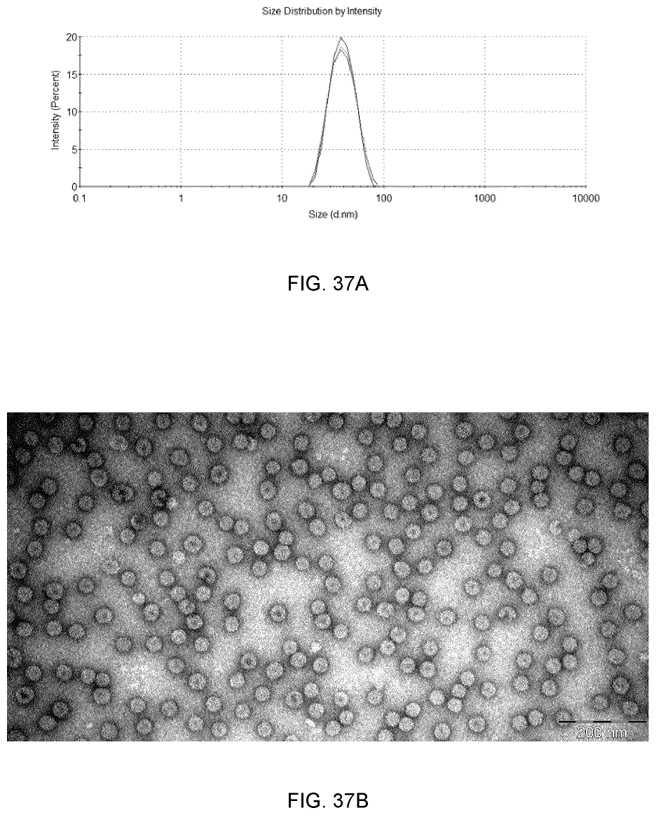

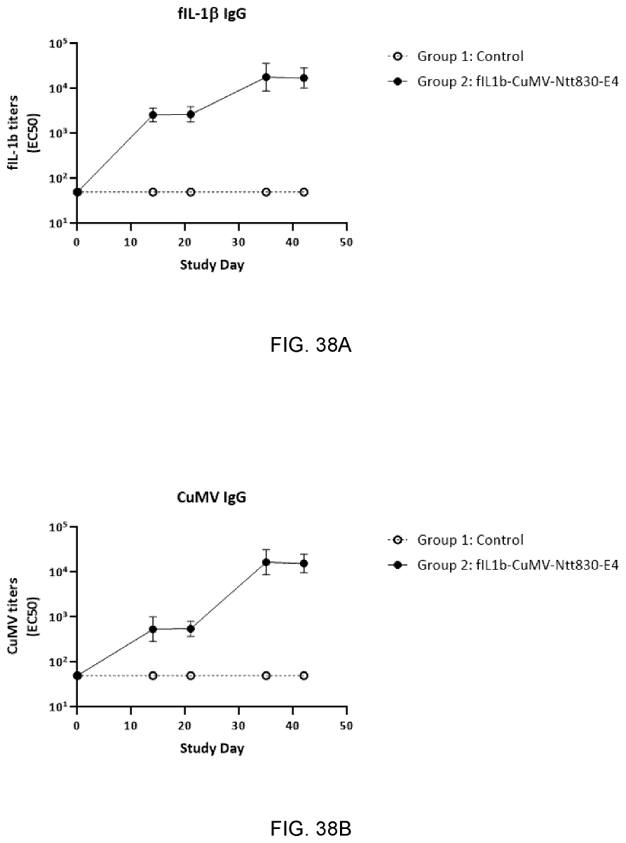

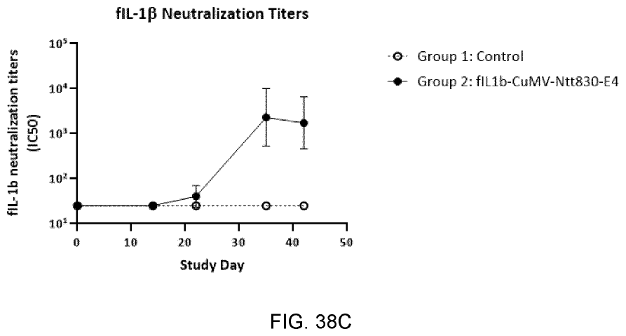

The biomass was suspended in lysis buffer (40 mM Tris-HCl (pH 8.0), 200 mM NaCl, 1 mM PMSF, 1 mM DTT and 1% Triton X-100) and cells lysed by sonication, using a UP200S (Hielscher) ultrasound device. The resultant sonicate was centrifuged for 40 min at 15 557 g. The supernatant was discarded and lysis buffer was added to the pellet which was Re-suspended by sonication. The suspension was centrifuged for 15 min at 15 557 g and the supernatant again discarded. This washing step was repeated three more times. The pellet was washed a final time with 50% lysis buffer and 3.5 M urea. After resuspension and centrifugation the pellet was solubilized with 8 M guanidine hydrochloride and 0.1 M dithiothreitol. The suspension was homogenized by sonication for 10 minutes then centrifuged for 25 min at 15 557 g. The supernatant (containing solubilized denatured NGF) was collected and filtered using a 45 m filter then added dropwise into refolding buffer (0.75 M L-arginine, 0.1 M Tris, 1 mM EDTA, 5 mM reduced glutathione and 0.5 mM oxidized glutathione pH 9.5) at 7° C. with constant stirring to a final concentration of 5 ml of NGF solution per 100 ml of refolding buffer. After overnight incubation the refolding solution was centrifuged at 10 000 g for 10 minutes and the supernatant collected and incubated for one week at +7° C. The solution was diluted threefold with deionized water, warmed to room temperature and the pH adjusted to 6.8 with acetic acid. The solution was then centrifuged at 7000 g for 10 minutes at room temperature to remove precipitates and loaded on a 5 ml Capto S cation exchange column, previously equilibrated with 50 mM sodium phosphate buffer (pH 6.5). The proteins were then eluted with a gradient of 0-1 M NaCl in 50 mM sodium phosphate buffer (pH 6.5). The eluted fractions were analyzed with SDS-PAGE and those containing proNGF were pooled and concentrated with ultrafiltration to 2-3 mg/ml. The renatured proNGF was digested with TrypZean (Sigma-Aldrich, cat no. T3449) trypsin solution for 4 hours at room with volume ratio of 30:1. The reaction was stopped by adding PMSF to final concentration of 1 mM, then loaded onto a Superdex 200 10/300 GL size exclusion column equilibrated with 0.5 M NaCl and 30 mM phosphate (pH 6.8). Fractions were collected and analysed with SDS-PAGE (FIG. 15A; shown for cDNA construct of SEQ ID NO:28 and resulting amino acid sequence of the full-length feline NGF pro-peptide of SEQ ID NO:29) and those containing the mature NGF were pooled and concentrated by ultrafiltration to a concentration of 2 mg/ml.

The authenticity of the recombinant canine mature NGF was confirmed using a bioassay which showed the canine mature NGF and mouse mature NGF (commercially produced by R&D systems) were similarly active at inducing neurite (FIG. 15B: shown for cDNA construct of SEQ ID NO:28 and resulting amino acid sequence of the full-length feline NGF pro-peptide of SEQ ID NO:29); a known function of properly folded and biologically active mature NGF.

### Example 5

### Coupling of Recombinant Canine Mature NGF to Modified CMV VLPs

Various NGF antigens comprising canine mature NGF (SEQ ID NO:30) were covalently linked to the various modified CMV VLPs prepared as described above. The linking was effected in accordance with the method described in Schmitz N, et al, J Exp Med (2009) 206: 1941-1955). Briefly, purified CMV-Ntt830, CMV-Ntt830-E4, CMV-Ntt830-E8 or CMV-Ntt830-E8* VLPs were diluted to 1.5 mg/ml and reacted with heterobifunctional chemical cross-linker succinimidyl-6-(b-maleimidopropionamide) hexanoate (SMPH) for 1 hour at room temperature (RT). SMPH contains a NHS ester which reacts with the lysine on the surface of the VLP. The amount of SMPH added was approximately 5× molar excess over one VLP coat protein monomer. Cross-linker which did not react with the VLP was removed by centrifugation using an Amicon-Ultra-0.5, 100K centrifugal filter (Merck-Millipore, #UFC910024). The SMPH-derivatized VLPs were then washed 3 times with 5 mM Na2HPO4, 2 mM EDTA (pH 7.5).

In detail, and described for the coupling of cNGF antigens having SEQ ID NO:174 to CMV-Ntt830-E4 VLPs: A solution of CMV-Ntt830-E4 VLPs in 5 mM NaHPO4 pH 7.5, 2 mM EDTA,—with a protein concentration of 7.43 mg/ml BCA Protein Assay Kit (TFS, Cat. No. 23225) was diluted to a working concentration of 1.5 mg/ml with 5 mM NaHPO4 pH 7.5, 2 mM EDTA pH 8.0 in 3×44 ml sample volume in 50 ml tubes (Sarstedt, sterile, Cat. No. 62.559.001), thus the total volume for derivatization was 132 ml. 50 mM (19 mg/ml) SMPH solution in DMSO was prepared directly before use.

For derivatization of CMV-Ntt830-E4 VLPs with SMPH, 264 μl of 50 mM SMPH solution in DMSO was added to each of previously prepared three tubes containing 44 ml of CMV-Ntt830-E4 VLPs. Mixture was vortexed for 5 seconds and incubated at RT for 1 h. To remove excess SMPH mixture was further centrifugated on Amicon-Ultra-15 100K units (Merck-Millipore, Cat. No. UFC910024) for 7 min at 3214 g in Eppendorf 5810R centrifuge. The buffer was exchanged to 5 mM NaHPO4 pH 7.5, 2 mM EDTA by 3 more centrifuge runs with same parameters. After the last centrifugation run the total volume was adjusted to 132 ml (same as before derivatization). UV absorption at 260 nm was measured and concentration of derivatized CMV-Ntt830-E4 VLPs was estimated at 1.5 mg/ml.

Next, and briefly, cNGF antigens were added to the VLPs in an about 0.5:1 to 1:1 molar ratio, with respect to the respective chimeric CMV polypeptide monomer, to the previously SMPH derivatized surface charge modified CMV VLPs for typically 3 hours at RT while shaking. The engineered free cysteine of the cNGF antigen reacted with the maleimide of the cross-linker SMPH bound to the VLPs to form a stable covalent linkage.

In detail, and described for the coupling of cNGF antigens having SEQ ID NO:174 to CMV-Ntt830-E4 VLPs: The coupling reaction were performed in six 50 ml tubes (Sarstedt, sterile, Cat. No. 62.559.001). In each tube 22 ml of derivatized CMV-Ntt830-E4 VLPs (1.5 mg/ml, 60 M in respect to CMV monomers) was mixed with 3.82 ml of buffer-exchanged cNGF of SEQ ID NO:174 (2.33 mg/ml, 172.6 μM). This yielded to a molar ratio of CMV monomers:NGF monomers=1:0.5. The reaction mix was incubated at RT by end-over-end rotation with DSG Titertek (Flow Laboratories). Uncoupled cNGF was removed by gel-filtration on Superdex 200 column (run buffer 20 mM NaHPO4 pH 7.5, 2 mM EDTA). 10 ml of the solution comprising cNGF-CMV-Ntt830-E4 VLPs was loaded on a HiLoad 26/600 Superdex 200 prep grade column equilibrated in 20 mM NaHPO4 pH 7.5, 2 mM EDTA. The fractions containing cNGF-CMV-Ntt830-E4 VLPs were pooled and filtrated with filtered through a 0.2 m filter (Sarstedt, Cat. No. 83.1826.001). Collected sample volume after gel-filtration was 280 ml. The sample was further concentrated by Amicon-Ultra-15, 100K (Merck-Millipore, Cat. No. UFC910024) to 230 ml and filtered through a 0.2 m filter (Sarstedt, Cat. No. 83.1826.001). The concentration was measured by Qubit and the final concentration was adjusted to 0.7 mg/ml with sterile 20 mM NaHPO4 pH 7.5, 2 mM EDTA buffer. UV absorbance at 260 nm was measured (A230=6.628 and A260=3.722).

To demonstrate covalent conjugation of cNGF antigens to VLPs, coupling reactions were analyzed by SDS-PAGE. Prominent conjugation bands were observed following chemical coupling of cNGF with CMV-Ntt830, CMV-Ntt830-E4, CMV-Ntt830-E8 and CMV-Ntt830-E8* VLPs (FIG. 16A and FIG. 16B). However, cNGF-CMV-Ntt VLPs formed large aggregates (1400-1700 nm) (FIG. 16C) and rapidly and completely precipitated from solution.

In contrast, after the covalent conjugation of cNGF to the CMV-Ntt830-E4, CMV-Ntt830-E8 and CMV-Ntt830-E8* VLPs, the resultant modified VLP conjugates remained soluble and did not precipitate from solution. Analysis by dynamic light scattering (DLS) (FIG. 16D, FIG. 16E and FIG. 16F) and electron microscopy (FIG. 16G and FIG. 16H) showed the modified VLP conjugates were not aggregated and were stable in solution.

### Example 6

Induction of Neutralizing Antibodies by Immunizing with Various Inventive Conjugates of Canine Mature NGF Coupled to Modified CMV VLPs

### Immunization of Mice

Balb/c mice were assigned to two groups (n=4 per group). The first group was immunized twice 14 days apart with 150 μl of canine mature NGF-CMV-Ntt830-E8* VLP formulated to a concentration of 100 μg/ml in 20 mM NaP, 2 mM EDTA, pH 7.5. The second group was similarly treated with canine mature NGF-CMV-Ntt830-E8* VLP formulated to a concentration of 100 μg/ml in 20 mM NaP, 2 mM EDTA, pH 7.5 and 100 μg/ml Quil-A adjuvant (InvivoGen vac-quil). Before each immunization, blood was taken as well as on days 21, 28, 35 and 42 after the first vaccination. Serum was prepared by spinning the blood samples in serum tubes at 10,000×g for 10 min. Sera were stored at ca. −20° C. until assay.

### Immunization of Dogs

Six male Beagles aged 22-26 months at the time of first dosing (obtained from Marshall US) were assigned across 2 groups (n=3 per group) by randomization. The first group was immunized three times with 1.0 ml of cNGF-CMV-Ntt830-E8* VLP formulated to a concentration of 250 μg/ml in Na phosphate buffer, pH 7.5. The second group was similarly treated with cNGF-CMV-Ntt830-E8* VLP formulated to a concentration of 250 μg/ml in Na phosphate buffer, pH 7.5 and 100 μg/ml Quil-A® adjuvant (In vivoGen vac-quil). Blood specimens were drawn from the jugular vein with single use needles and syringes of each animal 24 hours before the first (day 0), second (Day 21) and third (Day 42) immunization. Blood was also drawn on days 63, 84 and 105. Six ml samples of blood were collected in inert tubes and left at ambient temperature. After clot formation, the tubes were centrifuged and serum collected into inert tubes and stored at ca. −20° C. until IgG purification and/or assayed.

In a further study, 10 adult Beagle dogs over the age of 9 months at inclusion were allocated into 2 groups. For immunization, cNGF-CMV-Ntt830-E4 VLPs comprising cNGF antigens of SEQ ID NO:33 were used. Thus, the first group of 5 dogs were treated with 250 μg cNGF-CMV-Ntt830-E4 VLPs/dose formulated with 1.7 mg aluminium hydroxide, while the second group of 5 dogs were treated with 250 μg cNGF-CMV-Ntt830-E4 VLPs/dose without aluminium hydroxide. Dogs were administered subcutaneously on two occasions on study days 0 and 21. Serum samples were also collected throughout the study on days 42, 71 and 91.

### Measurement of NGF and CMV-VLP-Specific IgG Antibodies

For mice and dogs immunized with cNGF-CMV-Ntt830-E8* VLP, anti-NGF- and CMV-Ntt830-E8*-VLP specific IgG antibodies in sera were measured by ELISA. For dogs immunized with cNGF-CMV-Ntt830-E4 VLP, anti-NGF-specific IgG antibodies in sera were measured by ELISA.

As described in detail for the immunization with cNGF-CMV-Ntt830-E8* VLPs, Maxisorp ELISA plates were coated with recombinant canine mature NGF protein or CMV-Ntt830-E8*-VLP in 0.1 M Na Carbonate buffer, pH 9.6 at a concentration of 1 μg/ml overnight at 4° C. Plates were washed and SuperBlock™ (PBS) Blocking buffer (Thermo Fisher/Life Technologies Europe) added for 2 hours at RT then washed again. Serum samples were pre-diluted 1:9 in 2% BSA in PBS with 0.05% Tween 20, transferred to the ELISA plates and subjected to ten 3-fold serial dilutions. Following incubation for 2 hours at RT and washing, Horse-radish peroxidase- (HRP-)labelled goat anti-mouse IgG, Fc gamma fragment specific (Jackson ImmunoResearch Europe Ltd) or HRP-labelled rabbit anti-dog IgG (H+L)-RP, (Jackson ImmunoResearch Europe Ltd) diluted 1:2000 or 1:2500 respectively in 2% BSA in PBS (PBS pH 7.4 (1×) Gibco) with 0.05% Tween-20 was added. After incubation and washing, Pierce™ TMB Substrate Kit (Thermo Fisher/Life Technologies Europe)) was used for colorimetric development. The enzymatic reaction was stopped by the addition of 5% H₂SO₄ and the absorbance at 450 nm measured by spectrophotometry using an ELISA reader (Tecan Spark 10). An OD50 titer describes the reciprocal of the dilution, which reaches half of the maximal OD value.

### Neutralization Assay PC12 Cells.

An in vitro assay measuring mature NGF-mediated neurite outgrowth in rat adrenal phaeochromocytoma cell cultures (PC-12) was used to determine the bioactivity of recombinantly produced canine mature NGF and assess the neutralizing ability of antibodies induced by immunization of mice. Type-I collagen (Thermo Fisher/Life Technologies Europe) (10 μg/ml) coated 24-well tissue culture plates were seeded in duplicate with 5×104 PC-12 cells/well in assay media comprising RPMI 1640 (Sigma-Aldrich Switzerland), 2 mM L-Glutamine (Gibco), 2.4 g/L HEPES (AppliChem GmbH Germany)) 2.5 g/L Glucose (Sigma-Aldrich, Switzerland), further supplemented with 10% heat-inactivated Fetal Bovine Serum (FBS Premium, PAN Biotech, Germany), 10% Horse Serum (kindly provided by Evax, Switzerland), 1× Antibiotic-Antimycotic (A/A) (Gibco, Thermo Fisher/Life Technologies Europe) and 1 mM sodium pyruvate (Sigma-Aldrich, Switzerland) and incubated overnight at 37° C., 5% CO₂. The following day, media from wells were replaced with assay media (RPMI 1640, 1× A/A, 1 mM sodium pyruvate, 2 mM L-Glutamine, 0.5% FBS) containing final concentrations of 12.5 ng/ml mouse (R&D, 1156-NG-100), human mature NGF (R&D, 256-GF-100/CF) or recombinantly produced canine mature NGF, with human mature NGF polyclonal antibody (R&D AF-256-NA), human mature NGF monoclonal antibody (R&D MAB256-500) or purified IgG from vaccinated mice. NGF was omitted from negative control wells (starvation medium alone) and antibodies were omitted from positive control (12.5 ng/ml NGF in starvation medium) wells. Cells were stained with 0.05% w/v crystal violet solution after 5 days and inspected by microscopy. Brightfield images from several fields of view were captured on an inverted microscope Leica DM IL LED (Leica Microsystems (UK) Ltd), HI PLAN I 20× objective, using Q-Capture Pro 7software. Cells with and without neurite outgrowth (defined as extending cell body width) were counted and the proportion of neurite positive cells for each treatment was determined.

### Neutralization Assay TF-1 Cells

The neutralizing ability of sera from dogs immunized with cNGF-CMV-Ntt830-E8* VLP and cNGF-CMV-Ntt830-E4 VLP was determined using a bioactivity assay that involved measuring proliferation of the TF-1 erythroblastoma cell line (American Type Culture Collection (ATCC), Manassas, VA).

For the immunization with cNGF-CMV-Ntt830-E8* VLPs, TF-1 cells were harvested, washed three times in PBS (PBS pH 7.4 (1×) Gibco) and cultured overnight in starvation medium (RPMI 1640 Medium (ATCC modification) supplemented with heat inactivated 10% FBS, 1× A/A) at a cell density of 10⁵ cells/ml. 10⁴ TF-1 cells were seeded in a total of 100 μl assay medium (phenol-red free RPMI containing 10% FBS, 2 mM GlutaMax, 10 mM HEPES, 1 mM sodium pyruvate, 4500 mg/L glucose, 1500 mg/L sodium bicarbonate, 100 U/mL Penicillin, 100 μg/mL streptomycin, 25 μg/mL Amphotericin B) per well of a 96-well flat-bottom plate.

To test the in vitro neutralizing activity of antibodies raised by immunization with cNGF-CMV-Ntt830-E8* VLP, sera of immunized dogs were collected and total IgG purified according to manufacturer's instruction using Invitrogen Dynabeads™ Protein G (Thermo Fisher/Life Technologies Europe) for mouse IgG purification and Pierce Protein A Magnetic Beads (Thermo Fisher/Life Technologies Europe) for dog. The capacity of purified total IgGs to neutralize the bioactivity of NGF was tested by incubating a constant concentration of 5 ng/ml human mature NGF (R&D, 256-GF-100/CF) with increasing concentrations of purified dog total IgGs (625-20000 ng/mL), human mature NGF polyclonal antibody (R&D AF-256-NA) or human mature NGF monoclonal antibody (R&D MAB256-500) for 1 hour at room temperature. The NGF-antibody solution was then added to 10⁴ TF-1 cells starved overnight and cell proliferation was quantified over the last 24 hour period of the total 72 hour incubation time using the BrdU based cell Proliferation ELISA (Roche). Manufacturer's instruction were followed and color development was stopped with 5% sulfuric acid. Absorbance was measured at 450 nm with a reference wavelength of 690 nm.

The percent proliferation for each IgG dilution was calculated in relation to the proliferation measured for IgG purified from sera collected at baseline prior to infection (day 0). Data was expressed as percent proliferation versus IgG concentration. GraphPad Prism (version 8.0.0 for Windows, GraphPad Software, San Diego, California USA, www.graphpad.com) was used to fit a sigmoidal 4PL curve to determine the IgG concentration required to achieve 50% inhibition of proliferation (50% Neutralization Titer NT50).

NGF neutralizing antibodies in dogs after the immunization with cNGF-CMV-Ntt830-E4 VLP were determined as follows: TF-1 cells were harvested and washed 3 times with PBS prior to resuspension in starvation medium (Phenol-red free RPMI (Sigma) containing 10% HI-FBS, 2 mM GlutaMax (Gibco), 10 mM HEPES (Sigma), 1 mM sodium pyruvate (Sigma), 4500 mg/L glucose (Gibco), 1500 mg/L sodium bicarbonate, 100 U/mL Penicillin, 100 μg/mL streptomycin, 25 μg/mL Amphotericin B (100× anti-anti Gibco) at a cell density of 2×10⁵ cells/mL. Serum samples were heat inactivated for 30 minutes at 56° C. then diluted 1:25 (4-time final concentration of 1:100) in starvation medium and 2-fold serial dilution was performed. hNGF was diluted to 20 ng/mL (4-times final concentration of 5 ng/mL) and 25 μL added to wells containing 25 μL prediluted serum or 25 μL starvation medium (positive control wells). Instead of hNGF, 50 μL of starvation medium was added to negative control wells. hNGF-serum/antibody mix was incubated for 1 hour at room temperature. Serum starved TF-1 cells were collected, and 50 μL cell suspension were added at a cell density of 1×10⁴ cells/well of a flat bottom 96 well plate. The final sample volume per plate was 100 μL/well. Cell culture plates were incubated for approximately 68 hours at +37° C. in a 5% CO₂ cell culture incubator. Viability of cells was quantitated by the Promega CellTiter 96 Aqueous One Solution Cell Proliferation Assay (Promega). 20 μL of CellTiter 96® Aqueous One Solution Reagent was added per well. Plates were incubated for 7 hours at +37° C. in a humidified, 5% CO₂ incubator. Absorbance at 490 nm with a reference wavelength at 700 nm was recorded. To determine the IC50 values, titration curves were generated by plotting the OD values versus the dilution factor of the serum sample using GraphPad prism software (GraphPad Prism version 8 and 9 for Windows, GraphPad Software, San Diego, California USA). Using a 4-PL regression curve fit model the IC50 values, the dilution factor corresponding to half maximum OD values, were determined. Serum titers of samples at different time points were defined and depicted as the IC50 values of the curve fit.

### Results

### Murine Experiments

For mice immunized with cNGF-CMV-Ntt830-E8* VLPs with Quil A, cNGF-specific IgG antibodies were detected in sera collected from day 14 onwards (FIG. 17A). A further increase in the antibody titers was measured in day 21 sera 7 days after administration of the second injection on day 14. Titers remained high until termination of the experiment on day 42. After two immunizations of cNGF-CMV-Ntt830-E8* VLPs vaccine without adjuvant, NGF-specific IgG antibodies were detected in the sera isolated from day 21 onwards. The co-administration of Quil A adjuvant had an immune-enhancing effect and boosted the specific antibody response by a factor of approximately 10.

To test if anti-NGF IgG antibodies induced by immunization with cNGF-CMV-Ntt830-E8* VLPs were neutralizing, they were tested in a PC12 based bioassay where NGF acts as a neurotrophic factor inducing differentiation and neurite outgrowth. IgG was purified from pooled sera collected prior to immunization (ms pIgG NAÏVE) and at days 21, 28 and 35 after cNGF-CMV-Ntt830-E8* VLPs/QuilA boost (ms pIgGNGF vacc). FIG. 17B shows that IgG purified from immune sera neutralized NGF whereas IgG from naïve mice did not.

### Canine Experiments

In animals receiving cNGF-CMV-Ntt830-E8* VLPs in the absence of adjuvant, detectable anti-NGF IgG titers were observed in sera collected at day 21 (FIG. 18A) after a single administration of the vaccine. NGF-specific IgG titers were highest in day 42 sera 3 weeks following the second administration of the vaccine. After the third injection on day 42, the titers remained constantly high until day 63 and declined gradually thereafter in all animals. The magnitude of the anti-CMV IgG titers was similar to those measured against canine mature NGF but the kinetic of the response was slightly different (FIG. 18C). The anti-CMV IgG antibodies were somewhat delayed and only unequivocally detectable from day 42 onwards after the second immunization and peak titers were measured in day 63 sera following the third immunization where after the titers declined.

For animals immunized with cNGF-CMV-Ntt830-E8* VLPs in combination with adjuvant Quil A, anti-NGF IgG antibodies were first detected in day 21 sera after a single administration of vaccine on day 0 (FIG. 18B). The second and third doses of vaccine increased the titers in two out of three animals with peak titers measured in sera collected at day 63. The third animal achieved its peak titer at day 42 suggesting the third dose of vaccine may not have increased the antibody response. The kinetics and magnitude of the anti-CMV IgG antibody titers were similar to those measured against canine mature NGF (FIG. 18D).

In animals receiving cNGF-CMV-Ntt830-E8* VLPs in the absence of adjuvant, detectable anti-NGF IgG titers were observed in 4 out of 5 study animals in sera collected at day 21 (FIG. 18E) after a single administration of the vaccine. Highest titers were observed 21 days after second dose on day 42.

For animals immunized with cNGF-CMV-Ntt830-E4 VLPs in combination with aluminum hydroxide, anti-NGF IgG antibodies were detected all animals 3 weeks after a single administration of vaccine on day 0 (FIG. 18F). The second dose of vaccine increased the mean group titer.

The neutralizing ability of anti-NGF IgG antibodies induced in response to the vaccination with cNGF-CMV-Ntt830-E8* VLP was analysed using a bioassay based on NGF mediated proliferation of TF-1 cells. IgG antibodies purified from immunized dogs inhibited mature NGF induced proliferation in a concentration dependent manner whereas IgG antibodies purified from pre-immune sera of the same animals failed to do so (FIG. 19A). Vaccination with cNGF-CMV-Ntt830-E8* VLP induced high neutralization titers that could be further increased by co-administration of the vaccine with Quil A adjuvant (FIG. 19B). This observation reflects the anti-NGF ELISA IgG titers in these dogs described above. A clear correlation between the anti-NGF titers and the neutralization capacity was observed (FIG. 19C). IgG purified from sera with high vaccine specific titers had increased potency with respect to inhibition of NGF mediated proliferation of TF-1 cells.

Vaccination with cNGF-CMV-Ntt830-E4 VLP induced neutralizing anti NGF antibody titers. High levels of neutralizing anti-NGF antibodies in the sera collected from dogs immunized twice with cNGF-CMV-Ntt830-E4 in presence of aluminium hydroxide were observed at day 42 (FIG. 19D).

These results show that conjugates of canine mature NGF coupled to modified VLPs comprising chimeric CMV polypeptides in accordance with the present invention are able to overcome immune tolerance to the endogenous target antigen and induce NGF-specific IgG antibodies in dogs, the target species. Moreover, these antibodies were able to efficiently neutralize canine mature NGF activity in vitro.

### Example 7

### Cloning, Expression and Purification of Recombinant Feline IL-5 Dimer Antigen

### Cloning of Recombinant Feline IL-5 Dimer Antigen

A codon optimized nucleotide sequence, encoding feline IL-5 monomer with a hexa-histidine tag and a glycine-cysteine comprising linker at the C-terminus, cloned into Bam H1 and Spe I restriction sites of a modified pET42 expression plasmid was used as a template to synthesize a cDNA construct consisting of feline full-length IL-5 dimer with a flexible linker inserted between the C terminus and N terminus of the tandem IL5 sequences and a C-terminal hexa-histidine glycine-cysteine comprising motif.

The optimized nucleotide sequence of the template DNA has the sequence as set forth in SEQ ID NO:34, which encodes the amino acid sequence as set forth in SEQ ID NO:35.

The DNA construct was used as template DNA rather than for expression purposes because very low levels of target antigen were obtained with it. Since native feline IL-5 is a homo-dimer, it was decided to instead make a head-tail linked feline IL-5 dimer. This was achieved by PCR using the above construct as a template and the following pairs of oligonucleotide primers:

1^{st} reaction:
(SEQ ID NO: 36)
2xIL5-BamF
(SEQ ID NO: 37)
2xIL5-gsKpnR
2^{nd} reaction:
(SEQ ID NO: 38)
2xIL5-gsKpnF
(SEQ ID NO: 39)
2xIL5-SpeR

Both PCR products were directly sub-cloned into pTZ57 helper vectors (Fermentas). The resulting plasmids were amplified in E. coli XL1 Blue cells and sequenced using a BigDye cycle sequencing kit and an ABI Prism 3100 Genetic analyzer (Applied Biosystems, Carlsbad, USA). After identification of plasmid clones without PCR errors, the IL5 containing plasmid generated from the 2^{nd} PCR reaction was cut with Kpn2/EcoRI enzymes and ligated into the plasmid containing the product of the 1st PCR reaction cut with the same enzymes. The resulting pTZ-derived plasmid contained both feline IL-5 genes connected with a sequence encoding a (GGGGS)₃-linker (SEQ ID NO:40).

This construct encoding the head-tail linked feline IL-5 dimer was then cloned into a Bam HI/Spe I site of a modified pET42 vector. The map of the resulting expression vector pET42NBS-2×flIL5-C6Hcg is shown in FIG. 20.

The resulting amino acid sequence of the provided antigen comprising such feline IL-5 dimer head-tail linked via the flexible (GGGGS)₃-linker (SEQ ID NO:41) and further comprising the C-terminal hexa-histidine glycine-cysteine comprising motif is set forth in SEQ ID NO:42. The corresponding nucleotide sequence is set forth in SEQ ID NO:43.

### Expression and Purification of Recombinant Feline IL-5 Dimer Antigen

To obtain recombinant feline IL-5 dimer antigen, E. coli BL21 (DE3) cells were transformed with the pET42NBS-2×flIL5-C6Hcg plasmid. After selection of clones expressing the highest levels of target protein, E. coli cultures were grown in auto-induction medium (50 mM NaP pH 7.0, 50 mM KP pH 7.6, 25 mM (NH₄)₂SO₄, 2 mM MgCl₂, 0.05% Glucose, 0.5% Glycerol, 0.1% Lactose, 1.2% yeast extract, 0.75% tryptone, 0.375% NaCl, kanamycin 25 mg/l) on a rotary shaker (210 rev/min) at 20° C. for 24 h. The resulting biomass was collected by low-speed centrifugation and was frozen at −20° C. until the purification.

The recombinant feline IL-5 dimer antigen was purified using a USB PrepEase Kit (Affymetrix, High Wycombe, UK; #78803-1-KT) according to the manufacturer's recommendations. After thawing on ice, E. coli cells from 100 ml culture (approx. 0.75 g) were suspended in 1×LEW buffer and disrupted by sonication (UP200S; period 0.5; intensity 70% for 16 min). Insoluble proteins and cell debris were removed by centrifugation (13,000 rpm, 30 min at 5° C.). The clarified soluble fraction was applied to a USB PrepEase column, washed twice with the same buffer and eluted with 3×1.5 ml of imidazole containing 1×E buffer. Fractions containing the recombinant feline IL-5 dimer antigen were identified by SDS/PAGE and Western blot, pooled and dialyzed against 200 volumes of buffer (5 mM Na phosphate, 2 mM EDTA, pH 7.5) using a Spectra/Por membrane with MWCO 3.5 kDa (Spectrum Laboratories, #132720). The protein concentration was estimated using a QuBit fluorometer in accordance with the manufacturer's recommendations (Invitrogen, Eugene, USA). The purity and identity and of the purified recombinant feline IL-5 dimer antigen was demonstrated by SDS-PAGE and Western Blot (FIG. 21, Lanes 5) and mass spectrometric analysis.

### Example 8

### Coupling of Recombinant Feline IL-5 Dimer Antigen to Modified CMV VLPs

Recombinant feline IL-5 dimer antigen (SEQ ID NO:42) was covalently linked to the various modified CMV VLPs prepared as described above. The linking was effected in accordance with the method described in Schmitz N, et al, J. Exp. Med (2009) 206: 1941-1955). Briefly, purified CMV-Ntt830, CMV-Ntt830-E4 or CMV-Ntt830-E8* VLPs were diluted to 1.5 mg/ml and reacted with a 5× molar excess (over one VLP coat protein monomer) of the heterobifunctional chemical cross-linker succinimidyl-6-(b-maleimidopropionamide) hexanoate (SMPH) for 1 hour at room temperature (RT). SMPH contains a NHS ester which reacts with the lysine on the surface of the VLP. Cross-linker which did not react with the VLP was removed by centrifugation using an Amicon-Ultra-0.5, 100K centrifugal filter (Merck-Millipore, #UFC910024). The SMPH-derivatized VLPs were then washed 3 times with 5 mM Na2HPO4, 2 mM EDTA (pH 7.5).

Next, recombinant feline IL-5 dimer antigen was pre-treated with a 10× molar excess of TCEP for 10 minutes at RT, at a concentration of 1.87 mg/ml then added in a 1:1 molar ratio, with respect to the VLP coat protein monomer, to the previously SMPH derivatized CMV VLPs for 3 hours at RT while shaking. The engineered free cysteines of the recombinant feline IL-5 dimer antigen reacted with the maleimide group of the cross-linker SMPH bound to the VLP to form a stable covalent linkage.

To demonstrate covalent conjugation of antigen to VLPs, various stages of the coupling reactions were analyzed by SDS-PAGE and Western blot. This is exemplified in FIG. 21 where samples of SMPH-derivatized CMV-Ntt830-E8* VLPs, recombinant feline IL-5 dimer antigen and the recombinant feline IL-5 dimer antigen-CMV-E8* conjugated VLPs were run in parallel. These analyses demonstrated the presence of bands representing the recombinant feline IL-5 dimer antigen-VLP coat protein conjugates. A band consisting of one CMV-Ntt830-E8* monomer covalently linked to one recombinant feline IL-5 dimer antigen molecule was observed in both Coomassie blue stained gels and Western blot analysis. A higher molecular weight band of approximately representing CMV-Ntt830-E8* VLP coat protein dimer conjugated with one recombinant feline IL-5 dimer antigen was also observed.

Prominent conjugation bands were observed following chemical coupling of recombinant feline IL-5 dimer antigen with CMV-Ntt830, CMV-Ntt830-E4 or CMV-E8* VLPs (FIG. 22). However, analysis by agarose gel electrophoresis and dynamic light scattering (DLS) clearly demonstrated that recombinant feline IL-5 dimer antigen-CMV-Ntt830 VLPs formed large aggregates >1000 nm (FIG. 23A, FIG. 23B and FIG. 24A). Furthermore the vaccine was not stable and precipitated.

In contrast, analysis of the covalent conjugation of recombinant feline IL-5 dimer antigen to CMV-Ntt830-E4 and CMV-Ntt830-E8* VLP variants by native agarose gels, dynamic light scattering (DLS) and EM analysis clearly demonstrate the VLPs were not aggregated and had typical VLP structures (FIG. 23A, FIG. 23B, FIG. 24B, FIG. 24C and FIG. 24D). Moreover, the vaccines were soluble and did not precipitate from solution.

### Example 9

Induction of Neutralizing Antibodies Upon Immunization with Modified CMV VLPs to which Recombinant Feline IL-5 Dimer Antigens are Coupled

### Immunization of Mice

Balb/c mice (n=4) were immunized subcutaneously twice 14 days apart with 30 μg of 2×flIL5-CMV-Ntt830-E8* VLPs formulated in 5 mM NaP, 2 mM EDTA, pH 7.5. Before each immunization as well as on days 7 and 28 after the first vaccination, blood was collected into serum microtainer tubes (BD). Serum was prepared by spinning the collection tubes at 8000×g for 10 min. Sera were stored at ca. −20° C. until assay.

### Immunization of Cats

Domestic cats (n=3) were immunized subcutaneously three times with a 3 weekly interval with 250 μg of 2×flIL5-CMV-E8* vaccine. Blood was collected before each immunization and on weeks 9, 12 and 15 after the first immunization. Serum was prepared and stored at ca. −20° C. until assaying. Whole blood containing EDTA was used for manual counting of eosinophils.

For mice and cats immunized with 2×flIL5-CMV-Ntt830-E8* VLPs, IL-5 and CMV-VLP specific IgG antibodies in sera were measured by ELISA. Maxisorp ELISA plates were coated with recombinantly produced feline IL-5 protein or CMV-VLP in 0.1 M Na Carbonate buffer, pH 9.6 at a concentration of 1 μg/ml overnight at 4° C. Plates were washed and SuperBlock™ (PBS) blocking buffer (Thermo Fisher/Life Technologies Europe) added for a minimum of 30 minutes at RT then washed again. Serum samples were pre-diluted 10 fold in 2% BSA in PBS with 0.05% Tween 20, transferred to the ELISA plates and subjected to 7 or ten×3-fold serial dilutions. Following incubation for 1 or 1.5 hours at RT and washing, horse-radish peroxidase- (HRP-)labelled goat anti-mouse IgG, Fc gamma fragment specific (Jackson ImmunoResearch Europe Ltd) or HRP-labelled goat anti-cat IgG (H+L)-HRP, (Jackson ImmunoResearch Europe Ltd) diluted 1:2000 or 1:1000 respectively in 2% BSA in PBS (PBS pH 7.4 (1×) Gibco) with 0.05% Tween-20 was added. After incubation and washing, OPD substrate (Sigma-Aldrich) was used for colorimetric development. The enzymatic reaction was stopped by the addition of 5% H₂SO₄ and the absorbance at 490 nm measured by spectrophotometry using an ELISA reader (Tecan Spark 10). Serial dilutions were used to calculate the OD50 titers. An OD50 titer describes the reciprocal of the dilution, which reaches half of the maximal OD value.

The neutralizing ability of sera from mice and cats immunized with 2×fIL5-CMV-Ntt830-E8* VLPs was determined using a bioactivity assay which involved measuring proliferation of a human TL5 adapted TF-1 erythroblastoma cell line (American Type Culture Collection (ATCC), Manassas, VA) in response to feline IL-5.

Briefly, an TL-5 responsive TF-1 cell lines was generated by sub-culturing and sub-cloning the TF-1 erythroblastoma cell line (American Type Culture Collection (ATCC) in the presence of 10 ng/mL human IL-5 (Peprotech) in RPMI 1640 Medium (ATCC modification) supplemented with heat inactivated 10% FBS and antibiotics. On the day of the assay set up, IL-5 responsive TF-1 cells were harvested, washed three time in PBS (PBS pH 7.4 (1×) Gibco) and seeded in a total of 100 μl starvation medium (phenol-red free RPMI containing 10% FBS, 2 mM GlutaMax, 10 mM HEPES, 1 mM sodium pyruvate, 4500 mg/L glucose, 1500 mg/L sodium bicarbonate, 100 U/mL Penicillin, 100 μg/mL streptomycin, 25 μg/mL Amphotericin B per well of a 96-well flat-bottom plate. To test the in vitro neutralizing activity of antibodies raised in response to immunization with 2×fIL5-CMV-Ntt830-E8* VLPs, heat-inactivated, serial diluted sera were incubated with a constant concentration of 10 ng/mL recombinant feline IL-5 dimer. Goat anti-feline IL-5 polyclonal antibody (ThermoScientific/Invitrogen PA5-47994) was used as a positive control. Serial diluted sera or antibodies were pre-incubated with feline IL-5 for 1 hour at room temperature. The IL-5-antibody solution was then added to 10⁴ TF-1 cells and incubated for 3 days. 10 ng/ml of feline IL-5 in absence of antibodies was used a positive control. Neither antibodies nor cytokine was added to negative control wells. Cell viability was quantified over the last 6 to 7 hour incubation period using CellTiter 96 AQueous One Solution Cell Proliferation Assay reagent (Promega). Absorbance was measured using a microplate reader (TecanSpark) at a wavelength of 490 nm and a reference wavelength of 700 nm.

Titration curves were generated by plotting the difference in absorbance (490 nm-700 nm) versus the dilution factor of the serum sample. Using a 4-Parameter logistic regression curve fit model the neutralization titers, NT50 values, the dilution factor corresponding to half maximum OD values, were determined. Serum titers of samples at different time points were defined and depicted as the NT50 values of the curve fit (left graph).

### Results

### Murine Experiments

For all mice immunized with 2×fIL5-CMV-Ntt830-E8* VLPs, IL5-specific IgG antibodies were detected in sera collected from day 7 onwards (FIG. 25A). Antibody titers had increased further by day 14 and following administration of the second injection, also on day 14, titers increased further still until termination of the experiment on day 28.

To test if the anti-IL5 antibodies induced by immunization with 2×fIL5-CMV-Ntt830-E8* VLPs were neutralizing, a TF-1 cell based bioassay where feline IL-5 acts as a growth factor inducing cell survival and proliferation was employed. Sera collected at day 0 and 28 were incubated with feline IL-5. FIG. 25B shows that immune sera neutralized feline IL-5. There was no neutralization with pre-immune (day 0) sera.

### Feline Experiments

In animals immunized with 2×fIL5-CMV-Ntt830-E8* VLPs, detectable anti-IL5 IgG titers were observed in sera collected at day 21 (FIG. 26A) after a single administration of the vaccine. Peak IL-5-specific IgG titers were highest in either day 42 or day 63 sera 3 weeks following the second or third administration of vaccine respectively. After day 63, the titers declined gradually in all animals until study termination at day 105. The magnitude and kinetic of the anti-CMV IgG titers were similar to the IL5 titers FIG. 26B).

The neutralizing ability of anti-IL5 sera induced in response to the vaccination with 2×fIL5-CMV-Ntt830-E8* VLPs was analyzed using a bioassay based on IL-5 mediated proliferation of TF-1 cells. Immune sera collected on days 42, 63 and 105 inhibited IL-5 induced proliferation of TF-1 cells (FIG. 26C). Pre-immune sera (day 0) of the same animals failed to do so. A clear correlation between the high anti-IL-5 IgG titers and high neutralization capacity of sera was observed (FIG. 26D).

Concomitantly with the induction in IL-5 neutralizing antibodies, the number of blood eosinophils decreased from day 21 throughout the course of the study in all three animals after immunization. (FIG. 26E).

These results show that 2×fIL5-CMV-Ntt830-E8* VLPs is able to overcome immune tolerance to the endogenous target antigen and induce IL5-specific IgG antibodies. Moreover, these antibodies were able to efficiently neutralize IL5 activity in vitro and reduce circulating eosinophil numbers in vivo.

### Example 10

### Cloning, Expression and Purification of Recombinant Canine IL-1β

### Cloning of Recombinant Canine IL-1β

The canine IL-1β protein sequence with flanking BamHI/SpeI restriction sites and without a “stop”-codon was produced by oligonucleotide directed gene synthesis (BioCat GmbH). After synthesis of the gene, it was excised from a pUC57 helper plasmid and sub-cloned into the Bam HI/Spe I sites of a modified pET42 vector. The map of the resulting expression vector pET42NBS-cIL1b-C6Hcg (FIG. 27).

The resulting amino acid sequence of the so prepared canine IL-1β antigen used for coupling to modified CMV VLPs in accordance with the present invention is provided as SEQ ID NO:44. This amino acid sequence comprise the canine IL-1β sequence (SEQ ID NO:45) to which a His6-tag and a four amino acid linker (SEQ ID NO:46) at the C-terminus are added. The corresponding nucleotide sequence of this canine IL-1b antigen is described in SEQ ID NO:47.

### Expression and Purification of Recombinant IL-1β

The expression vector pET42NBS-cIL1b-C6Hcg was transformed into E. coli C2566 cells (New England Biolabs, Ipswich, USA). Clones expressing the highest levels of target protein were selected. Expression of the canine IL-1β antigen of SEQ ID NO:44 was performed in the following way: Cultures of E. coli harboring the expression plasmid were grown in 2×TY medium containing kanamycin (25 mg/L) on a rotary shaker (200 rev/min; Infers, Bottmingen, Switzerland) at 30° C. to an OD600 of 0.8-1.0. The expression of the canine IL-1β antigen of SEQ ID NO:44 was then induced by adding 0.2 mM IPTG. The medium was additionally supplemented with 5 mM MgCl2. Incubation was continued on a rotary shaker at 20° C. for 18 h. The resulting biomass was collected by low-speed centrifugation and frozen at −20° C. until purification.

The canine IL-1β antigen was purified using the USB PrepEase His-tagged protein purification kit (Affymetrix, High Wycombe, UK) according to manufacturer's instructions. After thawing on ice, E. coli cells (approx. 1.0 g) were suspended in 1×LEW buffer disrupted by ultrasonication (16 min; amplitude 70%, pulse 0.5; Hielscher UP200S device). Insoluble proteins and cell debris were removed by centrifugation (13,000 rpm, 30 min at 5° C.). The clarified soluble fraction was applied to a USB PrepEase column, washed twice with the same buffer and eluted with 3×1.5 ml of imidazole containing 1×E buffer. Fractions containing the canine IL-1β antigen were identified by SDS/PAGE (FIG. 28, lanes 5), and dialyzed against 200 volumes of the buffer (5 mM Na phosphate, 2 mM EDTA, pH 7.5) using a Spectra/Por membrane with MWCO 3.5 kDa (Spectrum Laboratories, #132720). The protein concentration was then estimated using a QuBit fluorometer in accordance with the manufacturer's recommendations (Invitrogen, Eugene, USA). The purity and identity and of the purified Can.IL1b.H6GGCG was demonstrated by SDS-PAGE and Western Blot (FIG. 28, Lane 5) and mass spectrometric analysis (data not shown).

### Example 11

### Coupling of Recombinant Canine IL-1β Antigen to Modified CMV VLPs

The recombinant canine IL-1β antigen of SEQ ID NO:44 was covalently linked to the various modified CMV VLPs prepared as described above. The linking was effected in accordance with the method described in Schmitz N, et al, J Exp Med (2009) 206: 1941-1955). Purified CMV-Ntt830, CMV-Ntt830-E4, and CMV-Ntt830-E8* VLPs were diluted to 1.5 mg/ml and reacted with a 5× molar excess (over one VLP coat protein monomer) of the heterobifunctional chemical cross-linker succinimidyl-6-(b-maleimido-propionamide) hexanoate (SMPH) for 1 hour at room temperature (RT). Cross-linker which did not react with the VLP was removed by centrifugation using an Amicon-Ultra-0.5, 100K centrifugal filter (Merck-Millipore, #UFC910024). The SMPH-derivatized VLPs were then washed 3 times with 5 mM Na2HPO4, 2 mM EDTA (pH 7.5).

Then, the canine IL-1β antigen was treated with a 10× molar ratio of TCEP for 10 min at RT and added in a 0.5:1 molar ratio, with respect to the VLP coat protein monomer, to the previously SMPH derivatized CMV VLPs for 3 hours at RT while shaking. The engineered free cysteine of the canine IL-1β antigen reacted with the cross-linker SMPH and formed a stable covalent linkage.

To demonstrate covalent conjugation of the canine IL-1β antigen to the modified CMV VLPs, coupling reactions were analyzed by SDS-PAGE and Western blot. This is exemplified in FIG. 28 where samples of CMV-Ntt830-E8* VLPs, SMPH-derivatized CMV-Ntt830-E8* VLPs, recombinant canine IL-1β antigen of SEQ ID NO:44 and the cIL1b-CMV-Ntt830-E8* VLPs were run in parallel. The analyses demonstrated the presence of bands representing canine IL-1β antigen covalently conjugated to either CMV VLP coat protein monomer or dimer in both Coomassie blue stained gels and Western blot analysis.

Prominent conjugation bands were observed following chemical coupling of the canine IL-1β antigen with CMV-Ntt830, CMV-Ntt830-E4 or CMV-Ntt830-E8* VLPs (FIG. 29). However, cIL1b-CMV-Ntt830 VLPs rapidly and completely precipitated from solution.

In contrast, analysis of the covalent conjugation of the canine IL-1β antigen to the CMV-Ntt830-E4 and CMV-Ntt830-E8* VLP variants by native agarose gels (FIG. 30A and FIG. 30B), dynamic light scattering (DLS) and EM analysis clearly demonstrate the VLPs were not aggregated and had typical VLP structures (FIG. 31A, FIG. 31B, FIG. 32A and FIG. 32B). Moreover, these vaccines remained soluble and did not precipitate from solution after completion of the conjugation reaction.

### Example 12

Induction of Neutralizing Antibodies Upon Immunization with Modified CMV VLPs to which Recombinant Canine IL-1b Antigens are Coupled

### Immunization of Mice

Two groups (n=5/group) of Balb/c mice were immunized subcutaneously twice 14 days apart with 30 μg of cIL1b-CMV-Ntt830-E4 or cIL1b-CMV-Ntt830-E8* formulated in 5 mM Na phosphate, 2 mM EDTA, pH 7.5. Before each immunization as well as on days 7 and 28 after the first vaccination, blood was collected into serum microtainer tubes (BD). Serum was prepared by spinning the collection tubes at 8000×g for 10 min. Sera were stored at ca. −20° C. until assaying.

Canine IL1b and CMV-VLP specific IgG antibodies in sera were measured by ELISA. Maxisorp ELISA plates were coated with recombinant canine IL-1b protein or CMV-VLP in 0.1 M Na Carbonate buffer, pH 9.6 at a concentration of 1 μg/ml overnight at 4° C. Plates were washed and SuperBlock™ (PBS) blocking buffer (Thermo Fisher/Life Technologies Europe) added for at least 30 minutes at RT then washed again. Serum samples were pre-diluted 100 fold in 2% BSA in PBS with 0.05% Tween 20, transferred to the ELISA plates and subjected to seven 3-fold serial dilutions. Following incubation for 1 hour at RT and washing, horse-radish peroxidase- (HRP-)labelled goat anti-mouse IgG, Fc gamma fragment specific (Jackson ImmunoResearch Europe Ltd) in 2% BSA in PBS (PBS pH 7.4 (1×) Gibco) with 0.05% Tween-20 was added. After incubation and washing, OPD substrate (Sigma-Aldrich) was used for colorimetric development. The enzymatic reaction was stopped by the addition of 5% H₂SO₄ and the absorbance at 490 nm measured by spectrophotometry using an ELISA reader (Tecan Spark 10). Serial dilutions were used to calculate the OD50 titers. An OD50 titer describes the reciprocal of the dilution, which reaches half of the maximal OD value. Titers below the detection limit of the assay were set to 50, the half of the lowest dilution factor (1:100) used in the assay.

The neutralizing ability of sera from mice immunized with cIL1b-CMV-E4 or cIL1b-CMV-E8* was determined using a bioactivity assay that measures IL-1β mediated secretion of IL-6 from HeLa cells. Briefly, serum were serial 2-fold serial diluted in assay medium starting at a final concentration of 1 in 50. A sheep anti canine IL-1β neutralizing antibody (R&D Systems) was used as a standard at a concentration range of 600 to 0.823 pg/mL. Canine IL-1β (R&D Systems) at a constant final concentration of 30 pg/mL was added to wells containing diluted sera or standard. Positive control wells containing 30 μg/mL IL-1β and negative containing assay medium alone were prepared. 1 hour later 5.2×10⁴ HeLa cells were added per well. After a 3 hour incubation period 10 μl supernatant of the final volume of 100 μl per well was used to quantitate IL-6 concentration in the solution using the IL-6 DuoSet ELISA kit (R&D Systems #DY206) according to the manufacturer's instructions. The TMB Substrate Kit (ThermoScientific) was used as a substrate reagent. After stopping enzymatic reaction by addition of 5% H₂SO₄, the absorbance at 450 nm was measured by spectrophotometry using an ELISA reader (Tecan Spark 10). Titration curves were generated by plotting the OD450 nm values versus the dilution factor of the serum sample. Using a 4-Parameter logistic regression curve fit model the IC50 values, the dilution factor corresponding to half maximum OD450 nm values, were determined. Neutralization titers below the detection limit of the assay were set to 25, the half of the lowest dilution factor (1:50) used in the assay.

### Results.

For animals immunized with cIL1b-CMV-E4 or cIL1b-CMV-E8*, IL-1β-specific IgG antibodies were detected in sera collected from day 7 onwards (FIG. 33A). Antibody titers increased further by day 14 and following administration of the second injection (day 14), titers increased again until termination of the experiment on day 28. The kinetic of the anti-CMV IgG titers was slightly delayed with respect to the anti-IL-1β titers and the magnitude of titers was slightly lower (FIG. 33B).

The neutralizing ability of the immune sera induced in response to the vaccination was demonstrated using an in vitro cellular assay, which measured secretion of IL-6 by HeLa cells in response to IL-1β stimulation. Day 28 sera from mice immunized with cIL1b-CMV-E4 or cIL1b-CMV-E8* inhibited IL-1β mediated IL-6 secretion from HeLa cells with mean group neutralization titers of 3000 and 130 respectively (FIG. 33C). Pre-immune sera collected from all animals prior to immunization (day 0) did not inhibit IL-6 secretion.

These results show that immunization of mice with either cIL1b-CMV-E4 or cIL1b-CMV-E8* induces IL-1β-specific IgG antibodies, which are capable of efficiently neutralizing IL-1β activity in vitro.

### Example 13

### Cloning, Expression and Purification of Recombinant Feline IL-1β

### Cloning of Recombinant Feline IL-1β

The gene coding for the feline IL-1β protein without neither a “start”—nor a “stop”—codon and with flanking BamHI/SpeI restriction sites was obtained by oligonucleotide directed gene synthesis in a pUC57 helper plasmid (BioCat GmbH, Heidelberg, Germany). The gene was then sub-cloned into the Bam HI/Spe I sites of a modified pET42 vector resulting in the production of the expression vector pET42NBS-fIL1b-c6Hcg (FIG. 34).

The resulting amino acid sequence of the so prepared feline IL-1β antigen used for coupling to modified CMV VLPs in accordance with the present invention is provided as SEQ ID NO:171. This amino acid sequence comprise the feline IL-1β sequence (SEQ ID NO:121) to which a His6-tag and a four amino acid linker (SEQ ID NO:46) at the C-terminus are added. The corresponding nucleotide sequence of this feline IL-1b antigen is described in SEQ ID NO:172.

### Expression and Purification of Recombinant Feline IL-1β

E. coli C2566 cells (New England Biolabs, Ipswich, USA) were transformed with the pET42NBS-fIL1b-c6Hcg plasmid. After selection of the clone with the highest target-protein expression, expression of the feline IL-1β antigen of SEQ ID NO:171 was performed in the following way: selected clone was cultivated in 2×TY medium containing kanamycin (25 mg/L) on a rotary shaker (200 rev/min; Infers, Bottmingen, Switzerland) at 30° C. to an OD600 of 0.8-1.0. The expression of the feline IL-1β antigen of SEQ ID NO:171 was then induced by adding 0.2 mM IPTG, and the medium was supplemented with 5 mM MgCl₂. Incubation was continued on a rotary shaker at 20° C. for 18 h. The resulting biomass was collected by low-speed centrifugation and frozen at −20° C. until processed further.

The feline IL-1β antigen was purified using the Protino, Ni-IDA 2000 His-Tag kit, (Macherei-Nagel, Duren, Germany) according to manufacturer's instructions. After thawing on ice, the E. coli biomass was were suspended in 1×LEW buffer disrupted by ultrasonication (16 min; amplitude 70%, pulse 0.5; Hielscher UP200S device). Insoluble proteins and cell debris were removed by centrifugation (11,000 rpm, 20 min at +4° C.). The clarified soluble fraction was applied to a Protino HisTag column, washed twice and eluted. Fractions containing the feline IL-1β antigen were identified by SDS/PAGE (FIG. 35A, lanes 8 and 9) and combined. Combined elution fractions containing feline IL-1β protein were purified by gel filtration (Superdex 75; running buffer 5 mM Na phosphate, 2 mM EDTA). Fractions containing feline IL-1β were concentrated using an Amicon® Ultra-15 Centrifugal Filter Unit (Merk, Darmstadt, Deutschland). Protein concentration was determined using a Qubit protein quantification kit in accordance with the manufacturer's recommendations (Invitrogen, Eugene, USA). The purity and identity and of the purified Fel.IL1b.H6GGCG was demonstrated by SDS-PAGE (FIG. 35A, Lanes 8 and 9) and feline IL-1 beta/IL-1F2 DuoSet ELISA (biotechne, Abingdon, United Kingdome) according to manufacturer's instruction (data not shown).

### Example 14

### Coupling of Recombinant Feline IL-1β Antigen to Modified CMV VLPs

The recombinant feline IL-1β antigen of SEQ ID NO:171 was covalently linked to the various modified CMV VLPs prepared as described above. The linking was effected in accordance with the method described in Schmitz N, et al, J Exp Med (2009) 206: 1941-1955). Purified CMV-Ntt830, and CMV-Ntt830-E4 VLPs were diluted to 1.5 mg/ml and reacted with a 5× molar excess (over one VLP coat protein monomer) of the heterobifunctional chemical cross-linker succinimidyl-6-(b-maleimido-propionamide) hexanoate (SMPH) for 1 hour at room temperature (RT). Cross-linker which did not react with the VLP was removed was removed by 4 buffer exchanges using Amicon-Ultra-15 (100K) filtration units (Merck, Darmstadt, Deutschland).

Meanwhile, the feline IL-1β antigen was treated with a 10× molar ratio of TCEP for 10 min at RT and added in equal molar ratio, with respect to the VLP coat protein monomer, to the previously SMPH derivatized CMV VLPs for 2 hours at RT while shaking. The engineered free cysteine of the feline IL-1β antigen reacted with the cross-linker SMPH and formed a stable covalent linkage. The solution was clarified by centrifugation and feline IL-13 not closely associated with the VLP was removed by gel filtration (Superdex200) or by centrifugation using Amicon-Ultra-15 (100K) filtration units (Merck, Darmstadt, Deutschland).

The covalent conjugation of the feline IL-1β antigen to the CMV VLPs, was demonstrated by analyzing the intermediate products of the coupling reactions by SDS-PAGE. This is exemplified in FIG. 35A where samples of CMV-Ntt830-E4 VLPs, SMPH-derivatized CMV-Ntt830-E4 VLPs, recombinant feline IL-1β antigen of SEQ ID NO:171, the fIL1b-CMV-Ntt830-E4 VLPs before and after clarification and removal of feline IL-1, that was not associated with the VLP after conjugation, were run in parallel. The analyses demonstrated the presence of bands representing feline IL-1β antigen covalently conjugated to either CMV VLP coat protein monomer or dimer in Coomassie blue stained gels.

Prominent conjugation bands were observed following chemical coupling of the feline IL-1β antigen with CMV-Ntt830-E4 (FIG. 35A) or CMV-Ntt830 VLPs (FIG. 35B). However, fIL1b-CMV-Ntt830 VLPs rapidly precipitated from solution.

In contrast, analysis of the covalent conjugation of the feline IL-1β antigen to the CMV-Ntt830-E4 VLP variants by native agarose gels (FIG. 36A and FIG. 36B), dynamic light scattering (DLS) and EM analysis clearly demonstrate the VLPs were not aggregated and had typical VLP structures (FIG. 37A, FIG. 37B). Moreover, this vaccine remained soluble and did not precipitate from solution after completion of the conjugation reaction.

### Example 15

Induction of Neutralizing Antibodies Upon Immunization with Modified CMV VLPs to which Recombinant Feline IL-1b Antigens are Coupled

### Immunization of Mice

Two groups (n=5/group) of Balb/c mice were immunized subcutaneously twice 21 days apart with 30 μg of fIL1b-CMV-Ntt830-E4 formulated in 5 mM Na phosphate, 2 mM EDTA, pH 7.5 (Group 2: fIL1b-CMV-Ntt830-E4 group) or formulation buffer control (Group 1: control group). Before each immunization as well as on days 14, 35 and 42 after the first vaccination, blood was collected into serum microtainer tubes (BD). Serum was prepared by spinning the collection tubes at 8000×g for 10 min. Sera were stored at ca. −20° C. until assaying.

Feline IL-1b and CMV-VLP specific IgG antibodies in sera were measured by ELISA. Maxisorp ELISA plates were coated with recombinant feline IL-1b protein or CMV-VLP in 0.1 M Na Carbonate buffer, pH 9.6 at a concentration of 1 μg/ml and 10 μg/ml, respectively, overnight at 4° C. Plates were washed and SuperBlock™ blocking buffer (Thermo Fisher/Life Technologies Europe) added for at least 60 minutes at RT then washed again. Serum samples were pre-diluted 100 fold in 2% BSA in PBS with 0.05% Tween 20, transferred to the ELISA plates and subjected to seven 3-fold serial dilutions. Following incubation for 1.5 hours at RT and washing, horse-radish peroxidase- (HRP-)labelled anti-mouse IgG (subclasses 1+2a+2b+3) specific (Jackson ImmunoResearch Europe Ltd) dilute 2000-fold in 2% BSA in PBS with 0.05% Tween-20 was added. After incubation and washing, OPD substrate (Sigma-Aldrich) was used for colorimetric development. The enzymatic reaction was stopped by the addition of 2M H₂SO₄ and the absorbance at 490 nm measured by spectrophotometry using an ELISA reader (Tecan Spark 10). Serial dilutions were used to calculate the OD50 titers. An OD50 titer describes the reciprocal of the dilution, which reaches half of the maximal OD value. Titers below the detection limit of the assay were set to 50, the half of the lowest dilution factor (1:100) used in the assay.

The neutralizing ability of sera from mice immunized with fIL1b-CMV-Ntt830-E4 VLPs was determined using a bioactivity assay based on the HEK-Blue IL-1beta reporter cells (InvivoGen hkb-il1bv2). Briefly, HEK-Blue IL-1b cells were grown and plated at a final concentration of 5×10⁴ cells per well. Sera were heat inactivated for 30 min at +56° C., then diluted 1:12.5 in assay medium (final dilution in assay 1:50). A 3-fold serial dilution was prepared for a total of 7 dilutions of each serum, prior to addition of equivalent volume of feline IL-1β (R&D Systems) at 2000 pg/mL (final concentration in assay 500 pg/mL). Serum/cytokine solution was incubated for 1 hour at room temperature. 50 μL of serum/cytokine mixture was added to 50 μL of a cell suspension prepared to 5×10⁴ cells per well. Plates were incubated at 5% CO₂, +37° C. for approximately 20 hours. After 20 hours incubation, cell viability was assessed visually. 40 μL/well of cell supernatant was added to 160 μL/well QUANTI-Blue Solution (Invivogen). Color development was monitored by measuring OD620 nm over time.

To determine neutralization titers, results were analyzed using Microsoft Excel and GraphPad prism. Titration curves were generated by plotting the OD620 nm values versus the dilution factor of the serum sample using GraphPad prism software (GraphPad Prism version 8.0.0 for Windows, GraphPad Software, San Diego, California USA). Using a 4-Parameter logistic regression curve fit model the IC50 values, the dilution factor corresponding to half maximum OD values, were determined. Serum fIL-1b neutralization titers of samples at different time points were defined and depicted as the IC50 values of the curve fit.

### Results

For animals immunized with fIL1b-CMV-Ntt830-E4 VLPs, IL-1β-specific IgG antibodies were detected in sera collected from day 14 onwards (FIG. 38A). Antibody titers were stable to day 21 and following administration of the second injection (day 21), titers increased again until termination of the experiment on day 42. The kinetic and the magnitude of the anti-CMV IgG titers were comparable to the anti-IL-1β titers (FIG. 38B).

The neutralizing ability of the immune sera induced in response to the vaccination was demonstrated using an in vitro cellular assay based on a human IL-1β reporter cell line, which measured secretion of alkaline phosphates in response to IL-1β stimulation. Day 42 sera from mice immunized with fIL1b-CMV-Ntt830-E4 VLPs inhibited IL-1β mediated secretion of alkaline phosphatase by the reporter cells with geometric mean group neutralization titers of 1730 (FIG. 38C). Pre-immune sera collected from all animals prior to immunization (day 0) and from control group did not inhibit IL-1β.

These results show that immunization of mice with fIL1b-CMV-Ntt830-E4 VLPs induces IL-1β-specific IgG antibodies, which are capable of efficiently neutralizing IL-1β activity in vitro.

1. A modified virus-like particle (VLP) of cucumber mosaic virus (CMV) comprising at least one chimeric CMV polypeptide, wherein said at least one chimeric CMV polypeptide comprises, preferably consists of
(i) a CMV polypeptide, wherein said CMV polypeptide comprises a coat protein of CMV or an amino acid sequence having a sequence identity of at least 75% with SEQ ID NO:48; and
(ii) a polypeptide comprising, preferably consisting of, a stretch of consecutive negative amino acids, wherein said negative amino acids are independently selected from aspartic acid or glutamic acid, wherein said polypeptide is inserted between any amino acid residue of said CMV polypeptide corresponding to any amino acid residue between position 75 and position 85 of SEQ ID NO:48.

2. The modified VLP of CMV of claim 1, wherein said chimeric CMV polypeptide further comprises a T helper cell epitope, wherein said T helper cell epitope replaces a N-terminal region of said CMV polypeptide, wherein said N-terminal region of said CMV polypeptide corresponds to amino acids 2-12 of SEQ ID NO:48.

3. The modified VLP of CMV of claim 2, wherein said T helper cell epitope is derived from tetanus toxin or is a PADRE sequence, and wherein preferably said Th cell epitope comprises the amino acid sequence of SEQ ID NO:50 or SEQ ID NO:51.

4. The modified VLP of CMV of any one of the preceding claims, wherein said CMV polypeptide is a coat protein of CMV or an amino acid sequence having a sequence identity of at least 90%, preferably 95% with SEQ ID NO:48.

5. The modified VLP of CMV of any one of the preceding claims, wherein said CMV polypeptide comprises, preferably consists of, the amino acid sequence of SEQ ID NO:5, wherein said polypeptide comprising said stretch of consecutive negative amino acids is inserted between amino acid residues of position 88 and position 89 of said SEQ ID NO:5.

6. The modified VLP of CMV of any one of the preceding claims, wherein said stretch of consecutive negative amino acids has a length of 3 to 10 amino acids.

7. The modified VLP of CMV of any one of the preceding claims, wherein said stretch of consecutive negative amino acids consists solely of glutamic acids.

8. The modified VLP of CMV of any one of the preceding claims, wherein said polypeptide comprising said stretch of consecutive negative amino acids further comprises a first amino acid linker and a second amino acid linker, wherein said first amino acid linker is positioned at the N-terminus of said stretch of consecutive negative amino acids, and said second amino acid linker is positioned at the C-terminus of said stretch of consecutive negative amino acids, and wherein said first and said second amino acid linker is independently selected from the group consisting of:
(a.) a polyglycine linker (G-linker) having an amino acid sequence (Gly)ₙ of a length of n=2-10;
(b.) a glycine-serine linker (GS-linker) comprising at least one glycine and at least one serine, wherein preferably said GS linker has an amino acid sequence of (GS)ᵣ(GₛS)ₜ(GS)ᵤ with r=0 or 1, s=1-5, t=1-5 and u=0 or 1; and
(c.) an amino acid linker (GS*-linker) comprising at least one Gly, at least one Ser, and at least one amino acid selected from Thr, Ala, Lys, and Cys.

9. The modified VLP of CMV of any one of the preceding claims, wherein said polypeptide comprises, preferably consists of, SEQ ID NO:62, SEQ ID NO:63 or SEQ ID NO:64.

10. The modified VLP of CMV of claim 1, wherein said chimeric CMV polypeptide comprises, preferably consists of, the amino acid sequence of SEQ ID NO:10, SEQ ID NO:11 or SEQ ID NO:12.

11. A composition comprising
(a) a modified VLP of CMV of any one of the claims 1 to 10, and wherein said modified VLP of CMV comprises at least one first attachment site; and
(b) at least one antigen, wherein said antigen comprises at least one second attachment site;
wherein (a) and (b) are linked through said at least one first and said at least one second attachment site via at least one covalent non-peptide bond.

12. The composition of claim 11, wherein said at least one first attachment site is not comprised or part of the polypeptide comprising said stretch of consecutive negative amino acid.

13. The composition of claim 11 or claim 12, wherein said first attachment site is an amino group, preferably an amino group of a lysine residue, and wherein said at least one second attachment site is a sulfhydryl group, preferably a sulfhydryl group of a cysteine residue.

14. The composition of any one of the claims 11 to 13, wherein said antigen is an allergen, a self antigen, a tumor antigen, a hormone, a growth factor, a cytokine, a chemokine, or a polypeptide of a viral, bacterial or pathogen.

15. The composition of any one of the claims 11 to 14, wherein said antigen is a growth factor or an interleukin, wherein said growth factor is selected from vascular endothelial growth factor, vascular endothelial growth factor receptor, hepatocyte growth factor, epidermal growth factor, epidermal growth factor receptor and nerve growth factor, and wherein said interleukin is selected from interleukin-1α, interleukin-1β, interleukin-4, interleukin-5, interleukin-6, interleukin-8, interleukin-13 interleukin-15, interleukin-23, interleukin-25, interleukin-31 and interleukin-33.


SEQUENCE LISTING (175 sequences)

SEQ ID NO: 1 - protein, 4 aa, synthetic construct
EEEE

SEQ ID NO: 2 - protein, 8 aa, synthetic construct
EEEEEEEE

SEQ ID NO: 3 - protein, 12 aa, synthetic construct
EEEEEEEEEEEE

SEQ ID NO: 4 - protein, 7 aa, synthetic construct
CGGGSGS

SEQ ID NO: 5 - protein, 222 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 6 - protein, 14 aa, synthetic construct
QYIKANSKFIGITE

SEQ ID NO: 7 - DNA, 672 bp, synthetic construct
  source 1..672: mol_type = other DNA
atgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtgggtcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataa

SEQ ID NO: 8 - protein, 4 aa, synthetic construct
GGGS

SEQ ID NO: 9 - protein, 6 aa, synthetic construct
GGGSGS

SEQ ID NO: 10 - protein, 236 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 11 - protein, 240 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 12 - protein, 241 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEECGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 13 - protein, 244 aa, synthetic construct
MGQYIKANSKFIGITERRRRPRRGSRSAPSSADANFRVLSQQLSRLNKTLAAGRPTINHPTFVGSERCKPGYTFTSITLKPPKIDRGSGGGSEEEEEEEEEEEEGGGSGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 14 - DNA, 720 bp, synthetic construct
  source 1..720: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 15 - DNA, 732 bp, synthetic construct
  source 1..732: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggaagaggaagaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 16 - DNA, 736 bp, synthetic construct
  source 1..736: mol_type = other DNA
catatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaagaggaagaagaggaagaagagtgcgggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 17 - DNA, 744 bp, synthetic construct
  source 1..744: mol_type = other DNA
ccatgggccagtatattaaggccaactccaaatttatcgggattaccgagcgtcgacgtcgtccgcgtcgtggttcccgctccgccccctcctccgcggatgctaactttagagtcttgtcgcagcagctttcgcgacttaataagacgttagcagctggtcgtccaactattaaccacccaacctttgtagggagtgaacgctgtaaacctgggtacacgttcacatctatcaccctaaagccaccaaaaatagaccgtggatccggtggcggtagcgaggaagaagaggaagaagaggaagaagaggaagaagggggtggttcgggttcttattatggtaaaaggttgttattacctgattcagtcacggaatatgataagaaacttgtttcgcgcattcaaattcgagttaatcctttgccgaaatttgattcaaccgtgtgggtgacagtccgtaaagttcctgcctcttcggacttatccgttgccgccatttctgctatgtttgcggacggagcctcaccggtactggtttatcagtacgctgcatctggagtccaagctaacaacaaactgttgtatgatctttcggcgatgcgcgctgatataggcgacatgagaaagtacgccgtcctcgtgtattcaaaagacgatgcactcgagacagacgagttagtacttcatgttgacgtcgagcaccaacgtattcccacatctggggtgctcccagtttgataagctt

SEQ ID NO: 18 - DNA, 51 bp, synthetic construct
  source 1..51: mol_type = other DNA
tgcgggggtggttcgggttcttattatggtaaaaggttgttattacctgat

SEQ ID NO: 19 - DNA, 62 bp, synthetic construct
  source 1..62: mol_type = other DNA
tggatccggtggcggtagcgaagaggaagaagaggaagaagagtgcgggggtggttcgggtt

SEQ ID NO: 20 - DNA, 37 bp, synthetic construct
  source 1..37: mol_type = other DNA
caaagcttatcaaactgggagcaccccagatgtggga

SEQ ID NO: 21 - DNA, 24 bp, synthetic construct
  source 1..24: mol_type = other DNA
aggatcgagatctcgatcccgcga

SEQ ID NO: 22 - DNA, 35 bp, synthetic construct
  source 1..35: mol_type = other DNA
accataataggatccacggtctatttttggtggct

SEQ ID NO: 23 - DNA, 40 bp, synthetic construct
  source 1..40: mol_type = other DNA
agaccgtggatcctattatggtaaaaggttgttattacct

SEQ ID NO: 24 - DNA, 25 bp, synthetic construct
  source 1..25: mol_type = other DNA
agtaccggtgaggctccgtccgcaa

SEQ ID NO: 25 - DNA, 51 bp, synthetic construct
  source 1..51: mol_type = other DNA
ggatccggtggcggtagcgaagaggaagaagggggtggttcgggttcttat

SEQ ID NO: 26 - DNA, 63 bp, synthetic construct
  source 1..63: mol_type = other DNA
ggatccggtggcggtagcgaggaagaggaagaagaggaagaagggggtggttcgggttcttat

SEQ ID NO: 27 - DNA, 75 bp, synthetic construct
  source 1..75: mol_type = other DNA
ggatccggtggcggtagcgaggaagaagaggaagaagaggaagaagaggaagaagggggtggttcgggttcttat

SEQ ID NO: 28 - DNA, 698 bp, synthetic construct
  source 1..698: mol_type = other DNA
ccatggccgaacctcacccagagtcaaacgttcccgcgggacatgcgattccacaggcgcactggacaaaacttcaacattcgttagacacagctcttcgccgtgcccgcagcacccctgcgggcgcaatcgcagcccgtgtagcgggtcaaacccgtaatattacagtggatccgaaattgtttaaaaagcgccgtctgcgtagtccccgcgtgttattctccacgcatcccccccctgttgccgcggatacccaaggcttagatctggaagctgggggagctgcatctttcaaccgcacgcaccgctcgaaacgcagtagcagtcacccagtgtttcaccgtggtgaattttctgtctgcgactccgtctctgtgtgggtaggggataagacgactgctaccgacatcaaggggaaggaagtgatggttttaggggaggtgaatattaacaattcggtcttcaagcaatacttcttcgaaacaaaatgccgcgatccaacgccagtggatagtggttgtcgcggtattgactcgaagcattggaactcatactgcaccacaacccatacctttgtgaaggcattgacaatggatggcaaacaggccgcctggcgttttattcgcattgacaccgcctgtgtctgtgtccttagtcgcaaagctggtcgccgcgcgggtggttgcgggtaactgcag

SEQ ID NO: 29 - protein, 229 aa, synthetic construct
MAEPHPESNVPAGHAIPQAHWTKLQHSLDTALRRARSTPAGAIAARVAGQTRNITVDPKLFKKRRLRSPRVLFSTHPPPVAADTQGLDLEAGGAASFNRTHRSKRSSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGGCG

SEQ ID NO: 30 - protein, 121 aa, synthetic construct
SSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAG

SEQ ID NO: 31 - protein, 124 aa, synthetic construct
SSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGGCG

SEQ ID NO: 32 - DNA, 62 bp, synthetic construct
  source 1..62: mol_type = other DNA
atattctagaaataattttgtttaactttaagaaggagatataccatggccgaacctcaccc

SEQ ID NO: 33 - DNA, 37 bp, synthetic construct
  source 1..37: mol_type = other DNA
cattaagcttgtcgacctgcagttacccgcaaccacc

SEQ ID NO: 34 - DNA, 792 bp, synthetic construct
  source 1..792: mol_type = other DNA
catatgattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaaatgcggtggtgaacgttggcgtgtgaaaaaattcctggattatctgcaggtttttcatatgattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaaatgcggtggtgaacgttggcgtgtgaaaaaattcctggattatctgcaggtttttctgggcgtgattaataccgaatggaccatggaaagccatcatcatcaccatcacggtggttgtggttaatgataactcgagctgggcgtgattaataccgaatggaccatggaaagccatcatcatcaccatcacggtggttgtggttaatgataactcgag

SEQ ID NO: 35 - protein, 126 aa, synthetic construct
MIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESHHHHHHGGCG

SEQ ID NO: 36 - DNA, 22 bp, synthetic construct
  source 1..22: mol_type = other DNA
aggaggtgacgaggatggatcc

SEQ ID NO: 37 - DNA, 62 bp, synthetic construct
  source 1..62: mol_type = other DNA
ctccggaaccaccgccaccgctaccaccaccaccgctttccatggtccattcggtattaatc

SEQ ID NO: 38 - DNA, 44 bp, synthetic construct
  source 1..44: mol_type = other DNA
ttccggaggtggcggtagcattgcagttcagagcccgatgaatc

SEQ ID NO: 39 - DNA, 21 bp, synthetic construct
  source 1..21: mol_type = other DNA
tcctccatcctcgtcactagt

SEQ ID NO: 40 - protein, 15 aa, synthetic construct
GGGGSGGGGSGGGGS

SEQ ID NO: 41 - protein, 245 aa, synthetic construct
IAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESGGGGSGGGGSGGGGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 42 - protein, 260 aa, synthetic construct
MGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESGGGGSGGGGSGGGGSIAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMESTSHHHHHHGGCG

SEQ ID NO: 43 - DNA, 799 bp, synthetic construct
  source 1..799: mol_type = other DNA
catatgggatccattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaagtgtggtggtgaacgttggcgtgtgaaaaaattcctggattatctgcaggtttttctgggcgtgattaataccgaatggaccatggaaagcggtggtggtggtagcggtggcggtggttccggaggtggcggtagcattgcagttcagagcccgatgaatcgtctggttgcagaaaccctggcactgctgagcacccatcgtaccctgctgattggtgatggtaatctgatgattccgacaccggaacataataatcatcagctgtgtatcgaagaagtgtttcagggcattgataccctgaaaaatcgtaccgttccgggtgatgcagttgaaaaactgtttcgtaatctgagcctgatcaaagaacatatcgatcgccagaaaaaaaagtgtggtggtgaacgttggcgtgtgaaaaaattcctggattatctgcaggtttttctgggcgtgattaataccgaatggaccatggaaagcactagtcatcatcatcaccatcatggtggttgtggataataagcttctcgag

SEQ ID NO: 44 - protein, 167 aa, synthetic construct
MGSAAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSSTSHHHHHHGGCG

SEQ ID NO: 45 - protein, 152 aa, synthetic construct
AAMQSVDCKLQDISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 46 - protein, 4 aa, synthetic construct
GGCG

SEQ ID NO: 47 - DNA, 520 bp, synthetic construct
  source 1..520: mol_type = other DNA
catatgggatccgcagccatgcaatcggtggactgcaagttacaggacataagccacaaatacctggtgctgtctaactcttatgagcttcgggctctccacctcaatggggaaaatgtgaacaaacaagtggtgttccacatgagctttgtgcacggggatgaaagtaataacaagatacctgtggtcttgggcatcaaacaaaagaatctgtacctgtcctgtgtgatgaaggatggaaagcccaccctacagctagagaaggtagaccccaaagtctacccaaagaggaagatggaaaagcgatttgtcttcaacaagatagaaatcaagaacacagtggaatttgagtcttctcagtaccctaactggtacatcagcacctctcaagtcgaaggaatgcctgtcttcctaggaaataccagaggtggccaggatataactgacttcacgatggaattctcttccactagtcatcatcatcaccatcatggtggttgtggataataagcttctcgag

SEQ ID NO: 48 - protein, 218 aa, synthetic construct
MDKSESTSAGRSRRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 49 - protein, 27 aa, synthetic construct
DKSESTSAGRSRRRRPRRGSRSAPSSA

SEQ ID NO: 50 - protein, 14 aa, synthetic construct
QYIKANSKFIGITE

SEQ ID NO: 51 - protein, 13 aa, synthetic construct
AKFVAAWTLKAAA

SEQ ID NO: 52 - protein, 13 aa, synthetic construct
PKYVKQNTLKLAT

SEQ ID NO: 53 - protein, 20 aa, synthetic construct
PHHTALRQAILCWGELMTLA

SEQ ID NO: 54 - protein, 21 aa, synthetic construct
DIEKKIAKMEKASSVFNVVNS

SEQ ID NO: 55 - protein, 16 aa, synthetic construct
YSGPLKAEIAQRLEDV

SEQ ID NO: 56 - protein, 21 aa, synthetic construct
FNNFTVSFWLRVPKVSASHLE

SEQ ID NO: 57 - protein, 220 aa, synthetic construct
MAKFVAAWTLKAAARRRRPRRGSRSAPSSADANFRVLSQQLSRLNKTLAAGRPTINHPTFVGSERCKPGYTFTSITLKPPKIDRGSYYGKRLLLPDSVTEYDKKLVSRIQIRVNPLPKFDSTVWVTVRKVPASSDLSVAAISAMFADGASPVLVYQYAASGVQANNKLLYDLSAMRADIGDMRKYAVLVYSKDDALETDELVLHVDVEHQRIPTSGVLPV

SEQ ID NO: 58 - protein, 70 aa, synthetic construct
EICPAVKRDVDLFLTGTPDEYVEQVAQYKALPVVLENARILKNCVDAKMTEEDKENALSLLDKIYTSPLC

SEQ ID NO: 59 - protein, 92 aa, synthetic construct
VKMAETCPIFYDVFFAVANGNELLLDLSLTKVNATEPERTAMKKIQDCYVENGLISRVLDGLVMTTISSSKDCMGEAVQNTVEDLKLNTLGR

SEQ ID NO: 60 - protein, 90 aa, synthetic construct
VKMAETCPIFYDVFFAVANGNELLLDLSLTKVNATEPERTAMKKIQDCYVENGLISRVLDGLVMIAINEYCMGEAVQNTVEDLKLNTLGR

SEQ ID NO: 61 - protein, 78 aa, synthetic construct
VKMAETCPIFYDVFFAVANGNELLLDLSLTKVNATEPERTAMKKIQDCYVENGLISRVLDGLVMPSTNIAWVKQFRTP

SEQ ID NO: 62 - protein, 19 aa, synthetic construct
GGGSEEEEEEEECGGGSGS

SEQ ID NO: 63 - protein, 14 aa, synthetic construct
GGGSEEEEGGGSGS

SEQ ID NO: 64 - protein, 18 aa, synthetic construct
GGGSEEEEEEEEGGGSGS

SEQ ID NO: 65 - DNA, 10 bp, synthetic construct
  source 1..10: mol_type = other DNA
gacgatcgtc

SEQ ID NO: 66 - DNA, 30 bp, synthetic construct
  source 1..30: mol_type = other DNA
gggggggggggacgatcgtcgggggggggg

SEQ ID NO: 67 - protein, 514 aa, synthetic construct
MAMKFIAPMAFVAMQLIIMAAAEDQSAQIMLDSDIEQYLRSNRSLRKVEHSRHDAINIFNVEKYGAVGDGKHDCTEAFSTAWQAACKKPSAMLLVPGNKKFVVNNLFFNGPCQPHFTFKVDGIIAAYQNPASWKNNRIWLQFAKLTGFTLMGKGVIDGQGKQWWAGQCKWVNGREICNDRDRPTAIKFDFSTGLIIQGLKLMNSPEFHLVFGNCEGVKIIGISITAPRDSPNTDGIDIFASKNFHLQKNTIGTGDDCVAIGTGSSNIVIEDLICGPGHGISIGSLGRENSRAEVSYVHVNGAKFIDTQNGLRIKTWQGGSGMASHIIYENVEMINSENPILINQFYCTSASACQNQRSAVQIQDVTYKNIRGTSATAAAIQLKCSDSMPCKNINLSDISLKLTSGKIASCLNDNANGYFSGHVIPACKNLSPSAKRKESKSHKHPKTVMVENMGAYDKGNRTRILLGSRPPNCTNKCHGCSPCKAKLVIVHRIMPQEYYPQRWMCSCHGKIYHP

SEQ ID NO: 68 - protein, 371 aa, synthetic construct
AEDLQEILPVNETRRLTTSGAYNIIDGCWRGKADWAENRKALADCAQGFGKGTVGGKDGDIYTVTSDLDDDVANPKEGTLRFGAAQNRPLWIIFERDMVIRLDKEMVVNSDKTIDGRGAKVEIINAGFTLNGVKNVIIHNINMHDVKVNPGGLIKSNDGPAAPRAGSDGDAISISGSSQIWIDHCSLSKSVDGLVDAKLGTTRLTVSNSLFTQHQFVLLFGAGDENIEDRGMLATVAFNTFTDNVDQRMPRCRHGFFQVVNNNYDKWGSYAIGGSASPTILSQGNRFCAPDERSKKNVLGRHGEAAAESMKWNWRTNKDVLENGAIFVASGVDPVLTPEQSAGMIPAEPGESALSLTSSAGVLSCQPGAPC

SEQ ID NO: 69 - protein, 130 aa, synthetic construct
GIAFPQNPGCRNTEDKNFPQHVKVNLNILNRNTNSRRPSDYYNRSTSPWNLHRNEDPERYPSVIWEAKCRHLGCVNNEGNINYHMNSVPIQQEILVLRRESQHCPHSFRLEKMLVAVGCTCVTPIVRHVA

SEQ ID NO: 70 - protein, 130 aa, synthetic construct
GIAFPQNPGCPTTEDKNFPQHVKVNVNILNGNKSSRRPLDYYRRSTSPWSLHRNEDPERYPSVIWEAKCLHWGCVNTEGKEDHHMNSVPIQQEILVLRRESRHCPHSFRLEKMLVTVGCTCVTPIVRHVV

SEQ ID NO: 71 - protein, 130 aa, synthetic construct
GIVIPQNPECPNTGDKNFPQNVKINLNVLNRKTNSRRASDYHNRSTSPWNLHRNEDPERYPSVIWEAKCRHLGCVNAEGKVDFHMNSVPIQQEILVLRRESQNCPHSFQLEKMLVAVGCTCVTPIVRHMG

SEQ ID NO: 72 - protein, 130 aa, synthetic construct
GVIIPQSPGCPPTEDKNFPQHVRVNLNIVNRSTNSRRPTDYHKRSTSPWTLHRNEDPERYPSVIWEAKCSHSGCINAEGKVDHHMNSVTIQQEILVLRRESQHCPHSFRLEKMLVAVGCTCVTPIVRHLA

SEQ ID NO: 73 - protein, 130 aa, synthetic construct
GIMIPQSPGCPKTEDKNFPQHVRVNLNILNRSTPARRPSDYSKRFTSPWTLQRNEDPERYSSVIWEAKCSHSGCINAEGKEDHHMNSVPIQQEILVLRREPRHCPNSFRLEKVMVTVGCTCVTPIVRHIS

SEQ ID NO: 74 - protein, 115 aa, synthetic construct
IPTEIPTSALVKETLALLSTHRTLLIANETLRIPVPVHKNHQLCTEEIFQGIGTLESQTVQGGTVERLFKNLSLIKKYIDGQKKKCGEERRRVNQFLDYLQEFLGVMNTEWIIES

SEQ ID NO: 75 - protein, 114 aa, synthetic construct
AVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPES

SEQ ID NO: 76 - protein, 115 aa, synthetic construct
FAVENPMNRLVAETLTLLSTHRTWLIGDGNLMIPTPENKNHQLCIKEVFQGIDTLKNQTAHGEAVDKLFQNLSLIKEHIERQKKRCAGERWRVTKFLDYLQVFLGVINTEWTPES

SEQ ID NO: 77 - protein, 114 aa, synthetic construct
AVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 78 - protein, 115 aa, synthetic construct
IAVQSPMNRLVAETLALLSTHRTLLIGDGNLMIPTPEHNNHQLCIEEVFQGIDTLKNRTVPGDAVEKLFRNLSLIKEHIDRQKKKCGGERWRVKKFLDYLQVFLGVINTEWTMES

SEQ ID NO: 79 - protein, 109 aa, synthetic construct
IAVQSPMNRLVAETLALLSTHRTLLIGDGNLMIPTPEHNNHQLCIEEVFQGIDTLKNRTVPGDAVEKLFRNLSLIKEHIDRQKKNFGGKKWKVKNFLNYLQFFLGLLNT

SEQ ID NO: 80 - protein, 115 aa, synthetic construct
LAVESPMNRLVAETLTLLSTHRTLLIGDGNLMIPTPEHKNHQLCIEEVFQGIDTLKNQTVQGDAVAKLFQNLSLIKGYIDLQKKKCGGERWRVKQFLDYLQEFLGVINTEWTIEG

SEQ ID NO: 81 - protein, 113 aa, synthetic construct
VESTMNRLVAETLTLLSSHRTLLIGDGNLMIPTPQHTNHQLCIEEVFQGIDTLKNQTAQGDAVKKIFQNLSLIKEYIDLQKRKCGGERWRVKQFLDYLQVFLGVINTEWTMES

SEQ ID NO: 82 - protein, 113 aa, synthetic construct
VENTMNRLVAETLTLLSIHRTLLIGDGNLMISTPVHTNHQLCIEEVFQGIDTLKNQTARGDAVEKLFQNLSLIKEYIDRQKKNCGGERWRVTQFLDYLQVFLGVINTEWTMES

SEQ ID NO: 83 - protein, 129 aa, synthetic construct [sequence omitted]

SEQ ID NO: 84 - protein, 109 aa, synthetic construct
GHNFNITIKEIIKMLNILTARNDSCMELTVKDVFTAPKNTSDKEIFCRAATVLRQIYTHNCSNRYLRGLYRNLSSMANKTCSMNEIKKSTLKDFLERLKVIMQKKYYRH

SEQ ID NO: 85 - protein, 109 aa, synthetic construct
QNFNNTLKEIIKTLNILTARNDSCMELTVMDVLAAPKNTSDKEIFCRATTVLRQIYTHHNCSTKFLKGLDRNLSSMANRTCSVNEVKKCTLKDFLERLKAIMQKKYSKH

SEQ ID NO: 86 - protein, 122 aa, synthetic construct
LTCLGGFASPGPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 87 - protein, 113 aa, synthetic construct
SPSPVTPSPTLKELIEELVNITQNQASLCNGSMVWSVNLTAGMYCAALESLINVSDCSAIQRTQRMLKALCSQKPAAGQISSERSRDTKIEVIQLVKNLLTYVRGVYRHGNFR

SEQ ID NO: 88 - protein, 113 aa, synthetic construct
SPGPHSRRELKELIEELVNITQNQVSLCNGSMVWSVNLTTGMQYCAALESLINVSDCTAIQRTQRMLKALCTQKPSAGQTASERSRDTKIEVIQLVKNLLNHLRRNFRHGNFK

SEQ ID NO: 89 - protein, 111 aa, synthetic construct
PGPHSRRELKELIEELVNITQNQVSLCNGSMVWSVNLTTGMYCAALESLINVSDCTAIQRTQRMLKALCTQKPSAGQTASERSRDTKIEVIQLVKNLLNHLRRNFRHGNFK

SEQ ID NO: 90 - protein, 121 aa, synthetic construct
CLGGLASPAPLPSSMALKELIKELVNITQNQAPLCNGSMVWSVNLTADTYCRALESLSNVSTCSAIQNTRKMLTKLCPHQLSAGQVSSERARDTKIEVIVLVKDLLKNLRKIFHGGKHVDA

SEQ ID NO: 91 - protein, 113 aa, synthetic construct
APLPSSMALKELIKELVNITQNQAPLCNGSMVWSVNLTADTYCRALESLSNVSTCSAIQNTRKMLTKLCPHQLSAGQVSSERARDTKIEVIVLVKDLLKNLRKIFHGGKHVDA

SEQ ID NO: 92 - protein, 114 aa, synthetic construct
SPSPVPSATALKELIEELVNITQNQKVPLCNGSMVWSLNLTSSMYCAALDSLISISNCSVIQRTKRMLNALCPHKPSAKQVSSEYVRDTKIEVAQFLKDLLRHSRIVFRNERFN

SEQ ID NO: 93 - protein, 113 aa, synthetic construct
SPGPVPPHSTALKELIEELVNITQNQKTPLCNGSMVWSVNLTTSMQYCAALESLINISDCSAIQKTQRMLSALCSHKPPSEQVPGKHIRDTKIEVAQFVKDLLKHLRMIFRHG

SEQ ID NO: 94 - protein, 159 aa, unidentified
SAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 95 - protein, 153 aa, synthetic construct
LSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 96 - protein, 157 aa, synthetic construct
SVAYNFHNNEKYNYIRIIKSQFILNDNLNQSIVRQTGGNYLMTAALQNLDDAVKFDMGAYTSEDSKLPVTLRISKTRLFVSAQNEDEPVLLKEMPETPKTIRDETNLLFFWERHGSKHYFKSVAQPKLFIATQERKLVHMARGQPSITDFRLLETQP

SEQ ID NO: 97 - protein, 158 aa, synthetic construct
SVAPNFYSSEKYNYQKIIKSQFILNDNLSQSVIRKAGGKYLAAAALQNLDDAVKFDMGAYTSKEDSKLPVTLRISKTRLFVSAQNEDEPVLLKEMPETPKTIRDETNLLFFWERHGSKNYFKSVAHPKLFIATQEEQLVHMARGLPSVTDFQILETQS

SEQ ID NO: 98 - protein, 157 aa, synthetic construct
SVAPNFYSSEKYNYQKIIKSQFILNDNLSQSVIRKAGGKYLAAAALQNLDDAVKFDMGAYTSKEDSKLPVTLRISKTRLFVSAQNEDEPVLLKEMPETPKTIRDETNLLFFWERHGSKNYFKSVAHPKLFIATQEEQLVHMARGLPSVTDFQILETQ

SEQ ID NO: 99 - protein, 158 aa, synthetic construct
SVHYNFQSNTKYNFMRIVNHQCTLNDALNQSVIRDTSGQYLATAALNNLDDAVKFDMGAYTSEEDSQLPVTLRISKTRLFVSAQNEDEPVLLKEMPDTPKTIKDETNLLFFWERHGSKNYFKSVAHPKLFIATKQGKLVHMARGQPSITDFQILDNQF

SEQ ID NO: 100 - protein, 157 aa, synthetic construct
SVHYNFQSNTKYNFMRIVNHQCTLNDALNQSVIRDTSGQYLATAALNNLDDAVKFDMGAYTSEEDSQLPVTLRISKTRLFVSAQNEDEPVLLKEMPDTPKTIKDETNLLFFWERHGSKNYFKSVAHPKLFIATKQGKLVHMARGQPSITDFQILDNQ

SEQ ID NO: 101 - protein, 153 aa, synthetic construct
SAHYSFQSNVKYNFMRVIHQECILNDALNQSIIRDMSGPYLTAATLNNLEEAVKFDMVAYVSEEDSQLPVTLRISKTQLFVSAQNEDEPVLLKEMPETPKIIKDETNLLFFWEKHGSMDYFKSVAHPKLFIATKQEKLVHMASGPPSITDFQI

SEQ ID NO: 102 - protein, 157 aa, synthetic construct
SATYSFQSNMKYNFMRVINHQCILNDARNQSIIRDPSGQYLMAAVLNNLDEAVKFDMAAYTSNDDSQLPVTLRISETRLFVSAQNEDEPVLLKELPETPKTIKDETSLLFFWEKHGNMDYFKSAAHPKLLIATRQEKLVHMARGLPSVTDFQILENQ

SEQ ID NO: 103 - protein, 175 aa, synthetic construct
FGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 104 - protein, 176 aa, synthetic construct
AFGISGVQKYTRALHDSSITGISPITEYLASLSTYNDQSITFALEDESYEIYVEDLKKDEKKDKVLLSYYESQHPSNESGDGVDGKMLMVTLSPTKDFWLHANNKEHSVELHKCEKPLPDQAFFVLHNMHSNCVSFECKTDPGVFIGVKDNHLALIKVDSSENLCTENILFKLSET

SEQ ID NO: 105 - protein, 151 aa, synthetic construct
EYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 106 - protein, 163 aa, synthetic construct
MASGRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 107 - protein, 154 aa, synthetic construct
SIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 108 - protein, 160 aa, synthetic construct
GRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 109 - protein, 169 aa, synthetic construct
DVPMLQKCFGRANVPSIQEYSASLSTYNDQSITFVFEDGSYEIYVEDLRKGQEKDKVLFRYYDSQSPSHETGDDVDGQTLLVNLSPTKDKDFLLHANNEEHSVELQKCENQLPDQAFFLLHRKSSECVSFECKNNPGVFIGVKDNHLALIKVGDQTKDSYIEKTIFKLS

SEQ ID NO: 110 - protein, 170 aa, synthetic construct
FGVPMVQKCFGTTDVPSIQEYSASLSTYNDQSITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECKNDPGVFIGVKDNHLALIKVEDQTEYFSTENIIFKLS

SEQ ID NO: 111 - protein, 171 aa, synthetic construct
AFGVPMVQKCFGTTDVPSIQEYSASLSTYNDQSITFVFEDGSYEIYVEDLGKDQEKDKVLFRYYDFQSPSRETGDADDGHTLLVNLSPTKDKDFLLHANNKEHSVELQKCKKPLPDQAFFRLHRKSSKCVSFECKNDPGVFIGVKDNHLALIKVEDQTEYFSTENIIFKLS

SEQ ID NO: 112 - protein, 170 aa, synthetic construct
FDVPMIQRCARSTAGPIFEEDFASLSTYNDQSISFVIEDGSYEIYIEDLSGQQEKDPVLFRCYPSQCSSSETAGGADGQKLMVNLSPKKYKDFLLHANNKEHSVELQKCENPFPDQAFFLMHKTSSRCVSFECKNNPGVFIGVKDKHLALIKVGEQTEPSSRQNIIFKLS

SEQ ID NO: 113 - protein, 145 aa, synthetic construct
YSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 114 - protein, 147 aa, synthetic construct
KDCTHWPNCCPSKRQDPTHEWLKRDTVLKFPGETTSLTHHPESCKASEDGPLNSRSISPWKYELDRDLNRLPQDLYHARCLCQHCVSLQTGSHMDPLGNSELLYHNQTVFYRRPCPGEQGAPDGYCLEQRLYRVSLACVCVRPRVMA

SEQ ID NO: 115 - protein, 147 aa, synthetic construct
KDCIHWSSCCPSKGQNPTHEWLTENTVLMTPPESASLIHSLESCRASEDGPLNSRSISPWRYELDRDLNRLPQDLYHARCLCPHCVSLQTGSHMDPLGNSELLYHNQTVFYRRPCPGEQGTRDGYCLEQRLYRVSLACVCVRPRVMA

SEQ ID NO: 116 - protein, 147 aa, synthetic construct
KECTHWPSCCPKKGQDPIEEWLKWSTAHVPPPETANLAHHPESCRASEDGPLNSRSISPWRYELDRDLNRLPQDLYHARCLCPHCVSLQTGSHMDPLGNSELLYHNQTVFYRRPCPGKRGAHDGYCLERRLYRVSLACVCVRPRVMA

SEQ ID NO: 117 - protein, 153 aa, synthetic construct
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 118 - protein, 154 aa, synthetic construct
DAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 119 - protein, 152 aa, synthetic construct
AAMQSVDCKLQGISHKYLVLSNSYELRALHLNGENVNKQVVFHMSFVHGDESNNKIPVVLGIKQKNLYLSCVMKDGKPTLQLEKVDPKVYPKRKMEKRFVFNKIEIKNTVEFESSQYPNWYISTSQVEGMPVFLGNTRGGQDITDFTMEFSS

SEQ ID NO: 120 - protein, 152 aa, synthetic construct
AALPSVDCTLQDINHKYLVLSNSYELRALHLNGENVNKQVVFHMSFVHGDESNYKIPVVLGIKQKNLYLSCVMKDGKPTLQLEKVDPKVYPKRKMEKRFVFNKIEIKNTVEFESSQYPNWYISTSQVEGMPVFLGNTKGGQDITDFTMEFSS

SEQ ID NO: 121 - protein, 152 aa, synthetic construct
AAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESAS

SEQ ID NO: 122 - protein, 153 aa, synthetic construct
AAMHSVNCRLRDIYHKSLVLSGACELQAVHLNGENTNQQVVFCMSFVQGEEETDKIPVALGLKEKNLYLSCGMKDGKPTLQLETVDPNTYPKRKMEKRFVFNKMEIKGNVEFESAMYPNWYISTSQAEKSPVFLGNTRGGRDITDFIMEITSA

SEQ ID NO: 123 - protein, 152 aa, synthetic construct
PVQSIKCKLQDREQKSLVLASPCVLKALHLLSQEMNREVVFCMSFVQGEERDNKIPVALGIKDKNLYLSCVKKGDTPTLQLEEVDPKVYPKRNMEKRFVFYKTEIKNTVEFESVLYPNWYISTSQIEERPVFLGHFRGGQDITDFRMETLSP

SEQ ID NO: 124 - protein, 154 aa, synthetic construct
DANVQSMECKLQDKDHKSLVLAGPHMLKALHLLTGDLKREVVFCMSFVQGDDSNNKIPVTLGIKGKNLYLSCVMKDNTPTLQLEDIDPKRYPKRDMEKRFVFYKTEIKNRVEFESALYPNWYISTSQAEQKPVFLGNSKGRQDITDFTMEVLSP

SEQ ID NO: 125 - protein, 141 aa, synthetic construct [sequence omitted]

SEQ ID NO: 126 - protein, 136 aa, synthetic construct
SHMAPTHQLPPSDVRKIILELQPLSRGLLEDYQKKETGVPESNRTLLLCLTSDSQPPRLNSSAILPYFRAIRPLSDKNIIDKIIEQLDKLKFQHEPETEISVPADTFECKSFILTILQQFSACLESVFKSLNSGPQ

SEQ ID NO: 127 - protein, 136 aa, synthetic construct
SHMAPAHRLQPSDVRKIILELRPMSKGLLQDYLKKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 128 - protein, 133 aa, synthetic construct
APAHRLQPSDVRKIILELRPMSKGLLQDYVSKEIGLPESNHSSLPCLSSDSQLPHINGSAILPYFRAIRPLSDKNTIDKIIEQLDKLKFQREPEAKVSMPADNFERKNFILAVLQQFSACLEHVLQSLNSGPQ

SEQ ID NO: 129 - protein, 130 aa, synthetic construct
SHTGPIYQLQPKEIQAIIVELQNLSKKLLDDYLNKEKGVQKFDSDLPSCFTSDSQAPGNINSSAILPYFKAISPSLNNDKSLYIIEQLDKLNFQNAPETEVSMPTDNFERKRFILTILRWFSNCLEHRAQ

SEQ ID NO: 130 - protein, 130 aa, synthetic construct
GPIYQLQPKEIQAIIVELQNLSKKLLDDYVSALETSILSCFFKTDLPSCFTSDSQAPGNINSSAILPYFKAISPSLNNDKSLYIIEQLDKLNFQNAPETEVSMPTDNFERKRFILTILRWFSNCLEHRAQ

SEQ ID NO: 131 - protein, 129 aa, synthetic construct
SFSKLMEEYQKEKGVAESCTCRMQCFSVSPQPPHNINSSAILPYFRAIKPFFKDTNNFSDVMDHLKKLELQRAPDIAVPTATYPSKMFVKCVLETFSKCLKLVGTEPPKVTEDHLVDPVPFDWQECKEP

SEQ ID NO: 132 - protein, 134 aa, synthetic construct
SVPSPEEQKGIIKELQASVKKVLKDYEEKEKGVQEQERFQIPCLTFNPQSPHNIESSAILPYLRAIKRSFDNDDDIIDIINHLHKLKFQQGPETTVPVPTNSFEGKSFTLSILKAFSKCLTSDFNSHDPRRGQS

SEQ ID NO: 133 - protein, 131 aa, synthetic construct
YDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 134 - protein, 127 aa, synthetic construct
YNFIDCDFEKIRWKYQEVIYQALEKYMDGTRSTEFSHPVYCADPPDCLARIERLTLHRIRGCASGAREAFAEGTVAALAAECPGYAAAPINNTQAKKKRKKRGVTTNKCREQVAHLIGLWRRFSRIS

SEQ ID NO: 135 - protein, 127 aa, synthetic construct
YNFTDCDFGKIRKKYQEVIYQALNKYMNGVKSTEFNNYIYCEDPPDCLAKIDLITFYPTHGCTTLAKEIFAIRTNATLTLQCPGYSGTQINNTQAKKKRKKRQVTTNKCREQVSHLMELWRRFSRIS

SEQ ID NO: 136 - protein, 134 aa, synthetic construct
VSRSPSPPPPPPESSLVSYGLSFFVSFRCLFFLAFSPAFPPFLVNLAAREQPDCLAKIDLITFYPTHGCTTLAKEIFAIRTNATLTLQCPGYSGTQINNTQAKKKRKKRQVTTNKCREQVSHLMELWRRFSRIS

SEQ ID NO: 137 - protein, 125 aa, synthetic construct
YDFTDCNFAKIEEEYQNVIFLALKEYMNGIKSTQFNHTVYCGDRPSCLTEIERLTFKSSQCLSLAKKIVTATNATLNSHCPGHSGIQINNTQAMKKRKKREVTTNKCLKQVSNLIELWRYFSRSQ

SEQ ID NO: 138 - protein, 120 aa, synthetic construct
SSSHPIFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAVRRA

SEQ ID NO: 139 - protein, 121 aa, synthetic construct; identical to SEQ ID NO: 30

SEQ ID NO: 140 - protein, 120 aa, synthetic construct
SSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKTGRKA

SEQ ID NO: 141 - protein, 120 aa, synthetic construct
SSSHPVFHRGEFSVCDSISVWVGDKTTATDIKGKEVMVLGEVNINNSVFKQYFFETKCRDPNPVDSGCRGIDAKHWNSYCTTTHTFVKALTMDGKQAAWRFIRIDTACVCVLSRKTGQRA

SEQ ID NO: 142 - protein, 120 aa, synthetic construct
SSSHPVFHRGEFSVCDSVSVWVGDKTTATDIKGKEVMVLGEVNINNSVFKQYFFETKCRDPNPVDSGCRGIDSKHWNSYCTTTHTFVKALTMDGKQAAWRFIRIDTACVCVLSRKAGRRA

SEQ ID NO: 143 - protein, 42 aa, synthetic construct [sequence omitted]

SEQ ID NO: 144 - protein, 6 aa, synthetic construct
DAEFRH

SEQ ID NO: 145 - protein, 7 aa, synthetic construct
DAEFRHD

SEQ ID NO: 146 - protein, 4 aa, synthetic construct
EFRH

SEQ ID NO: 147 - protein, 5 aa, synthetic construct
DAEFR

SEQ ID NO: 148 - protein, 5 aa, synthetic construct
AEFRH

SEQ ID NO: 149 - protein, 5 aa, synthetic construct
EFRHD

SEQ ID NO: 150 - protein, 10 aa, synthetic construct
EGYQDYEPEA

SEQ ID NO: 151 - protein, 8 aa, synthetic construct
KNEEGAPQ

SEQ ID NO: 152 - protein, 8 aa, synthetic construct
MDVFMKGL

SEQ ID NO: 153 - protein, 7 aa, synthetic construct
KNEEGAP

SEQ ID NO: 154 - protein, 12 aa, synthetic construct
MALDGSSGGGSN

SEQ ID NO: 155 - protein, 10 aa, synthetic construct
EHWSYGLRPG

SEQ ID NO: 156 - protein, 10 aa, synthetic construct
QHWSYGLRPG

SEQ ID NO: 157 - protein, 8 aa, synthetic construct
DRVYIHPF

SEQ ID NO: 158 - protein, 7 aa, synthetic construct
DRVYIHP

SEQ ID NO: 159 - protein, 10 aa, synthetic construct
DRVYIHPFHL

SEQ ID NO: 160 - protein, 95 aa, synthetic construct
CCRYPLTVDFEAFGWDWIIAPKRYKANYCSGECEFVFLQKYPHTHLVHQANPRGSAGPCCTPTKMSPINMLYFNGEGQIIYGKIPAMVVDRCGCS

SEQ ID NO: 161 - protein, 165 aa, synthetic construct
DRADGQPAGDRADGQPAGDRAAGQPAGDRAAGQPAGDRADGQPAGDRADGQPAGAPGANQEGGAAAPGANQEGGAAAPGANQEGGAAAPGANQEGGAAAPGANQEGGAAAPGANQEGGAAANGAGNQPGANGAGNQPGANGAGNQPGANGAGNQPGANGAGNQPG

SEQ ID NO: 162 - protein, 213 aa, synthetic construct
MARNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLLA

SEQ ID NO: 163 - protein, 215 aa, synthetic construct
MARNINELKIFVEKAKYYSIKLDAIYNECTGAYNDIMTYSEGTFSDQSKVNQAISIFKKDNKIVNKFKELEKIIEEYKPMFLSKLIDDFAIELDQAVDNDVSNARHVADSYKKLRKSVVLAYIESFDVISSKFVDSKFVEASKKFVNKAKEFVEENDLIALECIVKTIGDMVNDREINSRSRYNNFYKKEADFLGAAVELEGAYKAIKQTLLAAS

SEQ ID NO: 164 - protein, 117 aa, synthetic construct
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKMGSGA

SEQ ID NO: 165 - protein, 113 aa, synthetic construct
MDKLTLKGMSYVMCTGSFKLEKEVAETQHGTVLVQVKYEGTDAPCKIPFSTQDEKGVTQNGRLITANPIVTDKEKPVNIETEPPFGESYIVVGAGEKALKLSWFKKGSSIGKM

SEQ ID NO: 166 - protein, 24 aa, synthetic construct
SLLTEVETPIRNEWGCRCNDSSDG

SEQ ID NO: 167 - protein, 211 aa, synthetic construct
NITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 168 - protein, 72 aa, synthetic construct
NSNNLDSKVGGNYNYLYRLFRKSNLKPFERDISTEIYQAGSTPCNGVEGFNCYFPLQSYGFQPTNGVGYQPY

SEQ ID NO: 169 - protein, 211 aa, synthetic construct
QAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPK

SEQ ID NO: 170 - protein, 83 aa, synthetic construct
VPHNLTTITKPLKYSYINKCSRLLSDDRTEVPQLVNANQYSPCVSIVPSTVWEDGDYYRKQLSPLEGGGWLVASGSTVAMTEQ

SEQ ID NO: 171 - protein, 167 aa, synthetic construct
MGSAAIQSQDYTFRDISQKSLVLSGSYELRALHLNGQNMNQQVVFRMSFVHGEENSKKIPVVLCIKKNNLYLSCVMKDGKPTLQLEMLDPKVYPKKKMEKRFVFNKTEIKGNVEFESSQFPNWYISTSQAEEMPVFLGNTKGGQDITDFIMESASTSHHHHHHGGCG

SEQ ID NO: 172 - DNA, 520 bp, synthetic construct
  source 1..520: mol_type = other DNA
catatgggatccgcagccatacagtcacaggactacacgttccgagacataagccaaaagagcctggtgctgtctggctcatacgagcttcgggctctccacctcaatggacagaatatgaaccaacaagtggtgttccgcatgagctttgtgcacggggaggaaaatagtaagaagataccagtagtgttgtgcatcaagaaaaataacctgtacctgtcctgtgtgatgaaagacgggaaacccaccctacagctggagatgttagaccccaaagtttacccaaagaagaagatggaaaagcgatttgtcttcaacaagacagaaatcaagggcaatgtggaatttgagtcttcccagttccccaactggtacatcagcacctctcaagcagaagaaatgcctgtcttcctaggaaataccaaaggtggccaggatataactgacttcatcatggaaagcgcttccactagtcatcatcatcaccatcatggtggttgtggataataagcttctcgag

SEQ ID NO: 173 - DNA, 711 bp, synthetic construct
  source 1..711: mol_type = other DNA
atggccgaacctcacccagagtcaaacgttcccgcgggacatgcgattccacaggcgcactggacaaaacttcaacattcgttagacacagctcttcgccgtgcccgcagcacccctgcgggcgcaatcgcagcccgtgtagcgggtcaaacccgtaatattacagtggatccgaaattgtttaaaaagcgccgtctgcgtagtccccgcgtgttattctccacgcatcccccccctgttgccgcggatacccaaggcttagatctggaagctgggggagctgcatctttcaaccgcacgcaccgctcgaaacgcagtagcagtcacccagtgtttcaccgtggtgaattttctgtctgcgactccgtctctgtgtgggtaggggataagacgactgctaccgacatcaaggggaaggaagtgatggttttaggggaggtgaatattaacaattcggtcttcaagcaatacttcttcgaaacaaaatgccgcgatccaacgccagtggatagtggttgtcgcggtattgactcgaagcattggaactcatactgcaccacaacccatacctttgtgaaggcattgacaatggatggcaaacaggccgcctggcgttttattcgcattgacaccgcctgtgtctgtgtccttagtcgcaaagctggtcgccgcgcgggtggttgcgggaaacaccatcaccatcaccattaa

SEQ ID NO: 174 - protein, 131 aa, synthetic construct
SSSHPVFHRGEFSVCDSVSVWVGDKTTATDIKGKEVMVLGEVNINNSVFKQYFFETKCRDPTPVDSGCRGIDSKHWNSYCTTTHTFVKALTMDGKQAAWRFIRIDTACVCVLSRKAGRRAGGCGKHHHHHH

SEQ ID NO: 175 - protein, 6 aa, synthetic construct
HHHHHH